SEQUENCE LISTING (31 sequences)

SEQ ID NO: 1 - DNA, 4374 bp, synthetic construct
  source 1..4374: mol_type = other DNA
atgcagattgagctgtctacctgcttcttcctgtgcctgctgaggttctgcttctctgctaccaggaggtactacctgggggctgtggagctgagctgggattacatgcagtctgacctgggggagctgcctgtggatgccaggtttccccccagggtgcccaagagcttccccttcaatacctctgtggtgtataagaagaccctgtttgtggagttcactgatcatctgttcaacattgctaaacccaggcccccctggatggggctgctgggccctaccatccaggctgaggtgtatgacactgtggtgatcactctgaagaacatggctagccatcctgtgtctctgcatgctgtgggggtgagctactggaaggcttctgagggggctgagtatgatgatcagactagccagagggagaaggaggatgacaaggtgttccctgggggctctcacacctatgtctggcaggtgctgaaggagaatggccccatggcctctgatcctctgtgtctgacctatagctacctgagccatgtggacctggtgaaggacctgaactctggcctgattggggccctgctggtgtgtagggaggggagcctggccaaggagaagacccagaccctgcacaagttcattctgctgtttgctgtgtttgatgagggcaagagctggcattctgaaaccaagaacagcctgatgcaggacagggatgctgcctctgctagggcctggcccaagatgcacactgtgaatgggtatgtcaataggtctctgcctggcctgattggctgccacaggaagtctgtgtactggcatgtgattgggatgggcaccacccctgaggtgcacagcatctttctggagggccacaccttcctggtgaggaatcacagacaggccagcctggagatcagccccatcaccttcctgactgcccagaccctgctgatggacctgggccagtttctgctgttctgccacatctctagccaccagcatgatggcatggaggcctatgtgaaggtggactcctgccctgaggagccccagctgaggatgaagaataatgaggaggctgaggactatgatgatgacctgactgactctgagatggatgtggtgagatttgatgatgacaattctcccagcttcattcagatcaggtctgtggccaagaagcatcccaagacctgggtgcactacattgctgctgaggaggaggactgggactatgcccccctggtgctggcccctgatgacaggagctataagagccagtacctgaataatggcccccagaggattgggaggaagtataagaaggtgaggttcatggcctatactgatgaaaccttcaagaccagagaggccatccagcatgagtctgggatcctggggcccctgctgtatggggaggtgggggacaccctgctgatcatcttcaagaaccaggccagcaggccctacaacatctaccctcatggcatcactgatgtgaggcctctgtacagcagaaggctgcccaagggggtgaagcatctgaaggacttccccattctgcctggggagattttcaagtacaagtggactgtgactgtggaggatggcccaaccaagtctgaccctaggtgcctgactaggtactacagcagctttgtgaatatggagagggacctggcctctggcctgattggccccctgctgatctgctacaaggagtctgtggatcagaggggcaaccagatcatgtctgacaagaggaatgtgatcctgttctctgtgtttgatgagaacaggagctggtacctgactgagaacattcagaggtttctgcccaaccctgctggggtgcagctggaggaccctgaattccaggcctctaacatcatgcacagcattaatggctatgtgtttgacagcctgcagctgtctgtgtgcctgcatgaggtggcctactggtacattctgagcattggggcccagactgacttcctgtctgtgttcttctctggctacacctttaagcacaagatggtgtatgaggataccctgaccctgtttcctttctctggggagactgtgttcatgagcatggagaaccctggcctgtggatcctgggctgccacaactctgacttcaggaacagggggatgactgctctgctgaaggtgagcagctgtgataagaacactggggactactatgaggacagctatgaggacatctctgcctatctgctgagcaagaataatgctattgagcccaggagcttctctcagaacccccctgtgctgaagaggcaccagagggagatcaccagaactactctgcagtctgaccaggaggagattgactatgatgacaccatctctgtggagatgaagaaggaggattttgatatttatgatgaggatgaaaaccagagccccaggagctttcagaagaagactaggcactatttcattgctgctgtggagaggctgtgggactatggcatgtcttctagcccccatgtgctgaggaacagggcccagtctggctctgtgccccagttcaagaaggtggtgttccaggagttcactgatggcagcttcactcagcccctgtacaggggggagctgaatgagcacctggggctgctgggcccttatatcagggctgaggtggaggataacatcatggtgaccttcaggaaccaggccagcaggccctacagcttctactctagcctgatcagctatgaggaggaccagaggcagggggctgagcccaggaagaactttgtgaagcccaatgagaccaagacttatttctggaaggtgcagcaccatatggcccccaccaaggatgagtttgattgcaaagcctgggcctacttctctgatgtggacctggagaaggatgtgcactctgggctgattggccccctgctggtgtgccacaccaacactctgaaccctgcccatggcaggcaggtgactgtgcaggagtttgccctgttcttcaccatctttgatgagactaagagctggtacttcactgagaacatggagaggaactgcagggccccctgcaatatccagatggaggaccccacctttaaggaaaattataggtttcatgccattaatggctacatcatggacaccctgcctggcctggtgatggcccaggaccagaggatcaggtggtacctgctgagcatgggcagcaatgagaacattcacagcatccacttctctggccatgtgttcactgtgaggaagaaggaggagtacaagatggccctgtataatctgtaccctggggtgtttgagactgtggagatgctgcccagcaaggctggcatctggagggtggagtgcctgattggggagcacctgcatgctggcatgagcaccctgttcctggtgtattctaacaagtgtcagacccccctgggcatggcctctggccatatcagggacttccagatcactgcctctggccagtatgggcagtgggcccccaagctggccaggctgcattactctggcagcatcaatgcctggagcaccaaggagccattcagctggattaaggtggacctgctggctccaatgattatccatggcatcaagacccagggggccaggcagaagtttagcagcctgtacatctctcagtttatcatcatgtactctctggatggcaaaaagtggcagacctacaggggcaattctactggcactctgatggtgttctttggcaatgtggacagctctgggatcaagcacaacatctttaacccccctatcattgccaggtacattaggctgcaccccacccattacagcatcaggagcaccctgaggatggagctgatgggctgtgatctgaacagctgcagcatgcccctgggcatggagagcaaggctatctctgatgcccagattactgccagcagctacttcaccaatatgtttgccacctggagccccagcaaggccaggctgcacctgcagggcaggtctaatgcctggaggccccaggtgaacaaccccaaggagtggctgcaggtggacttccagaagaccatgaaggtgactggggtgaccacccagggggtgaagagcctgctgactagcatgtatgtgaaggagttcctgatcagcagcagccaggatggccatcagtggaccctgttcttccagaatggcaaggtgaaggtgttccagggcaatcaggacagcttcacccctgtggtgaacagcctggacccccccctgctgaccagatacctgaggatccacccccagagctgggtgcatcagattgccctgaggatggaggtgctggggtgtgaggcccaggacctgtactga

SEQ ID NO: 2 - DNA, 4374 bp, synthetic construct
  source 1..4374: mol_type = other DNA
atgcagattgagctgtctacctgctttttcctgtgtctgctgaggttctgcttctctgccactaggaggtactacctgggggctgtggagctgtcttgggattacatgcagtctgatctgggggagctgcctgtggatgccaggtttcctcccagggtgcccaagtctttccccttcaatacctctgtggtgtataagaagaccctgtttgtggagtttactgatcacctgttcaacattgccaagcccaggcccccttggatgggcctgctggggcccaccatccaggctgaggtgtatgacactgtggtgatcaccctgaagaacatggcctctcaccctgtgagcctgcatgctgtgggggtgagctactggaaggcctctgagggggctgagtatgatgaccagaccagccagagggagaaggaggatgataaggtgttccctggggggagccacacttatgtgtggcaggtgctgaaggagaatggcccaatggcctctgatcccctgtgcctgacctattcttacctgagccatgtggacctggtgaaggacctgaactctggcctgattggggccctgctggtgtgcagggagggctctctggctaaggagaagacccagaccctgcacaagttcatcctgctgtttgctgtgtttgatgaggggaagagctggcactctgagaccaagaacagcctgatgcaggacagggatgctgcctctgccagggcctggcccaaaatgcacactgtgaatggctatgtgaataggagcctgcctggcctgattggctgccacaggaagtctgtgtattggcatgtgattggcatgggcaccacccctgaggtgcactctatcttcctggagggccatactttcctggtgaggaatcataggcaggccagcctggagattagccccattacctttctgactgcccagaccctgctgatggacctgggccagttcctgctgttttgccacatcagctctcaccagcatgatggcatggaggcctatgtgaaggtggatagctgccctgaggagccccagctgaggatgaagaacaatgaggaggctgaggattatgatgatgatctgactgattctgaaatggatgtggtgaggtttgatgatgacaatagcccctctttcatccagatcaggtctgtggccaagaagcatcctaagacctgggtgcactacattgctgctgaggaggaggactgggactatgctcccctggtgctggcccctgatgacaggtcttacaagagccagtacctgaacaatggcccccagagaattgggaggaagtataagaaggtgagattcatggcttacactgatgagaccttcaagactagggaggccatccagcatgagtctggcattctgggccccctgctgtatggggaggtgggggacaccctgctgatcatcttcaagaaccaggcctctaggccctacaatatttacccccatgggatcactgatgtgaggcccctgtacagcaggaggctgcctaagggggtgaagcatctgaaggacttccccatcctgcctggggagatcttcaagtataagtggactgtgactgtggaagatggccccaccaagtctgaccctaggtgcctgaccaggtactactcttcttttgtgaacatggagagggacctggcctctggcctgattggccccctgctgatctgctacaaggagtctgtggaccagagggggaaccagattatgtctgacaagaggaatgtgattctgttctctgtgtttgatgagaacaggagctggtatctgactgagaacatccagaggttcctgcccaatcctgctggggtgcagctggaggaccctgagttccaggccagcaacatcatgcacagcatcaatgggtatgtgtttgattctctgcagctgtctgtgtgcctgcatgaggtggcctactggtacatcctgagcattggggctcagactgatttcctgtctgtgttcttttctggctacacctttaagcataagatggtgtatgaggacactctgaccctgtttcccttctctggggagactgtgtttatgagcatggagaaccctggcctgtggatcctgggctgccacaactctgatttcaggaacaggggcatgactgctctgctgaaggtgtcttcttgtgacaagaacactggggactattatgaggacagctatgaggacatctctgcctacctgctgagcaagaacaatgctattgagcccagatctttcagccagaacccccctgtgctgaagaggcaccagagggagatcactaggaccaccctgcagtctgaccaggaggagattgactatgatgacactatctctgtggagatgaagaaggaggactttgatatctatgatgaggatgagaaccagtctcccaggagcttccagaaaaagaccaggcactacttcattgctgctgtggagaggctgtgggactatggcatgtcttctagcccccatgtgctgaggaacagggcccagtctgggtctgtgccccagttcaagaaggtggtgttccaggagttcactgatgggagcttcacccagcctctgtacaggggggagctgaatgagcacctggggctgctgggcccttatattagggctgaggtggaggacaacatcatggtgactttcaggaatcaggcctctaggccctatagcttctacagctctctgatcagctatgaggaggatcagaggcagggggctgagcccaggaagaactttgtgaagcccaatgagaccaagacctacttctggaaggtgcagcaccacatggctcctaccaaggatgagtttgactgcaaggcctgggcctacttttctgatgtggacctggagaaggatgtgcactctggcctgattggccccctgctggtgtgtcataccaacaccctgaaccctgcccatggcaggcaggtgactgtgcaggagtttgccctgttcttcaccatctttgatgagaccaagagctggtactttactgagaacatggagaggaattgcagagccccttgcaacatccagatggaggacccaaccttcaaagagaactacaggttccatgccatcaatgggtacatcatggacaccctgcctggcctggtgatggctcaggaccagaggatcaggtggtatctgctgagcatgggcagcaatgagaatatccatagcattcacttctctggccatgtgttcactgtgaggaagaaggaggagtacaagatggccctgtataacctgtaccctggggtgtttgagactgtggagatgctgccaagcaaggctgggatttggagggtggagtgcctgattggggagcacctgcatgctggcatgtctaccctgttcctggtgtactccaataagtgccagacccccctgggcatggcctctggccacatcagggacttccagatcactgcctctggccagtatgggcagtgggccccaaagctggccaggctgcactattctgggagcatcaatgcttggagcaccaaggagcctttcagctggattaaggtggatctgctggcccccatgatcattcatggcatcaaaacccagggggctagacagaagttttctagcctgtacatcagccagttcatcatcatgtacagcctggatggcaagaagtggcagacttacaggggcaatagcactggcaccctgatggtgttttttggcaatgtggacagctctggcatcaagcacaacatctttaacccccccattattgccaggtatatcaggctgcatcccacccactattctattaggtctactctgagaatggagctgatgggctgtgacctgaacagctgtagcatgcccctggggatggagagcaaggctatctctgatgcccagatcactgccagctcttatttcaccaatatgtttgccacctggtctccctctaaggccaggctgcacctgcagggcaggagcaatgcttggaggccccaggtgaataaccccaaggagtggctgcaggtggacttccagaagaccatgaaggtgactggggtgactacccagggggtgaagtctctgctgactagcatgtatgtgaaggagttcctgatcagcagcagccaggatgggcatcagtggactctgttcttccagaatggcaaggtgaaggtcttccaggggaaccaggatagcttcactcctgtggtgaactctctggacccccccctgctgactaggtatctgaggatccacccccagagctgggtgcaccagattgccctgaggatggaggtgctgggctgtgaggcccaggacctgtattga

SEQ ID NO: 3 - DNA, 4374 bp, synthetic construct
  source 1..4374: mol_type = other DNA
atgcagattgaactgtctacttgtttcttcctgtgcctgctgaggttttgcttctctgctactaggaggtactatctgggggctgtggagctgtcttgggactatatgcagtctgacctgggggagctgcctgtggatgctaggtttccccccagggtgcccaagagcttcccctttaacacctctgtggtgtataagaagactctgtttgtggagttcactgaccatctgttcaacattgccaagccaaggcccccctggatgggcctgctgggccccaccatccaggctgaggtgtatgacactgtggtgattactctgaagaacatggccagccatcctgtgagcctgcatgctgtgggggtgtcttactggaaggcctctgagggggctgagtatgatgaccagacctctcagagggagaaggaggatgacaaggtgttccctgggggctctcatacctatgtgtggcaggtcctgaaggagaatgggcccatggcctctgaccccctgtgcctgacctactcttatctgtctcatgtggacctggtgaaggacctgaactctggcctgattggggccctgctggtgtgcagggagggcagcctggctaaggagaagacccagactctgcacaagttcatcctgctgtttgctgtgtttgatgagggcaagagctggcactctgagaccaagaacagcctgatgcaggacagggatgctgcctctgctagggcctggcccaagatgcacactgtgaatgggtatgtgaacaggagcctgccaggcctgattggctgccataggaagtctgtgtattggcatgtgattgggatggggactacccctgaggtccacagcattttcctggaggggcatacctttctggtgaggaaccacaggcaggcctctctggagatctctcccattactttcctgactgcccagaccctgctgatggacctgggccagttcctgctgttctgccacatcagcagccaccagcatgatggcatggaggcctatgtgaaggtggatagctgccctgaggagccccagctgaggatgaaaaacaatgaggaggctgaggattatgatgatgacctgactgattctgagatggatgtggtgaggtttgatgatgataacagccccagcttcatccagattaggtctgtggccaagaagcatcccaagacctgggtgcactacattgctgctgaggaggaggattgggactatgctcctctggtgctggcccctgatgacaggagctacaagagccagtacctgaataatggcccccagaggattggcaggaagtataagaaggtgaggttcatggcctacactgatgagacctttaagaccagggaggccatccagcatgaatctgggatcctgggccccctgctgtatggggaggtgggggacaccctgctgattatctttaagaaccaggctagcaggccctacaacatttacccccatggcattactgatgtgaggcccctgtacagcaggaggctgcccaagggggtgaagcacctgaaggatttccccattctgcctggggagatctttaagtacaaatggactgtgactgtggaggatggccctactaagtctgatcccaggtgtctgaccagatactacagcagctttgtgaatatggagagggacctggcttctggcctgattggccccctgctgatctgctacaaggagtctgtggaccagaggggcaatcagattatgtctgacaagaggaatgtgatcctgttctctgtgtttgatgagaacagaagctggtacctgactgagaacatccagaggttcctgcccaaccctgctggggtgcagctggaggaccctgagttccaggctagcaatatcatgcacagcattaatggctatgtgtttgacagcctgcagctgtctgtgtgcctgcatgaggtggcctattggtacattctgagcattggggcccagactgatttcctgtctgtgttcttttctggctacaccttcaagcacaagatggtgtatgaggatactctgaccctgtttcccttctctggggagactgtgttcatgagcatggagaaccctggcctgtggatcctgggctgtcacaactctgacttcaggaacaggggcatgactgccctgctgaaggtgagctcttgtgataagaacactggggactactatgaggactcttatgaggacatctctgcctacctgctgagcaagaacaatgctattgagcccaggagcttctctcagaatccccctgtgctgaagaggcatcagagggagatcactaggactaccctgcagtctgaccaggaagagattgactatgatgacaccatctctgtggaaatgaagaaggaggactttgatatctatgatgaggatgaaaaccagagccccaggagcttccagaagaagaccaggcattacttcattgctgctgtggagaggctgtgggactatgggatgagctcttctccccatgtgctgaggaatagggctcagtctggctctgtcccacagttcaagaaggtggtgtttcaggagttcactgatggcagcttcactcagcccctgtacaggggggagctgaatgagcatctgggcctgctggggccctacatcagggctgaggtggaggataacattatggtgactttcaggaaccaggcctctaggccctacagcttctacagcagcctgatcagctatgaggaggaccagaggcagggggctgagcccaggaagaactttgtgaagcccaatgagactaagacctatttctggaaggtgcagcatcacatggctcccactaaagatgagtttgactgcaaggcctgggcctacttctctgatgtggatctggagaaggatgtgcattctgggctgattggccctctgctggtctgccatactaacaccctgaatcctgcccatggcaggcaggtgactgtgcaggagtttgccctgttctttaccatctttgatgagaccaagtcttggtacttcactgagaacatggagaggaactgcagggccccctgtaacatccagatggaggaccccacctttaaggagaactacaggttccatgccatcaatggctacatcatggacactctgcctggcctggtgatggcccaggaccagaggatcaggtggtacctgctgtctatgggctctaatgagaacattcattctatccacttctctggccatgtgtttactgtgaggaagaaggaggagtacaagatggccctgtacaatctgtaccctggggtgtttgaaactgtggagatgctgccctctaaggctggcatctggagggtggagtgcctgattggggaacacctgcatgctggcatgagcaccctgttcctggtctatagcaataagtgccagacccccctggggatggcctctgggcatatcagagacttccagatcactgcctctggccagtatggccagtgggcccccaagctggccaggctgcactactctggcagcattaatgcctggagcaccaaggagcccttctcttggatcaaggtggacctgctggctcccatgatcatccatgggatcaagacccagggggccaggcagaagttcagcagcctgtacatctctcagttcatcatcatgtactctctggatggcaagaagtggcagacctacaggggcaatagcactgggaccctgatggtgttctttgggaatgtggacagctctggcatcaagcacaatatcttcaacccccccatcattgccaggtacatcagactgcaccccactcattacagcatcaggagcactctgaggatggagctgatgggctgtgacctgaatagctgctctatgcccctgggcatggagagcaaggccatttctgatgcccagattactgcctcttcttacttcactaatatgtttgccacctggagccccagcaaggccaggctgcatctgcaggggaggagcaatgcctggaggccccaggtgaacaaccccaaggagtggctgcaggtggacttccagaagactatgaaggtgactggggtgaccactcagggggtgaagagcctgctgaccagcatgtatgtgaaggagttcctgatctcttctagccaggatgggcaccagtggaccctgtttttccagaatgggaaggtgaaggtgtttcagggcaatcaggacagctttactcctgtggtgaacagcctggacccccccctgctgactaggtacctgaggattcacccccagagctgggtgcaccagattgccctgaggatggaggtgctgggctgtgaggcccaggatctgtactga

SEQ ID NO: 4 - DNA, 4374 bp, synthetic construct
  source 1..4374: mol_type = other DNA
atgcagattgagctgtctacctgcttctttctgtgcctgctgaggttctgtttctctgccactaggaggtattatctgggggctgtggagctgtcctgggactacatgcagtctgatctgggggagctgcctgtggatgccaggttccctcccagggtgcccaagtctttccctttcaatacctctgtggtgtacaagaagactctgtttgtggagtttactgatcacctgtttaacattgccaagcccaggcccccctggatggggctgctgggccccaccatccaggctgaggtgtatgacactgtggtgattactctgaagaatatggcttctcaccctgtgagcctgcatgctgtgggggtgagctactggaaggcctctgagggggctgagtatgatgaccagaccagccagagggagaaggaggatgacaaggtgttccctgggggcagccacacttatgtgtggcaggtgctgaaggagaatggcccaatggcctctgaccccctgtgcctgacctacagctatctgagccatgtggatctggtgaaggatctgaactctggcctgattggggccctgctggtgtgcagggagggctctctggccaaggagaagactcagactctgcacaagttcatcctgctgtttgctgtgtttgatgagggcaagagctggcactctgagaccaagaactctctgatgcaggatagggatgctgcttctgccagggcctggcccaagatgcacactgtgaatgggtatgtgaataggagcctgcctgggctgattgggtgtcacaggaagtctgtgtactggcatgtgattggcatgggcaccactcctgaggtgcacagcatctttctggagggccacacttttctggtgaggaatcacaggcaggccagcctggagatcagccccatcaccttcctgactgcccagaccctgctgatggatctgggccagttcctgctgttttgccatatcagcagccatcagcatgatgggatggaggcttatgtgaaggtggactcttgccctgaggagcctcagctgaggatgaagaataatgaagaggctgaggactatgatgatgatctgactgactctgagatggatgtggtgaggtttgatgatgacaacagccccagctttatccagattaggtctgtggccaagaagcaccccaagacctgggtgcattacattgctgctgaggaagaggattgggactatgcccccctggtgctggcccctgatgacaggagctacaagtctcagtacctgaacaatggccctcagaggattggcaggaagtacaagaaggtgaggttcatggcttacactgatgagaccttcaagaccagggaggccattcagcatgaatctgggatcctgggccccctgctgtatggggaggtgggggacaccctgctgattattttcaagaaccaggccagcaggccctacaacatttatcctcatggcattactgatgtgagacccctgtacagcaggaggctgcctaagggggtgaagcacctgaaggacttccccatcctgcctggggagatcttcaagtacaagtggactgtgactgtggaggatggccccactaagtctgaccccaggtgcctgactaggtactactccagctttgtgaacatggagagggacctggcctctggcctgattggccccctgctgatctgctacaaggagtctgtggatcagaggggcaaccagatcatgtctgacaagagaaatgtgatcctgttctctgtgtttgatgagaataggtcttggtacctgactgagaacatccagaggtttctgcctaatcctgctggggtgcagctggaggatcctgagttccaggcctctaacattatgcacagcatcaatgggtatgtgtttgacagcctgcagctgtctgtgtgcctgcatgaggtggcctactggtacatcctgagcattggggcccagactgactttctgtctgtgttcttctctggctacacctttaagcataagatggtgtatgaggacaccctgactctgttccccttctctggggagactgtgttcatgagcatggagaacccaggcctgtggatcctgggctgccacaactctgatttcaggaataggggcatgactgccctgctgaaggtgagcagctgtgataagaacactggggactattatgaggatagctatgaggacatctctgcctacctgctgagcaagaacaatgccattgagcccaggagcttcagccagaatcctcctgtgctgaagaggcaccagagggagatcaccaggaccaccctgcagtctgatcaggaggagattgactatgatgacactatctctgtggagatgaagaaggaggactttgacatctatgatgaggatgagaatcagagccccaggagcttccagaagaagactagacactactttattgctgctgtggagaggctgtgggactatggcatgagctcttctccccatgtgctgagaaacagggcccagtctggctctgtgccccagttcaagaaggtggtcttccaggagttcactgatggctctttcacccagcctctgtatagaggggagctgaatgagcacctgggcctgctgggcccttacatcagggctgaggtggaggacaatatcatggtgaccttcaggaaccaggctagcaggccctactctttctacagcagcctgatcagctatgaggaggaccagaggcagggggctgagcctaggaagaattttgtgaagcccaatgagaccaagacctacttctggaaggtgcagcaccacatggctcccactaaggatgagtttgactgcaaggcctgggcctacttttctgatgtggacctggagaaggatgtgcattctggcctgattggccccctgctggtctgccacaccaatactctgaaccctgctcatgggagacaggtgactgtgcaggagtttgccctgttcttcaccatctttgatgagaccaagtcctggtactttactgagaacatggagaggaattgcagggccccttgcaacatccagatggaggaccccaccttcaaggaaaattataggttccatgccatcaatggctacatcatggacaccctgcctggcctggtgatggcccaggaccagaggatcaggtggtatctgctgtctatgggctctaatgagaacatccacagcatccatttctctggccatgtgttcactgtgaggaagaaggaggagtataagatggctctgtacaacctgtaccctggggtctttgagactgtggagatgctgcccagcaaggctggcatttggagggtggagtgcctgattggggaacacctgcatgctgggatgagcaccctgttcctggtgtactctaacaagtgccagaccccactgggcatggcttctggccacatcagggatttccagattactgcctctggccagtatggccagtgggctcccaagctggctaggctgcactactctgggagcatcaatgcctggtctactaaggagcctttctcttggatcaaagtggacctgctggcccctatgatcatccatgggatcaagactcagggggccaggcagaagttcagcagcctgtacatctctcagttcatcattatgtacagcctggatggcaagaagtggcagacctacaggggcaacagcactggcaccctgatggtgttctttgggaatgtggacagctctgggattaagcacaacatctttaacccccccatcattgccaggtatatcaggctgcaccctacccactacagcattaggagcaccctgaggatggagctgatgggctgtgacctgaacagctgcagcatgcccctggggatggagagcaaggccatttctgatgctcagatcactgcttctagctacttcactaacatgtttgccacctggtctcccagcaaggctagactgcacctgcaggggaggagcaatgcctggaggccccaggtgaataatcccaaggagtggctgcaggtggatttccagaaaaccatgaaggtgactggggtgactacccagggggtgaagtctctgctgaccagcatgtatgtgaaggagttcctgatcagcagcagccaggatgggcatcagtggaccctgttctttcagaatgggaaggtgaaggtgtttcagggcaatcaggacagcttcacccctgtggtgaacagcctggacccccccctgctgaccaggtacctgaggatccacccccagagctgggtgcatcagattgccctgaggatggaggtgctgggctgtgaggcccaggacctgtactga

SEQ ID NO: 5 - DNA, 4374 bp, synthetic construct
  source 1..4374: mol_type = other DNA
atgcagattgagctgtctacttgcttcttcctgtgcctgctgaggttctgcttctctgccactaggaggtattacctgggggctgtggagctgagctgggactatatgcagtctgacctgggggagctgcctgtggatgccaggtttcctcccagggtgcctaagagcttccccttcaacacctctgtggtgtacaagaagactctgtttgtggagtttactgatcatctgttcaacattgccaagcccaggcctccttggatggggctgctgggccccaccatccaggctgaggtgtatgacactgtggtgattaccctgaagaatatggccagccatcctgtgagcctgcatgctgtgggggtgagctattggaaggcctctgagggggctgagtatgatgatcagactagccagagggagaaggaggatgacaaggtgttccctggggggagccatacctatgtgtggcaggtgctgaaggagaatggccccatggcctctgaccctctgtgcctgacttatagctacctgagccatgtggatctggtgaaggacctgaactctggcctgattggggccctgctggtgtgcagggagggcagcctggccaaggagaagactcagaccctgcacaagttcatcctgctgtttgctgtgtttgatgaggggaagtcctggcactctgagactaagaacagcctgatgcaggatagggatgctgcttctgccagggcctggcctaagatgcacactgtgaatggctatgtgaataggagcctgcctggcctgattggctgccataggaagtctgtgtactggcatgtgattgggatgggcaccacccctgaggtgcactctattttcctggagggccatactttcctggtgaggaaccataggcaggccagcctggagatcagccccatcactttcctgactgcccagactctgctgatggacctgggccagttcctgctgttctgccacatcagcagccatcagcatgatggcatggaggcttatgtgaaggtggacagctgccctgaggagcctcagctgaggatgaagaataatgaggaggctgaggactatgatgatgacctgactgactctgagatggatgtggtgaggtttgatgatgacaactctccctctttcatccagatcaggtctgtggccaagaagcaccctaagacctgggtgcactacattgctgctgaggaggaggattgggactatgcccccctggtgctggccccagatgacaggagctacaagtcccagtacctgaacaatggcccccagaggattggcaggaagtacaagaaggtgaggttcatggcttatactgatgagactttcaagaccagggaggccatccagcatgagtctggcatcctgggccctctgctgtatggggaggtgggggacaccctgctgattatcttcaagaaccaggcttctaggccctacaatatctaccctcatggcatcactgatgtgaggcccctgtacagcaggaggctgcccaagggggtgaagcatctgaaggatttccccatcctgcctggggagatctttaagtataagtggactgtgactgtggaggatggccccactaagtctgaccccaggtgcctgaccaggtattacagcagctttgtgaacatggagagggatctggcttctgggctgattggccccctgctgatctgctacaaggagtctgtggaccagaggggcaaccagatcatgtctgacaagaggaatgtgatcctgttctctgtgtttgatgagaataggagctggtacctgactgagaacatccagaggtttctgcccaatcctgctggggtgcagctggaggatcctgagtttcaggcctctaatatcatgcacagcatcaatggctatgtgtttgactctctgcagctgtctgtgtgcctgcatgaggtggcctattggtacatcctgagcattggggcccagactgactttctgtctgtgtttttttctggctacaccttcaagcacaagatggtgtatgaggatactctgactctgttccctttttctggggagactgtgttcatgtctatggagaaccctgggctgtggattctgggctgccacaattctgacttcaggaacagaggcatgactgctctgctgaaggtgagcagctgtgacaagaacactggggactactatgaggactcttatgaggacatttctgcctacctgctgagcaagaacaatgccattgagcccagaagcttttctcagaacccccctgtgctgaagaggcaccagagggagatcaccaggaccaccctgcagtctgaccaggaggagattgactatgatgatactatttctgtggagatgaagaaggaggactttgacatctatgatgaggatgagaaccagagccccaggtctttccagaagaagactaggcactactttattgctgctgtggagaggctgtgggactatgggatgtctagctctcctcatgtgctgaggaacagggcccagtctggctctgtgccccagtttaaaaaggtggtgttccaggaattcactgatggcagctttacccagcctctgtacaggggggagctgaatgagcacctggggctgctggggccttacattagggctgaggtggaggacaacatcatggtgaccttcaggaatcaggccagcaggccctactctttctacagcagcctgatctcttatgaggaggaccagaggcagggggctgaacccaggaagaactttgtgaagcccaatgagaccaagacctacttctggaaggtgcagcaccacatggctcccaccaaggatgagtttgattgcaaggcctgggcttacttctctgatgtggatctggagaaggatgtgcactctgggctgattggccccctgctggtgtgccacaccaacactctgaaccctgcccatggcagacaggtgactgtgcaggagtttgccctgttcttcactatctttgatgagactaagagctggtacttcactgagaacatggagaggaattgcagggccccttgcaacatccagatggaggaccccacctttaaggagaactacaggtttcatgccattaatggctacatcatggacaccctgcctggcctggtgatggcccaggaccagaggatcaggtggtacctgctgtctatggggagcaatgagaacatccacagcattcacttctctggccatgtgttcactgtgaggaagaaggaggagtacaagatggccctgtacaacctgtaccctggggtgtttgagactgtggagatgctgcccagcaaggctgggatctggagggtggagtgcctgattggggagcacctgcatgctgggatgagcaccctgttcctggtgtatagcaacaagtgccagacccccctgggcatggcctctggccacatcagagactttcagattactgcctctggccagtatgggcagtgggcccccaagctggccaggctgcactattctggctctattaatgcctggagcactaaggagcccttcagctggattaaggtggacctgctggctcccatgatcatccatggcatcaagactcagggggccaggcagaagttctcttctctgtacatcagccagttcattatcatgtactccctggatggcaagaagtggcagacctataggggcaacagcactggcaccctgatggtgttctttgggaatgtggacagctctggcatcaagcataatatcttcaatccccccatcattgctaggtacatcaggctgcaccccacccactactctattaggtctaccctgaggatggagctgatgggctgtgacctgaacagctgcagcatgcctctgggcatggagagcaaagccatctctgatgcccagatcactgccagcagctactttaccaacatgtttgctacttggagccccagcaaggccaggctgcacctgcaggggaggtctaatgcctggaggccccaggtgaacaaccccaaggagtggctgcaggtggacttccagaagactatgaaggtgactggggtgaccacccagggggtgaagagcctgctgacctctatgtatgtgaaggagttcctgattagcagcagccaggatggccaccagtggaccctgtttttccagaatgggaaggtgaaggtgtttcaggggaaccaggacagcttcactcctgtggtgaactctctggacccccccctgctgaccaggtatctgaggatccaccctcagagctgggtgcaccagattgccctgaggatggaggtgctgggctgtgaggcccaggacctgtactga

SEQ ID NO: 6 - DNA, 4374 bp, synthetic construct
  source 1..4374: mol_type = other DNA
atgcagattgagctgagcacctgcttcttcctgtgcctgctgaggttttgcttctctgccaccaggaggtactacctgggggctgtggagctgagctgggattacatgcagtctgacctgggggagctgcctgtggatgccaggttccctcccagggtgcccaagtctttccccttcaacacttctgtggtgtacaagaagaccctgtttgtggagtttactgaccacctgttcaacattgccaagcccaggcctccctggatgggcctgctgggccccaccattcaggctgaggtgtatgacactgtggtcatcaccctgaaaaatatggctagccaccctgtgtctctgcatgctgtgggggtgagctactggaaggcctctgagggggctgagtatgatgaccagactagccagagggagaaggaggatgacaaggtgttccctgggggcagccacacttatgtgtggcaggtgctgaaagagaatggccccatggcttctgatcccctgtgtctgacctatagctacctgagccatgtggatctggtgaaggacctgaactctggcctgattggggccctgctggtgtgcagggagggcagcctggctaaggagaagacccagaccctgcataagttcatcctgctgtttgctgtgtttgatgagggcaagagctggcactctgagactaagaacagcctgatgcaggatagggatgctgcttctgccagggcctggcccaagatgcacactgtgaatgggtatgtgaacaggagcctgcctggcctgattggctgccataggaagtctgtctattggcatgtgattggcatgggcactactcctgaggtgcacagcatctttctggagggccacaccttcctggtgaggaaccacaggcaggccagcctggagatctctcccatcactttcctgactgctcagaccctgctgatggacctgggccagttcctgctgttctgtcacatctctagccaccagcatgatggcatggaggcctatgtgaaggtggatagctgccctgaggaaccccagctgaggatgaagaacaatgaggaggctgaggattatgatgatgatctgactgattctgagatggatgtggtgaggtttgatgatgacaattctcctagcttcattcagatcagatctgtggccaaaaagcatcctaagacttgggtgcattatattgctgctgaggaggaggattgggattatgcccccctggtgctggctcctgatgataggagctacaagtctcagtacctgaataatgggccccagaggattggcaggaagtacaagaaggtgaggttcatggcctacactgatgagaccttcaagaccagggaggccattcagcatgagtctgggattctggggcccctgctgtatggggaggtgggggataccctgctgatcattttcaagaaccaggccagcaggccctacaacatctacccccatgggattactgatgtgaggcccctgtactctaggaggctgcctaagggggtgaagcacctgaaggattttcctatcctgcctggggaaatcttcaagtacaagtggactgtgactgtggaggatggccccactaagtctgatcccaggtgtctgaccaggtattatagctcttttgtgaacatggagagggatctggcctctgggctgattggccctctgctgatctgctacaaggagtctgtggaccagaggggcaaccagatcatgtctgacaagaggaatgtgatcctgttctctgtgtttgatgagaacaggagctggtatctgactgagaacatccagaggtttctgcccaatcctgctggggtgcagctggaggatcctgagttccaggctagcaacatcatgcacagcatcaatgggtatgtgtttgacagcctgcagctgtctgtgtgtctgcatgaggtggcctactggtatatcctgtctattggggcccagactgacttcctgtctgtgtttttttctgggtatacttttaagcacaagatggtgtatgaggacaccctgactctgttccccttctctggggagactgtgtttatgagcatggagaaccctggcctgtggatcctgggctgccacaattctgacttcaggaatagggggatgactgccctgctgaaggtgagcagctgtgataagaatactggggactactatgaggactcttatgaggacatttctgcctatctgctgtctaagaacaatgccattgaacccaggagcttctctcagaacccccctgtgctgaagaggcaccagagggaaatcaccagaactactctgcagtctgatcaggaggaaattgactatgatgacactatttctgtggagatgaagaaggaggactttgacatctatgatgaggatgagaaccagagcccaaggagcttccagaagaagactaggcactacttcattgctgctgtggagaggctgtgggactatggcatgagcagcagcccccatgtgctgagaaacagggcccagtctgggtctgtgccccagttcaagaaggtggtgttccaggagttcactgatgggagcttcacccagcccctgtataggggggagctgaatgagcacctgggcctgctgggcccctatattagggctgaggtggaggacaacatcatggtgaccttcaggaatcaggcctctaggccctacagcttctacagcagcctgattagctatgaggaggatcagaggcagggggctgaacccaggaagaactttgtgaagcccaatgagaccaagacctatttctggaaggtgcagcatcacatggcccccaccaaggatgagtttgactgcaaggcctgggcctacttctctgatgtggatctggagaaggatgtgcactctggcctgattggccccctgctggtgtgccacaccaacaccctgaaccctgctcatggcaggcaggtgactgtgcaggagtttgccctgttcttcaccatctttgatgagactaagtcttggtacttcactgagaatatggagaggaattgcagggccccctgcaatattcagatggaagaccccaccttcaaggagaattacaggttccatgccattaatggctacatcatggataccctgcctggcctggtgatggcccaggatcagaggatcaggtggtacctgctgagcatgggcagcaatgagaacatccactctatccacttctctggccatgtgtttactgtgaggaagaaggaggagtataagatggccctgtacaacctgtaccctggggtctttgagactgtggagatgctgccttctaaggctggcatttggagggtggagtgcctgattggggaacacctgcatgctggcatgtctaccctgttcctggtgtacagcaataagtgccagacccccctgggcatggcctctgggcatatcagggatttccagatcactgcctctggccagtatggccagtgggccccaaagctggctaggctgcactactctgggagcatcaatgcctggagcactaaggagcccttcagctggatcaaggtggacctgctggcccccatgattatccatgggattaagactcagggggccaggcagaagttcagcagcctgtacatcagccagttcattatcatgtacagcctggatggcaagaagtggcagacctataggggcaactctactgggaccctgatggtgttctttgggaatgtggatagctctgggatcaagcacaatatcttcaacccccccatcattgccaggtatatcaggctgcaccccacccactacagcattaggtctaccctgaggatggagctgatgggctgtgatctgaacagctgtagcatgcctctgggcatggagtctaaggccatttctgatgcccagattactgctagcagctacttcaccaacatgtttgccacctggtctcccagcaaggccaggctgcatctgcagggcaggtctaatgcttggaggccccaggtgaacaacccaaaggagtggctgcaggtggatttccagaagactatgaaggtgactggggtgaccactcagggggtgaagtctctgctgacctctatgtatgtgaaggagttcctgatctctagcagccaggatggccatcagtggaccctgttcttccagaatggcaaggtgaaagtgttccagggcaatcaggatagcttcactccagtggtgaacagcctggatccccctctgctgactaggtacctgaggatccacccccagagctgggtgcaccagattgccctgaggatggaggtgctgggctgtgaggcccaggacctgtactga

SEQ ID NO: 7 - DNA, 4374 bp, synthetic construct
  source 1..4374: mol_type = other DNA
atgcagattgagctgagcacctgcttcttcctgtgtctgctgaggttctgcttctctgccaccaggaggtattacctgggggctgtggagctgagctgggactatatgcagtctgacctgggggagctgcctgtggatgctaggttcccccccagggtgcccaagagcttcccctttaacacttctgtggtgtacaagaagaccctgtttgtggagttcactgaccacctgttcaacattgccaagcccaggcccccctggatggggctgctggggcccaccatccaggctgaggtgtatgacactgtggtgatcaccctgaagaacatggccagccaccctgtgagcctgcatgctgtgggggtgagctactggaaggcttctgagggggctgagtatgatgaccagactagccagagggagaaggaggatgacaaggtgtttcctgggggcagccatacctatgtgtggcaggtgctgaaggagaatggccccatggcctctgaccccctgtgcctgacctacagctacctgtctcatgtggacctggtgaaggacctgaactctggcctgattggggctctgctggtgtgtagggagggcagcctggctaaggaaaagacccagaccctgcataagtttatcctgctgtttgctgtgtttgatgagggcaagagctggcactctgagaccaagaacagcctgatgcaggatagggatgctgcctctgccagggcttggcctaagatgcacactgtgaatgggtatgtgaataggagcctgcctggcctgattggctgccacaggaagtctgtgtactggcatgtgattgggatgggcaccacccctgaggtccatagcatcttcctggagggccacactttcctggtgaggaaccacagacaggcctctctggagatctctcccatcaccttcctgactgctcagactctgctgatggacctgggccagttcctgctgttttgccatattagcagccaccagcatgatgggatggaggcctatgtgaaggtggatagctgccctgaggagcctcagctgaggatgaagaacaatgaggaggctgaagactatgatgatgacctgactgattctgagatggatgtggtgaggtttgatgatgacaatagccccagcttcattcagatcaggtctgtggccaagaaacaccccaagacctgggtgcactacattgctgctgaggaagaggactgggactatgctcccctggtgctggcccctgatgataggtcttataagagccagtacctgaacaatgggccccagaggattggcaggaagtacaagaaggtgaggttcatggcctacactgatgaaaccttcaaaaccagggaggccattcagcatgagtctggcatcctgggccctctgctgtatggggaggtgggggacaccctgctgatcatcttcaagaaccaggccagcaggccctacaacatctatcctcatggcatcactgatgtgaggcccctgtacagcaggaggctgcccaagggggtgaagcacctgaaagacttccccatcctgcctggggagatctttaagtataagtggactgtgactgtggaggatggccctaccaagtctgaccccaggtgtctgaccaggtactattctagctttgtgaacatggagagggacctggcctctggcctgattgggcccctgctgatctgctacaaggagtctgtggaccagaggggcaaccagatcatgtctgacaagaggaatgtgatcctgttttctgtgtttgatgagaataggagctggtacctgactgagaacatccagaggtttctgcccaatcctgctggggtgcagctggaggatcctgagttccaggccagcaatatcatgcatagcatcaatggctatgtgtttgacagcctgcagctgtctgtgtgcctgcatgaggtggcctactggtacatcctgagcattggggcccagactgactttctgtctgtgttcttttctggctataccttcaagcacaagatggtgtatgaggataccctgaccctgttccccttctctggggagactgtgttcatgagcatggagaatcctgggctgtggatcctggggtgccacaactctgattttaggaacagggggatgactgccctgctgaaggtgtctagctgtgataagaacactggggactactatgaggacagctatgaggacatttctgcttatctgctgtctaagaataatgccattgagcccagaagcttcagccagaatccccctgtgctgaagagacatcagagggagatcaccagaactaccctgcagtctgatcaggaggagattgactatgatgacactatctctgtggagatgaagaaggaggactttgacatctatgatgaggatgagaatcagtctcccaggagctttcagaagaagaccagacattacttcattgctgctgtggagaggctgtgggactatggcatgagctctagccctcatgtgctgaggaacagggcccagtctggctctgtgccccagttcaagaaggtggtgttccaggaattcactgatggcagcttcacccagcccctgtacaggggggagctgaatgagcacctgggcctgctggggccttatatcagggctgaggtggaggataatattatggtgactttcaggaaccaggccagcaggccctactctttctatagcagcctgatctcttatgaggaggatcagaggcagggggctgagcctaggaagaactttgtgaagcccaatgagactaagacctacttctggaaggtccagcaccacatggcccctaccaaggatgagtttgactgcaaggcctgggcctatttctctgatgtggatctggagaaggatgtccattctgggctgattggccccctgctggtgtgccacactaacactctgaatcctgcccatggcaggcaggtgactgtccaggagtttgccctgttcttcactatctttgatgagaccaagagctggtactttactgagaacatggagaggaactgcagagctccttgcaatattcagatggaggaccccaccttcaaggagaattacaggttccatgccattaatgggtacatcatggacaccctgcctggcctggtgatggctcaggaccagaggatcaggtggtacctgctgagcatgggctctaatgagaatatccacagcatccacttctctgggcatgtgttcactgtgaggaagaaggaggagtacaagatggctctgtataatctgtaccctggggtgtttgaaactgtggagatgctgccctctaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactattctggcagcatcaatgcctggagcaccaaggagcccttcagctggatcaaggtggacctgctggcccccatgatcattcatggcatcaagacccagggggccaggcagaagttcagctctctgtacatctctcagttcatcatcatgtactctctggatgggaagaagtggcagacctacaggggcaacagcactggcaccctgatggtgttctttgggaatgtggactcttctggcatcaagcacaacatcttcaatccccccatcattgctaggtatattaggctgcatcccacccactacagcatcaggtctaccctgaggatggagctgatgggctgtgacctgaactcttgcagcatgcccctgggcatggagtctaaggccatctctgatgcccagattactgccagcagctacttcaccaacatgtttgccacctggagcccctctaaggccaggctgcatctgcaggggaggagcaatgcctggaggcctcaggtgaacaaccccaaggagtggctgcaggtggatttccagaagaccatgaaggtgactggggtgaccacccagggggtcaagagcctgctgaccagcatgtatgtgaaggagttcctgatcagcagcagccaggatggccaccagtggactctgttctttcagaatgggaaggtgaaggtgtttcagggcaatcaggactctttcacccctgtggtgaacagcctggacccccccctgctgaccagatacctgaggatccacccccagtcttgggtgcatcagattgccctgaggatggaggtgctgggctgtgaggctcaggatctgtactga

SEQ ID NO: 8 - DNA, 4374 bp, synthetic construct
  source 1..4374: mol_type = other DNA
atgcagattgagctgagcacttgcttttttctgtgcctgctgaggttttgtttttctgccaccaggaggtactacctgggggctgtggagctgagctgggactatatgcagtctgatctgggggagctgcctgtggatgccaggttcccccccagggtgcccaagtcttttcccttcaacacctctgtggtgtataagaagaccctgtttgtggagttcactgaccacctgttcaacattgctaagcctaggcccccctggatgggcctgctgggccctaccattcaggctgaggtgtatgacactgtggtgatcaccctgaagaacatggccagccatcctgtgagcctgcatgctgtgggggtctcttactggaaggcctctgagggggctgagtatgatgaccagaccagccagagagagaaggaggatgacaaggtcttccctgggggctctcacacctatgtgtggcaggtgctgaaggaaaatggccccatggcctctgaccccctgtgcctgacctacagctatctgagccatgtggatctggtgaaggacctgaattctggcctgattggggccctgctggtgtgcagggagggcagcctggccaaggagaagacccagaccctgcacaagtttatcctgctgtttgctgtgtttgatgagggcaagtcttggcactctgagactaagaacagcctgatgcaggacagggatgctgcctctgccagggcctggcccaagatgcacactgtgaatggctatgtgaacaggagcctgcctgggctgattggctgccacaggaagtctgtgtactggcatgtgattggcatgggcaccacccctgaggtgcacagcatcttcctggaaggccacactttcctggtgaggaaccataggcaggccagcctggagatcagccctatcaccttcctgactgcccagaccctgctgatggatctggggcagttcctgctgttctgccacatctctagccaccagcatgatgggatggaggcctatgtgaaggtggacagctgcccagaggagcctcagctgaggatgaaaaacaatgaagaggctgaggattatgatgatgatctgactgactctgagatggatgtggtgagatttgatgatgacaatagccctagctttattcagatcaggtctgtggctaagaagcaccccaagacctgggtgcattacattgctgctgaggaggaggactgggattatgctcctctggtgctggcccctgatgataggagctacaagagccagtacctgaataatggccctcagaggattggcaggaagtacaagaaggtgaggttcatggcttacactgatgagaccttcaagactagggaggccatccagcatgagtctgggatcctggggcccctgctgtatggggaggtgggggacaccctgctgatcatcttcaagaaccaggctagcaggccttacaacatctatccccatgggatcactgatgtgagacctctgtacagcaggaggctgcccaagggggtcaagcatctgaaagacttccccatcctgcctggggagatctttaagtataagtggactgtgactgtggaggatgggcccaccaagtctgaccccaggtgcctgaccaggtattacagcagctttgtgaacatggagagggatctggcctctgggctgattggccccctgctgatctgttacaaggaatctgtggatcagaggggcaatcagatcatgtctgacaagaggaatgtgatcctgttctctgtgtttgatgagaataggtcttggtacctgactgaaaacatccagaggttcctgcccaaccctgctggggtccagctggaggatcctgagttccaggctagcaacatcatgcacagcatcaatgggtatgtgtttgatagcctgcagctgtctgtgtgcctgcatgaggtggcctactggtacatcctgtctattggggcccagactgacttcctgtctgtgttcttttctggctacaccttcaagcacaagatggtgtatgaggacaccctgaccctgttccccttctctggggagactgtctttatgagcatggagaaccctgggctgtggatcctgggctgccacaactctgatttcaggaataggggcatgactgctctgctgaaggtgagctcttgtgacaagaacactggggattactatgaggacagctatgaggacatttctgcctacctgctgagcaagaacaatgccattgagcctaggagctttagccagaatcctcctgtcctgaagaggcaccagagggagatcaccaggaccaccctgcagtctgaccaggaggagattgactatgatgataccatctctgtggagatgaagaaggaggactttgacatctatgatgaggatgagaatcagtctcccaggagcttccagaagaagaccaggcactatttcattgctgctgtggagaggctgtgggactatggcatgagcagctctcctcatgtgctgaggaatagggctcagtctggctctgtgccccagttcaagaaagtggtgtttcaggagttcactgatggctctttcacccagcctctgtataggggggagctgaatgagcacctggggctgctgggcccctatatcagggctgaggtggaggataacatcatggtgaccttcaggaaccaggcctctaggccctacagcttctatagcagcctgatcagctatgaggaggaccagaggcagggggctgagcccaggaagaactttgtgaagcccaatgagaccaagacttacttctggaaggtgcagcatcacatggcccccaccaaggatgagtttgactgtaaggcctgggcctacttctctgatgtggatctggagaaggatgtgcactctggcctgattggccccctgctggtgtgccataccaatactctgaaccctgctcatggcaggcaggtgactgtgcaggagtttgctctgttcttcactatctttgatgagaccaagtcttggtatttcactgagaatatggagaggaactgcagggccccctgcaacatccagatggaggaccccacctttaaggagaactataggtttcatgccatcaatggctacatcatggacaccctgcctggcctggtgatggcccaggatcagaggatcaggtggtacctgctgagcatggggtctaatgagaacatccacagcatccacttctctggccatgtgtttactgtgagaaagaaggaggagtacaagatggctctgtacaatctgtaccctggggtctttgagactgtggagatgctgcctagcaaggctgggatctggagggtggagtgcctgattggggaacatctgcatgctgggatgtctactctgttcctggtgtacagcaacaagtgccagacccccctgggcatggcttctggccatatcagggactttcagattactgcctctgggcagtatggccagtgggcccccaagctggctaggctgcattattctggcagcatcaatgcctggtctactaaggagcccttcagctggatcaaggtggatctgctggcccccatgatcatccatggcatcaagacccagggggccaggcagaagtttagctctctgtacattagccagttcatcatcatgtacagcctggatgggaagaagtggcagacctacaggggcaattctactggcaccctgatggtgttctttggcaatgtggacagctctggcatcaagcacaacatctttaacccccctatcattgctaggtacatcaggctgcatcccacccattacagcatcaggagcaccctgaggatggagctgatgggctgtgacctgaactcttgcagcatgcccctgggcatggagagcaaggccatttctgatgcccagattactgccagcagctacttcactaacatgtttgccacctggtctcccagcaaggccaggctgcacctgcagggcaggagcaatgcctggaggccccaggtgaacaaccccaaggagtggctgcaggtggatttccagaagaccatgaaggtgactggggtgaccacccagggggtgaagagcctgctgactagcatgtatgtgaaggagttcctgatcagctctagccaggatggccaccagtggactctgtttttccagaatggcaaggtgaaggtgttccagggcaaccaggactctttcactcctgtggtgaacagcctggacccccccctgctgaccaggtatctgaggattcacccccagtcttgggtgcatcagattgccctgaggatggaggtgctgggctgtgaggcccaggatctgtactga

SEQ ID NO: 9 - DNA, 4374 bp, synthetic construct
  source 1..4374: mol_type = other DNA
atgcagattgagctgagcacctgcttcttcctgtgtctgctgagattttgcttttctgccactaggaggtattacctgggggctgtggagctgtcttgggactacatgcagtctgatctgggggagctgcctgtggatgccaggttcccacctagggtgcctaagagctttcccttcaatacctctgtggtgtacaagaagaccctgtttgtggagttcactgaccacctgttcaacattgccaagcctaggcccccctggatgggcctgctgggccctaccatccaggctgaagtgtatgacactgtggtgatcaccctgaagaacatggccagccaccctgtgagcctgcatgctgtgggggtgtcttactggaaggcctctgagggggctgagtatgatgatcagaccagccagagggagaaggaagatgacaaggtgttccctgggggcagccacacctatgtctggcaggtgctgaaggagaatggccccatggcctctgatcccctgtgcctgacctactcttacctgagccatgtggacctggtgaaggatctgaattctggcctgattggggccctgctggtgtgcagggagggcagcctggccaaggagaagacccagaccctgcataagttcatcctgctgtttgctgtgtttgatgaagggaagagctggcactctgagactaagaacagcctgatgcaggacagggatgctgcttctgccagggcctggcccaagatgcacactgtgaatggctatgtgaatagaagcctgcctggcctgattgggtgccacaggaagtctgtgtactggcatgtgattgggatgggcactacccctgaggtgcatagcatcttcctggaaggccataccttcctggtgaggaatcataggcaggcttctctggaaatttctcccatcactttcctgactgctcagaccctgctgatggacctgggccagttcctgctgttctgccacatcagctctcaccagcatgatgggatggaggcctatgtgaaggtggacagctgtcctgaggagccccagctgaggatgaagaacaatgaggaggctgaggactatgatgatgacctgactgactctgagatggatgtggtcaggtttgatgatgacaatagcccctctttcatccagatcaggtctgtggccaagaagcaccccaagacttgggtgcactacattgctgctgaggaggaggattgggattatgcccctctggtgctggcccctgatgacaggagctataagtctcagtacctgaataatggcccccagaggattgggaggaagtataagaaggtgaggtttatggcctacactgatgagaccttcaagaccagggaggccatccagcatgagtctggcatcctgggccccctgctgtatggggaggtgggggataccctgctgatcatcttcaagaaccaggcctctaggccctacaatatctaccctcatggcatcactgatgtgagacccctgtatagcaggaggctgcctaagggggtgaagcacctgaaggacttccccatcctgcctggggagatcttcaagtataagtggactgtgactgtggaggatggccccaccaagtctgaccccaggtgcctgaccaggtattacagctcttttgtgaacatggagagggatctggcctctgggctgattggcccactgctgatctgctacaaggagtctgtggatcagaggggcaatcagatcatgtctgacaagaggaatgtgatcctgttttctgtgtttgatgaaaataggtcttggtatctgactgagaacatccagaggtttctgcccaatcctgctggggtgcagctggaggatcctgagtttcaggcctctaatatcatgcattctatcaatggctatgtgtttgacagcctgcagctgtctgtgtgcctgcatgaggtggcctactggtacatcctgagcattggggctcagactgacttcctgtctgtgttcttttctggctatactttcaagcacaagatggtgtatgaggacactctgaccctgttccccttctctggggagactgtgttcatgtctatggaaaatcctgggctgtggattctgggctgccacaattctgacttcaggaatagggggatgactgccctgctgaaggtgtctagctgtgataagaacactggggattactatgaggactcttatgaagatatctctgcctatctgctgagcaagaacaatgccattgagcccaggagcttcagccagaacccccctgtgctgaagaggcaccagagggagatcaccaggaccactctgcagtctgatcaggaggagattgactatgatgacactatctctgtggagatgaagaaggaggattttgacatttatgatgaggatgagaaccagtctcccaggagcttccagaagaagaccaggcattactttattgctgctgtggagaggctgtgggactatgggatgagcagctctcctcatgtgctgaggaacagggcccagtctgggtctgtgccccagttcaagaaggtggtgttccaggagttcactgatgggagcttcacccagcccctgtataggggggagctgaatgagcacctgggcctgctgggcccctacatcagggctgaggtggaggataatatcatggtgaccttcaggaaccaggctagcaggccttacagcttttacagcagcctgatctcttatgaagaagaccagaggcagggggctgagcccaggaagaattttgtgaagcctaatgagaccaagacttatttttggaaggtgcagcatcacatggctcctaccaaggatgagtttgactgcaaggcctgggcctacttttctgatgtggatctggagaaggatgtgcactctggcctgattggccctctgctggtgtgccatactaacactctgaaccctgcccatgggaggcaggtgactgtgcaggagtttgccctgttcttcactatttttgatgagaccaagtcttggtatttcactgagaacatggagaggaactgcagggctccctgcaacatccagatggaagaccccaccttcaaggagaactataggttccatgccatcaatgggtacatcatggataccctgcctggcctggtgatggcccaggatcagaggattaggtggtatctgctgagcatgggctctaatgagaacatccacagcatccatttctctggccatgtgttcactgtgaggaagaaggaggagtacaagatggctctgtacaacctgtatcctggggtgtttgagactgtggagatgctgcccagcaaggctggcatctggagggtggaatgcctgattggggagcacctgcatgctggcatgagcactctgttcctggtgtatagcaacaagtgccagacccccctgggcatggcctctggccatatcagggatttccagatcactgcttctggccagtatggccagtgggcccccaagctggccaggctgcactattctggcagcatcaatgcctggagcactaaggagcctttttcttggatcaaggtggacctgctggcccctatgattattcatggcatcaagacccagggggccaggcagaagttctctagcctgtacatctctcagttcatcattatgtatagcctggatggcaagaagtggcagacctacaggggcaatagcactggcaccctgatggtgttttttgggaatgtggactcttctgggatcaagcacaacatctttaacccccccatcattgccaggtatattaggctgcaccccacccactacagcatcaggagcaccctgaggatggagctgatgggctgtgatctgaattcttgctctatgcccctgggcatggagagcaaggccatctctgatgcccagatcactgccagctcttacttcaccaacatgtttgccacctggtctcctagcaaggccaggctgcatctgcagggcaggagcaatgcctggaggccccaggtgaacaaccccaaggagtggctgcaggtggacttccagaagaccatgaaggtgactggggtgaccactcagggggtgaagagcctgctgacctctatgtatgtgaaggagttcctgatcagcagcagccaggatggccaccagtggactctgttcttccagaatgggaaggtgaaggtgttccagggcaaccaggatagctttacccctgtggtgaacagcctggaccctcctctgctgaccagatacctgaggatccatcctcagagctgggtgcaccagattgccctgaggatggaggtgctgggctgtgaggcccaggatctgtactga

SEQ ID NO: 10 - DNA, 4374 bp, synthetic construct
  source 1..4374: mol_type = other DNA
atgcagattgagctgagcacttgcttcttcctgtgcctgctgaggttctgcttttctgctactaggaggtactacctgggggctgtggagctgagctgggattacatgcagtctgacctgggggagctgccagtggatgccaggttcccccccagggtgcccaagtcttttcctttcaacacctctgtggtgtacaagaagaccctgtttgtggagttcactgaccacctgttcaacattgccaagcccaggcccccctggatggggctgctggggcccaccatccaggctgaggtgtatgacactgtggtgattaccctgaagaacatggctagccaccctgtgagcctgcatgctgtgggggtgagctattggaaggcctctgagggggctgagtatgatgatcagaccagccagagggaaaaggaggatgacaaggtgttccctgggggcagccatacttatgtgtggcaggtgctgaaggagaatgggcccatggcctctgaccccctgtgcctgacttacagctatctgagccatgtggacctggtgaaggatctgaactctggcctgattggggctctgctggtgtgcagggagggcagcctggctaaggagaagactcagactctgcataagttcatcctgctgtttgctgtgtttgatgaaggcaagagctggcactctgagaccaagaactctctgatgcaggatagggatgctgcctctgccagggcttggcccaagatgcacactgtgaatggctatgtgaacaggagcctgcctggcctgattgggtgccacaggaagtctgtgtactggcatgtgattggcatgggcaccacccctgaggtgcacagcattttcctggagggccacaccttcctggtgaggaatcacaggcaggccagcctggagatcagccccatcaccttcctgactgcccagaccctgctgatggacctggggcagtttctgctgttctgccacatcagcagccatcagcatgatggcatggaggcctatgtgaaggtggactcttgccctgaggagccccagctgaggatgaagaacaatgaggaggctgaggattatgatgatgacctgactgactctgagatggatgtggtgaggtttgatgatgacaatagccccagcttcatccagattaggtctgtggccaagaagcaccctaagacctgggtgcactacattgctgctgaggaggaggattgggattatgcccccctggtgctggctcctgatgacaggtcttataagagccagtacctgaacaatgggccccagaggattggcaggaagtacaagaaggtgaggttcatggcttacactgatgagaccttcaagactagggaggccatccagcatgagtctggcatcctgggccccctgctgtatggggaggtgggggataccctgctgatcatcttcaagaaccaggccagcaggccctacaacatttaccctcatggcatcactgatgtgaggcccctgtacagcaggagactgcccaagggggtgaagcacctgaaggattttcccattctgcctggggagatcttcaagtacaagtggactgtgactgtggaggatggccccaccaagtctgatcccaggtgcctgactaggtactactcttcttttgtgaatatggagagggatctggcctctggcctgattggccccctgctgatctgctacaaggagtctgtggaccagaggggcaaccagatcatgtctgacaagaggaatgtgatcctgttctctgtgtttgatgagaataggagctggtacctgactgagaatatccagaggttcctgcctaatcctgctggggtccagctggaggatcctgagttccaggctagcaacattatgcacagcatcaatggctatgtgtttgattctctgcagctgtctgtgtgcctgcatgaggtggcttactggtacatcctgtctattggggcccagactgatttcctgtctgtgttcttctctggctacactttcaagcataagatggtgtatgaggataccctgaccctgttccccttctctggggagactgtgttcatgtctatggagaaccctggcctgtggatcctgggctgtcataactctgacttcagaaacaggggcatgactgccctgctgaaggtgagcagctgtgacaagaacactggggactactatgaggacagctatgaggatatctctgcttatctgctgagcaagaataatgccattgagcccaggagcttcagccagaacccccctgtgctgaagaggcaccagagggagatcactaggactaccctgcagtctgatcaggaggagattgactatgatgacaccatctctgtggagatgaagaaggaggactttgacatctatgatgaggatgagaaccagtcccccaggtctttccagaagaagaccaggcactacttcattgctgctgtggagaggctgtgggactatggcatgagctctagcccccatgtgctgaggaacagggctcagtctggctctgtgccccagttcaagaaggtggtcttccaggagttcactgatggctcttttacccagcctctgtacagaggggagctgaatgagcacctgggcctgctgggcccctacatcagggctgaggtggaggataatatcatggtgaccttcagaaaccaggcctctaggccctacagcttctacagcagcctgatctcttatgaggaggatcagaggcagggggctgagcccaggaagaactttgtgaagcccaatgagaccaagacctacttctggaaggtgcagcaccatatggcccctactaaggatgagtttgactgcaaggcctgggcttatttttctgatgtggacctggagaaggatgtgcactctgggctgattggccccctgctggtgtgccacaccaacaccctgaaccctgcccatggcaggcaggtgactgtgcaggagtttgccctgttcttcactatctttgatgagaccaagagctggtacttcactgagaacatggagagaaattgtagggctccctgcaatatccagatggaggaccccaccttcaaagaaaattacagattccatgccatcaatgggtacatcatggataccctgcctgggctggtgatggctcaggaccagaggatcaggtggtacctgctgagcatggggtctaatgagaacatccactctatccatttctctggccatgtgttcactgtgagaaagaaggaggagtataagatggctctgtacaacctgtacccaggggtgtttgagactgtggaaatgctgcccagcaaagctgggatctggagggtggagtgcctgattggggagcacctgcatgctggcatgtctaccctgttcctggtgtacagcaacaagtgccagactcccctgggcatggcctctgggcacatcagggattttcagatcactgcctctggccagtatggccagtgggcccccaagctggccaggctgcactactctggcagcattaatgcttggagcactaaggagcccttcagctggatcaaggtggatctgctggcccccatgatcatccatggcatcaagacccagggggccaggcagaagttctctagcctgtacatttctcagttcatcatcatgtacagcctggatgggaagaagtggcagacctacagggggaacagcactgggaccctgatggtgttctttggcaatgtggatagctctggcatcaagcacaatatcttcaatccccccattattgccaggtacattaggctgcatcctactcactactctattaggagcaccctgaggatggagctgatggggtgtgacctgaacagctgttctatgcccctgggcatggagtctaaggctatctctgatgcccagatcactgccagcagctacttcactaatatgtttgccacctggagccctagcaaggccagactgcacctgcagggcaggagcaatgcctggaggccccaggtgaacaaccccaaggagtggctgcaggtggacttccagaagaccatgaaggtgactggggtgaccactcagggggtgaagagcctgctgaccagcatgtatgtgaaggagttcctgatcagcagcagccaggatggccaccagtggaccctgttcttccagaatgggaaggtgaaggtgttccagggcaaccaggactctttcacccctgtggtgaacagcctggatcctcccctgctgaccaggtacctgaggatccacccccagagctgggtgcaccagattgctctgaggatggaagtgctgggctgtgaggcccaggatctgtactga

SEQ ID NO: 11 - DNA, 4374 bp, synthetic construct
  source 1..4374: mol_type = other DNA
atgcagattgagctgagcacctgcttcttcctgtgcctgctgaggttttgcttctctgctaccaggaggtactacctgggggctgtggagctgagctgggactatatgcagtctgacctgggggagctgcctgtggatgctaggttccctcccagggtgcccaagagcttcccctttaatacctctgtggtgtacaagaaaaccctgtttgtggagttcactgaccatctgttcaacattgccaagcccaggcccccttggatgggcctgctgggccccaccattcaggctgaggtgtatgacactgtggtcattaccctgaagaacatggcttctcaccctgtgagcctgcatgctgtgggggtgagctactggaaggcctctgagggggctgagtatgatgaccagaccagccagagggagaaggaggatgataaggtgttccctgggggcagccacacctatgtgtggcaggtgctgaaggagaatggccccatggcctctgatcccctgtgcctgacctactcttatctgtctcatgtggacctggtgaaggacctgaactctggcctgattggggctctgctggtgtgcagggagggctctctggccaaggagaagacccagaccctgcacaagtttattctgctgtttgctgtctttgatgagggcaagagctggcattctgagaccaagaacagcctgatgcaggacagggatgctgcctctgccagggcctggcccaaaatgcacactgtgaatggctatgtgaacaggagcctgcctggcctgattggctgccacaggaagtctgtgtactggcatgtgattggcatgggcaccacccctgaggtgcacagcatcttcctggagggccacacctttctggtgaggaatcacaggcaggccagcctggagattagccccatcaccttcctgactgcccagaccctgctgatggacctgggccagttcctgctgttctgccacatcagcagccaccagcatgatggcatggaggcctatgtgaaggtggatagctgccctgaggagccccagctgaggatgaaaaacaatgaggaggctgaggattatgatgatgacctgactgactctgagatggatgtggtgaggtttgatgatgacaatagccccagctttattcagattaggtctgtggctaagaagcaccccaagacttgggtgcactacattgctgctgaggaggaggattgggactatgcccctctggtcctggcccctgatgataggtcttacaagagccagtatctgaacaatggcccccagaggattggcaggaagtacaagaaggtgaggttcatggcctacactgatgagacctttaagaccagggaggccattcagcatgagtctgggatcctgggccccctgctgtatggggaggtgggggacactctgctgatcatcttcaagaaccaggccagcaggccttataacatctaccctcatgggatcactgatgtgaggcccctgtactctagaaggctgcccaagggggtcaagcacctgaaggattttcccatcctgcctggggagattttcaagtacaagtggactgtgactgtggaggatggccccaccaagtctgaccctaggtgcctgaccaggtactacagctcttttgtgaacatggagagggacctggcctctggcctgattggccctctgctgatttgctacaaggagtctgtggaccagaggggcaaccagatcatgtctgacaagaggaatgtgatcctgttttctgtgtttgatgagaacaggtcttggtacctgactgagaacatccagaggttcctgcctaacccagctggggtgcagctggaggatcctgagttccaggccagcaatattatgcatagcattaatggctatgtgtttgatagcctgcagctgtctgtgtgcctgcatgaggtggcctactggtacatcctgagcattggggcccagactgactttctgtctgtgttcttctctggctacaccttcaagcataagatggtgtatgaggacaccctgactctgttccctttttctggggagactgtgtttatgagcatggagaatcctggcctgtggatcctgggctgccataattctgacttcaggaacaggggcatgactgccctgctgaaagtgagcagctgtgacaagaatactggggactactatgaagacagctatgaggacatctctgcctacctgctgagcaagaacaatgccattgagcccaggagcttcagccagaaccccccagtgctgaagaggcaccagagagagatcaccaggactaccctgcagtctgaccaggaggagattgactatgatgacaccatttctgtggagatgaagaaggaggactttgacatttatgatgaggatgagaatcagagccccaggagcttccagaagaagactaggcactattttattgctgctgtggagaggctgtgggactatggcatgagcagctctccccatgtgctgaggaatagggcccagtctggctctgtgcctcagttcaagaaggtggtgttccaggagttcactgatggcagctttacccagcccctgtataggggggagctgaatgagcacctgggcctgctgggcccctatatcagggctgaggtggaggacaatattatggtgacctttaggaaccaggccagcaggccctactctttctatagcagcctgatcagctatgaggaggaccagaggcagggggctgagcccaggaagaattttgtgaagcctaatgagaccaagacctacttctggaaggtgcagcatcacatggcccccaccaaggatgagtttgactgcaaggcttgggcctatttctctgatgtggacctggagaaggatgtgcactctggcctgattggccccctgctggtgtgccacactaacactctgaatcctgcccatggcaggcaggtgactgtgcaggagtttgccctgttcttcaccatctttgatgagaccaagagctggtacttcactgagaacatggagaggaactgcagggccccctgcaacatccagatggaggatcccaccttcaaggagaactacaggtttcatgccatcaatggctacatcatggacactctgcctggcctggtgatggcccaggatcagaggatcaggtggtacctgctgagcatgggctctaatgagaatatccatagcatccacttctctggccatgtgttcactgtcaggaagaaggaggagtacaagatggctctgtataatctgtaccctggggtgtttgagactgtggagatgctgcccagcaaggctggcatctggagggtggagtgcctgattggggagcacctgcatgctgggatgagcaccctgtttctggtgtactctaacaagtgccagacccccctgggcatggcctctgggcacatcagggatttccagatcactgcttctggccagtatggccagtgggcccccaagctggccaggctgcactactctggcagcatcaatgcctggtctaccaaggagcccttttcttggattaaggtggacctgctggcccccatgatcatccatggcatcaagacccagggggccaggcagaagttcagcagcctgtacatcagccagttcatcatcatgtacagcctggatggcaaaaagtggcagacctacaggggcaatagcactgggactctgatggtgttctttggcaatgtggacagctctgggatcaagcacaatatcttcaaccctcccatcattgctaggtacatcaggctgcaccccacccactatagcatcaggtctaccctgaggatggagctgatgggctgtgacctgaactcttgcagcatgcccctgggcatggagtccaaagctatctctgatgcccagattactgccagcagctacttcaccaacatgtttgccacctggtctccctctaaggccaggctgcacctgcagggcaggagcaatgcctggaggccccaggtgaacaatcccaaggagtggctgcaggtggatttccagaaaactatgaaggtgactggggtgaccacccagggggtgaagtctctgctgaccagcatgtatgtgaaggagttcctgatctcttctagccaggatggccaccagtggactctgttcttccagaatggcaaggtgaaggtgttccagggcaaccaggacagcttcacccctgtggtgaactctctggatccccccctgctgaccaggtacctgaggattcatccccagagctgggtgcaccagattgctctgagaatggaggtgctggggtgtgaggctcaggacctgtattga

SEQ ID NO: 12 - DNA, 4374 bp, synthetic construct
  source 1..4374: mol_type = other DNA
atgcagattgagctgtctacttgtttttttctgtgcctgctgaggttctgcttctctgccaccaggaggtattacctgggggctgtggagctgagctgggattacatgcagtctgatctgggggagctgcctgtggatgccaggttcccccccagggtgcccaagagcttccccttcaacacctctgtggtgtataagaagaccctgtttgtggagttcactgatcatctgtttaacattgccaagcccaggcccccctggatgggcctgctgggcccaactatccaggctgaggtgtatgacactgtggtcatcaccctgaagaatatggccagccatcctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacttatagctacctgtctcatgtggacctggtgaaggacctgaactctggcctgattggggccctgctggtctgtagggaaggcagcctggccaaggagaagacccagaccctgcacaagtttattctgctgtttgctgtgtttgatgaaggcaagagctggcactctgagaccaagaattctctgatgcaggatagggatgctgcctctgccagggcctggcccaagatgcatactgtgaatggctatgtgaacagaagcctgcctggcctgattggctgccataggaagtctgtgtattggcatgtgattgggatgggcactacccctgaagtgcacagcattttcctggagggccacactttcctggtgaggaaccacaggcaggcctctctggagatcagccccattactttcctgactgcccagaccctgctgatggatctgggccagttcctgctgttctgccacatctctagccaccagcatgatggcatggaggcctatgtgaaggtggacagctgccctgaggagccccagctgaggatgaagaataatgaggaggctgaggattatgatgatgacctgactgactctgagatggatgtggtgaggtttgatgatgataatagccccagcttcatccagatcaggtctgtggccaagaagcatcccaagacctgggtgcactatattgctgctgaagaggaggactgggactatgcccctctggtgctggctcctgatgacaggagctataagagccagtatctgaacaatgggccccagaggattgggaggaagtacaagaaggtgaggttcatggcctacactgatgagacctttaagaccagggaggccatccagcatgagtctggcattctggggcccctgctgtatggggaggtgggggacactctgctgatcattttcaagaaccaggccagcaggccctacaatatttacccccatggcatcactgatgtgaggcccctgtacagcaggaggctgcccaagggggtgaagcacctgaaggacttccccatcctgcctggggagatcttcaagtacaagtggactgtgactgtggaggatggccctaccaagtctgaccctaggtgtctgactaggtactacagcagctttgtgaacatggagagagacctggcttctggcctgattggccccctgctgatctgctacaaggagtctgtggatcagaggggcaaccagattatgtctgataagaggaatgtcatcctgttctctgtgtttgatgagaacaggagctggtatctgactgagaacattcagaggttcctgcccaaccctgctggggtgcagctggaggaccctgagttccaggccagcaacatcatgcattctattaatggctatgtgtttgacagcctgcagctgtctgtgtgcctgcatgaggtggcctactggtacatcctgagcattggggcccagactgactttctgtctgtgtttttctctgggtacaccttcaagcacaagatggtctatgaggacaccctgaccctgttccccttttctggggaaactgtgtttatgagcatggagaaccctgggctgtggatcctgggctgccacaactctgactttaggaataggggcatgactgccctgctgaaggtgagcagctgtgacaagaatactggggattactatgaggacagctatgaggatatctctgcctacctgctgagcaagaacaatgccattgagcctaggagcttcagccagaacccccctgtgctgaagaggcaccagagggagatcaccaggaccaccctgcagtctgatcaggaggagattgactatgatgacaccatctctgtggagatgaagaaggaggactttgatatttatgatgaggatgagaaccagagccccaggagcttccagaagaagaccaggcactatttcattgctgctgtggagaggctgtgggactatggcatgagctctagcccccatgtgctgaggaacagggcccagtctggctctgtgccccagttcaagaaggtggtgttccaggaatttactgatggcagctttacccagcccctgtacagaggggagctgaatgagcacctgggcctgctgggcccctacatcagggctgaggtggaggataatatcatggtgacctttaggaaccaggcctctaggccctattctttttacagcagcctgatcagctatgaggaggaccagaggcagggggctgagcctaggaagaactttgtgaagcccaatgagaccaagacctacttttggaaagtgcagcaccacatggcccccactaaggatgagtttgattgcaaggcctgggcctatttctctgatgtggacctggagaaggatgtgcactctggcctgattggccccctgctggtgtgccacaccaacactctgaaccctgcccatggcaggcaggtgactgtgcaggagtttgccctgttctttaccatctttgatgagactaagagctggtatttcactgagaacatggagaggaactgcagagccccttgcaacatccagatggaggaccctaccttcaaggagaactataggttccatgccatcaatgggtacatcatggataccctgcctggcctggtgatggctcaggaccagaggatcaggtggtacctgctgagcatggggagcaatgagaacattcatagcatccacttctctgggcatgtgttcactgtgaggaagaaggaggagtataagatggccctgtacaacctgtaccctggggtgtttgagactgtggagatgctgcccagcaaggctggcatctggagggtggagtgcctgattggggagcacctgcatgctggcatgagcactctgttcctggtgtacagcaacaagtgccagacccccctgggcatggcctctggccacatcagggacttccagattactgcctctgggcagtatgggcagtgggcccccaagctggccaggctgcactactctgggtctatcaatgcttggagcaccaaggagcctttcagctggatcaaggtggatctgctggcccccatgatcattcatgggatcaagacccagggggccaggcagaagttcagcagcctgtatatttctcagttcatcatcatgtattctctggatggcaaaaagtggcagacctatagagggaacagcactgggaccctgatggtgttttttggcaatgtggatagctctggcatcaagcacaatatcttcaacccccccattattgccaggtacatcaggctgcaccccacccactactctatcaggagcaccctgaggatggagctgatgggctgtgatctgaacagctgctctatgcctctggggatggaaagcaaggccatctctgatgcccagatcactgccagcagctatttcaccaatatgtttgccacttggagccctagcaaggctaggctgcatctgcagggcaggtctaatgcctggaggccccaggtgaacaaccccaaggagtggctgcaggtggacttccagaagactatgaaagtgactggggtgaccacccagggggtgaaaagcctgctgaccagcatgtatgtgaaggagttcctgattagcagcagccaggatggccaccagtggaccctgttcttccagaatgggaaggtgaaggtgtttcagggcaatcaggatagcttcaccccagtggtgaacagcctggacccccccctgctgaccaggtacctgaggatccacccccagagctgggtgcaccagattgccctgaggatggaggtgctgggctgtgaggcccaggatctgtactga

SEQ ID NO: 13 - DNA, 4374 bp, synthetic construct
  source 1..4374: mol_type = other DNA
atgcagattgagctgagcacctgctttttcctgtgcctgctgaggttctgcttctctgctaccaggaggtactacctgggggctgtggagctgtcttgggattacatgcagtctgacctgggggagctgcctgtggatgccaggtttccccccagggtgcccaagtctttcccctttaacacctctgtggtgtataagaagactctgtttgtggagttcactgatcacctgttcaatattgccaagcccaggcccccttggatgggcctgctgggccccactatccaggctgaggtgtatgacactgtggtcatcaccctgaagaacatggccagccaccctgtgagcctgcatgctgtgggggtgagctactggaaggcctctgagggggctgagtatgatgaccagaccagccagagggagaaggaggatgacaaggtgttcccaggggggtctcacacttatgtgtggcaggtgctgaaggagaatgggcccatggcctctgaccctctgtgcctgacttatagctacctgtctcatgtggatctggtgaaggacctgaactctggcctgattggggccctgctggtgtgcagggaggggagcctggccaaggagaagacccagaccctgcacaagttcatcctgctgtttgctgtgtttgatgaggggaagagctggcactctgagaccaagaatagcctgatgcaggacagggatgctgcttctgctagggcctggcctaagatgcacactgtgaatggctatgtgaacaggagcctgcctggcctgattgggtgtcacaggaagtctgtgtactggcatgtgattggcatggggactactccagaagtgcacagcatcttcctggaggggcacaccttcctggtgaggaatcacaggcaggccagcctggagatttctcccatcactttcctgactgcccagaccctgctgatggatctggggcagttcctgctgttctgccacatcagcagccatcagcatgatgggatggaggcctatgtgaaggtggacagctgccctgaggagcctcagctgaggatgaagaacaatgaggaggctgaggactatgatgatgatctgactgactctgagatggatgtggtgaggtttgatgatgacaactctcccagcttcatccagatcaggtctgtggccaagaagcaccccaagacctgggtgcactacattgctgctgaggaggaggattgggattatgctcccctggtgctggctcctgatgataggagctacaagagccagtatctgaataatgggccccagaggattggcaggaagtataagaaggtgaggttcatggcctacactgatgagacctttaagaccagggaggctattcagcatgagtctggcatcctgggccccctgctgtatggggaggtgggggacaccctgctgatcattttcaagaaccaggccagcaggccctataacatctatccccatgggatcactgatgtgaggcccctgtactctaggaggctgcccaagggggtcaagcacctgaaggacttccccatcctgcctggggagatcttcaagtacaagtggactgtgactgtggaggatggccccactaagtctgaccccaggtgcctgactaggtactacagcagctttgtgaacatggagagagatctggcctctggcctgattggccccctgctgatctgctacaaagagtctgtggatcagaggggcaaccagatcatgtctgacaagaggaatgtgatcctgttctctgtgtttgatgagaacagaagctggtacctgactgagaacattcagaggtttctgcccaaccctgctggggtccagctggaggaccctgagtttcaggccagcaacatcatgcacagcatcaatgggtatgtgtttgacagcctgcagctgtctgtgtgcctgcatgaggtggcctactggtatatcctgagcattggggcccagactgatttcctgtctgtgttcttctctggctacactttcaagcacaagatggtgtatgaggataccctgaccctgttccctttctctggggaaactgtgttcatgagcatggagaaccctgggctgtggatcctggggtgccacaattctgatttcaggaacagaggcatgactgctctgctgaaggtgtctagctgtgacaagaacactggggactactatgaggacagctatgaggacatctctgcctacctgctgagcaagaacaatgctattgaacccaggtctttcagccagaacccccctgtgctgaagaggcaccagagggagatcactaggaccaccctgcagtctgatcaggaggagattgactatgatgacaccatctctgtggagatgaagaaggaggactttgacatctatgatgaggatgagaatcagtctcccaggagcttccagaagaagactaggcattacttcattgctgctgtggagaggctgtgggactatggcatgagctctagccctcatgtgctgaggaacagggcccagtctggctctgtgccccagttcaagaaggtggtgtttcaggagttcactgatggcagcttcacccagcccctgtacaggggggagctgaatgagcatctgggcctgctgggcccctacatcagggctgaggtggaggacaacatcatggtgaccttcagaaatcaggctagcaggccctacagcttctacagcagcctgatctcttatgaggaggaccagaggcagggggctgagcccaggaagaactttgtgaagcccaatgagaccaagacctatttctggaaggtgcagcaccacatggcccccaccaaggatgagtttgattgcaaggcctgggcctacttctctgatgtggacctggagaaggatgtgcattctgggctgattggccctctgctggtgtgccacaccaacaccctgaatcctgcccatggcaggcaggtgactgtgcaggagtttgccctgttctttactatctttgatgagaccaagtcttggtattttactgagaacatggagaggaactgcagggccccctgcaacatccagatggaggaccccaccttcaaggagaactacagattccatgccatcaatggctacattatggacactctgcctggcctggtgatggcccaggaccagaggatcaggtggtacctgctgtctatgggcagcaatgagaacattcactctatccacttctctgggcatgtgttcactgtgaggaagaaggaggagtacaagatggccctgtacaacctgtaccctggggtgtttgagactgtggagatgctgcctagcaaggctgggatctggagggtggagtgcctgattggggagcacctgcatgctggcatgtctaccctgttcctggtgtacagcaacaagtgccagacccccctgggcatggcctctggccacatcagagattttcagatcactgcctctggccagtatggccagtgggctcctaagctggccaggctgcactactctggcagcatcaatgcctggagcaccaaggagccctttagctggatcaaggtggacctgctggcccccatgatcatccatggcatcaagactcagggggccaggcagaagttctctagcctgtacattagccagttcatcatcatgtatagcctggatggcaagaagtggcagacctacaggggcaacagcactgggaccctgatggtgttctttgggaatgtggacagctctgggatcaagcacaatatcttcaacccccccattattgccaggtatattaggctgcaccccactcactacagcattaggagcaccctgaggatggagctgatgggctgtgatctgaacagctgcagcatgcccctgggcatggagtctaaggccatctctgatgcccagatcactgccagctcttacttcaccaacatgtttgccacttggagccccagcaaggccaggctgcacctgcagggcaggagcaatgcctggaggccccaggtgaacaaccccaaggagtggctgcaggtggatttccagaagactatgaaggtgactggggtgaccactcagggggtgaagagcctgctgactagcatgtatgtgaaggagttcctgatcagctctagccaggatggccaccagtggaccctgttctttcagaatggcaaggtgaaggtgttccagggcaaccaggactctttcacccctgtggtgaattctctggaccctcccctgctgactaggtatctgaggattcatccccagagctgggtgcatcagattgccctgaggatggaggtgctgggctgtgaggcccaggacctgtattga

SEQ ID NO: 14 - DNA, 4374 bp, synthetic construct
  source 1..4374: mol_type = other DNA
atgcagattgagctgagcacctgcttcttcctgtgcctgctgaggttttgcttttctgccactaggaggtactacctgggggctgtggagctgtcttgggattacatgcagtctgacctgggggagctgccagtggatgccaggttccccccaagggtgcccaagtcttttcccttcaatacctctgtggtgtacaagaagaccctgtttgtggagtttactgatcatctgtttaacattgccaagcccaggcccccctggatggggctgctgggccccaccatccaggctgaggtgtatgatactgtggtgattaccctgaagaatatggccagccatcctgtgtctctgcatgctgtgggggtgtcttattggaaggcctctgagggggctgagtatgatgatcagaccagccagagggagaaggaggatgataaggtgttccctgggggctctcacacctatgtgtggcaggtgctgaaggagaatgggcctatggcctctgacccactgtgcctgacttacagctatctgagccatgtggacctggtgaaggacctgaactctgggctgattggggccctgctggtgtgcagggagggcagcctggccaaggagaagactcagaccctgcacaagttcatcctgctgtttgctgtgtttgatgagggcaagtcttggcactctgagaccaagaacagcctgatgcaggatagggatgctgcctctgccagggcctggcccaagatgcacactgtgaatggctatgtgaacaggtctctgcctggcctgattggctgccacaggaagtctgtgtactggcatgtgattggcatgggcaccacccctgaggtgcatagcattttcctggagggccacaccttcctggtgaggaaccacaggcaggctagcctggagatcagccccatcactttcctgactgcccagaccctgctgatggacctgggccagttcctgctgttctgccacatctctagccaccagcatgatggcatggaggcctatgtgaaggtggactcttgtcctgaggagccccagctgaggatgaagaacaatgaggaggctgaggattatgatgatgatctgactgattctgagatggatgtggtgaggtttgatgatgacaacagcccctctttcatccagatcaggtctgtggccaagaagcaccccaagacctgggtgcactacattgctgctgaggaggaggattgggattatgcccccctggtgctggcccctgatgacaggagctataagtctcagtacctgaacaatggcccccagagaattggcaggaagtacaagaaggtgaggttcatggcctatactgatgagaccttcaaaaccagggaggccattcagcatgagtctggcatcctggggcccctgctgtatggggaggtgggggacaccctgctgatcatcttcaagaaccaggctagcaggccttacaacatctacccccatgggatcactgatgtgaggcccctgtacagcaggaggctgcctaagggggtgaagcacctgaaggactttcccattctgcctggggagatcttcaagtataagtggactgtgactgtggaggatgggcccaccaagtctgaccccaggtgcctgactaggtactactctagctttgtgaacatggagagggacctggcctctgggctgattggccccctgctgatctgttacaaggagtctgtggaccagaggggcaaccagatcatgtctgataagaggaatgtgatcctgttctctgtgtttgatgagaacaggagctggtacctgactgagaacatccagagattcctgcccaaccctgctggggtgcagctggaggatcctgagttccaggccagcaacatcatgcattctatcaatgggtatgtgtttgatagcctgcagctgtctgtgtgtctgcatgaggtggcctactggtacattctgagcattggggcccagactgacttcctgtctgtgttcttctctggctacactttcaaacacaagatggtgtatgaggacaccctgaccctgttccccttctctggggagactgtgtttatgagcatggagaaccctgggctgtggattctgggctgccacaactctgacttcagaaacaggggcatgactgccctgctgaaggtgtcttcttgtgataagaacactggggactattatgaagacagctatgaggacatctctgcctacctgctgagcaagaataatgctattgagcccaggtctttctctcagaacccccctgtgctgaagaggcaccagagggagatcaccaggaccaccctgcagtctgatcaggaggagattgactatgatgacactatttctgtggagatgaagaaggaagactttgatatctatgatgaggatgagaaccagagccctaggagcttccagaagaagactaggcattacttcattgctgctgtggagaggctgtgggactatggcatgagcagcagcccccatgtgctgaggaatagggctcagtctggctctgtgcctcagttcaagaaggtggtgttccaggaattcactgatggcagcttcactcagcccctgtacaggggggagctgaatgagcacctggggctgctgggcccttacatcagggctgaggtggaggacaatatcatggtgacctttaggaaccaggcctctaggccttacagcttctactctagcctgatctcttatgaagaggaccagaggcagggggctgagcccaggaagaactttgtgaagcccaatgagactaagacttacttctggaaggtgcagcaccacatggctcccaccaaggatgagtttgactgcaaggcttgggcctacttctctgatgtggacctggagaaggatgtgcactctgggctgattgggcccctgctggtgtgccacactaacactctgaatcctgcccatggcagacaggtgactgtgcaggagtttgccctgttttttaccatctttgatgagactaagtcttggtacttcactgagaacatggagaggaactgcagggccccctgcaacatccagatggaggatcccaccttcaaggagaactacaggtttcatgccatcaatggctacatcatggacaccctgcctggcctggtgatggctcaggaccagaggattaggtggtatctgctgagcatgggcagcaatgagaatatccactctatccacttctctgggcatgtgttcactgtgaggaagaaggaggagtacaagatggccctgtataacctgtatcctggggtgtttgagactgtggagatgctgcccagcaaggctggcatctggagagtggagtgcctgattggggagcacctgcatgctggcatgagcactctgtttctggtgtatagcaacaagtgtcagacccctctgggcatggcctctgggcacattagggactttcagatcactgcttctggccagtatgggcagtgggctcccaagctggccaggctgcactattctggcagcattaatgcctggagcaccaaggagcctttcagctggatcaaggtggacctgctggcccccatgatcatccatgggatcaagacccagggggctaggcagaagttcagcagcctgtacatcagccagtttatcatcatgtattctctggatggcaagaagtggcagacctacaggggcaattctactggcactctgatggtgttctttgggaatgtggatagctctgggatcaagcataatatcttcaatccccccattattgctaggtatatcaggctgcaccccacccactatagcatcaggagcaccctgaggatggagctgatggggtgtgacctgaacagctgcagcatgcccctgggcatggagagcaaggctatttctgatgcccagatcactgccagcagctactttactaatatgtttgccacctggagccccagcaaggccagactgcacctgcagggcaggtctaatgcctggaggcctcaggtgaataaccccaaggagtggctgcaggtggacttccagaaaaccatgaaggtgactggggtgactacccagggggtgaagtctctgctgaccagcatgtatgtgaaggagttcctgatctcttctagccaggatggccaccagtggaccctgttctttcagaatgggaaggtgaaggtcttccagggcaaccaggatagcttcacccctgtggtgaatagcctggatcctcctctgctgaccaggtatctgaggatccacccccagagctgggtgcatcagattgccctgaggatggaggtgctgggctgtgaggctcaggacctgtactga

SEQ ID NO: 15 - DNA, 4374 bp, synthetic construct
  source 1..4374: mol_type = other DNA
atgcagattgagctgagcacctgtttcttcctgtgcctgctgaggttctgtttctctgccactaggaggtactacctgggggctgtggagctgagctgggactatatgcagtctgacctgggggagctgcctgtggatgccaggttcccccccagggtgcctaagagcttccccttcaatacttctgtggtgtacaagaagactctgtttgtggagtttactgaccacctgttcaacattgctaagcccaggcctccctggatggggctgctgggccccaccatccaggctgaggtgtatgatactgtggtgattaccctgaagaacatggcctctcatccagtgagcctgcatgctgtgggggtgagctactggaaggcctctgaaggggctgagtatgatgaccagaccagccagagggagaaggaggatgacaaggtgttccctgggggcagccacacctatgtgtggcaggtgctgaaggagaatggcccaatggcctctgaccccctgtgcctgacttatagctacctgagccatgtggatctggtgaaggacctgaattctggcctgattggggccctgctggtgtgcagagagggctctctggctaaggagaagacccagactctgcacaagttcatcctgctgtttgctgtgtttgatgagggcaagagctggcactctgagactaagaatagcctgatgcaggacagggatgctgcttctgccagggcctggcccaagatgcatactgtgaatggctatgtgaacaggagcctgcctggcctgattggctgtcacaggaaatctgtctactggcatgtgattgggatgggcactacccctgaggtgcactctatcttcctggagggccataccttcctggtgaggaaccacaggcaggccagcctggagatctctcccattaccttcctgactgcccagaccctgctgatggatctgggccagttcctgctgttctgccacatcagcagccaccagcatgatgggatggaggcttatgtgaaggtggatagctgccctgaggagccccagctgaggatgaagaacaatgaggaggctgaggactatgatgatgacctgactgactctgagatggatgtggtgaggtttgatgatgacaactctcccagctttattcagatcaggtctgtggctaagaagcaccccaagacttgggtgcactacattgctgctgaggaggaggactgggactatgcccctctggtgctggctcctgatgacaggtcttacaagtctcagtacctgaataatggccctcagaggattggcaggaagtacaagaaggtgaggttcatggcctacactgatgagaccttcaagaccagggaggccatccagcatgagtctggcatcctgggccccctgctgtatggggaggtgggggataccctgctgatcatcttcaagaatcaggccagcaggccctacaacatctacccccatggcatcactgatgtgaggccactgtacagcaggaggctgcccaagggggtgaagcatctgaaggacttccccattctgcctggggagatcttcaagtacaaatggactgtgactgtggaggatggccctaccaagtctgaccccaggtgtctgaccaggtactacagcagctttgtgaatatggagagggacctggcctctggcctgattggccccctgctgatctgctacaaggagtctgtggaccagaggggcaatcagatcatgtctgataagaggaatgtgattctgttctctgtgtttgatgagaacaggagctggtacctgactgagaacatccagaggttcctgcccaatcctgctggggtgcagctggaggaccctgagttccaggccagcaatatcatgcacagcatcaatggctatgtctttgacagcctgcagctgtctgtgtgcctgcatgaggtggcttactggtatattctgagcattggggcccagactgatttcctgtctgtgttcttttctggctatacctttaagcacaagatggtgtatgaggacaccctgaccctgttccccttctctggggagactgtgttcatgtctatggagaaccctgggctgtggatcctgggctgccacaactctgacttcaggaacagggggatgactgccctgctgaaggtgtctagctgtgataagaacactggggactattatgaggacagctatgaggacatctctgcttacctgctgagcaagaacaatgccattgagcccaggtctttcagccagaatccccctgtgctgaagaggcatcagagggagatcaccaggaccaccctgcagtctgatcaggaggagattgattatgatgacactatctctgtggaaatgaagaaggaggactttgacatctatgatgaggatgagaaccagagccccaggagcttccagaagaagaccaggcactacttcattgctgctgtggagaggctgtgggattatggcatgagcagctctccccatgtgctgaggaacagagcccagtctggctctgtgcctcagttcaagaaggtggtcttccaggagttcactgatggctctttcacccagcccctgtacaggggggagctgaatgagcacctgggcctgctggggccctacattagggctgaggtggaggataacatcatggtgactttcagaaaccaggccagcaggccttacagcttttactcttctctgattagctatgaggaggatcagaggcagggggctgagcctaggaagaactttgtgaagcccaatgagaccaagacctatttctggaaggtgcagcaccacatggctcccactaaggatgagtttgactgcaaggcttgggcctacttctctgatgtggacctggagaaggatgtgcactctggcctgattgggcccctgctggtgtgccacaccaacaccctgaaccctgcccatggcaggcaggtgactgtgcaggagtttgccctgttcttcaccatctttgatgagactaagagctggtacttcactgagaacatggagaggaactgcagggccccctgcaacatccagatggaggaccccaccttcaaggagaattacaggttccatgccatcaatggctacattatggacaccctgcctggcctggtgatggcccaggatcagaggatcaggtggtatctgctgagcatgggctctaatgagaacatccacagcatccacttctctggccatgtgtttactgtgaggaagaaggaggaatacaagatggctctgtataacctgtaccctggggtgtttgagactgtggagatgctgcccagcaaggctgggatctggagggtggagtgcctgattggggagcacctgcatgctgggatgagcaccctgttcctggtgtatagcaataagtgccagacccccctgggcatggcttctggccacatcagggatttccagatcactgcttctggccagtatggccagtgggctcccaagctggctaggctgcattactctgggtctatcaatgcctggagcactaaggagcccttcagctggatcaaggtggacctgctggcccccatgatcattcatggcatcaagacccagggggctaggcagaagttcagcagcctgtacatcagccagttcatcattatgtacagcctggatggcaagaagtggcagacttacaggggcaatagcactgggactctgatggtgttctttggcaatgtggactcttctggcatcaagcacaacatcttcaaccctcccatcattgccaggtacattaggctgcaccctacccactactctatcaggagcaccctgaggatggagctgatggggtgtgatctgaactcttgcagcatgcctctgggcatggaaagcaaagccatctctgatgcccagatcactgcctctagctatttcaccaatatgtttgccacctggagccctagcaaggccaggctgcacctgcagggcagatctaatgcctggaggccccaggtgaacaatcccaaggagtggctgcaggtggacttccagaagaccatgaaggtgactggggtgaccactcagggggtgaagagcctgctgactagcatgtatgtgaaggagttcctgatctcttctagccaggatggccaccagtggaccctgttcttccagaatggcaaggtgaaagtgttccagggcaaccaggatagcttcactcctgtggtgaactctctggaccctcccctgctgactaggtacctgaggattcatccccagagctgggtgcaccagattgccctgaggatggaggtgctgggctgtgaggcccaggatctgtactga

SEQ ID NO: 16 - DNA, 4374 bp, synthetic construct
  source 1..4374: mol_type = other DNA
atgcagattgagctgagcacctgcttcttcctgtgcctgctgaggttctgcttctctgccaccaggaggtactacctgggggctgtggagctgtcttgggactatatgcagtctgacctgggggagctgccagtggatgccaggttcccccccagggtgcccaagagctttcctttcaacacttctgtggtgtacaagaagaccctgtttgtggagttcactgaccacctgttcaatattgctaagcccaggccaccctggatgggcctgctgggccctaccattcaggctgaggtgtatgacactgtggtgattactctgaagaatatggccagccaccctgtgagcctgcatgctgtgggggtgtcttactggaaggcctctgagggggctgagtatgatgatcagacttctcagagggagaaggaggatgataaggtgttccctgggggctctcacacttatgtgtggcaggtgctgaaggagaatggccccatggcttctgatccactgtgcctgacctactcttacctgagccatgtggacctggtgaaggacctgaactctggcctgattggggccctgctggtgtgcagggagggcagcctggccaaggagaagacccagaccctgcataagttcatcctgctgtttgctgtgtttgatgaggggaagagctggcactctgagaccaagaattctctgatgcaggacagggatgctgcctctgccagggcctggcctaagatgcacactgtgaatggctatgtgaacaggtctctgcctggcctgattggctgccacaggaagtctgtgtactggcatgtgattggcatgggcactacccctgaggtgcacagcattttcctggagggccacaccttcctggtcaggaaccataggcaggcctctctggagatcagccccatcactttcctgactgcccagaccctgctgatggacctgggccagttcctgctgttctgccacattagcagccaccagcatgatggcatggaggcctatgtgaaggtggactcttgccctgaggagccccagctgaggatgaagaacaatgaggaagctgaggattatgatgatgacctgactgactctgagatggatgtggtgaggtttgatgatgacaacagccccagcttcatccagatcaggtctgtggccaagaagcaccccaagacctgggtgcactacattgctgctgaggaggaggattgggactatgctcccctggtgctggctcctgatgataggagctacaagtctcagtacctgaataatggcccccagaggattggcaggaagtacaagaaggtgaggttcatggcctacactgatgagaccttcaagaccagagaggctatccagcatgagtctgggatcctggggcccctgctgtatggggaggtgggggacaccctgctgatcatcttcaagaaccaggccagcagaccctacaacatctacccccatgggatcactgatgtgaggcccctgtacagcaggaggctgcctaagggggtgaagcacctgaaggacttccccatcctgcctggggagatcttcaagtataagtggactgtgactgtggaggatgggcccaccaagtctgaccctaggtgcctgactaggtactactctagctttgtgaacatggagagggacctggcctctggcctgattggccccctgctgatttgctacaaggagtctgtggatcagaggggcaatcagatcatgtctgacaagaggaatgtgatcctgttctctgtgtttgatgagaataggtcttggtacctgactgagaacatccagaggttcctgcctaatcctgctggggtgcagctggaggaccctgagtttcaggccagcaacatcatgcacagcatcaatggctatgtgtttgactctctgcagctgtctgtgtgcctgcatgaggtggcttactggtatatcctgagcattggggctcagactgacttcctgtctgtgttcttttctggctacacttttaagcacaagatggtgtatgaggacaccctgaccctgttccccttttctggggagactgtgttcatgtctatggagaaccctgggctgtggattctgggctgtcacaactctgacttcagaaacaggggcatgactgccctgctgaaggtgtctagctgtgacaagaatactggggactactatgaggacagctatgaggacatttctgcctatctgctgagcaagaacaatgccattgagcccaggagcttttctcagaatccccctgtgctgaagaggcaccagagagagatcaccaggaccactctgcagtctgatcaggaggagattgattatgatgacactatctctgtggagatgaagaaagaggactttgatatctatgatgaggatgagaatcagtctcccaggagcttccagaagaagactagacactacttcattgctgctgtggagaggctgtgggactatggcatgagctctagccctcatgtgctgaggaacagggcccagtctgggtctgtgccccagttcaagaaggtggtgttccaggagttcactgatggcagctttacccagcccctgtataggggggagctgaatgagcatctgggcctgctgggcccctatattagggctgaagtggaggacaacatcatggtgacctttaggaaccaggccagcaggccctacagcttttacagcagcctgattagctatgaggaggatcagagacagggggctgagcccaggaagaactttgtgaagcccaatgagaccaagacctacttctggaaggtgcagcaccacatggcccctaccaaggatgagtttgactgcaaggcctgggcttacttctctgatgtggacctggagaaagatgtgcactctggcctgattgggcccctgctggtgtgccacaccaacaccctgaaccctgcccatgggaggcaggtgactgtgcaggagtttgccctgtttttcaccatctttgatgagaccaagagctggtacttcactgagaacatggagaggaactgcagggccccctgtaacatccagatggaggatcctactttcaaggagaactacaggttccatgccattaatgggtacatcatggacaccctgcctgggctggtgatggcccaggatcagaggattaggtggtatctgctgtctatgggctctaatgagaacatccactctatccacttctctggccatgtgttcactgtgaggaagaaggaggagtacaagatggccctgtacaacctgtaccctggggtgtttgaaactgtggagatgctgccctctaaagctgggatctggagggtggagtgcctgattggggagcacctgcatgctggcatgagcaccctgttcctggtgtacagcaataagtgccagactcccctgggcatggcttctgggcacatcagggatttccagatcactgcctctggccagtatggccagtgggcccccaagctggctaggctgcactactctggcagcatcaatgcctggagcaccaaggagcccttctcttggattaaggtggacctgctggctcccatgatcattcatggcatcaagacccagggggccaggcagaagttttctagcctgtatattagccagttcatcatcatgtatagcctggatgggaagaagtggcagacctacagggggaatagcactggcaccctgatggtgttttttggcaatgtggattcttctggcatcaagcataacatcttcaatccccctatcattgccaggtacattaggctgcatcccacccattactctatcaggagcaccctgaggatggagctgatggggtgtgatctgaacagctgtagcatgcccctgggcatggagtccaaggctatctctgatgcccagatcactgccagcagctacttcaccaacatgtttgccacctggagccccagcaaggccaggctgcacctgcagggcaggtctaatgcctggaggccccaggtgaacaatcccaaggagtggctgcaggtggacttccagaagactatgaaggtgactggggtgaccactcagggggtgaagagcctgctgaccagcatgtatgtgaaggagttcctgatctcttctagccaggatgggcatcagtggaccctgttttttcagaatggcaaagtgaaggtgtttcaggggaatcaggacagctttacccctgtggtgaacagcctggatcctcctctgctgactagatacctgaggatccacccccagagctgggtccaccagattgctctgaggatggaggtgctggggtgtgaggctcaggacctgtactga

SEQ ID NO: 17 - DNA, 4374 bp, synthetic construct
  source 1..4374: mol_type = other DNA
atgcagattgagctgagcacctgcttctttctgtgcctgctgaggttctgcttctctgccaccaggaggtactacctgggggctgtggaactgagctgggactatatgcagtctgacctgggggagctgcctgtggatgccaggttcccccccagggtgcccaagtctttcccctttaacacttctgtggtgtacaagaagaccctgtttgtggagtttactgaccacctgttcaatattgccaagcccaggcccccctggatgggcctgctgggcccaaccatccaggctgaggtgtatgatactgtggtgatcaccctgaagaacatggccagccaccctgtgagcctgcatgctgtgggggtgagctattggaaggcttctgagggggctgagtatgatgaccagactagccagagggagaaggaggatgacaaggtgttccctggggggtctcatacctatgtgtggcaggtgctgaaggagaatggccccatggcctctgaccccctgtgcctgacctattcttacctgagccatgtggacctggtcaaggacctgaactctggcctgattggggctctgctggtgtgcagggagggcagcctggccaaggagaagactcagactctgcataagttcatcctgctgtttgctgtgtttgatgagggcaagagctggcactctgagaccaagaactctctgatgcaggatagggatgctgcctctgccagggcctggcccaagatgcacactgtgaatggctatgtgaataggtctctgcctggcctgattggctgccataggaagtctgtgtactggcatgtgattggcatgggcactacccctgaggtgcactctatcttcctggaggggcacaccttcctggtgaggaaccacaggcaggccagcctggagatctctcccatcaccttcctgactgcccagactctgctgatggacctgggccagttcctgctgttctgccatatcagcagccaccagcatgatggcatggaggcctatgtgaaggtggacagctgcccagaggaaccccagctgaggatgaagaacaatgaggaggctgaggactatgatgatgacctgactgactctgagatggatgtggtgaggtttgatgatgacaacagccccagctttattcagatcaggtctgtggccaagaagcaccccaagacctgggtgcactacattgctgctgaggaggaggactgggattatgcccccctggtgctggcccctgatgacaggtcttacaagtctcagtacctgaacaatggcccccagaggattgggaggaagtacaagaaggtgaggttcatggcctacactgatgagaccttcaagaccagggaggccatccagcatgagtctggcatcctggggcccctgctgtatggggaggtgggggataccctgctgattatcttcaagaaccaggctagcaggccctataacatctacccccatggcattactgatgtgaggcccctgtactctaggagactgcccaagggggtgaagcacctgaaagacttccccatcctgcctggggagatcttcaagtataagtggactgtgactgtggaggatggccccactaagtctgaccccaggtgcctgaccaggtattacagcagctttgtgaatatggagagggatctggcttctggcctgattgggcctctgctgatttgctacaaggagtctgtggatcagagggggaaccagattatgtctgacaagaggaatgtgattctgttctctgtgtttgatgagaacaggagctggtacctgactgagaatatccagaggttcctgcctaatcctgctggggtgcagctggaggaccctgagttccaggctagcaacattatgcacagcatcaatggctatgtgtttgacagcctgcagctgtctgtgtgcctgcatgaggtggcttactggtacattctgtctattggggcccagactgacttcctgtctgtgttcttctctggctacaccttcaagcacaagatggtgtatgaggacactctgaccctgttccccttctctggggagactgtgttcatgagcatggagaatcctgggctgtggattctggggtgccacaactctgatttcaggaacaggggcatgactgccctgctgaaggtgagcagctgtgacaagaacactggggattattatgaggacagctatgaggacatttctgcctacctgctgagcaagaacaatgccattgagcctaggagcttcagccagaatccccctgtgctgaagagacaccagagggagatcactaggaccactctgcagtctgatcaggaggagattgactatgatgacaccatttctgtggagatgaagaaggaggactttgatatttatgatgaggatgagaaccagagccccagaagcttccagaagaagaccaggcactacttcattgctgctgtggagaggctgtgggattatggcatgtcttctagcccccatgtgctgaggaacagggctcagtctggctctgtgcctcagttcaagaaggtggtgttccaggagttcactgatgggagcttcacccagcctctgtacaggggggagctgaatgaacatctgggcctgctggggccctacatcagggctgaggtggaggataatatcatggtgactttcaggaatcaggcctctaggccctacagcttctactctagcctgatcagctatgaggaggaccagaggcagggggctgagcctaggaagaattttgtgaaacccaatgagaccaagacctacttttggaaggtgcagcaccacatggcccctaccaaggatgagtttgactgtaaggcctgggcctacttctctgatgtggacctggagaaggatgtgcattctgggctgattggccccctgctggtgtgccacaccaacaccctgaaccctgcccatggcaggcaggtgactgtgcaggagtttgccctgttcttcaccatctttgatgagactaagagctggtatttcactgagaacatggagaggaactgtagggctccctgcaacatccagatggaggatccaactttcaaggagaactacaggttccatgccatcaatggctacatcatggacaccctgcctggcctggtgatggcccaggaccagaggattaggtggtacctgctgagcatgggctctaatgagaacatccactctatccacttctctggccatgtgtttactgtgaggaagaaggaggagtacaagatggctctgtacaacctgtaccctggggtgtttgagactgtggagatgctgcctagcaaggctggcatttggagagtggagtgtctgattggggagcacctgcatgctgggatgtctaccctgttcctggtgtactctaacaagtgccagacccccctggggatggcttctgggcacatcagagattttcagattactgcttctgggcagtatggccagtgggctcccaagctggccagactgcattactctggctctattaatgcttggagcaccaaggagcctttcagctggatcaaggtggacctgctggctcccatgatcatccatggcattaagactcagggggctaggcagaagttcagcagcctgtatatttctcagtttattatcatgtattctctggatggcaagaagtggcagacttacaggggcaacagcactggcaccctgatggtgttctttggcaatgtggacagctctgggatcaagcataacatcttcaacccccccattattgccaggtacatcaggctgcaccccacccactattctatcaggagcactctgaggatggagctgatggggtgtgacctgaacagctgctctatgcccctgggcatggagagcaaggccatctctgatgcccagatcactgccagctcttatttcaccaacatgtttgccacctggagccccagcaaggccaggctgcacctgcagggcagaagcaatgcctggaggccccaggtgaacaatcctaaggagtggctgcaggtggacttccagaagactatgaaggtgactggggtgactacccagggggtgaagagcctgctgaccagcatgtatgtgaaggagttcctgattagcagcagccaggatgggcatcagtggaccctgttcttccagaatgggaaggtgaaggtgttccagggcaatcaggacagcttcacccctgtggtgaacagcctggacccccccctgctgaccaggtacctgaggatccatccccagagctgggtgcaccagattgctctgagaatggaggtgctgggctgtgaggcccaggacctgtattga

SEQ ID NO: 18 - DNA, 4374 bp, synthetic construct
  source 1..4374: mol_type = other DNA
atgcagattgagctgtctacctgtttttttctgtgcctgctgaggttctgcttctctgctaccaggaggtattatctgggggctgtggagctgagctgggactacatgcagtctgacctgggggagctgcctgtggatgccaggtttcctcccagggtgcctaagagcttccccttcaacacctctgtggtgtacaagaagactctgtttgtggagttcactgaccacctgttcaacattgccaagcccaggcccccctggatggggctgctgggccccactatccaggctgaggtgtatgatactgtggtgattaccctgaagaacatggcctctcaccctgtgtctctgcatgctgtgggggtgagctactggaaggcttctgagggggctgaatatgatgatcagacctctcagagggagaaggaggatgacaaggtgtttcctgggggcagccacacctatgtgtggcaggtgctgaaggagaatgggcccatggcctctgatcccctgtgcctgacctacagctacctgagccatgtggacctggtgaaggacctgaactctggcctgattggggccctgctggtgtgcagggagggcagcctggccaaggaaaagacccagaccctgcataagttcatcctgctgtttgctgtgtttgatgagggcaagtcttggcactctgagaccaagaacagcctgatgcaggacagggatgctgcctctgctagggcctggcccaagatgcacactgtgaatgggtatgtgaacagatctctgcctggcctgattggctgccacaggaagtctgtgtactggcatgtgattggcatggggaccacccctgaggtgcatagcatcttcctggaggggcacaccttcctggtgagaaatcataggcaggccagcctggagattagccccatcaccttcctgactgcccagaccctgctgatggacctgggccagttcctgctgttctgccacatttctagccaccagcatgatggcatggaggcctatgtgaaggtggatagctgccctgaagagccccagctgaggatgaagaacaatgaggaggctgaggattatgatgatgatctgactgactctgagatggatgtggtgaggtttgatgatgacaacagccccagcttcatccagatcaggtctgtggccaagaagcaccctaagacctgggtgcactacattgctgctgaagaggaggactgggactatgcccccctggtgctggccccagatgacaggtcttacaagagccagtacctgaataatggcccccagaggattgggaggaagtataagaaagtgaggttcatggcttacactgatgagacctttaagactagggaggccattcagcatgagtctgggattctgggccctctgctgtatggggaggtgggggacaccctgctgatcattttcaagaaccaggccagcaggccctataatatttatccccatgggattactgatgtcaggcccctgtacagcaggaggctgcctaagggggtgaagcacctgaaggacttccccattctgcctggggagatcttcaagtataagtggactgtgactgtggaggatggccccaccaagtctgatcctaggtgcctgaccaggtactatagcagctttgtgaacatggagagggacctggcttctggcctgattggccccctgctgatctgctacaaggaatctgtggaccagaggggcaaccagattatgtctgacaagaggaatgtgatcctgttttctgtgtttgatgagaataggagctggtatctgactgagaacatccagaggttcctgcccaatcctgctggggtgcagctggaggaccctgagttccaggcttctaacatcatgcatagcatcaatgggtatgtgtttgactctctgcagctgtctgtgtgcctgcatgaggtggcctattggtacatcctgagcattggggcccagactgacttcctgtctgtgttcttctctggctacaccttcaagcacaagatggtgtatgaggacaccctgaccctgttccctttctctggggagactgtgttcatgagcatggagaaccctggcctgtggattctgggctgccataattctgacttcagaaacaggggcatgactgctctgctgaaggtgagcagctgtgacaagaatactggggactactatgaggactcttatgaggatatttctgcctacctgctgagcaagaacaatgctattgagcccaggagcttcagccagaacccccctgtcctgaagaggcatcagagggagatcactaggaccaccctgcagtctgatcaggaggagattgactatgatgacactatctctgtggaaatgaagaaggaggactttgatatctatgatgaggatgagaaccagagccccaggtctttccagaagaagaccaggcactacttcattgctgctgtggagaggctgtgggactatggcatgtctagcagcccccatgtgctgaggaacagagcccagtctggctctgtgccccagttcaagaaggtggtgtttcaggagttcactgatgggagcttcactcagcccctgtataggggggagctgaatgagcatctgggcctgctggggccctacatcagggctgaggtggaggataacatcatggtgaccttcaggaaccaggccagcaggccctactctttctactcttctctgatcagctatgaggaggatcagaggcagggggctgagcctaggaagaactttgtcaagcctaatgagactaagacctacttttggaaggtgcagcaccacatggctcccactaaggatgagtttgattgcaaggcctgggcctacttctctgatgtggacctggagaaggatgtgcactctggcctgattggccccctgctggtgtgtcacaccaataccctgaaccctgcccatggcaggcaggtcactgtgcaggagtttgccctgtttttcactatctttgatgagactaagtcttggtacttcactgagaacatggaaaggaattgcagggctccctgcaacatccagatggaggaccccaccttcaaggagaactacaggtttcatgccatcaatggctacatcatggacaccctgcctggcctggtgatggctcaggatcagaggattaggtggtatctgctgagcatgggcagcaatgagaacatccacagcatccacttttctggccatgtgttcactgtgaggaagaaggaggagtacaagatggctctgtacaatctgtaccctggggtgtttgagactgtggagatgctgcccagcaaggctgggatctggagggtggagtgcctgattggggaacacctgcatgctggcatgtctaccctgttcctggtgtactctaacaagtgccagactcccctgggcatggcctctgggcacatcagggacttccagatcactgcctctgggcagtatggccagtgggcccctaagctggctaggctgcattactctggcagcatcaatgcctggagcaccaaggagcccttcagctggatcaaggtggacctgctggcccctatgatcatccatggcatcaagacccagggggccagacagaagttctcttctctgtacatctctcagttcatcatcatgtactctctggatggcaagaagtggcagacctacagggggaattctactggcactctgatggtgttctttgggaatgtggatagctctgggatcaagcataatattttcaacccccccattattgctaggtacatcaggctgcacccaacccactactctattaggtctaccctgaggatggagctgatgggctgtgacctgaactcttgtagcatgcccctgggcatggagagcaaggctatctctgatgcccagatcactgccagcagctactttaccaacatgtttgctacttggagccccagcaaggccaggctgcacctgcagggcaggagcaatgcctggaggccccaggtgaacaaccccaaggagtggctgcaggtggattttcagaagaccatgaaggtgactggggtgaccactcagggggtgaaaagcctgctgactagcatgtatgtgaaggagtttctgatcagcagctctcaggatggccatcagtggaccctgttcttccagaatggcaaggtgaaggtgttccagggcaaccaggatagcttcacccctgtggtgaatagcctggacccccccctgctgaccaggtacctgaggatccatccccagagctgggtgcaccagattgccctgaggatggaggtgctgggctgtgaagcccaggacctgtactga

SEQ ID NO: 19 - DNA, 4374 bp, synthetic construct
  source 1..4374: mol_type = other DNA
atgcaaatagagctctccacctgcttctttctgtgccttttgcgattctgctttagtgccaccagaagatactacctgggtgcagtggaactgtcatgggactatatgcaaagtgatctcggtgagctgcctgtggacgcaagatttcctcctagagtgccaaaatcttttccattcaacacctcagtcgtgtacaaaaagactctgtttgtagaattcacggatcaccttttcaacatcgctaagccaaggccaccctggatgggtctgctaggtcctaccatccaggctgaggtttatgatacagtggtcattacacttaagaacatggcttcccatcctgtcagtcttcatgctgttggtgtatcctactggaaagcttctgagggagctgaatatgatgatcagaccagtcaaagggagaaagaagatgataaagtcttccctggtggaagccatacatatgtctggcaggtcctgaaagagaatggtccaatggcctctgacccactgtgccttacctactcatatctttctcatgtggacctggtaaaagacttgaattcaggcctcattggagccctactagtatgtagagaagggagtctggccaaggaaaagacacagaccttgcacaaatttatactactttttgctgtatttgatgaagggaaaagttggcactcagaaacaaagaactccttgatgcaggatagggatgctgcatctgctcgggcctggcctaaaatgcacacagtcaatggttatgtaaacaggtctctgccaggtctgattggatgccacaggaaatcagtctattggcatgtgattggaatgggcaccactcctgaagtgcactcaatattcctcgaaggtcacacatttcttgtgaggaaccatcgccaggcgtccttggaaatctcgccaataactttccttactgctcaaacactcttgatggaccttggacagtttctactgttttgtcatatctcttcccaccaacatgatggcatggaagcttatgtcaaagtagacagctgtccagaggaaccccaactacgaatgaaaaataatgaagaagcggaagactatgatgatgatcttactgattctgaaatggatgtggtcaggtttgatgatgacaactctccttcctttatccaaattcgctcagttgccaagaagcatcctaaaacttgggtacattacattgctgctgaagaggaggactgggactatgctcccttagtcctcgcccccgatgacagaagttataaaagtcaatatttgaacaatggccctcagcggattggtaggaagtacaaaaaagtccgatttatggcatacacagatgaaacctttaagactcgtgaagctattcagcatgaatcaggaatcttgggacctttactttatggggaagttggagacacactgttgattatatttaagaatcaagcaagcagaccatataacatctaccctcacggaatcactgatgtccgtcctttgtattcaaggagattaccaaaaggtgtaaaacatttgaaggattttccaattctgccaggagaaatattcaaatataaatggacagtgactgtagaagatgggccaactaaatcagatcctcggtgcctgacccgctattactctagtttcgttaatatggagagagatctagcttcaggactcattggccctctcctcatctgctacaaagaatctgtagatcaaagaggaaaccagataatgtcagacaagaggaatgtcatcctgttttctgtatttgatgagaaccgaagctggtacctcacagagaatatacaacgctttctccccaatccagctggagtgcagcttgaggatccagagttccaagcctccaacatcatgcacagcatcaatggctatgtttttgatagtttgcagttgtcagtttgtttgcatgaggtggcatactggtacattctaagcattggagcacagactgacttcctttctgtcttcttctctggatataccttcaaacacaaaatggtctatgaagacacactcaccctattcccattctcaggagaaactgtcttcatgtcgatggaaaacccaggtctatggattctggggtgccacaactcagactttcggaacagaggcatgaccgccttactgaaggtttctagttgtgacaagaacactggtgattattacgaggacagttatgaagatatttcagcatacttgctgagtaaaaacaatgccattgaaccaagaagcttctcccaaaacccaccagtcttgaaacgccatcaacgggaaataactcgtactactcttcagtcagatcaagaggaaattgactatgatgataccatatcagttgaaatgaagaaggaagattttgacatttatgatgaggatgaaaatcagagcccccgcagctttcaaaagaaaacacgacactattttattgctgcagtggagaggctctgggattatgggatgagtagctccccacatgttctaagaaacagggctcagagtggcagtgtccctcagttcaagaaagttgttttccaggaatttactgatggctcctttactcagcccttataccgtggagaactaaatgaacatttgggactcctggggccatatataagagcagaagttgaagataatatcatggtaactttcagaaatcaggcctctcgtccctattccttctattctagccttatttcttatgaggaagatcagaggcaaggagcagaacctagaaaaaactttgtcaagcctaatgaaaccaaaacttacttttggaaagtgcaacatcatatggcacccactaaagatgagtttgactgcaaagcctgggcttatttctctgatgttgacctggaaaaagatgtgcactcaggcctgattggaccccttctggtctgccacactaacacactgaaccctgctcatgggagacaagtgacagtacaggaatttgctctgtttttcaccatctttgatgagaccaaaagctggtacttcactgaaaatatggaaagaaactgcagggctccctgcaatatccagatggaagatcccacttttaaagagaattatcgcttccatgcaatcaatggctacataatggatacactacctggcttagtaatggctcaggatcaaaggattcgatggtatctgctcagcatgggcagcaatgaaaacatccattctattcatttcagtggacatgtgttcaccgtacgaaaaaaagaggagtataaaatggcactgtacaatctctatccaggtgtttttgagacagtggaaatgttaccatccaaagctggaatttggcgggtggaatgccttattggcgagcatctacatgctgggatgagcacactttttctggtgtacagcaataagtgtcagactcccctgggaatggcttctggacacattagagattttcagattacagcttcaggacaatatggacagtgggccccaaagctggccagacttcattattccggatcaatcaatgcctggagcaccaaggagcccttttcttggatcaaggtggatctgttggcaccaatgattattcacggcatcaagacccagggtgcccgtcagaagttctccagcctctacatctctcagtttatcatcatgtatagtcttgatgggaagaagtggcagacttatcgaggaaattccactggaaccttaatggtcttctttggcaatgtggattcatctgggataaaacacaatatttttaaccctccaattattgctcgatacatccgtttgcacccaactcattatagcattcgcagcactcttcgcatggagttgatgggctgtgatttaaatagttgcagcatgccattgggaatggagagtaaagcaatatcagatgcacagattactgcttcatcctactttaccaatatgtttgccacctggtctccttcaaaagctcgacttcacctccaagggaggagtaatgcctggagacctcaggtgaataatccaaaagagtggctgcaagtggacttccagaagacaatgaaagtcacaggagtaactactcagggagtaaaatctctgcttaccagcatgtatgtgaaggagttcctcatctccagcagtcaagatggccatcagtggactctcttttttcagaatggcaaagtaaaggtttttcagggaaatcaagactccttcacacctgtggtgaactctctagacccaccgttactgactcgctaccttcgaattcacccccagagttgggtgcaccagattgccctgaggatggaggttctgggctgcgaggcacaggacctctactga

SEQ ID NO: 20 - DNA, 4890 bp, synthetic construct
  source 1..4890: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcaccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 21 - DNA, 4374 bp, synthetic construct
  source 1..4374: mol_type = other DNA
atgcagattgagctgtcaacttgctttttcctgtgcctgctgagattttgtttttccgctactagaagatactacctgggggctgtggaactgtcttgggattacatgcagagtgacctgggagagctgccagtggacgcacgatttccacctagagtccctaaatcattccccttcaacaccagcgtggtctataagaaaacactgttcgtggagtttactgatcacctgttcaacatcgctaagcctcggccaccctggatgggactgctgggaccaacaatccaggcagaggtgtacgacaccgtggtcattacactgaaaaacatggcctcacaccccgtgagcctgcatgctgtgggcgtcagctactggaaggcttccgaaggggcagagtatgacgatcagacttcccagagagaaaaagaggacgataaggtgtttcctggcgggtctcatacctatgtgtggcaggtcctgaaagagaatggccccatggcttccgaccctctgtgcctgacctactcttatctgagtcacgtggacctggtcaaggatctgaacagcggactgatcggagcactgctggtgtgtagggaagggagcctggctaaggagaaaacccagacactgcataagttcattctgctgttcgccgtgtttgacgaaggaaaatcatggcacagcgagacaaagaatagtctgatgcaggaccgggatgccgcttcagccagagcttggcccaaaatgcacactgtgaacggctacgtcaatcgctcactgcctggactgatcggctgccaccgaaagagcgtgtattggcatgtcatcggaatgggcaccacacctgaagtgcactccattttcctggaggggcatacctttctggtccgcaaccaccgacaggcctccctggagatctctccaattaccttcctgacagctcagactctgctgatggatctgggacagttcctgctgttttgccacatcagctcccaccagcatgatggcatggaggcctacgtgaaagtggacagctgtcccgaggaacctcagctgaggatgaagaacaatgaggaagctgaagactatgacgatgacctgaccgactccgagatggatgtggtccgattcgatgacgataacagcccctcctttatccagattagatctgtggccaagaaacaccctaagacatgggtccattacatcgcagccgaggaagaggactgggattatgcaccactggtgctggcaccagacgatcgatcctacaaatctcagtatctgaacaatggaccacagcggattggcagaaagtacaagaaagtgaggttcatggcttataccgatgaaaccttcaagactcgcgaagcaatccagcacgagagcgggattctgggaccactgctgtacggagaagtgggggacaccctgctgatcatttttaagaaccaggccagcaggccttacaatatctatccacatggaattacagatgtgcgccctctgtacagccggagactgccaaagggcgtcaaacacctgaaggacttcccaatcctgcccggggaaatttttaagtataaatggactgtcaccgtcgaggatggccccactaagagcgaccctaggtgcctgacccgctactattctagtttcgtgaatatggaaagggatctggccagcggactgatcggcccactgctgatttgttacaaagagagcgtggatcagagaggcaaccagatcatgtccgacaagaggaatgtgattctgttcagtgtctttgacgaaaaccggtcatggtatctgaccgagaacatccagagattcctgcctaatccagccggagtgcagctggaagatcctgagtttcaggcttctaacatcatgcatagtattaatggctacgtgttcgacagtctgcagctgtcagtgtgtctgcacgaggtcgcttactggtatatcctgagcattggagcacagacagatttcctgagcgtgttcttttccggctacacttttaagcataaaatggtgtatgaggacacactgactctgttccccttcagcggcgaaaccgtgtttatgtccatggagaatcccgggctgtggatcctgggatgccacaacagcgatttcaggaatcgcgggatgactgccctgctgaaagtgtcaagctgtgacaagaacaccggagactactatgaagattcatacgaggacatcagcgcatatctgctgtccaaaaacaatgccattgaacccaggtcttttagtcagaatcctccagtgctgaagaggcaccagcgcgagatcacccgcactaccctgcagagtgatcaggaagagatcgactacgacgatacaatttctgtggaaatgaagaaagaggacttcgatatctatgacgaagatgagaaccagagtcctcgatcattccagaagaaaacccggcattactttattgctgcagtggagcgcctgtgggattatggcatgtcctctagtcctcacgtgctgcgaaatcgggcccagtcagggagcgtcccacagttcaagaaagtggtcttccaggagtttacagacggatcctttactcagccactgtaccggggcgaactgaacgagcacctggggctgctgggaccctatatcagagctgaagtggaggataacattatggtcaccttcagaaatcaggcatctaggccttacagtttttattcaagcctgatctcttacgaagaggaccagaggcagggagcagaaccacgaaaaaacttcgtgaagcctaatgagaccaaaacatacttttggaaggtgcagcaccatatggccccaacaaaagacgaattcgattgcaaggcatgggcctatttttctgacgtggatctggagaaggacgtccacagtggcctgatcgggccactgctggtgtgtcatactaacaccctgaatcccgcacacggcaggcaggtcactgtccaggaattcgccctgttctttaccatctttgatgagacaaaaagctggtacttcaccgaaaacatggagcgaaattgccgggctccatgtaatattcagatggaagaccccacattcaaggagaactaccgctttcatgccatcaatgggtatattatggatactctgcccggactggtcatggctcaggaccagagaatcaggtggtacctgctgagcatggggtccaacgagaatatccactcaattcatttcagcggacacgtgtttactgtccggaagaaagaagagtataaaatggccctgtacaacctgtatcccggcgtgttcgaaaccgtcgagatgctgcctagcaaggcagggatctggagagtggaatgcctgattggggagcacctgcatgccggaatgtctaccctgtttctggtgtacagtaataagtgtcagacacccctggggatggcttccggacatatccgggatttccagattaccgcatctggacagtacggccagtgggcccctaagctggctagactgcactattccgggtctatcaacgcttggtccacaaaagagcctttctcttggattaaggtggacctgctggcaccaatgatcattcatggcatcaaaactcagggggccaggcagaagttctcctctctgtacatctcacagtttatcatcatgtacagcctggatggcaagaaatggcagacataccgcggcaatagcacagggactctgatggtgttctttggcaacgtggacagttcagggatcaagcacaacattttcaatccccctatcattgctagatacatcaggctgcacccaacccattattctattcgaagtacactgcggatggaactgatggggtgcgatctgaacagttgttcaatgcccctgggaatggagtccaaggcaatctctgacgcccagattaccgctagctcctacttcactaatatgtttgctacctggagcccctccaaagcacgactgcatctgcagggacgaagcaacgcatggcgaccacaggtgaacaatcccaaggagtggctgcaggtcgattttcagaaaactatgaaggtgaccggagtcacaactcagggcgtgaaaagtctgctgacctcaatgtacgtcaaggagttcctgatctctagttcacaggacggccaccagtggacactgttctttcagaacggaaaggtgaaagtcttccagggcaatcaggattcctttacacctgtggtcaactctctggacccacccctgctgactcgctacctgcgaatccacccacagtcctgggtgcatcagattgcactgagaatggaagtcctgggctgcgaggcccaggacctgtattga

SEQ ID NO: 22 - DNA, 222 bp, synthetic construct
  source 1..222: mol_type = other DNA
gtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcct

SEQ ID NO: 23 - DNA, 5066 bp, synthetic construct
  source 1..5066: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtgtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggtttaaacgccaccatgcagattgagctgagcacctgcttcttcctgtgtctgctgaggttctgcttctctgccaccaggaggtattacctgggggctgtggagctgagctgggactatatgcagtctgacctgggggagctgcctgtggatgctaggttcccccccagggtgcccaagagcttcccctttaacacttctgtggtgtacaagaagaccctgtttgtggagttcactgaccacctgttcaacattgccaagcccaggcccccctggatggggctgctggggcccaccatccaggctgaggtgtatgacactgtggtgatcaccctgaagaacatggccagccaccctgtgagcctgcatgctgtgggggtgagctactggaaggcttctgagggggctgagtatgatgaccagactagccagagggagaaggaggatgacaaggtgtttcctgggggcagccatacctatgtgtggcaggtgctgaaggagaatggccccatggcctctgaccccctgtgcctgacctacagctacctgtctcatgtggacctggtgaaggacctgaactctggcctgattggggctctgctggtgtgtagggagggcagcctggctaaggaaaagacccagaccctgcataagtttatcctgctgtttgctgtgtttgatgagggcaagagctggcactctgagaccaagaacagcctgatgcaggatagggatgctgcctctgccagggcttggcctaagatgcacactgtgaatgggtatgtgaataggagcctgcctggcctgattggctgccacaggaagtctgtgtactggcatgtgattgggatgggcaccacccctgaggtccatagcatcttcctggagggccacactttcctggtgaggaaccacagacaggcctctctggagatctctcccatcaccttcctgactgctcagactctgctgatggacctgggccagttcctgctgttttgccatattagcagccaccagcatgatgggatggaggcctatgtgaaggtggatagctgccctgaggagcctcagctgaggatgaagaacaatgaggaggctgaagactatgatgatgacctgactgattctgagatggatgtggtgaggtttgatgatgacaatagccccagcttcattcagatcaggtctgtggccaagaaacaccccaagacctgggtgcactacattgctgctgaggaagaggactgggactatgctcccctggtgctggcccctgatgataggtcttataagagccagtacctgaacaatgggccccagaggattggcaggaagtacaagaaggtgaggttcatggcctacactgatgaaaccttcaaaaccagggaggccattcagcatgagtctggcatcctgggccctctgctgtatggggaggtgggggacaccctgctgatcatcttcaagaaccaggccagcaggccctacaacatctatcctcatggcatcactgatgtgaggcccctgtacagcaggaggctgcccaagggggtgaagcacctgaaagacttccccatcctgcctggggagatctttaagtataagtggactgtgactgtggaggatggccctaccaagtctgaccccaggtgtctgaccaggtactattctagctttgtgaacatggagagggacctggcctctggcctgattgggcccctgctgatctgctacaaggagtctgtggaccagaggggcaaccagatcatgtctgacaagaggaatgtgatcctgttttctgtgtttgatgagaataggagctggtacctgactgagaacatccagaggtttctgcccaatcctgctggggtgcagctggaggatcctgagttccaggccagcaatatcatgcatagcatcaatggctatgtgtttgacagcctgcagctgtctgtgtgcctgcatgaggtggcctactggtacatcctgagcattggggcccagactgactttctgtctgtgttcttttctggctataccttcaagcacaagatggtgtatgaggataccctgaccctgttccccttctctggggagactgtgttcatgagcatggagaatcctgggctgtggatcctggggtgccacaactctgattttaggaacagggggatgactgccctgctgaaggtgtctagctgtgataagaacactggggactactatgaggacagctatgaggacatttctgcttatctgctgtctaagaataatgccattgagcccagaagcttcagccagaatccccctgtgctgaagagacatcagagggagatcaccagaactaccctgcagtctgatcaggaggagattgactatgatgacactatctctgtggagatgaagaaggaggactttgacatctatgatgaggatgagaatcagtctcccaggagctttcagaagaagaccagacattacttcattgctgctgtggagaggctgtgggactatggcatgagctctagccctcatgtgctgaggaacagggcccagtctggctctgtgccccagttcaagaaggtggtgttccaggaattcactgatggcagcttcacccagcccctgtacaggggggagctgaatgagcacctgggcctgctggggccttatatcagggctgaggtggaggataatattatggtgactttcaggaaccaggccagcaggccctactctttctatagcagcctgatctcttatgaggaggatcagaggcagggggctgagcctaggaagaactttgtgaagcccaatgagactaagacctacttctggaaggtccagcaccacatggcccctaccaaggatgagtttgactgcaaggcctgggcctatttctctgatgtggatctggagaaggatgtccattctgggctgattggccccctgctggtgtgccacactaacactctgaatcctgcccatggcaggcaggtgactgtccaggagtttgccctgttcttcactatctttgatgagaccaagagctggtactttactgagaacatggagaggaactgcagagctccttgcaatattcagatggaggaccccaccttcaaggagaattacaggttccatgccattaatgggtacatcatggacaccctgcctggcctggtgatggctcaggaccagaggatcaggtggtacctgctgagcatgggctctaatgagaatatccacagcatccacttctctgggcatgtgttcactgtgaggaagaaggaggagtacaagatggctctgtataatctgtaccctggggtgtttgaaactgtggagatgctgccctctaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactattctggcagcatcaatgcctggagcaccaaggagcccttcagctggatcaaggtggacctgctggcccccatgatcattcatggcatcaagacccagggggccaggcagaagttcagctctctgtacatctctcagttcatcatcatgtactctctggatgggaagaagtggcagacctacaggggcaacagcactggcaccctgatggtgttctttgggaatgtggactcttctggcatcaagcacaacatcttcaatccccccatcattgctaggtatattaggctgcatcccacccactacagcatcaggtctaccctgaggatggagctgatgggctgtgacctgaactcttgcagcatgcccctgggcatggagtctaaggccatctctgatgcccagattactgccagcagctacttcaccaacatgtttgccacctggagcccctctaaggccaggctgcatctgcaggggaggagcaatgcctggaggcctcaggtgaacaaccccaaggagtggctgcaggtggatttccagaagaccatgaaggtgactggggtgaccacccagggggtcaagagcctgctgaccagcatgtatgtgaaggagttcctgatcagcagcagccaggatggccaccagtggactctgttctttcagaatgggaaggtgaaggtgtttcagggcaatcaggactctttcacccctgtggtgaacagcctggacccccccctgctgaccagatacctgaggatccacccccagtcttgggtgcatcagattgccctgaggatggaggtgctgggctgtgaggctcaggatctgtactgagcggccgcaataaaagatcagagctctagagatctgtgtgttggttttttgtgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 24 - DNA, 11976 bp, synthetic construct
  source 1..11976: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctacgcgtgtctgtctgcacatttcgtagagcgagtgttccgatactctaatctccctaggcaaggttcatattgacttaggttacttattctccttttgttgactaagtcaataatcagaatcagcaggtttggagtcagcttggcagggatcagcagcctgggttggaaggagggggtataaaagccccttcaccaggagaagccgtcacacagatccacaagctcctgctagcaggtaagtgccgtgtgtggttcccgcgggcctggcctctttacgggttatggcccttgcgtgccttgaattactgacactgacatccactttttctttttctccacaggtttaaacgccaccatgcagattgagctgagcacctgcttcttcctgtgtctgctgaggttctgcttctctgccaccaggaggtattacctgggggctgtggagctgagctgggactatatgcagtctgacctgggggagctgcctgtggatgctaggttcccccccagggtgcccaagagcttcccctttaacacttctgtggtgtacaagaagaccctgtttgtggagttcactgaccacctgttcaacattgccaagcccaggcccccctggatggggctgctggggcccaccatccaggctgaggtgtatgacactgtggtgatcaccctgaagaacatggccagccaccctgtgagcctgcatgctgtgggggtgagctactggaaggcttctgagggggctgagtatgatgaccagactagccagagggagaaggaggatgacaaggtgtttcctgggggcagccatacctatgtgtggcaggtgctgaaggagaatggccccatggcctctgaccccctgtgcctgacctacagctacctgtctcatgtggacctggtgaaggacctgaactctggcctgattggggctctgctggtgtgtagggagggcagcctggctaaggaaaagacccagaccctgcataagtttatcctgctgtttgctgtgtttgatgagggcaagagctggcactctgagaccaagaacagcctgatgcaggatagggatgctgcctctgccagggcttggcctaagatgcacactgtgaatgggtatgtgaataggagcctgcctggcctgattggctgccacaggaagtctgtgtactggcatgtgattgggatgggcaccacccctgaggtccatagcatcttcctggagggccacactttcctggtgaggaaccacagacaggcctctctggagatctctcccatcaccttcctgactgctcagactctgctgatggacctgggccagttcctgctgttttgccatattagcagccaccagcatgatgggatggaggcctatgtgaaggtggatagctgccctgaggagcctcagctgaggatgaagaacaatgaggaggctgaagactatgatgatgacctgactgattctgagatggatgtggtgaggtttgatgatgacaatagccccagcttcattcagatcaggtctgtggccaagaaacaccccaagacctgggtgcactacattgctgctgaggaagaggactgggactatgctcccctggtgctggcccctgatgataggtcttataagagccagtacctgaacaatgggccccagaggattggcaggaagtacaagaaggtgaggttcatggcctacactgatgaaaccttcaaaaccagggaggccattcagcatgagtctggcatcctgggccctctgctgtatggggaggtgggggacaccctgctgatcatcttcaagaaccaggccagcaggccctacaacatctatcctcatggcatcactgatgtgaggcccctgtacagcaggaggctgcccaagggggtgaagcacctgaaagacttccccatcctgcctggggagatctttaagtataagtggactgtgactgtggaggatggccctaccaagtctgaccccaggtgtctgaccaggtactattctagctttgtgaacatggagagggacctggcctctggcctgattgggcccctgctgatctgctacaaggagtctgtggaccagaggggcaaccagatcatgtctgacaagaggaatgtgatcctgttttctgtgtttgatgagaataggagctggtacctgactgagaacatccagaggtttctgcccaatcctgctggggtgcagctggaggatcctgagttccaggccagcaatatcatgcatagcatcaatggctatgtgtttgacagcctgcagctgtctgtgtgcctgcatgaggtggcctactggtacatcctgagcattggggcccagactgactttctgtctgtgttcttttctggctataccttcaagcacaagatggtgtatgaggataccctgaccctgttccccttctctggggagactgtgttcatgagcatggagaatcctgggctgtggatcctggggtgccacaactctgattttaggaacagggggatgactgccctgctgaaggtgtctagctgtgataagaacactggggactactatgaggacagctatgaggacatttctgcttatctgctgtctaagaataatgccattgagcccagaagcttcagccagaatccccctgtgctgaagagacatcagagggagatcaccagaactaccctgcagtctgatcaggaggagattgactatgatgacactatctctgtggagatgaagaaggaggactttgacatctatgatgaggatgagaatcagtctcccaggagctttcagaagaagaccagacattacttcattgctgctgtggagaggctgtgggactatggcatgagctctagccctcatgtgctgaggaacagggcccagtctggctctgtgccccagttcaagaaggtggtgttccaggaattcactgatggcagcttcacccagcccctgtacaggggggagctgaatgagcacctgggcctgctggggccttatatcagggctgaggtggaggataatattatggtgactttcaggaaccaggccagcaggccctactctttctatagcagcctgatctcttatgaggaggatcagaggcagggggctgagcctaggaagaactttgtgaagcccaatgagactaagacctacttctggaaggtccagcaccacatggcccctaccaaggatgagtttgactgcaaggcctgggcctatttctctgatgtggatctggagaaggatgtccattctgggctgattggccccctgctggtgtgccacactaacactctgaatcctgcccatggcaggcaggtgactgtccaggagtttgccctgttcttcactatctttgatgagaccaagagctggtactttactgagaacatggagaggaactgcagagctccttgcaatattcagatggaggaccccaccttcaaggagaattacaggttccatgccattaatgggtacatcatggacaccctgcctggcctggtgatggctcaggaccagaggatcaggtggtacctgctgagcatgggctctaatgagaatatccacagcatccacttctctgggcatgtgttcactgtgaggaagaaggaggagtacaagatggctctgtataatctgtaccctggggtgtttgaaactgtggagatgctgccctctaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactattctggcagcatcaatgcctggagcaccaaggagcccttcagctggatcaaggtggacctgctggcccccatgatcattcatggcatcaagacccagggggccaggcagaagttcagctctctgtacatctctcagttcatcatcatgtactctctggatgggaagaagtggcagacctacaggggcaacagcactggcaccctgatggtgttctttgggaatgtggactcttctggcatcaagcacaacatcttcaatccccccatcattgctaggtatattaggctgcatcccacccactacagcatcaggtctaccctgaggatggagctgatgggctgtgacctgaactcttgcagcatgcccctgggcatggagtctaaggccatctctgatgcccagattactgccagcagctacttcaccaacatgtttgccacctggagcccctctaaggccaggctgcatctgcaggggaggagcaatgcctggaggcctcaggtgaacaaccccaaggagtggctgcaggtggatttccagaagaccatgaaggtgactggggtgaccacccagggggtcaagagcctgctgaccagcatgtatgtgaaggagttcctgatcagcagcagccaggatggccaccagtggactctgttctttcagaatgggaaggtgaaggtgtttcagggcaatcaggactctttcacccctgtggtgaacagcctggacccccccctgctgaccagatacctgaggatccacccccagtcttgggtgcatcagattgccctgaggatggaggtgctgggctgtgaggctcaggatctgtactgagcggccgcaataaaagatcagagctctagagatctgtgtgttggttttttgtgtaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcagcttgaaggaaatactaaggcaaaggtactgcaagtgctcgcaacattcgcttatgcggattattgccgtagtgccgcgacgccgggggcaagatgcagagattgccatggtacaggccgtgcggttgatattgccaaaacagagctgtgggggagagttgtcgagaaagagtgcggaagatgcaaaggcgtcggctattcaaggatgccagcaagcgcagcatatcgcgctgtgacgatgctaatcccaaaccttacccaacccacctggtcacgcactgttaagccgctgtatgacgctctggtggtgcaatgccacaaagaagagtcaatcgcagacaacattttgaatgcggtcacacgttagcagcatgattgccacggatggcaacatattaacggcatgatattgacttattgaataaaattgggtaaatttgactcaacgatgggttaattcgctcgttgtggtagtgagatgaaaagaggcggcgcttactaccgattccgcctagttggtcacttcgacgtatcgtctggaactccaaccatcgcaggcagagaggtctgcaaaatgcaatcccgaaacagttcgcaggtaatagttagagcctgcataacggtttcgggattttttatatctgcacaacaggtaagagcattgagtcgataatcgtgaagagtcggcgagcctggttagccagtgctctttccgttgtgctgaattaagcgaataccggaagcagaaccggatcaccaaatgcgtacaggcgtcatcgccgcccagcaacagcacaacccaaactgagccgtagccactgtctgtcctgaattcattagtaatagttacgctgcggccttttacacatgaccttcgtgaaagcgggtggcaggaggtcgcgctaacaacctcctgccgttttgcccgtgcatatcggtcacgaacaaatctgattactaaacacagtagcctggatttgttctatcagtaatcgaccttattcctaattaaatagagcaaatccccttattgggggtaagacatgaagatgccagaaaaacatgacctgttggccgccattctcgcggcaaaggaacaaggcatcggggcaatccttgcgtttgcaatggcgtaccttcgcggcagatataatggcggtgcgtttacaaaaacagtaatcgacgcaacgatgtgcgccattatcgcctagttcattcgtgaccttctcgacttcgccggactaagtagcaatctcgcttatataacgagcgtgtttatcggctacatcggtactgactcgattggttcgcttatcaaacgcttcgctgctaaaaaagccggagtagaagatggtagaaatcaataatcaacgtaaggcgttcctcgatatgctggcgtggtcggagggaactgataacggacgtcagaaaaccagaaatcatggttatgacgtcattgtaggcggagagctatttactgattactccgatcaccctcgcaaacttgtcacgctaaacccaaaactcaaatcaacaggcgccggacgctaccagcttctttcccgttggtgggatgcctaccgcaagcagcttggcctgaaagacttctctccgaaaagtcaggacgctgtggcattgcagcagattaaggagcgtggcgctttacctatgattgatcgtggtgatatccgtcaggcaatcgaccgttgcagcaatatctgggcttcactgccgggcgctggttatggtcagttcgagcataaggctgacagcctgattgcaaaattcaaagaagcgggcggaacggtcagagagattgatgtatgagcagagtcaccgcgattatctccgctctggttatctgcatcatcgtctgcctgtcatgggctgttaatcattaccgtgataacgccattacctacaaagcccagcgcgacaaaaatgccagagaactgaagctggcgaacgcggcaattactgacatgcagatgcgtcagcgtgatgttgctgcgctcgatgcaaaatacacgaaggagttagctgatgctaaagctgaaaatgatgctctgcgtgatgatgttgccgctggtcgtcgtcggttgcacatcaaagcagtctgtcagtcagtgcgtgaagccaccaccgcctccggcgtggataatgcagcctccccccgactggcagacaccgctgaacgggattatttcaccctcagagagaggctgatcactatgcaaaaacaactggaaggaacccagaagtatattaatgagcagtgcagatagagttgcccatatcgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatggcaacctgagccattggtaaaaccttccatgtgatacgagggcgcgtagtttgcattatcgtttttatcgtttcaatctggtctgacctccttgtgttttgttgatgatttatgtcaaatattaggaatgttttcacttaatagtattggttgcgtaacaaagtgcggtcctgctggcattctggagggaaatacaaccgacagatgtatgtaaggccaacgtgctcaaatcttcatacagaaagatttgaagtaatattttaaccgctagatgaagagcaagcgcatggagcgacaaaatgaataaagaacaatctgctgatgatccctccgtggatctgattcgtgtaaaaaatatgcttaatagcaccatttctatgagttaccctgatgttgtaattgcatgtatagaacataaggtgtctctggaagcattcagagcaattgaggcagcgttggtgaagcacgataataatatgaaggattattccctggtggttgactgatcaccataactgctaatcattcaaactatttagtctgtgacagagccaacacgcagtctgtcactgtcaggaaagtggtaaaactgcaactcaattactgcaatgccctcgtaattaagtgaatttacaatatcgtcctgttcggagggaagaacgcgggatgttcattcttcatcacttttaattgatgtatatgctctcttttctgacgttagtctccgacggcaggcttcaatgacccaggctgagaaattcccggaccctttttgctcaagagcgatgttaatttgttcaatcatttggttaggaaagcggatgttgcgggttgttgttctgcgggttctgttcttcgttgacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccttagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaactctatctcgggctattcttttgatttagacctgcaggcatgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcttcgagcaagacgtttcccgttgaatatggctcataacaccccttgtattactgtttatgtaagcagacagttttattgttcatgatgatatatttttatcttgtgcaatgtaacatcagagattttgagacacaacgtggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctctcgacggagttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 25 - protein, 1457 aa, synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 26 - protein, 2351 aa, synthetic construct
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 27 - protein, 736 aa, synthetic construct
MAADGYLPDWLEDNLSEGIREWWALQPGAPKPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRPL

SEQ ID NO: 28 - protein, 738 aa, synthetic construct
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVESPVKTAPGKKRPVEPSPQRSPDSSTGIGKKGQQPAKKRLNFGQTGDSESVPDPQPIGEPPAAPSGVGPNTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 29 - protein, 14 aa, synthetic construct
SFSQNPPVLKRHQR

SEQ ID NO: 30 - DNA, 11 bp, synthetic construct
  source 1..11: mol_type = other DNA
tatttgtgtag

SEQ ID NO: 31 - DNA, 11 bp, synthetic construct
  source 1..11: mol_type = other DNA
tattgacttag

(19) United States
(12) Patent Application Publication
(10) Pub. No.: US 2025/0073353 A1
(43) Pub. Date: Mar. 6, 2025

(54) FACTOR VIII (FVIII) GENE THERAPY METHODS

(71) Applicant: SPARK THERAPEUTICS, INC., Philadelphia, PA (US)

(72) Inventor: Xavier ANGUELA, Philadelphia, PA (US)

(73) Assignee: SPARK THERAPEUTICS, INC., Philadelphia, PA (US)

(21) Appl. No.: 18/803,700

(22) Filed: Aug. 13, 2024

Related U.S. Application Data
(63) Continuation of application No. 16/635,957, filed on Jan. 31, 2020, now abandoned.
(60) Provisional application No. 62/540,053, filed on Aug. 1, 2017.
(60) Provisional application No. 63/583,890, filed on Sep. 20, 2023.
(60) Provisional application No. 62/596,535, filed on Dec. 8, 2017.
(60) Provisional application No. 62/596,670, filed on Dec. 8, 2017.

(51) Int. Cl. A61K 48/00; A61K 9/51; A61K 38/37; A61P 7/02; C12N 15/86

(52) U.S. Cl. CPC A61K 48/0058; A61K 9/5184; A61K 38/37; A61K 48/0075; A61K 48/0083; A61P 7/02; C12N 15/86; C12N 2750/14132; C12N 2750/14143

(57) ABSTRACT

Methods of using vectors comprising nucleic acid and nucleic acid variants encoding FVIII protein are disclosed. In particular embodiments, a method of treating a human having hemophilia A includes administering a recombinant adeno-associated virus (rAAV) vector comprising a nucleic acid encoding Factor VIII (FVIII) or nucleic acid variant encoding Factor VIII (FVIII) having a B domain deletion (hFVIII-BDD). In some aspects, a nucleic acid variant has 95% or greater identity to SEQ ID NO:7 and/or a nucleic acid variant has no more than 2 cytosine-guanine dinucleotides (CpGs). In other aspects, a rAAV vector is administered to the human at a dose of less than about 6×10¹² vector genomes per kilogram (vg/kg).

### RELATED APPLICATIONS

This patent application is a continuation of U.S. patent application Ser. No. 16/635,957, filed on Jan. 31, 2020, which was a 371 of International Application No. PCT/US2018/044892, filed Aug. 1, 2018, and claims the benefit of U.S. Provisional Patent Application No. 62/540,053, filed on Aug. 1, 2017; U.S. Provisional Patent Application No. 62/583,890, filed on Nov. 9, 2017; U.S. Provisional Patent Application No. 62/596,535, filed on Dec. 8, 2017; and U.S. Provisional Patent Application No. 62/596,670, filed Dec. 8, 2017. The entire content of the foregoing applications is incorporated herein by reference, including all text, tables and drawings.

### REFERENCE TO SEQUENCE LISTING SUBMITTED ELECTRONICALLY

This application contains a sequence listing, which is submitted electronically via EFS-Web. The contents of the electronic sequence listing (065830.38US6.xml; size: 142,749 bytes); and date of creation: Aug. 9, 2024) is incorporated herein by reference in its entirety.

### FIELD OF THE INVENTION

This invention relates to the fields of recombinant coagulation factor production and the treatment of medical disorders associated with aberrant hemostasis. More particularly, the invention provides methods for administering a nucleic acid encoding Factor VIII (FVIII) protein, and hemophilia A treatment methods.

### INTRODUCTION

Several publications and patent documents are cited throughout the specification in order to describe the state of the art to which this invention pertains. Each of these citations is incorporated herein by reference as though set forth in full.

Hemophilia is an X-linked bleeding disorder present in 1 in 5,000 males worldwide. Therapies aimed at increasing clotting factor levels just above 1% of normal are associated with substantial improvement of the severe disease phenotype. Recent clinical trials for AAV-mediated gene transfer for hemophilia B (HB) have demonstrated sustained long-term expression of therapeutic levels of factor IX (FIX) but established that the AAV vector dose may be limiting due to anti-AAV immune responses to the AAV capsid. While these data relate to hemophilia B, 80% of all hemophilia is due to FVIII deficiency, hemophilia A (HA).

Current treatment for this disease is protein replacement therapy that requires frequent infusion of the Factor VIII protein. There is an immediate need to achieve sustained therapeutic levels of Factor VIII expression so that patients no longer require such frequent protein treatments. Indeed, continuous Factor VIII expression would prevent bleeding episodes and may ensure that immune tolerance to the protein is established.

### SUMMARY

In accordance with the invention, methods of treating a human having hemophilia A or in need of Factor VIII (FVIII) are provided. In one embodiment, a method includes administering a recombinant adeno-associated virus (rAAV) vector wherein the vector genome comprises a nucleic acid variant encoding Factor VIII (FVIII) having a B domain deletion (hFVIII-BDD), wherein the nucleic acid variant has 95% or greater identity to SEQ ID NO:7. In another embodiment, a method includes administering a recombinant adeno-associated virus (rAAV) vector wherein the vector genome comprises a nucleic acid variant encoding Factor VIII (FVIII) having a B domain deletion (hFVIII-BDD), wherein the nucleic acid variant has no more than 2 cytosine-guanine dinucleotides (CpGs).

In a further embodiment, a method of treating a human having hemophilia A or in need of Factor VIII (FVIII) includes administering a recombinant adeno-associated virus (rAAV) vector wherein the vector genome comprises a nucleic acid encoding Factor VIII (FVIII) or encoding Factor VIII (FVIII) having a B domain deletion (hFVIII-BDD), wherein the dose of rAAV vector administered to the human is less than 6×10¹² vector genomes per kilogram (vg/kg).

Embodiments of the methods and uses include administering to the human a dose of rAAV vector between about 1×10⁹ to about 1×10¹⁴ vg/kg, inclusive.

Embodiments of the methods and uses include administering to the human a dose of rAAV vector between about 1×10¹⁰ to about 6×10¹³ vg/kg, inclusive.

Embodiments of the methods and uses include administering to the human a dose of rAAV vector between about 1×10¹⁰ to about 1×10¹³ vg/kg, inclusive.

Embodiments of the methods and uses include administering to the human a dose of rAAV vector between about 1×10¹⁰ to about 6×10¹² vg/kg, inclusive.

Embodiments of the methods and uses include administering to the human a dose of rAAV vector between about 1×10¹⁰ to about 5×10¹² vg/kg, inclusive.

The method of any of claims 1-3, wherein the dose of rAAV vector administered to the human is between about 1×10¹¹ to about 1×10¹² vg/kg, inclusive.

Embodiments of the methods and uses include administering to the human a dose of rAAV vector between about 2×10¹¹ to about 9×10¹¹ vg/kg, inclusive.

Embodiments of the methods and uses include administering to the human a dose of rAAV vector between about 3×10¹¹ to about 8×10¹² vg/kg, inclusive.

12. The method of any of claims 1-3, wherein the dose of rAAV vector administered to the human is between about 3×10¹¹ to about 7×10¹² vg/kg, inclusive.

Embodiments of the methods and uses include administering to the human a dose of rAAV vector between about 3×10¹¹ to about 6×10¹² vg/kg, inclusive.

Embodiments of the methods and uses include administering to the human a dose of rAAV vector between about 4×10¹¹ to about 6×10¹² vg/kg, inclusive.

Embodiments of the methods and uses include administering to the human a dose of rAAV vector between about 5×10¹¹ vg/kg or about 1×10¹² vg/kg.

Embodiments of the methods and uses include providing greater than expected amount of FVIII or hFVIII-BDD in humans based upon data obtained from non-human primate studies administered the rAAV vector. Amounts of FVIII or hFVIII-BDD expressed in the human, as reflected by clotting activity, for example, can be greater than predicted based upon a liner regression curve derived from non-human primate studies administered the rAAV vector.

In certain embodiments, the amount of FVIII or hFVIII-BDD expressed in the human, as reflected by clotting activity, is greater than predicted based upon data obtained from non-human primate studies administered the rAAV vector.

In certain embodiments, the amount of FVIII or hFVIII-BDD expressed in the human, as reflected by clotting activity, is 1-4 fold greater than predicted expression based upon a liner regression curve derived from non-human primate studies administered the rAAV vector.

In certain embodiments, the amount of FVIII or hFVIII-BDD expressed in the human, as reflected by clotting activity, is 2-4 fold greater than predicted based upon a liner regression curve derived from non-human primate studies administered the rAAV vector.

In certain embodiments, the amount of FVIII or hFVIII-BDD expressed in the human, as reflected by clotting activity, is 2-3 fold greater than predicted based upon a liner regression curve derived from non-human primate studies administered the rAAV vector.

In certain embodiments, the amount of FVIII or hFVIII-BDD expressed in the human, as reflected by clotting activity, is 1-2 fold greater than predicted based upon a liner regression curve derived from non-human primate studies administered the rAAV vector.

Non-human primates include the genus of Macaca. In a particular embodiment, a non-human primate is a cynomologus monkey (Macaca fascicularis).

In certain embodiments, the FVIII or hFVIII-BDD is expressed for a period of time that provides a short term, medium term or longer term improvement in hemostasis. In certain embodiments, the period of time is such that no supplemental FVIII protein or recombinant FVIII protein need be administered to the human in order to maintain hemostasis.

In certain embodiments, the FVIII or hFVIII-BDD is expressed for at least about 14 days after rAAV vector administration.

In certain embodiments, the FVIII or hFVIII-BDD is expressed for at least about 21 days after rAAV vector administration.

In certain embodiments, the FVIII or hFVIII-BDD is expressed for at least about 28 days after rAAV vector administration.

In certain embodiments, the FVIII or hFVIII-BDD is expressed for at least about 35 days after rAAV vector administration.

In certain embodiments, the FVIII or hFVIII-BDD is expressed for at least about 42 days after rAAV vector administration.

In certain embodiments, the FVIII or hFVIII-BDD is expressed for at least about 49 days after rAAV vector administration.

In certain embodiments, the FVIII or hFVIII-BDD is expressed for at least about 56 days after rAAV vector administration.

In certain embodiments, the FVIII or hFVIII-BDD is expressed for at least about 63 days after rAAV vector administration.

In certain embodiments, the FVIII or hFVIII-BDD is expressed for at least about 70 days after rAAV vector administration.

In certain embodiments, the FVIII or hFVIII-BDD is expressed for at least about 77 days after rAAV vector administration.

In certain embodiments, the FVIII or hFVIII-BDD is expressed for at least about 84 days after rAAV vector administration.

In certain embodiments, the FVIII or hFVIII-BDD is expressed for at least about 91 days after rAAV vector administration.

In certain embodiments, the FVIII or hFVIII-BDD is expressed for at least about 98 days after rAAV vector administration.

In certain embodiments, the FVIII or hFVIII-BDD is expressed for at least about 105 days after rAAV vector administration.

In certain embodiments, the FVIII or hFVIII-BDD is expressed for at least about 112 days after rAAV vector administration.

In certain embodiments, the FVIII or hFVIII-BDD is expressed for at least about 4 months after rAAV vector administration.

In certain embodiments, the FVIII or hFVIII-BDD is expressed for at least about 154 days.

In certain embodiments, the FVIII or hFVIII-BDD is expressed for at least about 210 days.

In certain embodiments, the FVIII or hFVIII-BDD is expressed for at least about 6 months after rAAV vector administration.

In certain embodiments, the FVIII or hFVIII-BDD is expressed for at least about 12 months after rAAV vector administration.

FVIII or hFVIII-BDD can be expressed in certain amounts for a period of time after rAAV vector administration. In certain embodiments, the amount is such that there is detectable FVIII or hFVIII-BDD or an amount of FVIII or hFVIII-BDD that provides a therapeutic benefit.

In certain embodiments, the amount of FVIII or hFVIII-BDD expressed in the human, as reflected by clotting activity, is about 3% or greater at 14 or more days after rAAV vector administration, is about 4% or greater at 21 or more days after rAAV vector administration, is about 5% or greater at 21 or more days after rAAV vector administration, is about 6% or greater at 21 or more days after rAAV vector administration, is about 7% or greater at 21 or more days after rAAV vector administration, is about 8% or greater at 28 or more days after rAAV vector administration, is about 9% or greater at 28 or more days after rAAV vector administration, is about 10% or greater at 35 or more days after rAAV vector administration, is about 11% or greater at 35 or more days after rAAV vector administration, is about 12% or greater at 35 or more days after rAAV vector administration.

In certain embodiments, the amount of FVIII or hFVIII-BDD expressed in the human, as reflected by clotting activity, averages over a continuous 14 day period, about 10% or greater.

In certain embodiments, the amount of FVIII or hFVIII-BDD expressed in the human, as reflected by clotting activity, averages over a continuous 4 week period, about 10% or greater.

In certain embodiments, the amount of FVIII or hFVIII-BDD expressed in the human, as reflected by clotting activity, averages over a continuous 8 week period, about 10% or greater.

In certain embodiments, the amount of FVIII or hFVIII-BDD expressed in the human, as reflected by clotting activity, averages over a continuous 12 week period, about 10% or greater.

In certain embodiments, the amount of FVIII or hFVIII-BDD expressed in the human, as reflected by clotting activity, averages over a continuous 16 week period, about 10% or greater.

In certain embodiments, the amount of FVIII or hFVIII-BDD expressed in the human, as reflected by clotting activity, averages over a continuous 6 month period, about 10% or greater.

In certain embodiments, the amount of FVIII or hFVIII-BDD expressed in the human, as reflected by clotting activity, averages over a continuous 7 month period, about 10% or greater.

In certain embodiments, the amount of FVIII or hFVIII-BDD expressed in the human, as reflected by clotting activity, averages over a continuous 14 day period, about 12% or greater.

In certain embodiments, the amount of FVIII or hFVIII-BDD expressed in the human, as reflected by clotting activity, averages from about 12% to about 100% for a continuous 4 week period, for a continuous 8 week period, for a continuous 12 week period, for a continuous 16 week period, for a continuous 6 month period, for a continuous 7 month period, or for a continuous 1 year period.

In certain embodiments, the amount of FVIII or hFVIII-BDD expressed in the human, as reflected by clotting activity, averages from about 20% to about 80% for a continuous 4 week period, for a continuous 8 week period, for a continuous 12 week period, for a continuous 16 week period, for a continuous 6 month period, or for a continuous 1 year period.

Steady-state FVIII expression can also be achieved after a certain period of time, e.g., 4-6, 6-8 or 6-12 weeks or longer, e.g., 6-12 months or even years after rAAV vector administration.

In certain embodiments, FVIII or hFVIII-BDD is produced in the human at a steady state wherein FVIII activity does not vary by more than 5-50% over 4, 6, 8 or 12 weeks or months.

In certain embodiments, FVIII or hFVIII-BDD is produced in the human at a steady state wherein FVIII activity does not vary by more than 25-100% over 4, 6, 8 or 12 weeks or months.

rAAV vector can be administered at doses that would be expected to provide expression of FVIII at certain amounts and for certain periods of time to provide sustained expression after administration.

In certain embodiments, rAAV vector is administered at a dose of between about 1×10⁹ to about 1×10¹⁴ vg/kg inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of between about 5×10⁹ to about 6×10¹³ vg/kg inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of between about 1×10¹⁰ to about 6×10¹³ vg/kg inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of between about 1×10¹⁰ to about 1×10¹³ vg/kg inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of between about 1×10¹⁰ to about 6×10¹² vg/kg inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of less than 6×10¹² vg/kg to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 1×10¹⁰ to about 5×10¹² vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 1×10¹¹ to about 1×10¹² vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 2×10¹¹ to about 9×10¹¹ vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 3×10¹¹ to about 8×10¹² vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 3×10¹¹ to about 7×10¹² vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 3×10¹¹ to about 6×10¹² vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 4×10¹¹ to about 6×10¹² vg/kg, inclusive to the human, and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

In certain embodiments, rAAV vector is administered at a dose of about 5×10¹¹ vg/kg or about 1×10¹² vg/kg and FVIII or hFVIII-BDD is produced in the human at levels averaging about 12% to about 100% activity for at least 1, 2, 3, 4, 5, 6, 7, 8, 9, 10, 11, 12, 13 or 14 continuous days, weeks or months after rAAV vector administration.

Humans according to the methods and uses include those that are sero-negative for or do not have detectable AAV antibodies.

In certain embodiments, AAV antibodies in the human are not detected prior to rAAV vector administration or wherein said human is sero-negative for AAV.

In certain embodiments, AAV antibodies against the FVIII or hFVIII-BDD are not detected for at least about 1, 2, 3, 4, 5, 6, 7, 8, 9, 10, 11 or months or longer after rAAV vector administration.

In certain embodiments, AAV antibodies against the rAAV vector are not detected for at least about 14 days, or for at least about 21 days, or for at least about 28 days, or for at least about 35 days, or for at least about 42 days, or for at least about 49 days, or for at least about 56 days, or for at least about 63 days, or for at least about 70 days, or for at least about 77 days, or for at least about 84 days, or for at least about 91 days, or for at least about 98 days, or for at least about 105 days, or for at least about 112 days, after rAAV vector administration.

Humans according to the methods and uses include those that have detectable AAV antibodies.

In certain embodiments, AAV antibodies in the human are at or less than about 1:5 prior to rAAV vector administration.

In certain embodiments, AAV antibodies in the human are at or less than about 1:3 prior to rAAV vector administration.

In certain methods and uses, a human administered the rAAV vector does not produce a cell mediated immune response against the rAAV vector.

In certain embodiments, the human administered the rAAV vector does not produce a cell mediated immune response against the rAAV vector for at least 1, 2, 3, 4, 5, 6, 7, 8, 9, 10, 11, 12, 13 or 14 continuous weeks or months after rAAV vector administration.

In certain embodiments, the human administered the rAAV vector does not develop a humoral immune response against the rAAV vector sufficient to decrease or block the FVIII or hFVIII-BDD therapeutic effect.

In certain embodiments, the human administered the rAAV vector does not produce detectable antibodies against the rAAV vector for at least about 1, 2, 3, 4, 5 or 6 months after rAAV vector administration.

In certain embodiments, the human administered the rAAV vector is not administered an immunusuppresive agent prior to, during and/or after rAAV vector administration.

In certain embodiments, the human administered the rAAV vector FVIII or hFVIII-BDD expressed in the human is achieved without administering an immunusuppresive agent.

In the case of a pre-existing or an immune response that develops after rAAV vector administration, a human may be administered an immunosuppressive agent prior to or after rAAV vector administration.

In certain embodiments, a method or use includes administering an immunosuppressive agent prior to administration of the rAAV vector.

In certain embodiments, a method or use includes administering an immunosuppressive agent after administration of the rAAV vector.

In certain embodiments, an immunosuppressive agent is administered from a time period within 1 hour to up to 45 days after the rAAV vector is administered.

In certain embodiments, an immunosuppressive agent immunosuppressive agent comprises a steroid, cyclosporine (e.g., cyclosporine A), mycophenolate, Rituximab or a derivative thereof.

In certain embodiments, nucleic acid variants have 90%, 91%, 92%, 93%, 94%, 95%, 96%, 97%, 98%, 99%, 99.5% or greater sequence identity to any of SEQ ID NOs:1-18. In certain embodiments, nucleic acid variants have 90-95% sequence identity to any of SEQ ID NOs:1-18. In certain embodiments, nucleic acid variants have 95%-100% sequence identity to any of SEQ ID NOs:1-18.

In certain embodiments, a nucleic acid variant encoding FVIII or hFVIII-BDD has a reduced CpG content compared to wild-type nucleic acid encoding FVIII. In certain embodiments, a nucleic acid variant has at least 20 fewer CpGs than wild-type nucleic acid encoding FVIII (SEQ ID NO:19). In certain embodiments, a nucleic acid variant has no more than 10 CpGs, has no more than 9 CpGs, has no more than 8 CpGs, has no more than 7 CpGs, has no more than 6 CPGs, has no more than 5 CpGs, has no more than 4 CpGs; has no more than 3 CpGs; has no more than 2 CpGs; or has no more than 1 CpG. In certain embodiments, a nucleic acid variant has at most 4 CpGs; 3 CpGs; 2 CpGs; or 1 CpG. In certain embodiments, a nucleic acid variant has no CpGs.

In certain embodiments, a nucleic acid variant encoding FVIII or hFVIII-BDD has a reduced CpG content compared to wild-type nucleic acid encoding FVIII, and such CpG reduced nucleic acid variants have 90% or greater sequence identity to any of SEQ ID NOs:1-18. In certain embodiments, CpG reduced nucleic acid variants have 91%, 92%, 93%, 94%, 95%, 96%, 97%, 98%, 99%, 99.5% or greater sequence identity to any of SEQ ID NOs:1-18. In certain embodiments, CpG reduced nucleic acid variants have 90-95% sequence identity to any of SEQ ID NOs:1-18. In certain embodiments, CpG reduced nucleic acid variants have 95%-100% sequence identity to any of SEQ ID NOs:1-18. In certain embodiments, FVIII encoding CpG reduced nucleic acid variants are set forth in any of SEQ ID NOs:1-18.

In certain embodiments, nucleic acid variants encoding FVIII or hFVIII-BDD protein are at least 75% identical to wild type human FVIII nucleic acid or wild type human FVIII nucleic acid comprising a B domain deletion. In certain embodiments, nucleic acid variants encoding FVIII protein are about 75-95% identical (e.g., about 75%, 76%, 77%, 78%, 79%, 80%, 81%, 82%, 83%, 84%, 85%, 86%, 87%, 88%, 89%, 90%, 91%, 92%, 93%, 94%, 95% identical) to wild type human FVIII nucleic acid or wild type human FVIII nucleic acid comprising a B domain deletion.

In certain embodiments, nucleic acids and variants encoding FVIII protein are mammalian, such as human. Such mammalian nucleic acids and nucleic acid variants encoding FVIII protein include human forms, which may be based upon human wild type FVIII or human wild type FVIII comprising a B domain deletion.

In certain embodiments, a recombinant adenovirus-associated virus (sAAV) vector comprises an AAV vector comprises an AAV serotype or an AAV pseudotype, such as AAV1, AAV2, AAV3, AAV4, AAV5, AAV6, AAV7, AAV8, AAV9, AAV10, AAV11, Rh10, Rh74 or AAV-2i8 AAV. In certain embodiments, an rAAV vector comprises any of SEQ ID Nos: 1-18, or comprises SEQ ID NO: 23 or 24.

In certain embodiments, an expression control element comprises a constitutive or regulatable control element, or a tissue-specific expression control element or promoter. In certain embodiments, an expression control element comprises an element that confers expression in liver. In certain embodiments, an expression control element comprises a TTR promoter or mutant TTR promoter, such as SEQ ID NO:22. In further particular aspects, an expression control element comprises a promoter set forth in PCT publication WO 2016/168728 (U.S. Ser. No. 62/148,696; 62/202,133; and 62/212,634), which are incorporated herein by reference in their entirety.

In certain embodiments, a rAAV vector comprises an AAV serotype or an AAV pseudotype comprising an AAV capsid serotype different from an ITR serotype. In additional embodiments, a rAAV vector comprises a VP1, VP2 and/or VP3 capsid sequence having 75% or more sequence identity (e.g., 80%, 85%, 90%, 95%, 96%, 97%, 98%, 99%, 99.1%, 99.2%, 99.3%, 99.4%, 99.5%, 99.6%, 99.7%, 99.8%, etc.) to any of AAV1, AAV2, AAV3, AAV4, AAV5, AAV6, AAV7, AAV8, AAV9, AAV10, AAV11, Rh10, Rh74 or AAV-218 AAV serotypes.

In certain embodiments, a rAAV vector comprises a VP1, VP2 and/or VP3 capsid sequence having 75% or more sequence identity (e.g., 80%, 85%, 90%, 95%, 96%, 97%, 98%, 99%, 99.1%, 99.2%, 99.3%, 99.4%, 99.5%, 99.6%, 99.7%, 99.8%, etc.) to any SEQ ID NO:27 or SEQ ID NO:28. In certain embodiments, a rAAV vector comprises a VP1, VP2 and/or VP3 capsid 100% identical to SEQ ID NO:27 or SEQ ID NO:28.

In certain embodiments, a rAAV vector further includes an intron, an expression control element, one or more AAV inverted terminal repeats (ITRs) (e.g., any of: AAV1, AAV2, AAV3, AAV4, AAV5, AAV6, AAV7, AAV8, AAV9, AAV10, AAV11, Rh10, Rh74 or AAV-218 AAV serotypes, or a combination thereof), a filler polynucleotide sequence and/or poly A signal.

In certain embodiments, an intron is within or flanks a nucleic acid or nucleic acid variant encoding FVIII or hFVIII-BDD, and/or an expression control element is operably linked to a nucleic acid or nucleic acid variant encoding FVIII or hFVIII-BDD, and/or an AAV ITR(s) flanks the 5′ or 3′ terminus of the nucleic acid or nucleic acid variant encoding FVIII, and/or a filler polynucleotide sequence flanks the 5′ or 3′terminus of the a nucleic acid or nucleic acid variant encoding FVIII or hFVIII-BDD.

In particular embodiments, an expression control element comprises a constitutive or regulatable control element, or a tissue-specific expression control element or promoter. In certain embodiments, an expression control element comprises an element that confers expression in liver (e.g., a TTR promoter or mutant TTR promoter).

In certain embodiments, a rAAV comprises a pharmaceutical composition. Such pharmaceutical compositions optionally include empty capsid AAV (e.g., lack vector genome comprising FVIII or hFVIII-BDD encoding nucleic acid or nucleic acid variant).

In certain embodiments, a nucleic acid or nucleic acid variant encoding FVIII or hFVIII-BDD protein, vectors, expression vectors, or virus or AAV vectors are encapsulated in a liposome or mixed with phospholipids or micelles.

Methods of the invention also include treating mammalian subjects (e.g., humans) such as humans in need of FVIII (the human produces an insufficient amount of FVIII protein, or a defective or aberrant FVIII protein) or that has hemophilia A.

In one embodiment, a human produces an insufficient amount of FVIII protein, or a defective or aberrant FVIII protein. In another embodiment, a human has mild, moderate or severe hemophilia A.

In certain embodiments, FVIII or hFVIII-BDD expressed by way of a rAAV vector administered is expressed at levels having a beneficial or therapeutic effect on the mammal.

Candidate subjects (e.g., a patient) and mammals (e.g., humans) for administration (e.g., delivery) of a rAAV comprising a nucleic acid or nucleic acid variant encoding FVIII or hFVIII-BDD include those having or those at risk of having a disorder such as: hemophilia A, von Willebrand diseases and bleeding associated with trauma, injury, thrombosis, thrombocytopenia, stroke, coagulopathy, disseminated intravascular coagulation (DIC) or over-anticoagulation treatment disorder.

Candidate subjects (e.g., a patient) and mammals (e.g., humans) for administration (e.g., delivery) of a a nucleic acid or nucleic acid variant encoding FVIII include those or sero-negative for AAV antibodies, as well as those having (seropositive) or those at risk of developing AAV antibodies. Such subjects (e.g., a patient) and mammals (e.g., humans) may be sero-negative or sero-positive for an AAV1, AAV2, AAV3, AAV4, AAV5, AAV6, AAV7, AAV8, AAV9, AAV10, AAV11, AAV-Rh10 or AAV-Rh74 serotype.

In certain embodiments, empty capsid of AAV1, AAV2, AAV3, AAV4, AAV5, AAV6, AAV7, AAV8, AAV9, AAV10, AAV11, AAV-12, AAV-Rh10 and/or AAV-Rh74 serotype is further administered to the mammal or patient alone or in combination with an rAAV vector comprising a nucleic acid or nucleic acid variant encoding FVIII.

Methods of administration (e.g., delivery) in accordance with the invention include any mode of contact or delivery, ex vivo or in vivo. In particular embodiments administration (e.g., delivery) is: intravenously, intraarterially, intramuscularly, subcutaneously, intra-cavity, intubation, or via catheter.

In certain embodiments, FVIII or hFVIII-BDD is expressed at levels without substantially increasing risk of thrombosis.

In certain embodiments, thrombosis risk is determined by measuring fibrin degradation products.

In certain embodiments, activity of the FVIII or hFVIII-BDD is detectable for at least 1, 2, 3 or 4 weeks, or at least 1, 2, 3, 4, 5, 6, 7, 8, 9, 10 or 11 months, or at least 1 year in the human.

In certain embodiments, a human is further analyzed or monitored for one or more of the following: the presence or amount of AAV antibodies, an immune response against AAV, FVIII or hFVIII-BDD antibodies, an immune response against FVIII or hFVIII-BDD, FVIII or hFVIII-BDD amounts, FVIII or hFVIII-BDD activity, amounts or levels of one or more liver enzymes or frequency, and/or severity or duration of bleeding episodes.

### DESCRIPTION OF DRAWINGS

FIG. 1 shows NHP Study design.

FIGS. 2A-2C show hFVIII antigen levels in NHPs following intravenous administration of either 2×10¹² (A), 5×10¹² (B) or 1×10¹³ vg/kg (C) of AAV-SPK-8005. Lines represent individual animals. Human FVIII plasma levels were assayed by ELISA and represent repeated measurements, obtained by serial bleeding, on the same group of animals during the course of the study (n=2-3 animals per cohort). Human FVIII levels measured in vehicle-treated animals are shown in open squares in all three graphs.

=Development of inhibitors against FVIII.

FIGS. 3A-3C show ALT levels in NHPs, at 2×10¹² (A), 5×10¹² (B) or 1×10¹³ vg/kg (C) of AAV-SPK-8005.

FIGS. 4A-4C show D-Dimer levels in NHPs. D-dimer antigen concentration in plasma of NHPs following intravenous administration of either 2×10¹² (A), 5×10¹² (B) or 1×10¹³ vg/kg (C) of AAV-SPK-8005. The dotted line indicates 500 ng/ml, the upper limit of normal for D-dimers in humans.

FIG. 5 shows a data summary of FVIII levels in the three doses of AAV-SPK-8005.

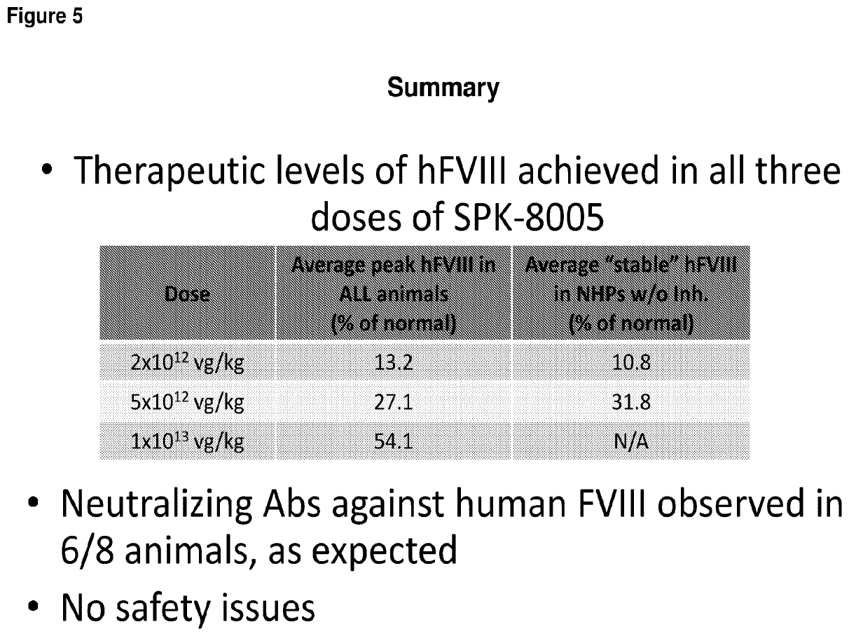

FIGS. 6A-6D show levels of hFVIII in plasma of cynomolgus macaques following intravenous administration of either 2×10¹² (A), 6×10¹² (B) or 2×10¹³ (vg/kg) (C) of AAV-SPK-8011 (LK03 capsid)-hFVIII (pilot study). Lines represent individual animals. hFVIII plasma levels were assayed by ELISA and represent repeated measurements, obtained by serial bleeding, on the same group of animals during the course of the study (n=3 animals per cohort). Human FVIII levels measured in vehicle-treated animals are shown in open squares (n=2). ε=Time when development of inhibitors against FVIII was detected in each individual animal.

FIG. 7 shows Human FVIII expression levels in cynomolgus macaques after administration of SPK-8011. Pilot study (squares) and GLP study (circles).

FIG. 8 shows a comparison of FVIII levels achieved with AAV-SPK-8011 (LK03 capsid)-hFVIII to the reported levels of FVIII delivered by way of AAV vectors with AAV5 and AAV8 capsids. http://www.biomarin.com/pdf/BioMarin_R&D_Day_4_20_2016.pdf, slide 16. AAV8: McIntosh J et al. Blood 2013; 121(17): 3335-44.

FIG. 9 shows AAV-SPK (SEQ ID NO:28) and AAV-LK03 (SEQ ID NO:27) tissue biodistribution in non-human primates, predominanyl in kidney, spleen and liver (3rd bar for each tissue).

FIG. 10 shows hepatic and splenic FVIII expression after systemic administration of AAV-SPK-8005 into mice.

FIG. 11 shows transduction efficiency of the AAV-LK03 capsid analyzed in vitro. X-axis, cynomolgus (left vertical bar), human (right vertical bar).

FIG. 12 shows human FVIII expression levels in cynomolgus macaques after administration of SPK-8011 follows a linear dose response. Panels A and B show SPK-8011 doses in a linear scale whereas panels C and D use a logarithmic X axis.

FIG. 13 shows analysis of linear regression using data from the low- and mid-dose cohorts only. Panels A and B show SPK-8011 doses in a linear scale whereas panels C and D use a logarithmic X axis.

FIG. 14 shows FVIII activity in 3 human subjects infused with AAV-LK03 (FVIII) vector. Subjects 1 and 2 (diamond, circle) were infused with 5×10¹¹ vg/kg AAV-LK03 (FVIII) vector. Subject 3 (triangle) was infused with 1×10¹² vg/kg AAV-LK03 (FVIII) vector.

FIG. 15 shows extended expression of FVIII activity at therapeutic levels in the same human subjects (Subjects 1 and 2, FIG. 14) infused with AAV-LK03 (FVIII) vector. Subjects 1 and 2 (circle, square) were infused with 5×10¹¹ vg/kg AAV-LK03 (FVIII) vector.

FIG. 16 shows 10 human subjects (Subjects 1-10) exhibiting therapeutic levels of FVIII. Subject 1 infused FVIII following emergency dental extraction in Week 6 post-infusion. FVIII shortly thereafter recorded 19% activity level; excluded from this chart due to FVIII infusion proximity. FVIII activity refers to FVIII:C values from local labs

FIG. 17 shows therapeutic levels of FVIII in Subject 1 infused with 5×10¹¹ vg/kg AAV-LK03 (FVIII) vector. Bottom graph shows results of the interferon-γ enzyme-linked immunosorbent spot (ELISPOT) assay regarding the reaction of the subject's peripheral blood mononuclear cells (PBMCs) to AAV capsid peptides (solid bar) and FVIII peptides (open circle). Results are shown as the number of spot-forming units (SFU) per 1 million PBMCs; values that are more than 50 SFU or that are above the media control (dotted line) by a factor of three are considered positive.

FIG. 18 shows therapeutic levels of FVIII in Subject 2 infused with 5×10¹¹ vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 19 shows therapeutic levels of FVIII in Subject 3 infused with 1×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 20 shows therapeutic levels of FVIII in Subject 4 infused with 1×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 21 shows therapeutic levels of FVIII in Subject 5 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 22 shows therapeutic levels of FVIII in Subject 6 infused with 1×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 23 shows therapeutic levels of FVIII in Subject 7 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 24 shows therapeutic levels of FVIII in Subject 8 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 25 shows therapeutic levels of FVIII in Subject 9 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 26 shows therapeutic levels of FVIII in Subject 10 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 27 shows therapeutic levels of FVIII in Subject 11 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

FIG. 28 shows therapeutic levels of FVIII in Subject 12 infused with 2×10¹² vg/kg AAV-LK03 (FVIII) vector. Bottom graph shows results of the interferon-γ ELISPOT assay regarding the reaction of the subject's PBMCs to AAV capsid peptides (solid bar) and FVIII peptides (open circle). Results are shown as the number of SFU per 1 million PBMCs; values that are more than 50 SFU or that are above the media control (dotted line) by a factor of three are considered positive.

### DETAILED DESCRIPTION

Disclosed herein are methods of treating a human having hemophilia A or in need of Factor VIII (FVIII) are provided. Such methods can be achieved using rAAV vectors with a geneome comprising nucleic acid or nucleic acid variants encoding FVIII or hFVIII-BDD, which can be expressed in cells and/or humans, which in turn can provide increased FVIII or hFVIII-BDD protein levels in vivo. Exemplary nucleic acid variants encoding FVIII or hFVIII-BDD can have reduced CpGs compared with a reference wild-type mammalian (e.g., human) FVIII or hFVIII-BDD and/or less than 100% sequence identity with a reference wild-type mammalian (e.g., human) FVIII or hFVIII-BDD. Such methods can also be achieved by administering a rAAV vector dose amount less than 6×10¹² vrAAV vector genomes per kilogram (vg/kg). rAAV vectors administered at dose amounts less than 6×10¹² vrAAV vector genomes per kilogram (vg/kg) can comprise a vector genome comprising a nucleic acid or nucleic acid variant encoding FVIII or hFVIII-BDD.

The terms “polynucleotide” and “nucleic acid” are used interchangeably herein to refer to all forms of nucleic acid, oligonucleotides, including deoxyribonucleic acid (DNA) and ribonucleic acid (RNA). Polynucleotides include genomic DNA, cDNA and antisense DNA, and spliced or unspliced mRNA, rRNA tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides include naturally occurring, synthetic, and intentionally modified or altered polynucleotides (e.g., variant nucleic acid). Polynucleotides can be single, double, or triplex, linear or circular, and can be of any length. In discussing polynucleotides, a sequence or structure of a particular polynucleotide may be described herein according to the convention of providing the sequence in the 5′ to 3′ direction.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular example of a modification or variant is a CpG reduced nucleic acid variant encoding FVIII.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of the protein (FVIII) encoded thereby, and some codons of the nucleic acid variant will be changed which in turn changes the amino acids of the protein (FVIII) encoded thereby.

The term “variant Factor VIII (FVIII)” refers to a modified FVIII which has been genetically altered as compared to unmodified wild-type FVIII (e.g., SEQ ID NO:19) or FVIII-BDD. Such a variant can be referred to as a “nucleic acid variant encoding Factor VIII (FVIII).” A particular example of a variant is a CpG reduced nucleic acid encoding FVIII or FVIII-BDD protein. The term “variant” need not appear in each instance of a reference made to CpG reduced nucleic acid encoding FVIII. Likewise, the term “CpG reduced nucleic acid” or the like may omit the term “variant” but it is intended that reference to “CpG reduced nucleic acid” includes variants at the genetic level.

FVIII and hFVIII-BDD constructs having reduced CpG content can exhibit improvements compared to wild-type FVIII or FVIII-BDD in which CpG content has not been reduced, and do so without modifications to the nucleic acid that result in amino acid changes to the encoded FVIII or FVIII-BDD protein. When comparing expression, if the CpG reduced nucleic acid encodes a FVIII protein that retains the B-domain, it is appropriate to compare it to wild-type FVIII expression; and if the CpG reduced nucleic acid encodes a FVIII protein without a B-domain, it is compared to expression of wild-type FVIII that also has a B-domain deletion.

A “variant Factor VIII (FVIII)” can also mean a modified FVIII protein such that the modified protein has an amino acid alteration compared to wild-type FVIII. Again, when comparing activity and/or stability, if the encoded variant FVIII protein retains the B-domain, it is appropriate to compare it to wild-type FVIII; and if the encoded variant FVIII protein has a B-domain deletion, it is compared to wild-type FVIII that also has a B-domain deletion.

A variant FVIII can include a portion of the B-domain. Thus, FVIII-BDD includes a portion of the B-domain. Typically, in FVIII-BDD most of the B-domain is deleted.

A variant FVIII can include an “SQ” sequence set forth as SFSQNPPVLKRHQR (SEQ ID NO:29). Typically, such a variant FVIII with an SQ (FVIII/SQ) has a BDD, e.g., at least all or a part of BD is deleted. Variant FVIII, such as FVIII-BDD can have all or a part of the “SQ” sequence, i.e. all or a part of SEQ ID NO:29. Thus, for example, a variant FVIII-BDD with an SQ sequence (SFSQNPPVLKRHQR, SEQ ID NO:29) can have all or just a portion of the amino acid sequence SFSQNPPVLKRHQR. For example, FVIII-BDD can have 1, 2, 3, 4, 5, 6, 7, 8, 9, 10, 11, 12 or 13 amino acid residues of SFSQNPPVLKRHQR included. Thus, SFSQNPPVLKRHQR with 1, 2, 3, 4, 5, 6, 7, 8, 9, 10, 11, 12 or 13 internal deletions as well as 1, 2, 3, 4, 5, 6, 7, 8, 9, 10, 11, 12 or 13 amino- or carboxy terminal deletions are included in the variant FVIII proteins set forth herein.

The “polypeptides,” “proteins” and “peptides” encoded by the “nucleic acid” or “polynucleotide” sequences,” include full-length native (FVIII) sequences, as with naturally occurring wild-type proteins, as well as functional subsequences, modified forms or sequence variants so long as the subsequence, modified form or variant retain some degree of functionality of the native full-length protein. For example, a CpG reduced nucleic acid encoding FVIII or hFVIII-BDD protein can have a B-domain deletion as set forth herein and retain clotting function. In methods and uses of the invention, such polypeptides, proteins and peptides encoded by the nucleic acid sequences can be but are not required to be identical to the endogenous protein that is defective, or whose expression is insufficient, or deficient in the treated mammal.

Non-limiting examples of modifications include one or more nucleotide or amino acid substitutions (e.g., 1-3, 3-5, 5-10, 10-15, 15-20, 20-25, 25-30, 30-40, 40-50, 50-100, 100-150, 150-200, 200-250, 250-500, 500-750, 750-850 or more nucleotides or residues). An example of a nucleic acid modification is CpG reduction. In certain embodiments, a CpG reduced nucleic acid encoding FVIII, such as human FVIII protein, has 10 or fewer CpGs compared to wild-type sequence encoding human Factor FVIII; or has 5 or fewer CpGs compared to wild-type sequence encoding human Factor FVIII; or has no more than 5 CpGs in the CpG reduced nucleic acid encoding FVIII.

An example of an amino acid modification is a conservative amino acid substitution or a deletion (e.g., subsequences or fragments) of a reference sequence, e.g. FVIII, such as FVIII with a B-domain deletion. In particular embodiments, a modified or variant sequence retains at least part of a function or activity of unmodified sequence.

All mammalian and non-mammalian forms of nucleic acid encoding proteins, including other mammalian forms of the CpG reduced nucleic acid encoding FVIII and hFVIII-BDD disclosed herein are expressly included, either known or unknown. Thus, the invention includes genes and proteins from non-mammals, mammals other than humans, and humans, which genes and proteins function in a substantially similar manner to the FVIII (e.g., human) genes and proteins described herein.

The term “vector” refers to small carrier nucleic acid molecule, a plasmid, virus (e.g., AAV vector), or other vehicle that can be manipulated by insertion or incorporation of a nucleic acid. Such vectors can be used for genetic manipulation (i.e., “cloning vectors”), to introduce/transfer polynucleotides into cells, and to transcribe or translate the inserted polynucleotide in cells. An “expression vector” is a specialized vector that contains a gene or nucleic acid sequence with the necessary regulatory regions needed for expression in a host cell. A vector nucleic acid sequence generally contains at least an origin of replication for propagation in a cell and optionally additional elements, such as a heterologous polynucleotide sequence, expression control element (e.g., a promoter, enhancer), intron, ITR(s), selectable marker (e.g., antibiotic resistance), polyadenylation signal.

A viral vector is derived from or based upon one or more nucleic acid elements that comprise a viral genome. Particular viral vectors include lentivirus, pseudo-typed lentivirus and parvo-virus vectors, such as adeno-associated virus (AAV) vectors.

The term “recombinant,” as a modifier of vector, such as recombinant viral, e.g., lenti- or parvo-virus (e.g., AAV) vectors, as well as a modifier of sequences such as recombinant polynucleotides and polypeptides, means that the compositions have been manipulated (i.e., engineered) in a fashion that generally does not occur in nature. A particular example of a recombinant vector, such as an AAV vector would be where a polynucleotide that is not normally present in the wild-type viral (e.g., AAV) genome is inserted within the viral genome. An example of a recombinant polynucleotide would be where a CpG reduced nucleic acid encoding a FVIII or hFVIII-BDD protein is cloned into a vector, with or without 5′, 3′ and/or intron regions that the gene is normally associated within the viral (e.g., AAV) genome. Although the term “recombinant” is not always used herein in reference to vectors, such as viral and AAV vectors, as well as sequences such as polynucleotides, recombinant forms including polynucleotides, are expressly included in spite of any such omission.

A recombinant viral “vector” or “AAV vector” is derived from the wild type genome of a virus, such as AAV by using molecular methods to remove the wild type genome from the virus (e.g., AAV), and replacing with a non-native nucleic acid, such as a CpG reduced nucleic acid encoding FVIII. Typically, for AAV one or both inverted terminal repeat (ITR) sequences of AAV genome are retained in the AAV vector. A “recombinant” viral vector (e.g., AAV) is distinguished from a viral (e.g., AAV) genome, since all or a part of the viral genome has been replaced with a non-native sequence with respect to the viral (e.g., AAV) genomic nucleic acid such as a CpG reduced nucleic acid encoding FVIII or hFVIII-BDD. Incorporation of a non-native sequence therefore defines the viral vector (e.g., AAV) as a “recombinant” vector, which in the case of AAV can be referred to as a “rAAV vector.”

A recombinant vector (e.g., lenti-, parvo-, AAV) sequence can be packaged-referred to herein as a “particle” for subsequent infection (transduction) of a cell, ex vivo, in vitro or in vivo. Where a recombinant vector sequence is encapsidated or packaged into an AAV particle, the particle can also be referred to as a “rAAV.” Such particles include proteins that encapsidate or package the vector genome. Particular examples include viral envelope proteins, and in the case of AAV, capsid proteins.

A vector “genome” refers to the portion of the recombinant plasmid sequence that is ultimately packaged or encapsidated to form a viral (e.g., AAV) particle. In cases where recombinant plasmids are used to construct or manufacture recombinant vectors, the vector genome does not include the portion of the “plasmid” that does not correspond to the vector genome sequence of the recombinant plasmid. This non vector genome portion of the recombinant plasmid is referred to as the “plasmid backbone,” which is important for cloning and amplification of the plasmid, a process that is needed for propagation and recombinant virus production, but is not itself packaged or encapsidated into virus (e.g., AAV) particles. Thus, a vector “genome” refers to the nucleic acid that is packaged or encapsidated by virus (e.g., AAV).

A “transgene” is used herein to conveniently refer to a nucleic acid that is intended or has been introduced into a cell or organism. Transgenes include any nucleic acid, such as a gene that encodes a polypeptide or protein (e.g., a CpG reduced nucleic acid encoding FVIII or hFVIII-BDD).

In a cell having a transgene, the transgene has been introduced/transferred by way of vector, such as AAV, “transduction” or “transfection” of the cell. The terms “transduce” and “transfect” refer to introduction of a molecule such as a nucleic acid into a cell or host organism. The transgene may or may not be integrated into genomic nucleic acid of the recipient cell. If an introduced nucleic acid becomes integrated into the nucleic acid (genomic DNA) of the recipient cell or organism it can be stably maintained in that cell or organism and further passed on to or inherited by progeny cells or organisms of the recipient cell or organism. Finally, the introduced nucleic acid may exist in the recipient cell or host organism extrachromosomally, or only transiently.

A “transduced cell” is a cell into which the transgene has been introduced. Accordingly, a “transduced” cell (e.g., in a mammal, such as a cell or tissue or organ cell), means a genetic change in a cell following incorporation of an exogenous molecule, for example, a nucleic acid (e.g., a transgene) into the cell. Thus, a “transduced” cell is a cell into which, or a progeny thereof in which an exogenous nucleic acid has been introduced. The cell(s) can be propagated and the introduced protein expressed, or nucleic acid transcribed. For gene therapy uses and methods, a transduced cell can be in a subject.

An “expression control element” refers to nucleic acid sequence(s) that influence expression of an operably linked nucleic acid. Control elements, including expression control elements as set forth herein such as promoters and enhancers, Vector sequences including AAV vectors can include one or more “expression control elements.” Typically, such elements are included to facilitate proper heterologous polynucleotide transcription and if appropriate translation (e.g., a promoter, enhancer, splicing signal for introns, maintenance of the correct reading frame of the gene to permit in-frame translation of mRNA and, stop codons etc.). Such elements typically act in cis, referred to as a “cis acting” element, but may also act in trans.

Expression control can be at the level of transcription, translation, splicing, message stability, etc. Typically, an expression control element that modulates transcription is juxtaposed near the 5′ end (i.e., “upstream”) of a transcribed nucleic acid. Expression control elements can also be located at the 3′ end (i.e., “downstream”) of the transcribed sequence or within the transcript (e.g., in an intron). Expression control elements can be located adjacent to or at a distance away from the transcribed sequence (e.g., 1-10, 10-25, 25-50, 50-100, 100 to 500, or more nucleotides from the polynucleotide), even at considerable distances. Nevertheless, owing to the length limitations of certain vectors, such as AAV vectors, expression control elements will typically be within 1 to 1000 nucleotides from the transcribed nucleic acid.

Functionally, expression of operably linked nucleic acid is at least in part controllable by the element (e.g., promoter) such that the element modulates transcription of the nucleic acid and, as appropriate, translation of the transcript. A specific example of an expression control element is a promoter, which is usually located 5′ of the transcribed sequence e.g., a CpG reduced nucleic acid encoding FVIII or hFVIII-BDD. A promoter typically increases an amount expressed from operably linked nucleic acid as compared to an amount expressed when no promoter exists.

An “enhancer” as used herein can refer to a sequence that is located adjacent to the heterologous polynucleotide. Enhancer elements are typically located upstream of a promoter element but also function and can be located downstream of or within a sequence (e.g., a CpG reduced nucleic acid encoding FVIII or hFVIII-BDD). Hence, an enhancer element can be located 100 base pairs, 200 base pairs, or 300 or more base pairs upstream or downstream of a CpG reduced nucleic acid encoding FVIII. Enhancer elements typically increase expressed of an operably linked nucleic acid above expression afforded by a promoter element.

An expression construct may comprise regulatory elements which serve to drive expression in a particular cell or tissue type. Expression control elements (e.g., promoters) include those active in a particular tissue or cell type, referred to herein as a “tissue-specific expression control elements/promoters.” Tissue-specific expression control elements are typically active in specific cell or tissue (e.g., liver). Expression control elements are typically active in particular cells, tissues or organs because they are recognized by transcriptional activator proteins, or other regulators of transcription, that are unique to a specific cell, tissue or organ type. Such regulatory elements are known to those of skill in the art (see, e.g., Sambrook et al. (1989) and Ausubel et al. (1992)).

The incorporation of tissue specific regulatory elements in the expression constructs of the invention provides for at least partial tissue tropism for the expression of a CpG reduced nucleic acid encoding FVIII or hFVIII-BDD. Examples of promoters that are active in liver are the TTR promoter, human alpha 1-antitrypsin (hAAT) promoter; albumin, Miyatake, et al. J. Virol., 71:5124-32(1997); hepatitis B virus core promoter, Sandig, et al., Gene Ther. 3:1002-9 (1996); alpha-fetoprotein (AFP), Arbuthnot, et al., Hum. Gene. Ther., 7:1503-14 (1996)], among others. An example of an enhancer active in liver is apolipoprotein E (apoE) HCR-1 and HCR-2 (Allan et al., J. Biol. Chem., 272:29113-19 (1997)).

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences and the other viral promoters/enhancers active in a variety of mammalian cell types, or synthetic elements that are not present in nature (see, e.g., Boshart et al, Cell, 41:521-530 (1985)), the SV40 promoter, the dihydrofolate reductase promoter, the cytoplasmic B-actin promoter and the phosphoglycerol kinase (PGK) promoter.

Expression control elements also can confer expression in a manner that is regulatable, that is, a signal or stimuli increases or decreases expression of the operably linked heterologous polynucleotide. A regulatable element that increases expression of the operably linked polynucleotide in response to a signal or stimuli is also referred to as an “inducible element” (i.e., is induced by a signal). Particular examples include, but are not limited to, a hormone (e.g., steroid) inducible promoter. Typically, the amount of increase or decrease conferred by such elements is proportional to the amount of signal or stimuli present; the greater the amount of signal or stimuli, the greater the increase or decrease in expression. Particular non-limiting examples include zinc-inducible sheep metallothionine (MT) promoter; the steroid hormone-inducible mouse mammary tumor virus (MMTV) promoter; the T7 polymerase promoter system (WO 98/10088); the tetracycline-repressible system (Gossen, et al., Proc. Natl. Acad. Sci. USA, 89:5547-5551 (1992)); the tetracycline-inducible system (Gossen, et al., Science. 268:1766-1769 (1995); see also Harvey, et al., Curr. Opin. Chem. Biol. 2:512-518 (1998)); the RU486-inducible system (Wang, et al., Nat. Biotech. 15:239-243 (1997) and Wang, et al., Gene Ther. 4:432-441 (1997)]; and the rapamycin-inducible system (Magari, et al., J. Clin. Invest. 100:2865-2872 (1997); Rivera, et al., Nat. Medicine. 2:1028-1032 (1996)). Other regulatable control elements which may be useful in this context are those which are regulated by a specific physiological state, e.g., temperature, acute phase, development.

Expression control elements also include the native elements(s) for the heterologous polynucleotide. A native control element (e.g., promoter) may be used when it is desired that expression of the heterologous polynucleotide should mimic the native expression. The native element may be used when expression of the heterologous polynucleotide is to be regulated temporally or developmentally, or in a tissue-specific manner, or in response to specific transcriptional stimuli. Other native expression control elements, such as introns, polyadenylation sites or Kozak consensus sequences may also be used.

The term “operably linked” means that the regulatory sequences necessary for expression of a coding sequence are placed in the appropriate positions relative to the coding sequence so as to effect expression of the coding sequence. This same definition is sometimes applied to the arrangement of coding sequences and transcription control elements (e.g. promoters, enhancers, and termination elements) in an expression vector. This definition is also sometimes applied to the arrangement of nucleic acid sequences of a first and a second nucleic acid molecule wherein a hybrid nucleic acid molecule is generated.

In the example of an expression control element in operable linkage with a nucleic acid, the relationship is such that the control element modulates expression of the nucleic acid. More specifically, for example, two DNA sequences operably linked means that the two DNAs are arranged (cis or trans) in such a relationship that at least one of the DNA sequences is able to exert a physiological effect upon the other sequence.

Accordingly, additional elements for vectors include, without limitation, an expression control (e.g., promoter/enhancer) element, a transcription termination signal or stop codon, 5′ or 3′ untranslated regions (e.g., polyadenylation (polyA) sequences) which flank a sequence, such as one or more copies of an AAV ITR sequence, or an intron.

Further elements include, for example, filler or stuffer polynucleotide sequences, for example to improve packaging and reduce the presence of contaminating nucleic acid. AAV vectors typically accept inserts of DNA having a size range which is generally about 4 kb to about 5.2 kb, or slightly more. Thus, for shorter sequences, inclusion of a stuffer or filler in order to adjust the length to near or at the normal size of the virus genomic sequence acceptable for AAV vector packaging into virus particle. In various embodiments, a filler/stuffer nucleic acid sequence is an untranslated (non-protein encoding) segment of nucleic acid. For a nucleic acid sequence less than 4.7 Kb, the filler or stuffer polynucleotide sequence has a length that when combined (e.g., inserted into a vector) with the sequence has a total length between about 3.0-5.5 Kb, or between about 4.0-5.0 Kb, or between about 4.3-4.8 Kb.

An intron can also function as a filler or stuffer polynucleotide sequence in order to achieve a length for AAV vector packaging into a virus particle. Introns and intron fragments that function as a filler or stuffer polynucleotide sequence also can enhance expression.

The phrase “hemostasis related disorder” refers to bleeding disorders such as hemophilia A, hemophilia A patients with inhibitory antibodies, deficiencies in coagulation Factors, VII, VIII, IX and X, XI, V, XII, II, von Willebrand factor, combined FV/FVIII deficiency, vitamin K epoxide reductase C1 deficiency, gamma-carboxylase deficiency; bleeding associated with trauma, injury, thrombosis, thrombocytopenia, stroke, coagulopathy, disseminated intravascular coagulation (DIC); over-anticoagulation associated with heparin, low molecular weight heparin, pentasaccharide, warfarin, small molecule antithrombotics (i.e. FXa inhibitors); and platelet disorders such as, Bernard Soulier syndrome, Glanzman thromblastemia, and storage pool deficiency.

The term “isolated,” when used as a modifier of a composition, means that the compositions are made by the hand of man or are separated, completely or at least in part, from their naturally occurring in vivo environment. Generally, isolated compositions are substantially free of one or more materials with which they normally associate with in nature, for example, one or more protein, nucleic acid, lipid, carbohydrate, cell membrane.

With reference to nucleic acids of the invention, the term “isolated” refers to a nucleic acid molecule that is separated from one or more sequences with which it is immediately contiguous (in the 5′ and 3′ directions) in the naturally occurring genome (genomic DNA) of the organism from which it originates. For example, the “isolated nucleic acid” may comprise a DNA or cDNA molecule inserted into a vector, such as a plasmid or virus vector, or integrated into the DNA of a prokaryote or eukaryote.

With respect to RNA molecules of the invention, the term “isolated” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the term “substantially pure” is defined below).

With respect to protein, the term “isolated protein” or “isolated and purified protein” is sometimes used herein. This term refers primarily to a protein produced by expression of an isolated nucleic acid molecule. Alternatively, this term may refer to a protein which has been sufficiently separated from other proteins with which it would naturally be associated, so as to exist in “substantially pure” form.

The term “isolated” does not exclude combinations produced by the hand of man, for example, a recombinant vector (e.g., rAAV) sequence, or virus particle that packages or encapsidates a vector genome and a pharmaceutical formulation. The term “isolated” also does not exclude alternative physical forms of the composition, such as hybrids/chimeras, multimers/oligomers, modifications (e.g., phosphorylation, glycosylation, lipidation) or derivatized forms, or forms expressed in host cells produced by the hand of man.

The term “substantially pure” refers to a preparation comprising at least 50-60% by weight the compound of interest (e.g., nucleic acid, oligonucleotide, protein, etc.). The preparation can comprise at least 75% by weight, or about 90-99% by weight, of the compound of interest. Purity is measured by methods appropriate for the compound of interest (e.g. chromatographic methods, agarose or polyacrylamide gel electrophoresis, HPLC analysis, and the like).

The phrase “consisting essentially of” when referring to a particular nucleotide sequence or amino acid sequence means a sequence having the properties of a given SEQ ID NO. For example, when used in reference to an amino acid sequence, the phrase includes the sequence per se and molecular modifications that would not affect the basic and novel characteristics of the sequence.

The term “oligonucleotide,” as used herein refers to primers and probes, and is defined as a nucleic acid molecule comprised of two or more ribo- or deoxyribonucleotides, such as more than three. The exact size of the oligonucleotide will depend on various factors and on the particular application for which the oligonucleotide is used.

The term “probe” as used herein refers to an oligonucleotide, polynucleotide or nucleic acid, either RNA or DNA, whether occurring naturally as in a purified restriction enzyme digest or produced synthetically, which is capable of annealing with or specifically hybridizing to a nucleic acid with sequences complementary to the probe. A probe may be either single-stranded or double-stranded. The exact length of the probe will depend upon many factors, including temperature, source of probe and method of use. For example, for diagnostic applications, depending on the complexity of the target sequence, the oligonucleotide probe typically contains 15-25 or more nucleotides, although it may contain fewer nucleotides.

The probes herein are selected to be “substantially” complementary to different strands of a particular target nucleic acid sequence. This means that the probes must be sufficiently complementary so as to be able to “specifically hybridize” or anneal with their respective target strands under a set of pre-determined conditions. Therefore, the probe sequence need not reflect the exact complementary sequence of the target. For example, a non-complementary nucleotide fragment may be attached to the 5′ or 3′ end of the probe, with the remainder of the probe sequence being complementary to the target strand. Alternatively, non-complementary bases or longer sequences can be interspersed into the probe, provided that the probe sequence has sufficient complementarity with the sequence of the target nucleic acid to anneal therewith specifically.

The term “specifically hybridize” refers to the association between two single-stranded nucleic acid molecules of sufficiently complementary sequence to permit such hybridization under pre-determined conditions generally used in the art (sometimes termed “substantially complementary”). In particular, the term refers to hybridization of an oligonucleotide with a substantially complementary sequence contained within a single-stranded DNA or RNA molecule of the invention, to the substantial exclusion of hybridization of the oligonucleotide with single-stranded nucleic acids of non-complementary sequence.

The term “primer” as used herein refers to an oligonucleotide, either RNA or DNA, either single-stranded or double-stranded, either derived from a biological system, generated by restriction enzyme digestion, or produced synthetically which, when placed in the proper environment, is able to act functionally as an initiator of template-dependent nucleic acid synthesis. When presented with an appropriate nucleic acid template, suitable nucleoside triphosphate precursors of nucleic acids, a polymerase enzyme, suitable cofactors and conditions such as a suitable temperature and pH, the primer may be extended at its 3′ terminus by the addition of nucleotides by the action of a polymerase or similar activity to yield a primer extension product.

The primer may vary in length depending on the particular conditions and requirements of the application. For example, in diagnostic applications, the oligonucleotide primer is typically 15-25 or more nucleotides in length. The primer must be of sufficient complementarity to the desired template to prime the synthesis of the desired extension product, that is, to be able to anneal with the desired template strand in a manner sufficient to provide the 3′ hydroxyl moiety of the primer in appropriate juxtaposition for use in the initiation of synthesis by a polymerase or similar enzyme. It is not required that the primer sequence represent an exact complement of the desired template. For example, a non-complementary nucleotide sequence may be attached to the 5′ end of an otherwise complementary primer. Alternatively, non-complementary bases may be interspersed within the oligonucleotide primer sequence, provided that the primer sequence has sufficient complementarity with the sequence of the desired template strand to functionally provide a template-primer complex for the synthesis of the extension product.

The term “identity,” “homology” and grammatical variations thereof, mean that two or more referenced entities are the same, when they are “aligned” sequences. Thus, by way of example, when two polypeptide sequences are identical, they have the same amino acid sequence, at least within the referenced region or portion. Where two polynucleotide sequences are identical, they have the same polynucleotide sequence, at least within the referenced region or portion. The identity can be over a defined area (region or domain) of the sequence. An “area” or “region” of identity refers to a portion of two or more referenced entities that are the same. Thus, where two protein or nucleic acid sequences are identical over one or more sequence areas or regions they share identity within that region. An “aligned” sequence refers to multiple polynucleotide or protein (amino acid) sequences, often containing corrections for missing or additional bases or amino acids (gaps) as compared to a reference sequence.

The identity can extend over the entire length or a portion of the sequence. In certain embodiments, the length of the sequence sharing the percent identity is 2, 3, 4, 5 or more contiguous nucleic acids or amino acids, e.g., 6, 7, 8, 9, 10, 11, 12, 13, 14, 15, 16, 17, 18, 19, 20, etc. contiguous nucleic acids or amino acids. In additional embodiments, the length of the sequence sharing identity is 21 or more contiguous nucleic acids or amino acids, e.g., 21, 22, 23, 24, 25, 26, 27, 28, 29, 30, 31, 32, 33, 34, 35, 36, 37, 38, 39, 40, etc. contiguous nucleic acids or amino acids. In further embodiments, the length of the sequence sharing identity is 41 or more contiguous nucleic acids or amino acids, e.g. 42, 43, 44, 45, 45, 47, 48, 49, 50, etc., contiguous nucleic acids or amino acids. In yet further embodiments, the length of the sequence sharing identity is 50 or more contiguous nucleic acids or amino acids, e.g., 50-55, 55-60, 60-65, 65-70, 70-75, 75-80, 80-85, 85-90, 90-95, 95-100, 100-150, 150-200, 200-250, 250-300, 300-500, 500-1,000, etc. contiguous nucleic acids or amino acids.

As set forth herein, nucleic acid variants such as CpG reduced variants encoding FVIII or hFVIII-BDD will be distinct from wild-type but may exhibit sequence identity with wild-type FVIII protein with, or without B-domain. In CpG reduced nucleic acid variants encoding FVIII or hFVIII-BDD, at the nucleotide sequence level, a CpG reduced nucleic acid encoding FVIII or hFVIII-BDD will typically be at least about 70% identical, more typically about 75% identical, even more typically about 80%-85% identical to wild-type FVIII encoding nucleic acid. Thus, for example, a CpG reduced nucleic acid encoding FVIII or hFVIII-BDD may have 75%-85% identity to wild-type FVIII encoding gene, or to each other, i.e., X01 vs. X02, X03 vs. X04, etc. as set forth herein.

At the amino acid sequence level, a variant such as a variant FVIII or hFVIII-BDD protein will be at least about 70% identical, more typically about 75% identical, or 80% identical, even more typically about 85 identity, or 90% or more identity. In other embodiments, a variant such as a variant FVIII or hFVIII-BDD protein has at least 90%, 91%, 92%, 93%, 94%, 95%, 96%, 97%, 98%, 99% or more identity to a reference sequence, e.g. wild-type FVIII protein with or without B-domain.

To determine identity, if the FVIII (e.g., CpG reduced nucleic acid encoding FVIII) retains the B-domain, it is appropriate to compare identity to wild-type FVIII. If the FVIII (e.g., CpG reduced nucleic acid encoding hFVIII-BDD) has a B-domain deletion, it is appropriate to compare identity to wild-type FVIII that also has a B-domain deletion.

The terms “homologous” or “homology” mean that two or more referenced entities share at least partial identity over a given region or portion. “Areas, regions or domains” of homology or identity mean that a portion of two or more referenced entities share homology or are the same. Thus, where two sequences are identical over one or more sequence regions they share identity in these regions. “Substantial homology” means that a molecule is structurally or functionally conserved such that it has or is predicted to have at least partial structure or function of one or more of the structures or functions (e.g., a biological function or activity) of the reference molecule, or relevant/corresponding region or portion of the reference molecule to which it shares homology.

The extent of identity (homology) or “percent identity” between two sequences can be ascertained using a computer program and/or mathematical algorithm. For purposes of this invention comparisons of nucleic acid sequences are performed using the GCG Wisconsin Package version 9.1, available from the Genetics Computer Group in Madison, Wisconsin. For convenience, the default parameters (gap creation penalty=12, gap extension penalty=4) specified by that program are intended for use herein to compare sequence identity. Alternately, the Blastn 2.0 program provided by the National Center for Biotechnology Information (found on the world wide web at ncbi.nlm.nih.gov/blast/; Altschul et al., 1990, J Mol Biol 215:403-410) using a gapped alignment with default parameters, may be used to determine the level of identity and similarity between nucleic acid sequences and amino acid sequences. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)). Programs for quantitating protein structural similarity using Delaunay-based topological mapping have also been developed (Bostick et al., Biochem Biophys Res Commun. 304:320 (2003)).

Nucleic acid molecules, expression vectors (e.g., vector genomes), plasmids, including nucleic acids and nucleic acid variants encoding FVIII or hFVIII-BDD of the invention may be prepared by using recombinant DNA technology methods. The availability of nucleotide sequence information enables preparation of isolated nucleic acid molecules of the invention by a variety of means. For example, CpG reduced nucleic acid variants encoding FVIII or hFVIII-BDD can be made using various standard cloning, recombinant DNA technology, via cell expression or in vitro translation and chemical synthesis techniques. Purity of polynucleotides can be determined through sequencing, gel electrophoresis and the like. For example, nucleic acids can be isolated using hybridization or computer-based database screening techniques. Such techniques include, but are not limited to: (1) hybridization of genomic DNA or cDNA libraries with probes to detect homologous nucleotide sequences; (2) antibody screening to detect polypeptides having shared structural features, for example, using an expression library; (3) polymerase chain reaction (PCR) on genomic DNA or cDNA using primers capable of annealing to a nucleic acid sequence of interest; (4) computer searches of sequence databases for related sequences; and (5) differential screening of a subtracted nucleic acid library.

Nucleic acids of the invention may be maintained as DNA in any convenient cloning vector. In a one embodiment, clones are maintained in a plasmid cloning/expression vector, such as pBluescript (Stratagene, La Jolla, CA), which is propagated in a suitable E. coli host cell. Alternatively, nucleic acids may be maintained in vector suitable for expression in mammalian cells. In cases where post-translational modification affects coagulation function, nucleic acid molecule can be expressed in mammalian cells.

Nucleic acids and nucleic acid variants encoding FVIII or hFVIII-BDD include cDNA, genomic DNA, RNA, and fragments thereof which may be single- or double-stranded. Thus, this invention provides oligonucleotides (sense or antisense strands of DNA or RNA) having sequences capable of hybridizing with at least one sequence of a nucleic acid of the invention. Such oligonucleotides are useful as probes for detecting FVIII or hFVIII-BDD expression.

Vectors such as those described herein (rAAV) optionally comprise regulatory elements necessary for expression of the DNA in the host cell positioned in such a manner as to permit expression of the encoded protein in the host cell. Such regulatory elements required for expression include, but are not limited to, promoter sequences, enhancer sequences and transcription initiation sequences as set forth herein and known to the skilled artisan.

Methods and uses of the invention of the invention include delivering (transducing) nucleic acid (transgene) into host cells, including dividing and/or non-dividing cells. The nucleic acids, rAAV vector, methods, uses and pharmaceutical formulations of the invention are additionally useful in a method of delivering, administering or providing a FVIII or hFVIII-BDD to a subject in need thereof, as a method of treatment. In this manner, the nucleic acid is transcribed and the protein may be produced in vivo in a subject. The subject may benefit from or be in need of the FVIII or hFVIII-BDD because the subject has a deficiency of FVIII, or because production of FVIII in the subject may impart some therapeutic effect, as a method of treatment or otherwise.

rAAV vectors comprising a genome with a nucleic acid or nucleic acid variant encoding FVIII or hFVIII-BDD permit the treatment of genetic diseases, e.g., a FVIII deficiency. For deficiency state diseases, gene transfer can be used to bring a normal gene into affected tissues for replacement therapy, as well as to create animal models for the disease using antisense mutations. For unbalanced disease states, gene transfer could be used to create a disease state in a model system, which could then be used in efforts to counteract the disease state. The use of site-specific integration of nucleic acid sequences to correct defects is also possible.

In particular embodiments, rAAV vectors comprising a genome with a nucleic acid or nucleic acid variant encoding FVIII or hFVIII-BDD may be used, for example, as therapeutic and/or prophylactic agents (protein or nucleic acid) which modulate the blood coagulation cascade or as a transgene in gene. For example, an encoded FVIII or hFVIII-BDD may have similar coagulation activity as wild-type FVIII, or altered coagulation activity compared to wild-type FVII. Cell-based strategies allow continuous expression of FVIII or hFVIII-BDD in hemophilia A patients. As disclosed herein, certain modifications of FVIII molecules (nucleic acid and protein) result in increased expression at the nucleic acid level, increased coagulation activity thereby effectively improving hemostasis.

Administration of FVIII or hFVIII-BDD-encoding rAAV vectors to a patient results in the expression of FVIII or hFVIII-BDD protein which serves to alter the coagulation cascade. In accordance with the invention, expression of FVIII or hFVIII-BDD protein as described herein, or a functional fragment, increases hemostasis.

rAAV vectors may be administered alone, or in combination with other molecules useful for modulating hemostasis. According to the invention, rAAV vectors or a combination of therapeutic agents may be administered to the patient alone or in a pharmaceutically acceptable or biologically compatible compositions.

Adeno-associated viruses” (AAV) are in the parvovirus family. AAV are viruses useful as gene therapy vectors as they can penetrate cells and introduce nucleic acid/genetic material so that the nucleic acid/genetic material may be stably maintained in cells. In addition, these viruses can introduce nucleic acid/genetic material into specific sites, for example. Because AAV are not associated with pathogenic disease in humans, rAAV vectors are able to deliver heterologous polynucleotide sequences (e.g., therapeutic proteins and agents) to human patients without causing substantial AAV pathogenesis or disease.

rAAV vectors possess a number of desirable features for such applications, including tropism for dividing and non-dividing cells. Early clinical experience with these vectors also demonstrated no sustained toxicity and immune responses were minimal or undetectable. AAV are known to infect a wide variety of cell types in vivo and in vitro by receptor-mediated endocytosis or by transcytosis. These vector systems have been tested in humans targeting retinal epithelium, liver, skeletal muscle, airways, brain, joints and hematopoietic stem cells.

It may be desirable to introduce a rAAV vector that can provide, for example, multiple copies of a desired gene and hence greater amounts of the product of that gene. Improved rAAV vectors and methods for producing these vectors have been described in detail in a number of references, patents, and patent applications, including: Wright J. F. (Hum Gene Ther 20:698-706, 2009) a technology used for the production of clinical grade vector at Children's Hospital of Philadelphia.

Accordingly, the invention provides virmethods for delivery of FVIII or hFVIII-BDD by way of a rAAV vector. For example, a recombinant AAV vector can include a nucleic acid variant encoding FVIII, where the encoded FVIII protein optionally has B-domain deletion. rAAV vector delivery or administration to a subject (e.g., mammal) therefore provides FVIII to a subject such as a mammal (e.g., human).

Direct delivery of vectors or ex-vivo transduction of human cells followed by infusion into the body will result in FVIII or hFVIII-BDD expression thereby exerting a beneficial therapeutic effect on hemostasis. In the context of invention Factor VIII described herein, such administration enhances pro-coagulation activity.

AAV vectors vectors do not typically include viral genes associated with pathogenesis. Such vectors typically have one or more of the wild type AAV genes deleted in whole or in part, for example, rep and/or cap genes, but retain at least one functional flanking ITR sequence, as necessary for the rescue, replication, and packaging of the recombinant vector into an AAV vector particle. For example, only the essential parts of vector e.g., the ITR elements, respectively are included. An AAV vector genome would therefore include sequences required in cis for replication and packaging (e.g., functional ITR sequences)

Recombinant AAV vector, as well as methods and uses thereof, include any viral strain or serotype. As a non-limiting example, a recombinant AAV vector can be based upon any AAV genome, such as AAV-1, -2, -3, -4, -5, -6, -7, -8, -9, -10, -11, -12, -rh74, -rh 10 or AAV-218, for example. Such vectors can be based on the same strain or serotype (or subgroup or variant), or be different from each other. As a non-limiting example, a recombinant AAV vector based upon one serotype genome can be identical to one or more of the capsid proteins that package the vector. In addition, a recombinant AAV vector genome can be based upon an AAV (e.g., AAV2) serotype genome distinct from one or more of the AAV capsid proteins that package the vector. For example, the AAV vector genome can be based upon AAV2, whereas at least one of the three capsid proteins could be a AAV1, AAV3, AAV4, AAV5, AAV6, AAV7, AAV8, AAV9, AAV10, AAV11, AAV12, Rh10, Rh74 or AAV-218 or variant thereof, for example.

In particular embodiments, adeno-associated virus (AAV) vectors include AAV1, AAV2, AAV3, AAV4, AAV5, AAV6, AAV7, AAV8, AAV9, AAV10, AAV11, AAV12, Rh10, Rh74 and AAV-218, as well as variants (e.g., capsid variants, such as amino acid insertions, additions, substitutions and deletions) thereof, for example, as set forth in WO 2013/158879 (International Application PCT/US2013/037170), WO 2015/013313 (International Application PCT/US2014/047670) and US 2013/0059732 (U.S. Pat. No. 9,169,299, discloses LK01, LK02, LK03, etc.).

AAV variants include variants and chimeras of AAV1, AAV2, AAV3, AAV4, AAV5, AAV6, AAV7, AAV8, AAV9, AAV10, AAV11, AAV12, Rh10, Rh74 and AAV-218 capsid. Accordingly, AAV vectors and AAV variants (e.g., capsid variants) that include (encapsidate or package) nucleic acid or nucleic acid variant encoding FVIII or hFVIII-BDD.

AAV and AAV variants (e.g., capsid variants) serotypes (e.g., VP1, VP2, and/or VP3 sequences) may or may not be distinct from other AAV serotypes, including, for example, AAV1-AAV12, Rh74 or Rh10 (e.g., distinct from VP1, VP2, and/or VP3 sequences of any of AAV1-AAV12, Rh74 or Rh10 serotypes).

As used herein, the term “serotype” is a distinction used to refer to an AAV having a capsid that is serologically distinct from other AAV serotypes. Serologic distinctiveness is determined on the basis of the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes). Despite the possibility that AAV variants including capsid variants may not be serologically distinct from a reference AAV or other AAV serotype, they differ by at least one nucleotide or amino acid residue compared to the reference or other AAV serotype.

Under the traditional definition, a serotype means that the virus of interest has been tested against serum specific for all existing and characterized serotypes for neutralizing activity and no antibodies have been found that neutralize the virus of interest. As more naturally occurring virus isolates of are discovered and/or capsid mutants generated, there may or may not be serological differences with any of the currently existing serotypes. Thus, in cases where the new virus (e.g., AAV) has no serological difference, this new virus (e.g., AAV) would be a subgroup or variant of the corresponding serotype. In many cases, serology testing for neutralizing activity has yet to be performed on mutant viruses with capsid sequence modifications to determine if they are of another serotype according to the traditional definition of serotype. Accordingly, for the sake of convenience and to avoid repetition, the term “serotype” broadly refers to both serologically distinct viruses (e.g., AAV) as well as viruses (e.g., AAV) that are not serologically distinct that may be within a subgroup or a variant of a given serotype.

AAV vectors therefore include gene/protein sequences identical to gene/protein sequences characteristic for a particular serotype. As used herein, an “AAV vector related to AAV1” refers to one or more AAV proteins (e.g., VP1, VP2, and/or VP3 sequences) that has substantial sequence identity to one or more polynucleotides or polypeptide sequences that comprise AAV1. Analogously, an “AAV vector related to AAV8” refers to one or more AAV proteins (e.g., VP1, VP2, and/or VP3 sequences) that has substantial sequence identity to one or more polynucleotides or polypeptide sequences that comprise AAV8. An “AAV vector related to AAV-Rh74” refers to one or more AAV proteins (e.g., VP1, VP2, and/or VP3 sequences) that has substantial sequence identity to one or more polynucleotides or polypeptide sequences that comprise AAV-Rh74. Such AAV vectors related to another serotype, e.g., AAV1, AAV2, AAV3, AAV4, AAV5, AAV6, AAV7, AAV8, AAV9, AAV10, AAV11, AAV12, Rh10, Rh74 or AAV-218, can therefore have one or more distinct sequences from AAV1, AAV2, AAV3, AAV4, AAV5, AAV6, AAV7, AAV8, AAV9, AAV10, AAV11, AAV12, Rh10, Rh74 and AAV-218, but can exhibit substantial sequence identity to one or more genes and/or proteins, and/or have one or more functional characteristics of AAV1, AAV2, AAV3, AAV4, AAV5, AAV6, AAV7, AAV8, AAV9, AAV10, AAV11, AAV12, Rh10, Rh74 or AAV-218 (e.g., such as cell/tissue tropism). Exemplary non-limiting AAV variants include capsid variants of any of VP1, VP2, and/or VP3.

In various exemplary embodiments, an AAV vector related to a reference serotype has a polynucleotide, polypeptide or subsequence thereof that includes or consists of a sequence at least 80% or more (e.g., 85%, 90%, 95%, 96%, 97%, 98%, 99%, 99.1%, 99.2%, 99.3%, 99.4%, 99.5%, etc.) identical to one or more AAV1, AAV2, AAV3, AAV4, AAV5, AAV6, AAV7, AAV8, AAV9, AAV10, AAV11, AAV12, Rh10, Rh74 or AAV-218 (e.g., such as an ITR, or a VP1, VP2, and/or VP3 sequences).

Compositions, methods and uses of the invention include AAV sequences (polypeptides and nucleotides), and subsequences thereof that exhibit less than 100% sequence identity to a reference AAV serotype such as AAV1, AAV2, AAV3, AAV4, AAV5, AAV6, AAV7, AAV8, AAV9, AAV10, AAV11, AAV12, Rh10, or AAV-218, but are distinct from and not identical to known AAV genes or proteins, such as AAV1, AAV2, AAV3, AAV4, AAV5, AAV6, AAV7, AAV8, AAV9, AAV10, AAV11, AAV12, Rh10, Rh74 or AAV-2i8, genes or proteins, etc. In one embodiment, an AAV polypeptide or subsequence thereof includes or consists of a sequence at least 75% or more identical, e.g., 80%, 85%, 85%, 87%, 88%, 89%, 90%, 91%, 92%, 93%, 94%, 95%, 96%, 97%, 98%, 99%, 99.1%, 99.2%, 99.3%, 99.4%, 99.5%, etc., up to 100% identical to any reference AAV sequence or subsequence thereof, such as AAV1, AAV2, AAV3, AAV4, AAV5, AAV6, AAV7, AAV8, AAV9, AAV10, AAV11, AAV12, Rh10, Rh74 or AAV-218 (e.g., VP1, VP2 and/or VP3 capsid or ITR). In certain embodiments, an AAV variant has 1, 2, 3, 4, 5, 5-10, 10-15, 15-20 or more amino acid substitutions.

Recombinant AAV vectors, including AAV1, AAV2, AAV3, AAV4, AAV5, AAV6, AAV7, AAV8, AAV9, AAV10, AAV11, AAV12, Rh10, Rh74 or AAV-218 and variant, related, hybrid and chimeric sequences, can be constructed using recombinant techniques that are known to the skilled artisan, to include one or more nucleic acid sequences (transgenes) flanked with one or more functional AAV ITR sequences.

In one embodiment of the invention, rAAV vector comprising a nucleic acid or variant encoding FVIII or hFVIII-BDD, may be administered to a patient via infusion in a biologically compatible carrier, for example, via intravenous injection. The rAAV vectors may optionally be encapsulated into liposomes or mixed with other phospholipids or micelles to increase stability of the molecule.

In accordance with the invention, rAAV vectors may be administered alone or in combination with other agents known to modulate hemostasis (e.g., Factor V, Factor Va or derivatives thereof).

Accordingly, rAAV vectors and other compositions, agents, drugs, biologics (proteins) can be incorporated into pharmaceutical compositions. Such pharmaceutical compositions are useful for, among other things, administration and delivery to a subject in vivo or ex vivo.

In particular embodiments, pharmaceutical compositions also contain a pharmaceutically acceptable carrier or excipient. Such excipients include any pharmaceutical agent that does not itself induce an immune response harmful to the individual receiving the composition, and which may be administered without undue toxicity.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. A “pharmaceutically acceptable” or “physiologically acceptable” composition is a material that is not biologically or otherwise undesirable, e.g., the material may be administered to a subject without causing substantial undesirable biological effects. Thus, such a pharmaceutical composition may be used, for example in administering a nucleic acid, vector, viral particle or protein to a subject.

Pharmaceutically acceptable excipients include, but are not limited to, liquids such as water, saline, glycerol, sugars and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles.

The pharmaceutical composition may be provided as a salt and can be formed with many acids, including but not limited to, hydrochloric, sulfuric, acetic, lactic, tartaric, malic, succinic, etc. Salts tend to be more soluble in aqueous or other protonic solvents than are the corresponding, free base forms. In other cases, a preparation may be a lyophilized powder which may contain any or all of the following: 1-50 mM histidine, 0.1%-2% sucrose, and 2-7% mannitol, at a pH range of 4.5 to 5.5, that is combined with buffer prior to use.

Pharmaceutical compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

Compositions suitable for parenteral administration comprise aqueous and non-aqueous solutions, suspensions or emulsions of the active compound, which preparations are typically sterile and can be isotonic with the blood of the intended recipient. Non-limiting illustrative examples include water, buffered saline, Hanks' solution, Ringer's solution, dextrose, fructose, ethanol, animal, vegetable or synthetic oils. Aqueous injection suspensions may contain substances which increase the viscosity of the suspension, such as sodium carboxymethyl cellulose, sorbitol, or dextran.

Additionally, suspensions of the active compounds may be prepared as appropriate oil injection suspensions. Suitable lipophilic solvents or vehicles include fatty oils such as sesame oil, or synthetic fatty acid esters, such as ethyl oleate or triglycerides, or liposomes. Optionally, the suspension may also contain suitable stabilizers or agents which increase the solubility of the compounds to allow for the preparation of highly concentrated solutions.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

After pharmaceutical compositions have been prepared, they may be placed in an appropriate container and labeled for treatment. Such labeling could include amount, frequency, and method of administration.

Pharmaceutical compositions and delivery systems appropriate for the compositions, methods and uses of the invention are known in the art (see, e.g., Remington: The Science and Practice of Pharmacy (2003) 20th ed., Mack Publishing Co., Easton, PA; Remington's Pharmaceutical Sciences (1990) 18th ed., Mack Publishing Co., Easton, PA; The Merck Index (1996) 12^{th} ed., Merck Publishing Group, Whitehouse, NJ; Pharmaceutical Principles of Solid Dosage Forms (1993), Technonic Publishing Co., Inc., Lancaster, Pa.; Ansel and Stoklosa, Pharmaceutical Calculations (2001) 11^{th} ed., Lippincott Williams & Wilkins, Baltimore, MD; and Poznansky et al., Drug Delivery Systems (1980), R. L. Juliano, ed., Oxford, N.Y., pp. 253-315).

An “effective amount” or “sufficient amount” refers to an amount that provides, in single or multiple doses, alone or in combination, with one or more other compositions (therapeutic or immunosupprosive agents such as a drug), treatments, protocols, or therapeutic regimens agents, a detectable response of any duration of time (long or short term), an expected or desired outcome in or a benefit to a subject of any measurable or detectable degree or for any duration of time (e.g., for minutes, hours, days, months, years, or cured).

Doses can vary and depend upon the type, onset, progression, severity, frequency, duration, or probability of the disease to which treatment is directed, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The dose to achieve a therapeutic effect, e.g., the dose in vector genomes/per kilogram of body weight (vg/kg), will vary based on several factors including, but not limited to: route of administration, the level of heterologous polynucleotide expression required to achieve a therapeutic effect, the specific disease treated, any host immune response to the viral vector, a host immune response to the heterologous polynucleotide or expression product (protein), and the stability of the protein expressed. One skilled in the art can determine a rAAV/vector genome dose range to treat a patient having a particular disease or disorder based on the aforementioned factors, as well as other factors.

Generally, doses will range from at least 1×10⁸, or more, for example, 1×10⁹, 1×10¹⁰, 1×10¹¹, 1×10¹², 1×10¹³ or 1×10¹⁴, or more, vector genomes per kilogram (vg/kg) of the weight of the subject, to achieve a therapeutic effect. AAV dose in the range of 1×10¹⁰-1×10¹¹ in mice, and 1×10¹²-1×10¹³ in dogs have been effective. Doses can be less, for example, a dose of less than 6×10¹² vector genomes per kilogram (vg/kg). More particularly, a dose of 5×10¹¹ vg/kg or 1×10¹² vg/kg.

Using hemophilia B as an example, generally speaking, it is believed that, in order to achieve a therapeutic effect, a blood coagulation factor concentration that is greater than 1% of factor concentration found in a normal individual is needed to change a severe disease phenotype to a moderate one. A severe phenotype is characterized by joint damage and life-threatening bleeds. To convert a moderate disease phenotype into a mild one, it is believed that a blood coagulation factor concentration greater than 5% of normal is needed.

FVIII levels in normal humans are about 150-200 ng/ml plasma, but may be less (e.g., range of about 100-150 ng/ml) or greater (e.g., range of about 200-300 ng/ml) and still considered normal due to functioning clotting as determined, for example, by an activated partial thromboplastin time (aPTT) one-stage clotting assay. Thus, a therapeutic effect can be achieved by expression of FVIII or hFVIII-BDD such that the total amount of FVIII in the subject/human is greater than 1% of the FVIII present in normal subjects/humans, e.g., 1% of 100-300 ng/ml.

rAAV vector doses can be at a level, typically at the lower end of the dose spectrum, such that there is not a substantial immune response against the FVIII or AAV vector. More particularly, a dose of up to but less than 6×10¹² vg/kg, such as about 5×10¹¹ to about 5×10¹² vg/kg, or more particularly, about 5×10¹¹ vg/kg or about 1×10¹² vg/kg.

The doses of an “effective amount” or “sufficient amount” for treatment (e.g., to ameliorate or to provide a therapeutic benefit or improvement) typically are effective to provide a response to one, multiple or all adverse symptoms, consequences or complications of the disease, one or more adverse symptoms, disorders, illnesses, pathologies, or complications, for example, caused by or associated with the disease, to a measurable extent, although decreasing, reducing, inhibiting, suppressing, limiting or controlling progression or worsening of the disease is a satisfactory outcome.

An effective amount or a sufficient amount can but need not be provided in a single administration, may require multiple administrations, and, can but need not be, administered alone or in combination with another composition (e.g., agent), treatment, protocol or therapeutic regimen. For example, the amount may be proportionally increased as indicated by the need of the subject, type, status and severity of the disease treated or side effects (if any) of treatment. In addition, an effective amount or a sufficient amount need not be effective or sufficient if given in single or multiple doses without a second composition (e.g., another drug or agent), treatment, protocol or therapeutic regimen, since additional doses, amounts or duration above and beyond such doses, or additional compositions (e.g., drugs or agents), treatments, protocols or therapeutic regimens may be included in order to be considered effective or sufficient in a given subject. Amounts considered effective also include amounts that result in a reduction of the use of another treatment, therapeutic regimen or protocol, such as administration of recombinant clotting factor protein (e.g., FVIII) for treatment of a clotting disorder (e.g., hemophilia A).

Accordingly, methods and uses of the invention also include, among other things, methods and uses that result in a reduced need or use of another compound, agent, drug, therapeutic regimen, treatment protocol, process, or remedy. For example, for a blood clotting disease, a method or use of the invention has a therapeutic benefit if in a given subject a less frequent or reduced dose or elimination of administration of a recombinant clotting factor protein to supplement for the deficient or defective (abnormal or mutant) endogenous clotting factor in the subject. Thus, in accordance with the invention, methods and uses of reducing need or use of another treatment or therapy are provided.

An effective amount or a sufficient amount need not be effective in each and every subject treated, nor a majority of treated subjects in a given group or population. An effective amount or a sufficient amount means effectiveness or sufficiency in a particular subject, not a group or the general population. As is typical for such methods, some subjects will exhibit a greater response, or less or no response to a given treatment method or use.

The term “ameliorate” means a detectable or measurable improvement in a subject's disease or symptom thereof, or an underlying cellular response. A detectable or measurable improvement includes a subjective or objective decrease, reduction, inhibition, suppression, limit or control in the occurrence, frequency, severity, progression, or duration of the disease, or complication caused by or associated with the disease, or an improvement in a symptom or an underlying cause or a consequence of the disease, or a reversal of the disease. For HemA, an effective amount would be an amount that reduces frequency or severity of acute bleeding episodes in a subject, for example, or an amount that reduces clotting time as measured by a clotting assay, for example.

Accordingly, pharmaceutical compositions of the invention include compositions wherein the active ingredients are contained in an effective amount to achieve the intended therapeutic purpose. Determining a therapeutically effective dose is well within the capability of a skilled medical practitioner using the techniques and guidance provided in the invention.

Therapeutic doses will depend on, among other factors, the age and general condition of the subject, the severity of the aberrant blood coagulation phenotype, and the strength of the control sequences regulating the expression levels of FVIII. Thus, a therapeutically effective amount in humans will fall in a relatively broad range that may be determined by a medical practitioner based on the response of an individual patient to vector-based FVIII treatment. Such doses may be alone or in combination with an immunosuppressive agent or drug.

Compositions such as pharmaceutical compositions may be delivered to a subject, so as to allow production of Factor VIII (FVIII). In a particular embodiment, pharmaceutical compositions comprising sufficient genetic material to enable a recipient to produce a therapeutically effective amount of a FVIII polypeptide can influence hemostasis in the subject.

The compositions may be administered alone. In certain embodiments, a recombinant AAV particle provides a therapeutic effect without an immunosuppressive agent. The therapeutic effect of FVIII optionally is sustained for a period of time, e.g., 2-4, 4-6, 6-8, 8-10, 10-14, 14-20, 20-25, 25-30, or 30-50 days or more, for example, 50-75, 75-100, 100-150, 150-200 days or more without administering an immunosuppressive agent. Accordingly, in certain embodiments CpG rAAV virus particle provide a therapeutic effect without administering an immunosuppressive agent for a period of time.

The compositions may be administered in combination with at least one other agent. In certain embodiments, rAAV vector is administered in conjunction with one or more immunosuppressive agents prior to, substantially at the same time or after administering a rAAV vector. In certain embodiments, e.g., 1-12, 12-24 or 24-48 hours, or 2-4, 4-6, 6-8, 8-10, 10-14, 14-20, 20-25, 25-30, 30-50, or more than 50 days following administering rAAV vector. Such administration of immunosuppressive agents after a period of time following administering rAAV vector if there is a decrease in FVIII after the initial expression levels for a period of time, e.g., 20-25, 25-30, 30-50, 50-75, 75-100, 100-150, 150-200 or more than 200 days following rAAV vector.

In certain embodiments, an immunosuppressive agent is an anti-inflammatory agent. In certain embodiments, an immunosuppressive agent is a steroid. In certain embodiments, an immunosuppressive agent is cyclosporine (e.g., cyclosporine A), mycophenolate, Rituximab or a derivative thereof. Additional particular agents include a stabilizing compound.

Compositions may be administered in any sterile, biocompatible pharmaceutical carrier, including, but not limited to, saline, buffered saline, dextrose, and water. The compositions may be administered to a patient alone, or in combination with other agents (e.g., co-factors) which influence hemostasis.

Protocols for the generation of adenoviral vectors and administration to patients have been described in U.S. Pat. Nos. 5,998,205; 6,228,646; 6,093,699; 6,100,242; and International Patent Application Nos. WO 94/17810 and WO 94/23744, which are incorporated herein by reference in their entirety. In particular, for example, AAV vectors are employed to deliver Factor VIII (FVIII) encoded by CpG reduced nucleic acid variants to a patient in need thereof.

Methods and uses of the invention include delivery and administration systemically, regionally or locally, or by any route, for example, by injection or infusion. Delivery of the pharmaceutical compositions in vivo may generally be accomplished via injection using a conventional syringe, although other delivery methods such as convection-enhanced delivery are envisioned (See e.g., U.S. Pat. No. 5,720,720). For example, compositions may be delivered subcutaneously, epidermally, intradermally, intrathecally, intraorbitally, intramucosally, intraperitoneally, intravenously, intra-pleurally, intraarterially, orally, intrahepatically, via the portal vein, or intramuscularly. Other modes of administration include oral and pulmonary administration, suppositories, and transdermal applications. A clinician specializing in the treatment of patients with blood coagulation disorders may determine the optimal route for administration of the adenoviral-associated vectors based on a number of criteria, including, but not limited to: the condition of the patient and the purpose of the treatment (e.g., enhanced or reduced blood coagulation).

Invention methods and uses can be combined with any compound, agent, drug, treatment or other therapeutic regimen or protocol having a desired therapeutic, beneficial, additive, synergistic or complementary activity or effect. Exemplary combination compositions and treatments include second actives, such as, biologics (proteins), agents (e.g., immunosuppressive agents) and drugs. Such biologics (proteins), agents, drugs, treatments and therapies can be administered or performed prior to, substantially contemporaneously with or following any other method or use of the invention, for example, a therapeutic method of treating a subject for a blood clotting disease such as HemA.

The compound, agent, drug, treatment or other therapeutic regimen or protocol can be administered as a combination composition, or administered separately, such as concurrently or in series or sequentially (prior to or following) delivery or administration of a nucleic acid, vector, recombinant vector (e.g., rAAV), or recombinant virus particle. The invention therefore provides combinations in which a method or use of the invention is in a combination with any compound, agent, drug, therapeutic regimen, treatment protocol, process, remedy or composition, set forth herein or known to one of skill in the art. The compound, agent, drug, therapeutic regimen, treatment protocol, process, remedy or composition can be administered or performed prior to, substantially contemporaneously with or following administration of a nucleic acid, vector, recombinant vector (e.g., rAAV), or recombinant virus particle of the invention, to a subject.

The invention is useful in animals including human and veterinary medical applications. Suitable subjects therefore include mammals, such as humans, as well as non-human mammals. The term “subject” refers to an animal, typically a mammal, such as humans, non-human primates (apes, gibbons, gorillas, chimpanzees, orangutans, macaques), a domestic animal (dogs and cats), a farm animal (poultry such as chickens and ducks, horses, cows, goats, sheep, pigs), and experimental animals (mouse, rat, rabbit, guinea pig). Human subjects include fetal, neonatal, infant, juvenile and adult subjects. Subjects include animal disease models, for example, mouse and other animal models of blood clotting diseases such as HemA and others known to those of skill in the art.

Subjects appropriate for treatment in accordance with the invention include those having or at risk of producing an insufficient amount or having a deficiency in a functional gene product (e.g., FVIII protein), or produce an aberrant, partially functional or non-functional gene product (e.g., FVIII protein), which can lead to disease. Subjects appropriate for treatment in accordance with the invention also include those having or at risk of producing an aberrant, or defective (mutant) gene product (protein) that leads to a disease such that reducing amounts, expression or function of the aberrant, or defective (mutant) gene product (protein) would lead to treatment of the disease, or reduce one or more symptoms or ameliorate the disease. Target subjects therefore include subjects having aberrant, insufficient or absent blood clotting factor production, such as hemophiliacs (e.g., hemophilia A).

Subjects can be tested for an immune response, e.g., antibodies against AAV. Candidate hemophilia subjects can therefore be screened prior to treatment according to a method of the invention. Subjects also can be tested for antibodies against AAV after treatment, and optionally monitored for a period of time after treatment. Subjects developing antibodies can be treated with an immunosuppressive agent, or can be administered one or more additional amounts of AAV vector.

Subjects appropriate for treatment in accordance with the invention also include those having or at risk of producing antibodies against AAV. rAAV vectors can be administered or delivered to such subjects using several techniques. For example, empty capsid AAV (i.e., AAV lacking a FVIII nucleic acid) can be delivered to bind to the AAV antibodies in the subject thereby allowing the AAV vector bearing nucleic acid or nucleic acid variant encoding FVIII and FVIII-BDD to transform cells of the subject.

Ratio of empty capsids to the rAAV vector can be between about 2:1 to about 50:1, or between about 2:1 to about 25:1, or between about 2:1 to about 20:1, or between about 2:1 to about 15:1, or between about 2:1 to about 10:1. Ratios can also be about 2:1, 3:1, 4:1, 5:1, 6:1, 7:1, 8:1, 9:1, or 10:1.

Amounts of empty capsid AAV to administer can be calibrated based upon the amount (titer) of AAV antibodies produced in a particular subject. Empty capsid can be of any AAV serotype, for example, AAV1, AAV2, AAV3, AAV4, AAV5, AAV6, AAV7, AAV8, AAV9, AAV10, AAV11, AAV12, Rh10, Rh74 or AAV-2i8.

Alternatively or in addition to, AAV vector can be delivered by direct intramuscular injection (e.g., one or more slow-twitch fibers of a muscle). In another alternative, a catheter introduced into the femoral artery can be used to delivery AAV vectors to liver via the hepatic artery. Non-surgical means can also be employed, such as endoscopic retrograde cholangiopancreatography (ERCP), to deliver AAV vectors directly to the liver, thereby bypassing the bloodstream and AAV antibodies. Other ductal systems, such as the ducts of the submandibular gland, can also be used as portals for delivering AAV vectors into a subject that develops or has preexisting anti-AAV antibodies.

Administration or in vivo delivery to a subject can be performed prior to development of an adverse symptom, condition, complication, etc. caused by or associated with the disease. For example, a screen (e.g., genetic) can be used to identify such subjects as candidates for invention compositions, methods and uses. Such subjects therefore include those screened positive for an insufficient amount or a deficiency in a functional gene product (e.g., FVIII protein), or that produce an aberrant, partially functional or non-functional gene product (e.g., FVIII protein).

Administration or in vivo delivery to a subject in accordance with the methods and uses of the invention as disclosed herein can be practiced within 1-2, 2-4, 4-12, 12-24 or 24-72 hours after a subject has been identified as having the disease targeted for treatment, has one or more symptoms of the disease, or has been screened and is identified as positive as set forth herein even though the subject does not have one or more symptoms of the disease. Of course, methods and uses of the invention can be practiced 1-7, 7-14, 14-21, 21-48 or more days, months or years after a subject has been identified as having the disease targeted for treatment, has one or more symptoms of the disease, or has been screened and is identified as positive as set forth herein.

A “unit dosage form” as used herein refers to physically discrete units suited as unitary dosages for the subject to be treated; each unit containing a predetermined quantity optionally in association with a pharmaceutical carrier (excipient, diluent, vehicle or filling agent) which, when administered in one or more doses, is calculated to produce a desired effect (e.g., prophylactic or therapeutic effect). Unit dosage forms may be within, for example, ampules and vials, which may include a liquid composition, or a composition in a freeze-dried or lyophilized state; a sterile liquid carrier, for example, can be added prior to administration or delivery in vivo. Individual unit dosage forms can be included in multi-dose kits or containers. Recombinant vector (e.g., rAAV) sequences, recombinant virus particles, and pharmaceutical compositions thereof can be packaged in single or multiple unit dosage form for ease of administration and uniformity of dosage.

Subjects can be tested for FVIII and FVIII-BDD amounts or FVIII and FVIII-BDD activity to determine if such subjects are appropriate for treatment according to a method of the invention. Candidate hemophilia subjects can be tested for FVIII and FVIII-BDD amounts or activity prior to treatment according to a method of the invention. Subjects also can be tested for amounts of FVIII and FVIII-BDD or FVIII and FVIII-BDD activity after treatment according to a method of the invention. Such treated subjects can be monitored after treatment for FVIII and FVIII-BDD amounts or FVIII and FVIII-BDD activity, periodically, e.g., every 1-4 weeks or 1-6 months.

Subjects can be tested for one or more liver enzymes for an adverse response or to determine if such subjects are appropriate for treatment according to a method of the invention. Candidate hemophilia subjects can therefore be screened for amounts of one or more liver enzymes prior to treatment according to a method of the invention. Subjects also can be tested for amounts of one or more liver enzymes after treatment according to a method of the invention. Such treated subjects can be monitored after treatment for elevated liver enzymes, periodically, e.g., every 1-4 weeks or 1-6 months.

Exemplary liver enzymes include alanine aminotransferase (ALT), aspartate aminotransferase (AST), and lactate dehydrogenase (LDH), but other enzymes indicative of liver damage can also be monitored. A normal level of these enzymes in the circulation is typically defined as a range that has an upper level, above which the enzyme level is considered elevated, and therefore indicative of liver damage. A normal range depends in part on the standards used by the clinical laboratory conducting the assay.

Subjects can be monitored for bleeding episodes to determine if such subjects are eligible for or responding to treatment, and/or the amount or duration of responsiveness. Subjects can be monitored for bleeding episodes to determine if such subjects are in need of an additional treatment, e.g., a subsequent AAV vector administration or administration of an immunosuppressive agent, or more frequent monitoring. Hemophilia subjects can therefore be monitored for bleeding episodes prior to and after treatment according to a method of the invention. Subjects also can be tested for frequency and severity of bleeding episodes during or after treatment according to a method of the invention.

The invention provides kits with packaging material and one or more components therein. A kit typically includes a label or packaging insert including a description of the components or instructions for use in vitro, in vivo, or ex vivo, of the components therein. A kit can contain a collection of such components, e.g., a nucleic acid, recombinant vector, virus (e.g., AAV) vector, or virus particle and optionally a second active, such as another compound, agent, drug or composition.

A kit refers to a physical structure housing one or more components of the kit. Packaging material can maintain the components sterilely, and can be made of material commonly used for such purposes (e.g., paper, corrugated fiber, glass, plastic, foil, ampules, vials, tubes, etc.).

Labels or inserts can include identifying information of one or more components therein, dose amounts, clinical pharmacology of the active ingredient(s) including mechanism of action, pharmacokinetics and pharmacodynamics. Labels or inserts can include information identifying manufacturer, lot numbers, manufacture location and date, expiration dates. Labels or inserts can include information identifying manufacturer information, lot numbers, manufacturer location and date. Labels or inserts can include information on a disease for which a kit component may be used. Labels or inserts can include instructions for the clinician or subject for using one or more of the kit components in a method, use, or treatment protocol or therapeutic regimen. Instructions can include dosage amounts, frequency or duration, and instructions for practicing any of the methods, uses, treatment protocols or prophylactic or therapeutic regimes described herein.

Labels or inserts can include information on any benefit that a component may provide, such as a prophylactic or therapeutic benefit. Labels or inserts can include information on potential adverse side effects, complications or reactions, such as warnings to the subject or clinician regarding situations where it would not be appropriate to use a particular composition. Adverse side effects or complications could also occur when the subject has, will be or is currently taking one or more other medications that may be incompatible with the composition, or the subject has, will be or is currently undergoing another treatment protocol or therapeutic regimen which would be incompatible with the composition and, therefore, instructions could include information regarding such incompatibilities.

Labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, such as a bar-coded printed label, a disk, optical disk such as CD- or DVD-ROM/RAM, DVD, MP3, magnetic tape, or an electrical storage media such as RAM and ROM or hybrids of these such as magnetic/optical storage media, FLASH media or memory type cards.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All patents, patent applications, publications, and other references, GenBank citations and ATCC citations cited herein are incorporated by reference in their entirety. In case of conflict, the specification, including definitions, will control.

Various terms relating to the biological molecules of the invention are used hereinabove and also throughout the specification and claims.

All of the features disclosed herein may be combined in any combination. Each feature disclosed in the specification may be replaced by an alternative feature serving a same, equivalent, or similar purpose. Thus, unless expressly stated otherwise, disclosed features (e.g., CpG reduced nucleic acid variants encoding FVIII, vector, plasmid, expression/recombinant vector (e.g., rAAV) sequence, or recombinant virus particle) are an example of a genus of equivalent or similar features.

As used herein, the singular forms “a”, “and,” and “the” include plural referents unless the context clearly indicates otherwise. Thus, for example, reference to “a nucleic acid” includes a plurality of such nucleic acids, reference to “a vector” includes a plurality of such vectors, and reference to “a virus” or “particle” includes a plurality of such viruses/particles.

As used herein, all numerical values or numerical ranges include integers within such ranges and fractions of the values or the integers within ranges unless the context clearly indicates otherwise. Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-850, includes ranges of 1-20, 1-30, 1-40, 1-50, 1-60, 10-30, 10-40, 10-50, 10-60, 10-70, 10-80, 20-40, 20-50, 20-60, 20-70, 20-80, 20-90, 50-75, 50-100, 50-150, 50-200, 50-250, 100-200, 100-250, 100-300, 100-350, 100-400, 100-500, 150-250, 150-300, 150-350, 150-400, 150-450, 150-500, etc.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

A number of embodiments of the invention have been described. Nevertheless, one skilled in the art, without departing from the spirit and scope of the invention, can make various changes and modifications of the invention to adapt it to various usages and conditions. Accordingly, the following examples are intended to illustrate but not limit the scope of the invention claimed in any way.

### Example 1

### CpG Reduced Factor VIII DNA Sequences and Certain Vector Constructs, Plasmid Constructs and AAV Vector Producing Cell Lines.

18 different CpG reduced nucleic acid variants encoding FVIII (SEQ ID NOs:1-18) were produced and assessed in expression assays. CpG reduced human FVIII cDNA constructs were generated with a mutant transthyretin (TTRmut) promoter (SEQ ID NO:22).

AAV-SPK-8011 expression cassette has the CpG reduced FVIII-X07 nucleic acid sequence and the LK03 capsid for packaging. LK03 capsid has substantial homology to AAV3, a non-pathogenic, naturally replication deficient single-stranded DNA virus.

Packaging plasmid pLK03 is a 7,484 bp plasmid construct that carries the AAV2 Rep and AAV-LK03 Cap genes under the control of AAV2 p5 promoter, bacterial origin of replication and gene conferring resistance to Kanamycin in bacterial cells. In this construct, the p5 rep promoter has been moved 3′ of the cap gene to reduce the potential for formation of wild-type or pseudo wild type AAV species, and to increase yield of the vector.

The cloned DNA for gene transfer is a gene expression cassette, packaged into the AAV-LK03 capsid as a single-stranded genome, encoding human coagulation factor VIII (hFVIII) under control of a liver-specific promoter. The expression plasmid is referred to as pAAV-TTRmut-hFVIII-X07. It was modified by the introduction of 4 point mutations in the TTR promoter, and the coding region optimized to increase expression of human FVIII. The AAV expression cassette contains the following elements:
AAV2 ITR
Transthyretin (TTR) promoter: A liver-specific transthyretin (TTR) promoter with 4 point mutations that increase gene expression compared with the wild type promoter (Costa et al. 1991)
Synthetic intron: Derived from human elongation factor EF-1 alpha gene
FVIII coding sequence: B-domain deleted, codon-optimized human FVIII coding sequence.
Rabbit beta globin poly A signal sequence (Levitt et al. 1989).
AAV2 ITR

Three DNA plasmid constructs are used to transfect human embryo kidney 293 cells to produce the SPK-8011 vector by a helper virus-free process (Matsushita et al. 1998):
The gene cassette (hFVIII coding sequence and associated regulatory elements) is cloned into a plasmid to give the vector plasmid, pAAV-TTRmut-hFVIII-X07.
The AAV viral genome (rep and cap) lacking the viral ITRs is cloned into a plasmid to give the AAV packaging plasmid, pLK03, providing the required AAV2 rep and AAV-LK03 cap genes in trans for AAV vector packaging. The viral promoter (p5) for the rep gene was relocated in the plasmid in order to prevent formation of replication competent AAV by non-homologous recombination.
Three genes from adenovirus-2 are cloned into a third plasmid (pCCVC-AD2HP) providing the necessary helper virus genes for vector production. Plasmid pCCVC-AD2HPv2 is an 11,832 bp plasmid construct that carries three adenovirus genes, E2A, E4 and the VA RNAs to provide ‘helper’ functions necessary for replication and encapsidation of AAV vector. Plasmid pCCVC-AD2HPv2 is a derivative of pCCVC-AD2HP in which the DrdI-DrdI 1882 bp restriction fragment containing the Amp^{R} gene and part of the pUC ori sequence has been removed and replaced with the DrdI-DrdI fragment from plasmid pAAV2-hRPE65v2 containing the entire Kan^{R} gene and part of the pUC ori sequence.

The cell substrate used for AAV vector production is a derivative of primary human embryonic kidney cells (HEK) 293. The HEK293 cell line is a permanent line transformed by sheared human adenovirus type 5 (Ad5) DNA (Graham et al. 1977). The Working Cell Bank is derived from a characterized HEK293 Master Cell Bank from the Center for Cellular and Molecular Therapeutics (CCMT) at The Children's Hospital of Philadelphia (CHOP).

### Example 2

Evaluation of AAV-SPK-8005 and AAV-SPK-8011 (LK03 capsid, FVIII-X07 (SEQ ID NO:7)) Vectors in Non-Human Primates (NHPs).

FVIII transgene constructs packaged into adeno-associated viral (AAV) vectors were delivered to non-human primates (NHPs). Both a pilot study and a GLP study were performed.

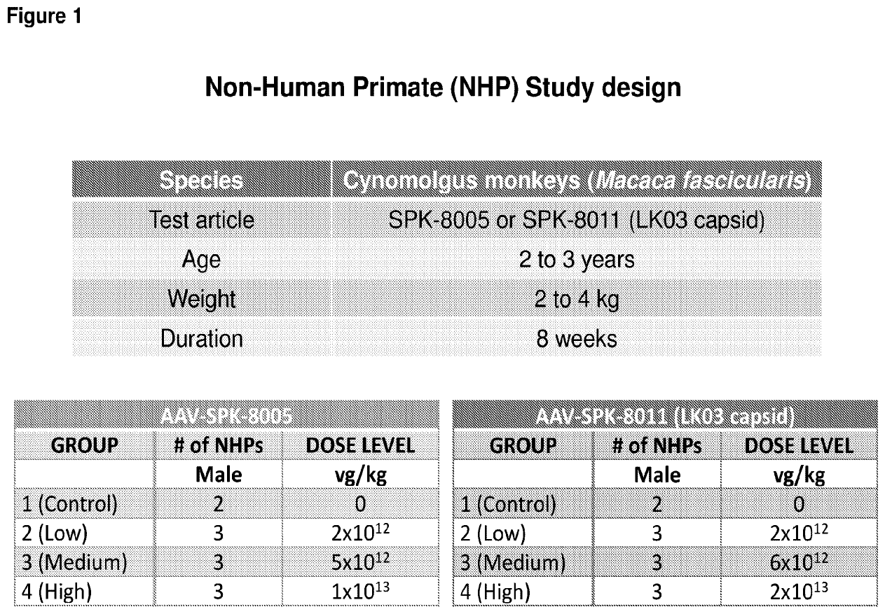

In brief, a dose-ranging study in male cynomolgus macaques administered a single intravenous infusion of AAV-SPK-8005 or AAV-SPK-8011 (LK03 capsid) was performed. Expression of hFVIII was evaluated over 8 weeks. The animal groups and dose levels of each vector (pilot study) are shown in FIG. 1.

NHPs received an intravenous infusion via the saphenous vein using a calibrated infusion pump over approximately 30 minutes. Macaques were prescreened for neutralizing antibodies against the AAV capsid. All treated animals were initially determined to have a <1:3 titer before vector administration. This was done to ensure successful hepatic transduction, as even low titers inhibit vector uptake by liver cells after systemic delivery (Jiang et al. 2006). All animals were also negative for the presence of neutralizing antibodies against FVIII before gene transfer.

Plasma levels of hFVIII were measured by a human-specific ELISA that does not detect the cynomolgus endogenous FVIII. All the animals in the study, with the exception of one macaque in the mid dose cohort, express hFVIII following vector delivery. Human factor VIII antigen levels peaked at around 1-2 weeks following vector administration. At one week after gene transfer, NHPs transduced with 2×10¹² vg/kg of AAV-SPK-8005 expressed hFVIII antigen levels of 13.2±3% (average±standard error of the mean). At one week after gene transfer, average hFVIII levels in two of the three animals in the next treatment cohort (5×10¹² vg/kg) were 27±0.2%. Human FVIII could not be detected in the third macaque in that cohort at any time point. Upon re-testing of baseline plasma samples it was determined that this animal was in fact positive for the presence of anti-AAV antibodies and that the initially determined titer of <1:3 was incorrect. Finally, at the highest tested dose of 1×10¹³ vg/kg, peak hFVIII antigen levels of 54.1±15.6% were observed after AAV infusion.

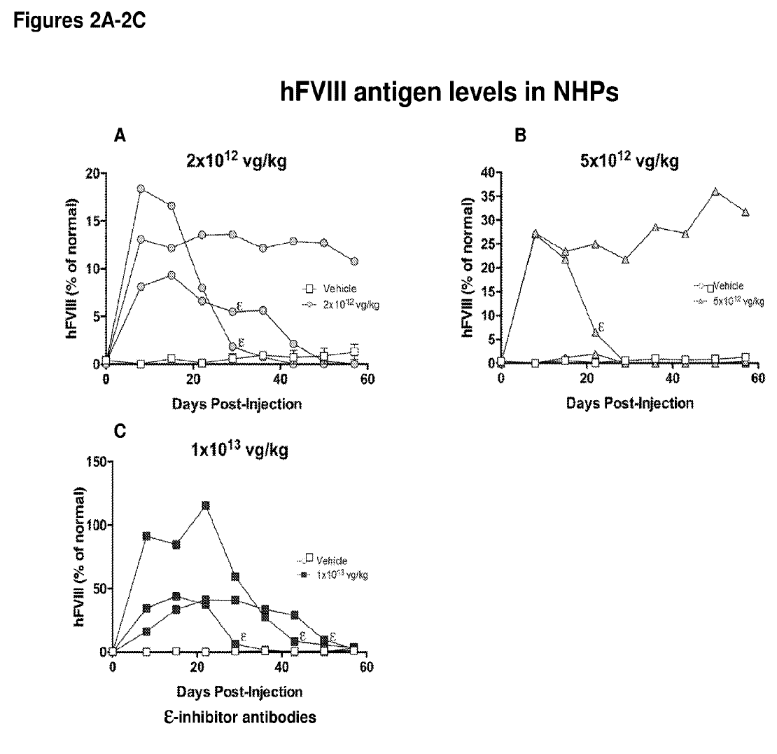

Human FVIII expression declined in approximately one third of the animals around week 4, concomitant with the appearance of inhibitor antibodies to hFVIII in these 3 macaques (labeled with a & symbol in FIG. 2). Development of species-specific antibodies to hFVIII has been previously documented in non-human primates, and is likely due to differences in several amino acid residues between the human transgene product and the endogenous cynomolgus FVIII (McIntosh, J. et al., Blood 121:3335-44 (2013)).

To assess potential thrombogenesis due to continuous expression of human FVIII, D-dimer antigen levels were measured in this study. It should be noted that reports on the clinical relevance or even the normal values of D-dimer antigen levels in cynomolgus macaques are scarce; as a reference, the normal range for D-dimers in humans is below 500 ng/ml. Since the animals express endogenous cynomolgus FVIII, production of hFVIII as a result of hepatic gene transfer will result in supraphysiological levels of FVIII activity.

The animal that was dosed at 5×10¹² vg/kg but did not express human FVIII had a peak of 863 ng/ml two weeks after AAV infusion. The rest of the animals did not show any significant increase in D-dimer antigen levels compared to baseline values. Taken together, these results suggest that expression of human FVIII, at the levels targeted in this study, is not associated with an increased risk of thrombosis.

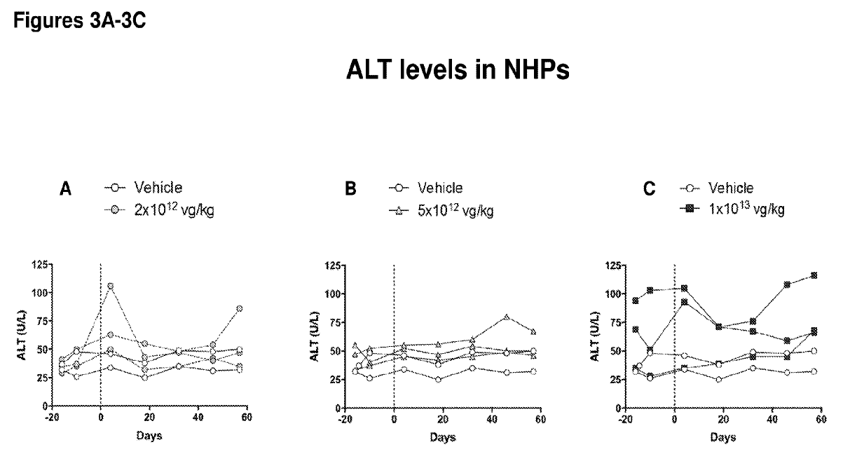

Four weeks after vector administration, no vector-related changes were apparent. Liver function tests showed normal values, with minor fluctuations that appeared to be unrelated to vector dose, as they were present prior to dosing in most cases (FIG. 3).

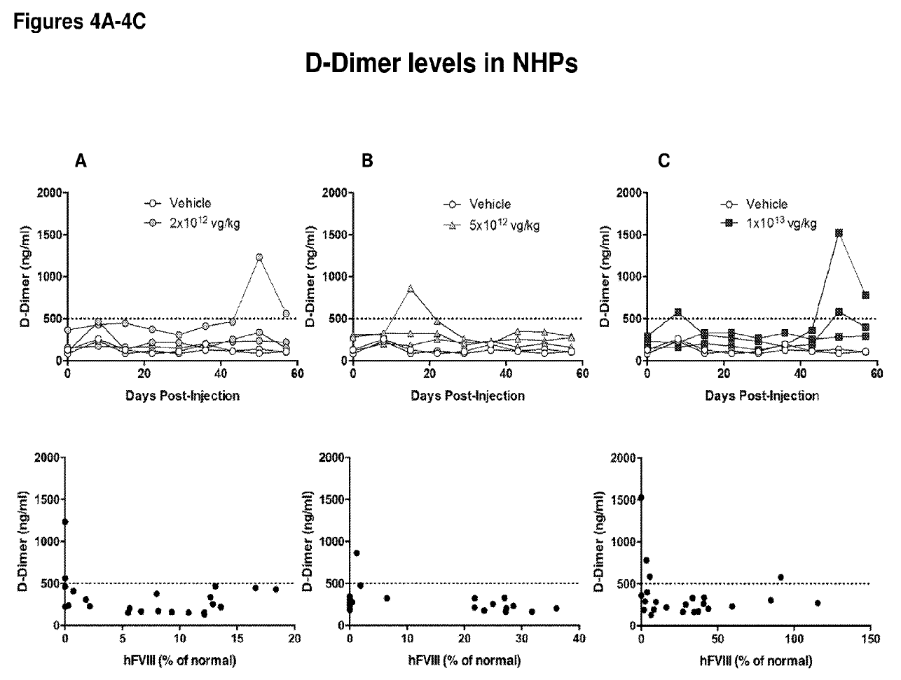

D-dimer levels up to week 5 are shown in FIG. 4. One animal in the high dose cohort had a slight (577 ng/ml), transient elevation in D-dimer levels one week after vector administration, when circulating human FVIII peaked at around 100%; the D-dimer levels rapidly returned to normal after this single elevate measurement. Notably, there was no correlation between D-dimer levels and hFVIII antigen levels (FIG. 4, bottom panels).

For AAV-SPK-8011 (LK03 capsid) vector in a pilot study, three cohorts of cynomolgus macaques (n=3) were treated with increasing doses of AAV-SPK-8011 (LK03 capsid) (2×10¹², 6×10¹² and 2×10¹³ (vg/kg); FIG. 1). In a GLP study, doses of 3×10¹², 6×10¹² and 2×10¹³ vg/kg (AAV-SPK-8011 (LK03 capsid)) vector were used.

A total of 11 NHPs were used in in each study. The pilot study had an observation period of 10 weeks in the absence of immunosuppression. This was followed by a 12-week immunosuppression phase, which was incorporated in order to eradicate the anti-hFVIII antibodies that were generated during the initial 10 weeks of the study. Subsequently, the animals were followed for an additional 20 weeks.

Animals were monitored for clinical observations, body weights clinical pathology (clinical chemistry, hematology, coagulation, urinalysis). In addition, hFVIII antigen levels, FVIII inhibitory antibodies and D-dimer levels were assessed throughout the study.

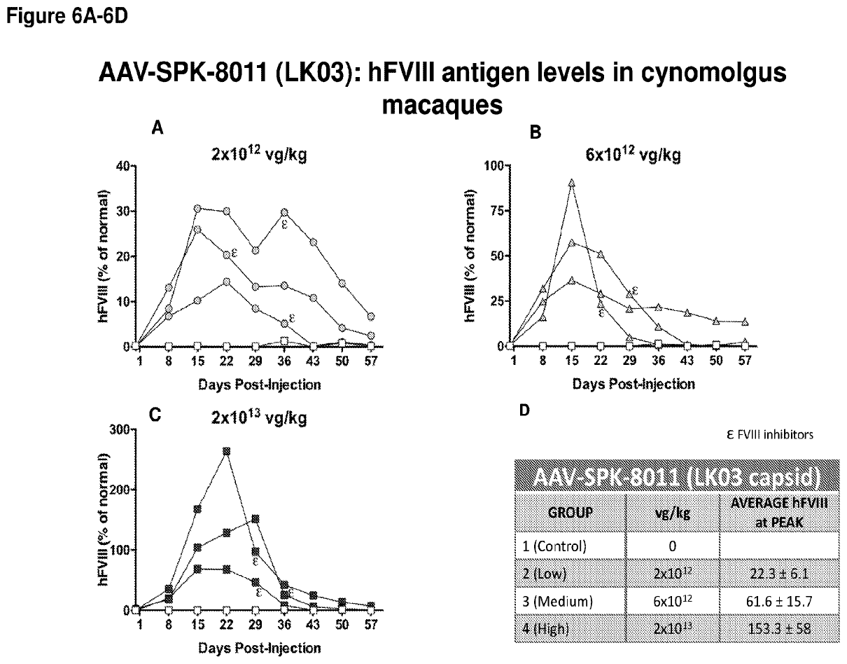

The hFVIII antigen pilot study data is shown in FIG. 6. Average hFVIII antigen levels peaked around week 2-3 with 22.3±6.2% hFVIII seen in the low dose cohort and 61.6±15.7% and 153±58.1% observed in the mid and high dose cohorts, respectively, using 150 ng/ml as the 100% normal hFVIII antigen level (FIGS. 6A-6D).

In the GLP toxicology study, hepatic gene transfer via peripheral vein infusion of SPK-8011 led to hFVIII expression in all animals as well. At the low dose of 3×10¹² vg/kg, hFVIII antigen levels ranged from 5-40% of normal, with an average peak level around week 2 after AAV administration of 20.3±11% (average±SEM). Average hFVIII antigen levels in the 6×10¹² vg/kg cohort were 40.7±4% of normal.

Thus, the LK03 AAV capsid serotype efficiently transduces NHP hepatocytes in vivo, unlike mouse liver. Despite the therapeutic hFVIII levels observed soon after gene transfer, in most animals the levels began to decline around week 4.

Humoral response to hFVIII in plasma of cynomolgus macaques was measured following administration of AAV-SPK-8011 (LK03 capsid). The animals were assessed for anti-hFVIII IgG antibodies by ELISA at baseline and at the indicated time points.

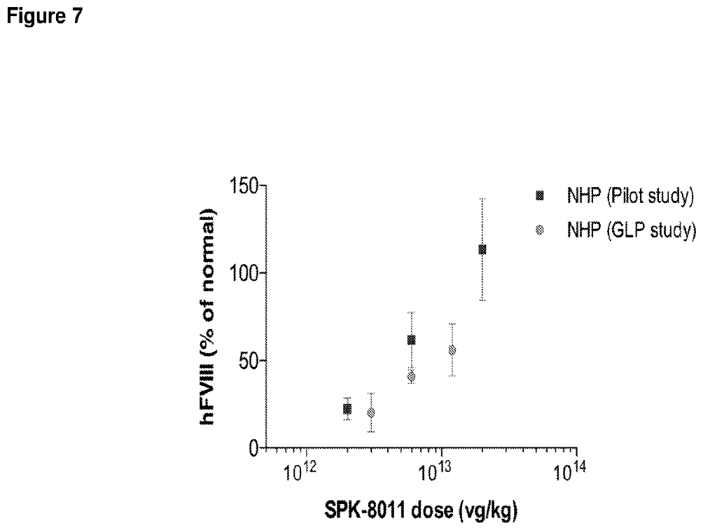

Most of the vector-treated animals in both pilot and GLP studies developed anti-FVIII neutralizing antibodies, an anticipated outcome based on preclinical cynomolgus macaques studies as well as reports by others (McIntosh, J. et al., Blood 121:3335-44 (2013)). Neutralizing antibodies against the human FVIII protein, which typically appear starting three weeks after AAV infusion in macaques, preclude detection of circulating hFVIII antigen. As a result, peak hFVIII antigen levels around weeks 2-3 (i.e. before the appearance of inhibitory antibodies against hFVIII) can be used to estimate the adequate starting vector dose in human subjects. The dose-response curves of SPK-8011 in the pilot and GLP NHP studies are shown in FIG. 7.

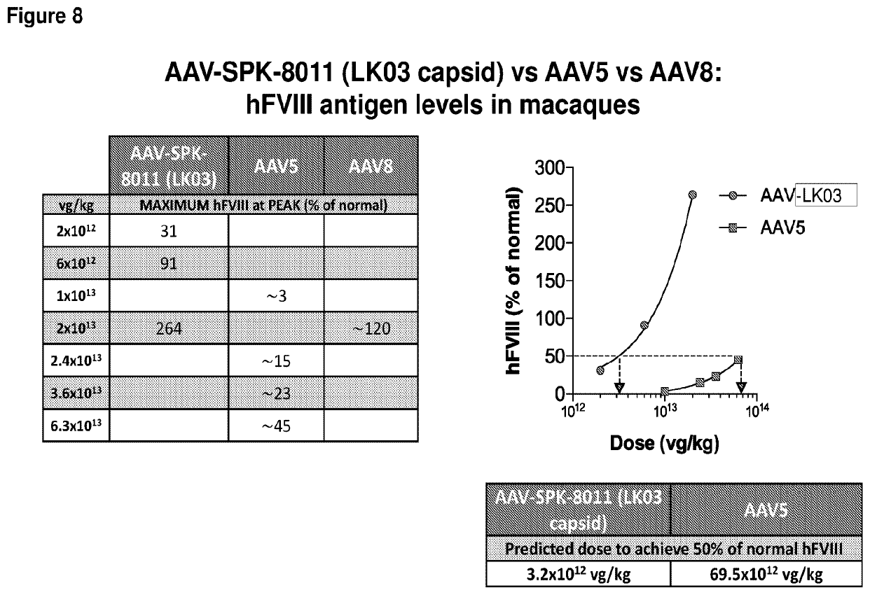

FVIII expression levels attained with AAV-SPK-8011 (LK03 capsid) were compared to reported levels of FVIII attained with AAV5 and AAV8 capsid based AAV vectors for delivery of FVIII. A comparison revealed that levels of FVIII achieved with AAV-SPK-8011 (LK03 capsid) were greater than the reported levels of FVIII delivered by way of AAV vectors with AAV5 and AAV8 capsids (FIG. 8).

### Example 3

### Biodistribution of AAV-LK03 Capsid in Non-Human Primates (NHPs).

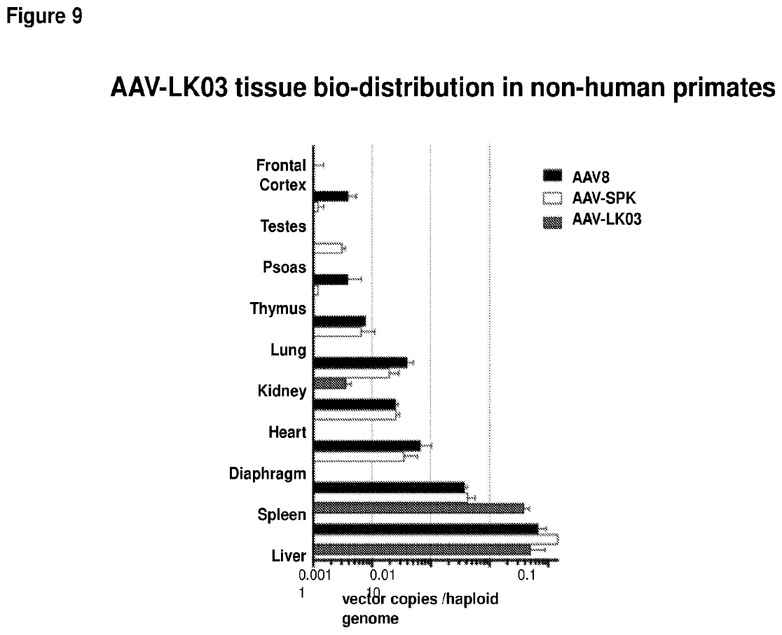

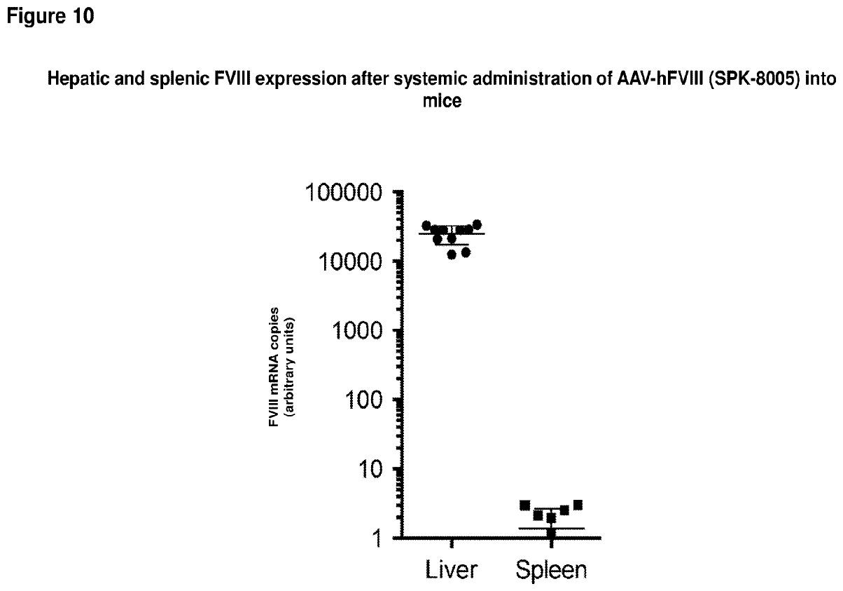

Biodistribution of the AAV-LK03 capsid in non-human primates was evaluated in a non-GLP study. Intravenous administration of an AAV-LK03-encapsidated vector encoding human coagulation factor IX (AAV-LK03-hFIX) showed that the two main target tissues are the liver and the spleen (FIG. 9). The splenic tropism is not a unique characteristic of AAV-LK03. For example, the AAV5 capsid, which has been used in several liver-directed gene therapy trials (e.g. NCT02396342, NCT02082860, NCT02576795) with a strong safety record, targets the spleen with the same if not higher efficacy than it targets the liver of non-human primates (Paneda et al. 2013). The SPK-8011 expression cassette uses the mouse transthyretin or TTR promoter, which is considered liver-specific (Costa, 1991). To further support the liver-specific nature of the promoter, a PCR-based expression analysis measured vector-derived FVIII expression in the livers and spleens of mice after administration of a different AAV vector packaging the same expression cassette as SPK-8011 (i.e. AAV-SPK-8005). As shown in FIG. 10, human FVIII expression in the spleen is several orders of magnitude lower compared with that derived from hepatocytes.

This is the first clinical study to use AAV-LK03, although studies have been conducted using other AAV vectors including several for hemophilia B (NCT02396342, NCT01620801 NCT00076557, NCT02484092, NCT02618915, NCT00979238, NCT01687608) and one for hemophilia A (NCT02576795). A study conducted by St. Jude Children's Research Hospital in collaboration with University College London utilized an AAV8 vector carrying a self-complementary genome encoding a codon-optimized human factor IX cDNA, scAAV2/8-LP1-hFIXco. Ten subjects who received the vector have had stable factor IX levels of 1-6% through a median of 3.2 years and all participants have either discontinued or reduced the use of prophylactic factor replacement (Nathwani et al. 2014). A clinical study for hemophilia A used an AAV5 encapsidated vector encoding human FVIII (NCT02576795). Preliminary data presented in 2016 demonstrate increases in FVIII activity after gene transfer in several subjects ranging from 2-60% with follow-up of up to 16 weeks (BioMarin, April 2016).

### Example 4

### Transduction Efficiency of AAV-LK03 Capsid Analyzed in an In Vitro Setting.

Primary hepatocytes from cynomolgus macaque and human origin were transduced with an AAV-LK03 vector expressing luciferase at four different multiplicities of infection (MOI) ranging from 500 to 62,500 vector genomes per cell. Seventy-two hours after transduction, luciferase expression was analyzed.

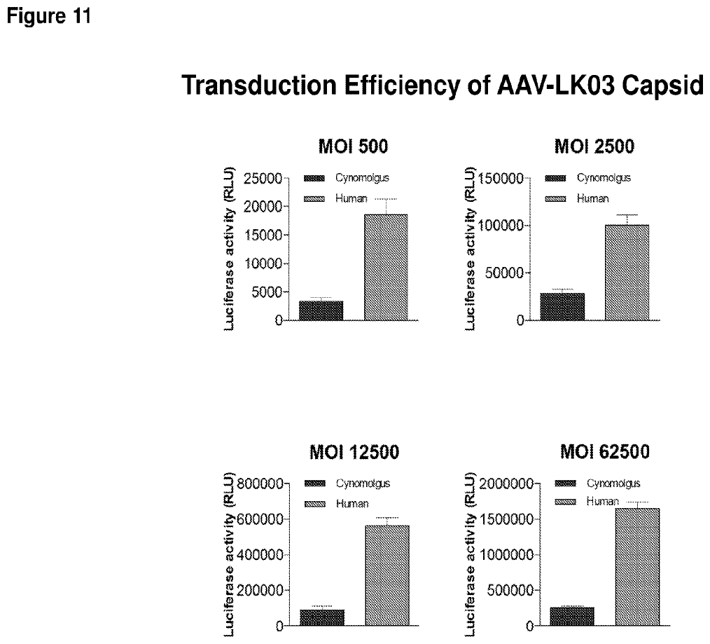

The AAV-LK03 capsid uniquely demonstrated significantly higher efficiency in transducing human hepatocytes in culture. In the representative example shown in FIG. 11, LK03 demonstrated approximately 5-fold higher efficiency in transducing human hepatocytes as compared to non-human primate hepatocytes in vitro. Importantly, these results are consistent across multiple MOIs and replicate studies.

### Example 5

### Human Clinical Trial Dose Calculations

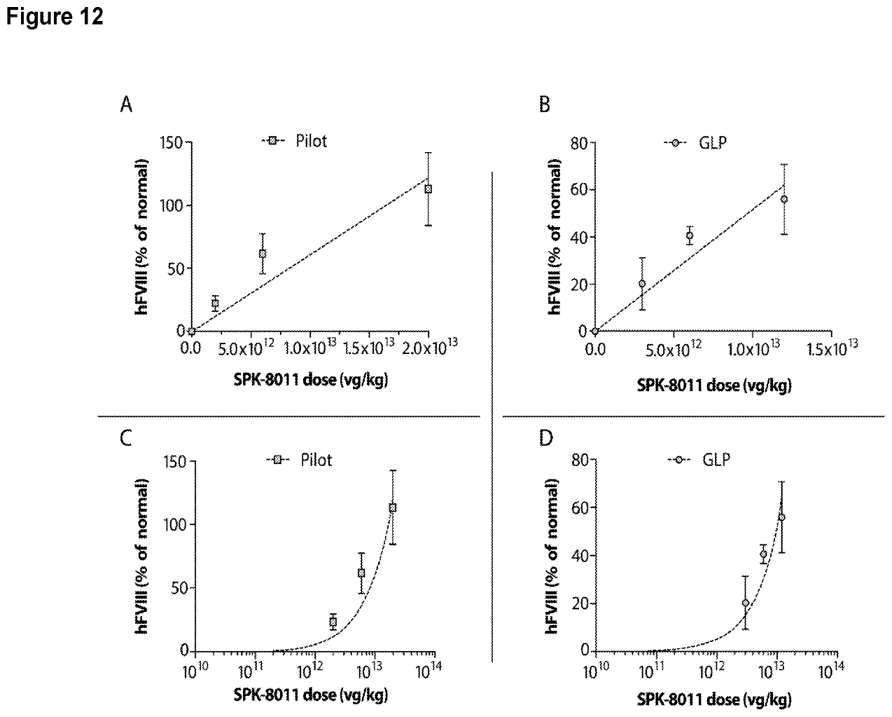

Based on hFVIII levels observed in non-human primates (NHPs), an estimate of the expected FVIII levels at the proposed starting dose of 5×10¹¹ vg/kg in humans was determined. Since different vector lots may have slightly different hepatic transduction efficacy, data from both the pilot and the GLP toxicology NHP studies were used to interpolate a range of FVIII concentrations after administration of 5×10¹¹ vg/kg. For this analysis, a linear regression model (FIG. 12), i.e. the relation between AAV dose and resulting hFVIII expression levels was not found to deviate significantly from linearity was used (Table 2).

TABLE 2

| | Pilot | GLP |
|---|---|---|
| Best-fit values | | |
| Slope | 6.099e−012 ± 7.962e−013 | 5.170e012 ± 6.421e−013 |
| Y-intercept when X = 0.0 | 0 | 0 |
| X-intercept when Y = 0.0 | 0 | 0 |
| 1/slope | 1.64E+11 | 1.934E+11 |
| 95% Confidence Intervals | | |
| Slope | 4.346e−012 to 7.851e−012 | 3.756e−012 to 6.583e−012 |
| Goodness of Fit | | |
| Sy.x | 28.93 | 15.29 |
| Is slope significantly non-zero? | | |
| t | 7.66 | 8.051 |
| DF | 11 | 11 |
| P value | <0.0001 | <0.0001 |
| Deviation from zero? | Significant | Significant |
| Data | | |
| Number of X values | 4 | 4 |
| Maximum number of Y | 3 | 3 |
| replicates | | |
| Total number of values | 12 | 12 |
| Number of missing values | 3 | 3 |
| Runs test | | |
| Points above line | 2 | 2 |
| Points below line | 1 | 1 |
| Number of runs | 2 | 2 |
| P value (runs test) | 0.6667 | 0.6667 |
| Deviation from linearity | Not Significant | Not Significant |
| Equation | Y = 6.099e−012*X − 0.0 | Y = 5.170e−012*X − 0.0 |

Using the linear regression model shown above, it was estimated that the average FVIII levels when infusing SPK-8011 at a dose of 5×10¹¹ vg/kg would be around 2.6% to 3.0% of normal. However, this linear regression curve appears to underestimate the actual values observed in low- and mid-dose animals when the equation in Table 2 is used to back calculate the expected FVIII expression values at 2×10¹² vg/kg, 3×10¹² vg/kg and 6×10¹² vg/kg (Table 3).

TABLE 3

| | | | |
|---|---|---|---|
| | FVIII | | Interpolated |
| | (Inter- | FVIII | vs |
| Dose | polated) | (Actual) | actual (%) |

Pilot

| | | | |
|---|---|---|---|
| 2E+12 | 12.2 | 22.3 | 54.8 |
| 6E+12 | 36.6 | 61.6 | 59.4 |
| 2E+13 | 122.0 | 113.5 | 107.5 |

GLP

| | | | |
|---|---|---|---|
| 3E+12 | 15.5 | 20.3 | 76.4 |
| 6E+12 | 31.0 | 40.7 | 76.2 |
| 1.2E+13 | 62.0 | 56.0 | 110.8 |

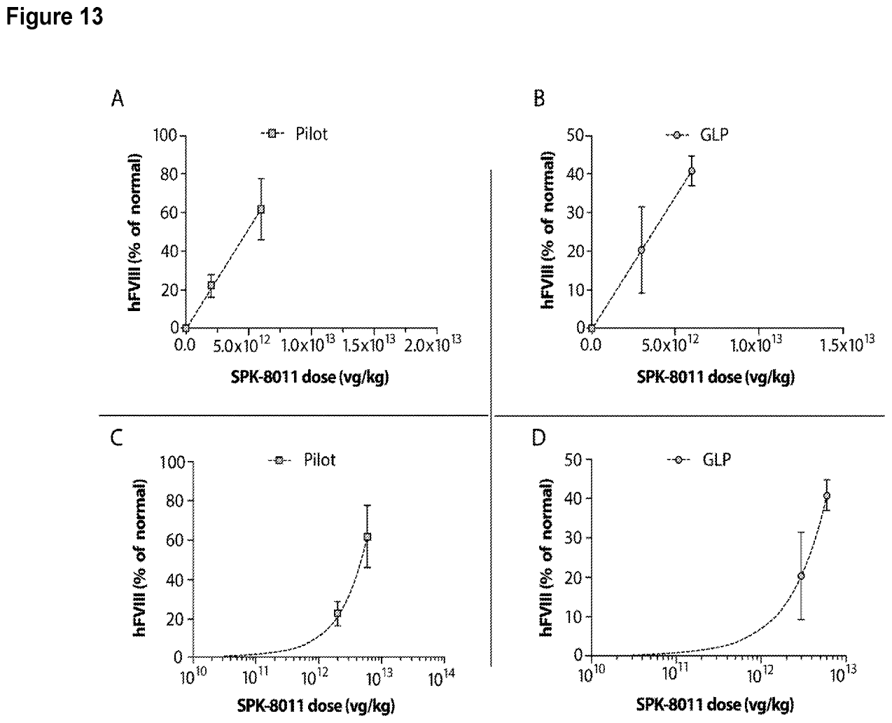

It is possible that hFVIII expression may follow a linear dose response at certain vector doses while reaching saturation as the AAV vector load is increased. The high dose cohort was removed from the previous analysis, the linear regression curve re-calculated and re-evaluated the predicted hFVIII expression levels at an SPK-8011 dose of 5×10¹¹ vg/kg determined (Table 4 and FIG. 13).

TABLE 4

| | Pilot | GLP |
|---|---|---|
| Best-fit values | | |
| Slope | 6.099e−012 ± 7.962e−013 | 5.170e−012 ± 6.421e−013 |
| Y-intercept when X = 0.0 | 0 | 0 |
| X-intercept when Y = 0.0 | 0 | 0 |
| 1/slope | 1.64E+11 | 1.934E+11 |
| 95% Confidence Intervals | | |
| Slope | 4.346e−012 to 7.851e−012 | 3.756e−012 to 6.583e−012 |
| Goodness of Fit | | |
| Sy.x | 28.93 | 15.29 |
| Is slope significantly non-zero? | | |
| t | 7.66 | 8.051 |
| DF | 11 | 11 |
| P value | <0.0001 | <0.0001 |
| Deviation from zero? | Significant | Significant |
| Data | | |
| Number of X values | 4 | 4 |
| Maximum number of Y | 3 | 3 |
| replicates | | |
| Total number of values | 12 | 12 |
| Number of missing values | 12 | 12 |
| Runs test | | |
| Points above line | 2 | 2 |
| Points below line | 1 | 1 |
| Number of runs | 2 | 2 |
| P value (runs test) | 0.6667 | 0.6667 |
| Deviation from linearity | Not Significant | Not Significant |
| Equation | Y = 6.099e−012*X-0.0 | Y = 5.170e−012*X-0.0 |

With the linear regression curves shown in FIG. 13, the average FVIII levels when infusing SPK-8011 at a dose of 5×10¹¹ vg/kg were estimated to be approximately between 3.4% to 5.2% of normal.

### Example 6

### Human Clinical Trial Design

### Eligibility

Ages Eligible for Study: 18 Years and older (Adult, Senior)
Sexes Eligible for Study: Male
Accepts Healthy Volunteers: No

### Criteria: Inclusion Criteria:

Males age 18 years or older
Confirmed diagnosis of hemophilia A as evidenced by their medical history with plasma FVIII activity levels ≤2% of normal
Have received >150 exposure days (EDs) to FVIII concentrates or cryoprecipitate
Have experienced >10 bleeding events over the previous 12 months only if receiving on-demand therapy and having FVIII baseline level 1-2% of normal
Have no prior history of allergic reaction to any FVIII product
Have no measurable inhibitor against factor VIII inhibitor as assessed by the central laboratory and have no prior history of inhibitors to FVIII protein
Agree to use reliable barrier contraception

### Criteria: Exclusion Criteria:

Evidence of active hepatitis B or C.
Currently on antiviral therapy for hepatitis B or C
Have significant underlying liver disease
Have serological evidence* of HIV-1 or HIV-2 with CD4 counts ≤200/mm3 (* participants who are HIV+ and stable with CD4 count >200/mm3 and undetectable viral load are eligible to enroll)
Have detectable antibodies reactive with AAV-Spark200 capsid
Participated in a gene transfer trial within the last 52 weeks or in a clinical trial with an investigational product within the last 12 weeks

### Example 7

### Predicted FVIII Levels at Different Doses of AAV-SPK-8011 (LK03 Capsid)-hFVIII

Clinical study NCT03003533 (‘A Gene Transfer Study for Hemophilia A’) is the first-in-human use of the AAV capsid known as LK03 (SEQ ID NO:27). Studies in non-human primates show that increasing doses of AAV-SPK-8011 (LK03 capsid)-hFVIII result in increasing levels of circulating human FVIII in a dose-dependent manner that, at least for some dose ranges, does not appear to significantly deviate from linearity. Mean steady-state FVIII levels (±standard error of the mean) in the first cohort were approximately 11.7±2.3% of normal. Given the n of two participants in this dose cohort, it is difficult to predict whether the relatively low variability in FVIII levels observed will be maintained as more participants are included in the study.

Recent experience using rAAV vectors to mediate expression of a coagulation factor in the liver, using investigational product rAAV-FIX for the treatment of hemophilia B (NCT02484092), may be a useful reference to estimate variability in a larger cohort of subjects. Steady-state FIX expression was reached by 12 weeks after rAAV-FIX vector infusion, resulting in a mean FIX activity (FIX:C) of approximately 33%. Importantly, the highest levels of FIX:C were around 79% (subject 9) and the lowest levels were around 14% (subject 7). Of note, interpretation of vector potency in subject 7 was confounded by the occurrence of an immune response against the rAAV-FIX vector capsid, which resulted in partial loss of FIX expression before a short course of steroids was initiated. Subject 6, however, in which no cellular immune response was detected, had steady state levels of approximately 18%. Thus, the difference between the highest and the lowest FIX:C levels in study NCT02484092 was approximately 4-fold. Other AAV clinical trials for the treatment of hemophilia have shown significantly higher variability. Pasi, et al. (2017) Thromb Haemost. 117(3): 508-518. Table 5 shows the predicted mean FVIII levels at different AAV-SPK-8011 (LK03 capsid)-hFVIII doses assuming a linear dose-response. The observed variability in the hemophilia B study was used as a conservative approach to estimate variability in the hemophilia A trial.

TABLE 5

| | Estimated | | Estimated |
|---|---|---|---|
| Dose | lowest | Estimated | highest |
| (vg/kg) | expresser | mean* | expresser |
| 5.00E+11 | 6 | 12 | 24 |
| 1.00E+12 | 12 | 24 | 48 |
| 2.00E+12 | 24 | 48 | 96 |
| 4.00E+12 | 48 | 96 | 192 |
| 6.00E+12 | 96 | 192 | 384 |

*Actual mean observed in the 5 × 10¹¹ vg/kg cohort.

### Example 8

### Human Clinical Trial Results

A dose escalation study was performed in twelve men with severe (N=11) or moderately severe (N=1) hemophilia A. Subjects ranged in age from 18-52. Prior to gene therapy, 8 of the 12 subjects were managed with prophylaxis, and 4 of the 12 subjects with episodic treatment. Subjects were enrolled in one of three dosing cohorts, and infused with SPK-8011 (AAV-hFVIII, LK03 capsid) at a dose of 5×10¹¹ vg/kg (N=2, Subjects 1 and 2), 1×10¹² vg/kg (N=3, Subjects 3, 4 and 6), or 2×10¹² vg/kg (N=7, Subjects 5 and 7-12).

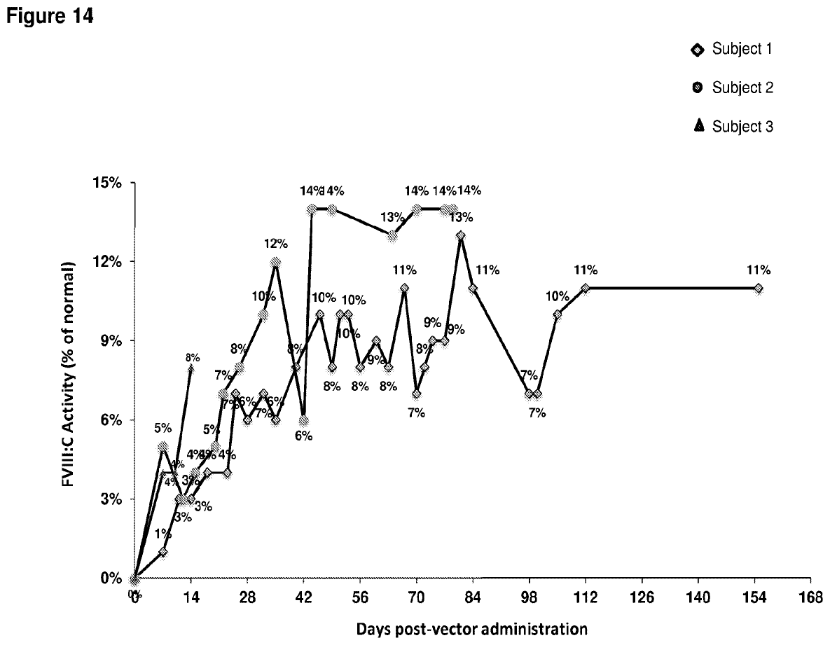

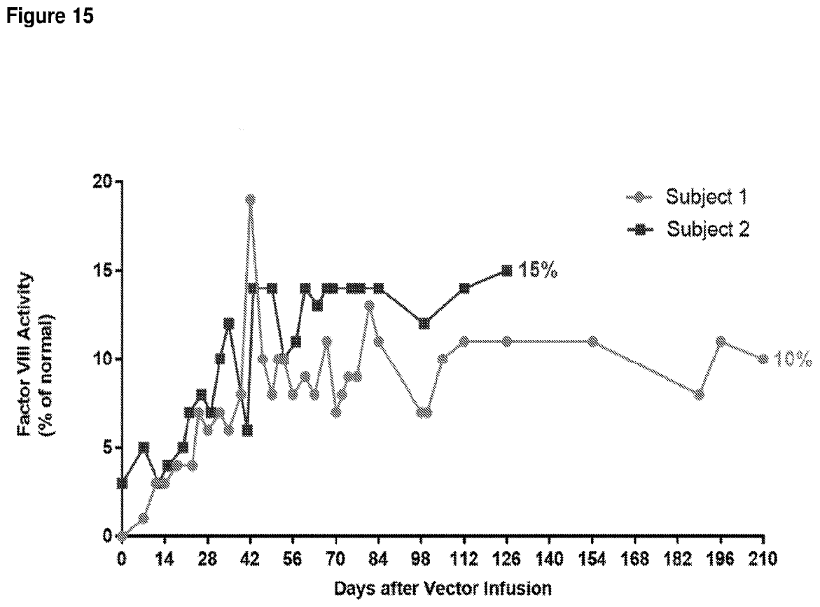

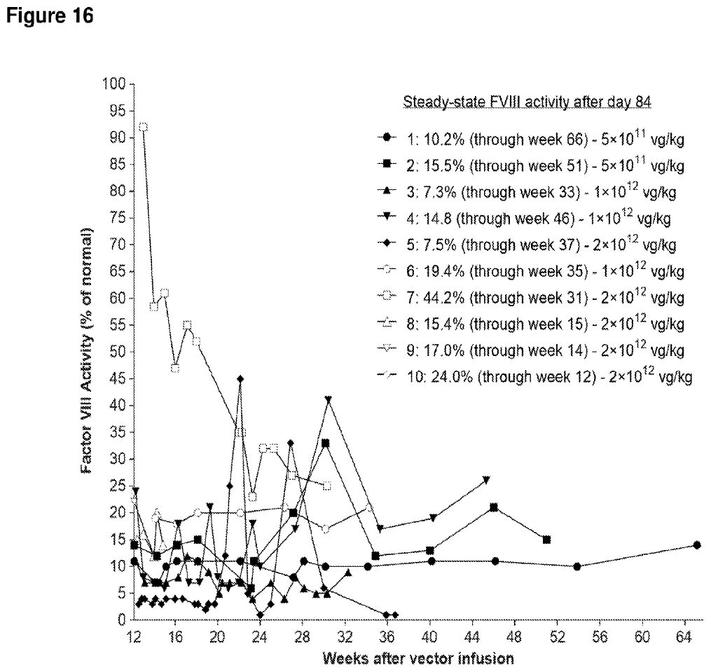

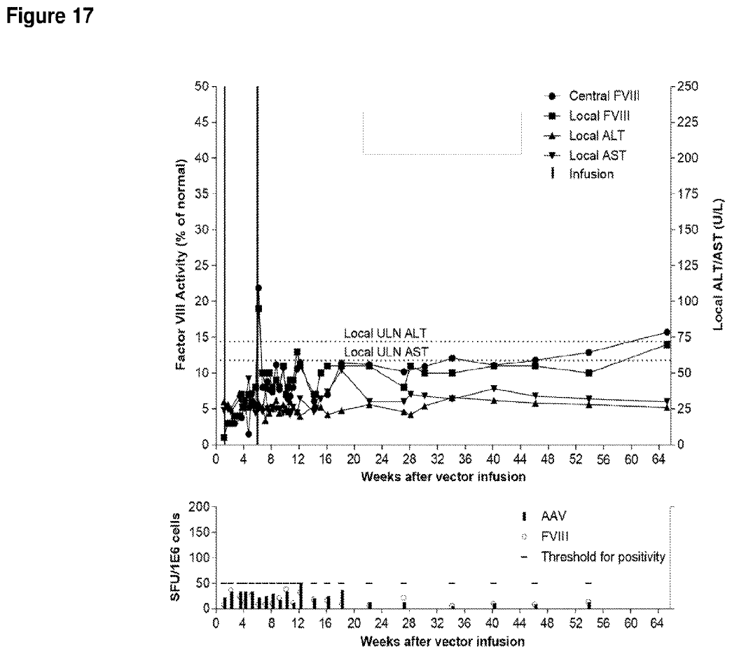

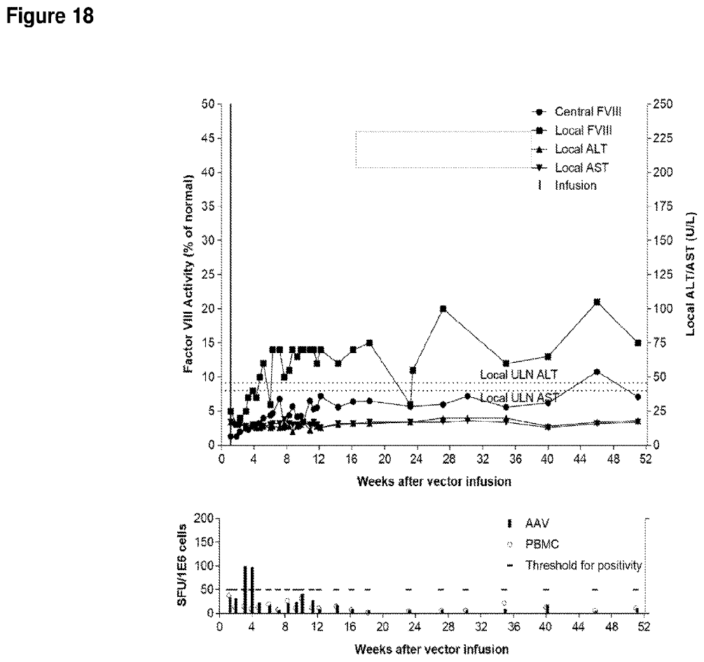

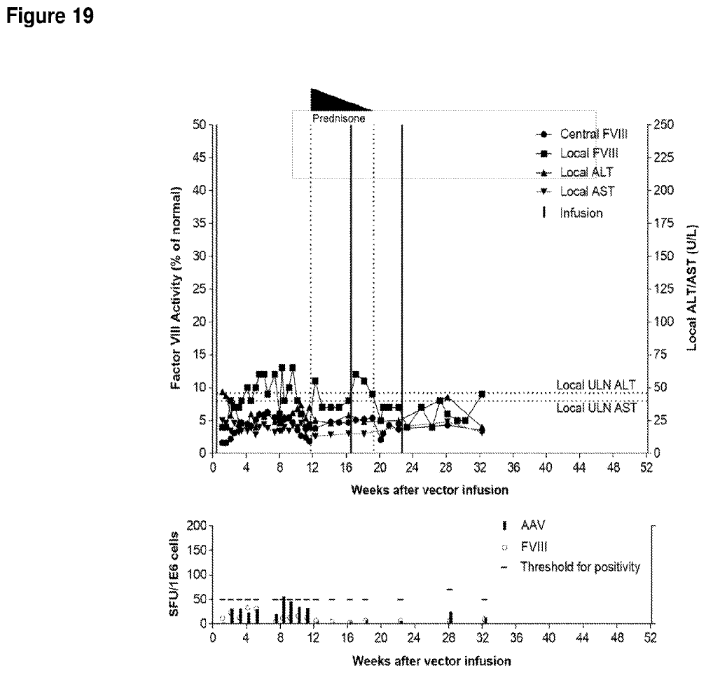

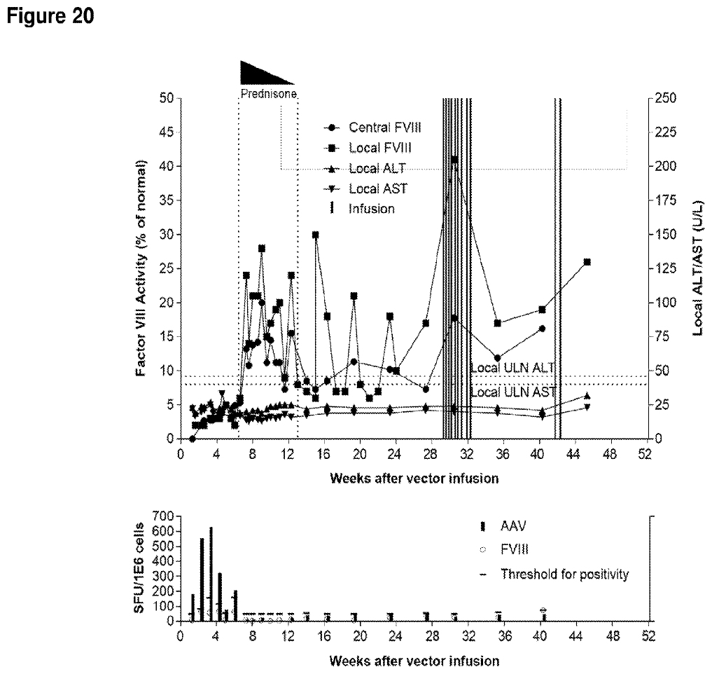

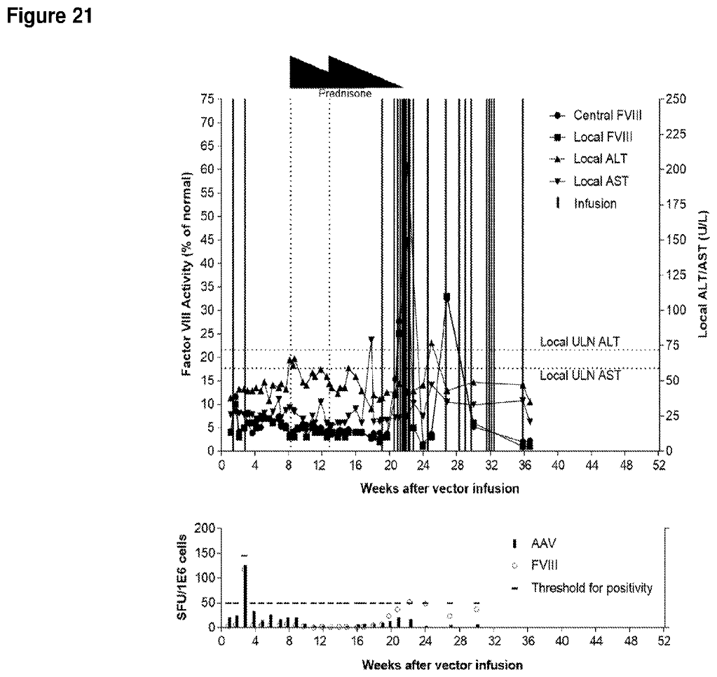

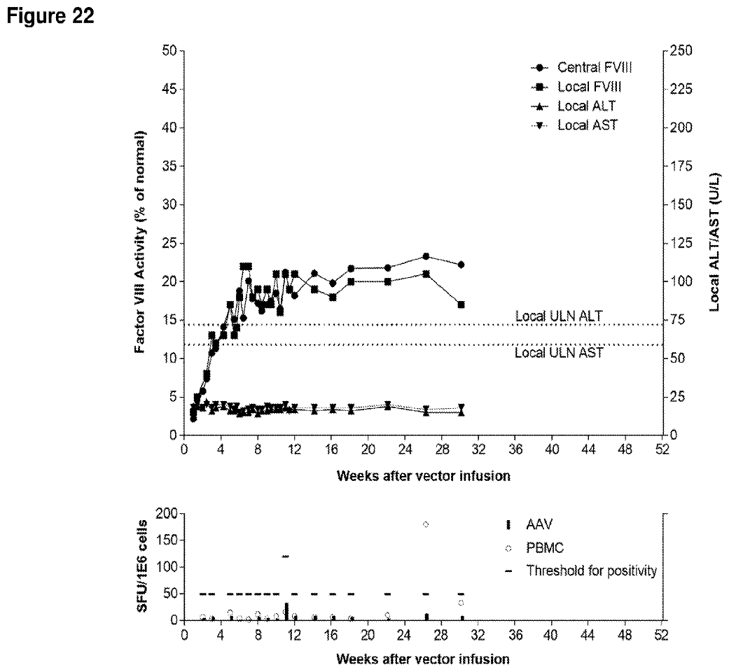

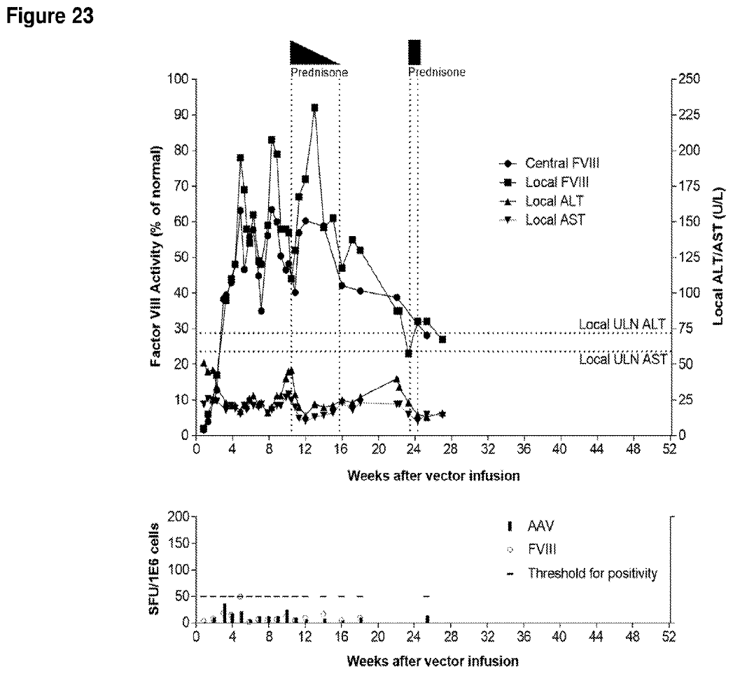

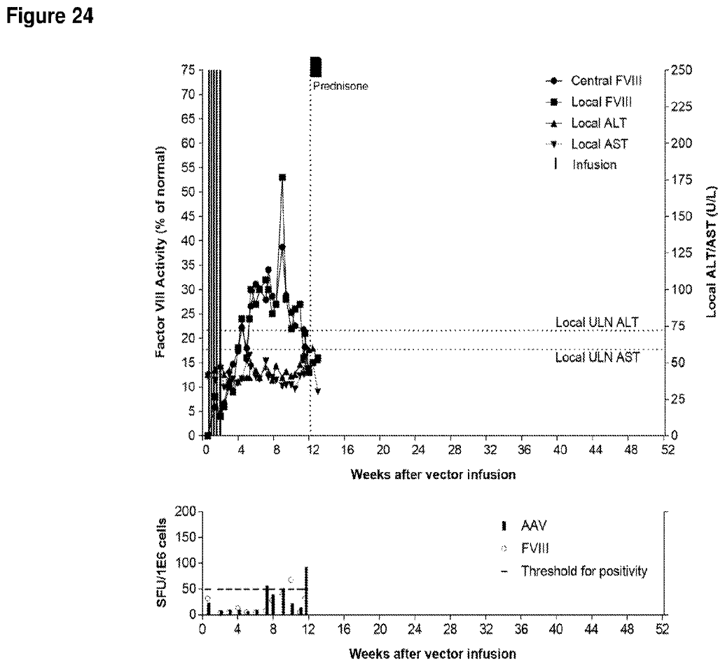

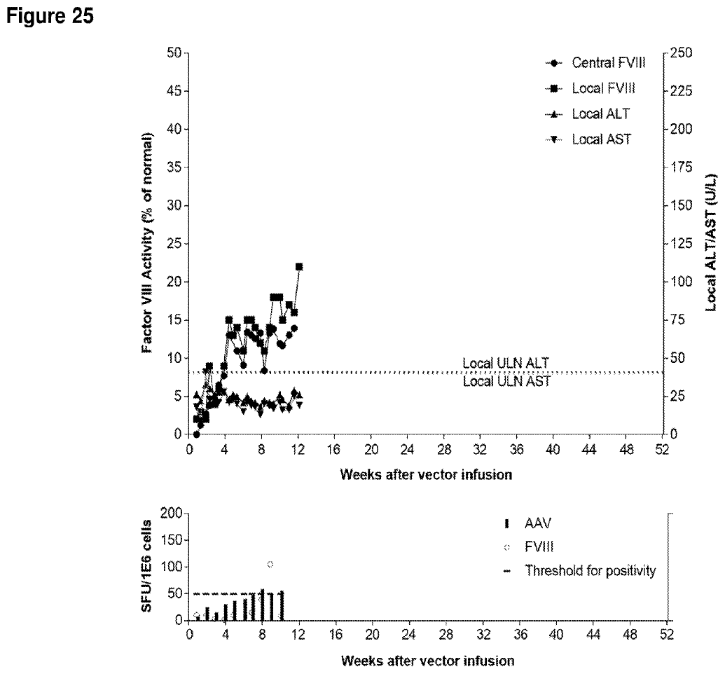

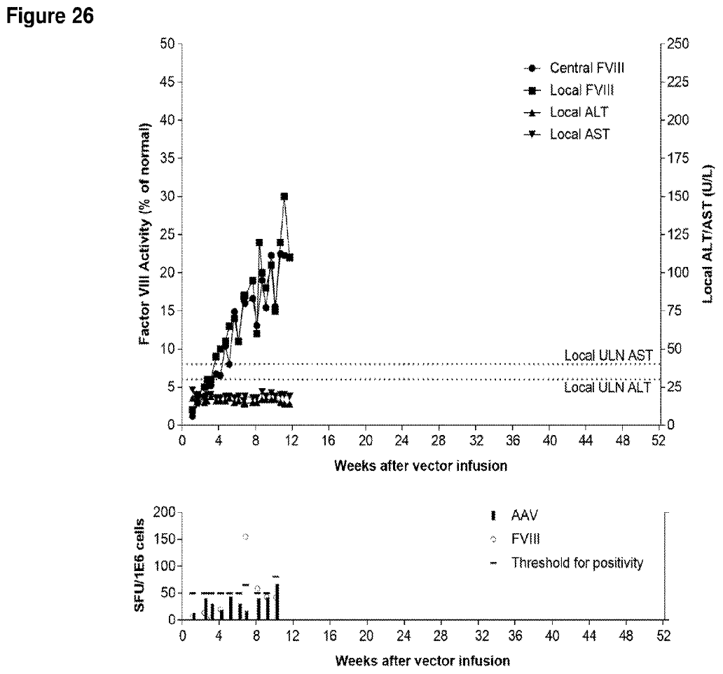

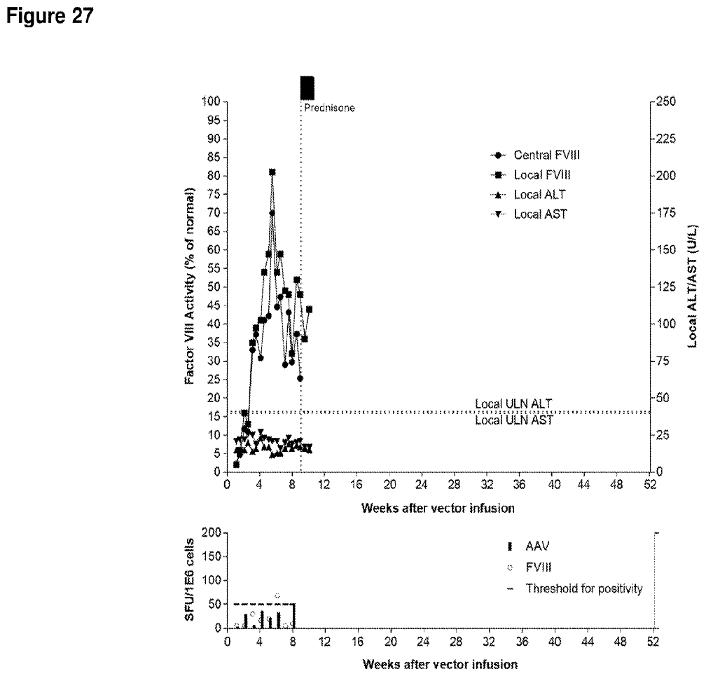

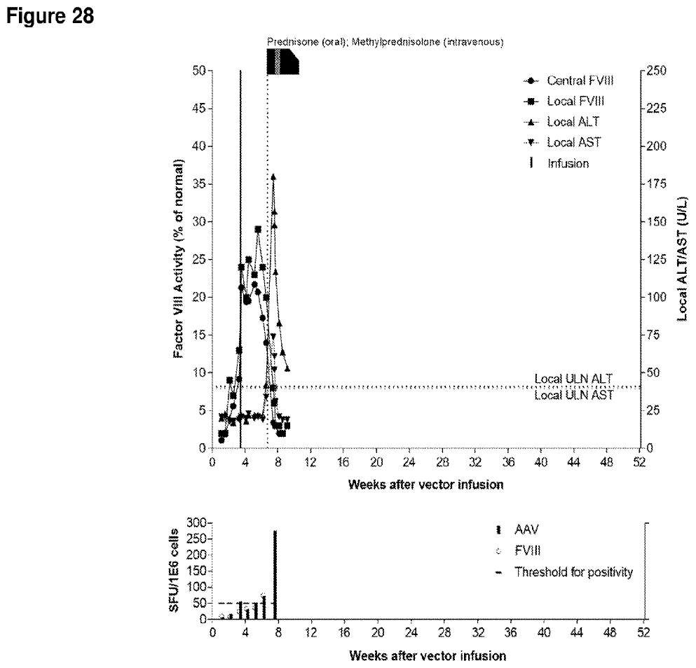

FIGS. 14-28 show dose response study data of the 12 human subjects administered the three different doses of AAV-SPK-8011 (LK03 capsid)-hFVIII. The values of FVIII activity determined in the subjects is relative to 100% FVIII in normal plasma. Typically, plasma is pooled from a large number (say 50 or 100) normal volunteers and the FVIII activity in this “normal pooled plasma” is defined as 100%. Dilutions of this plasma are used to make a standard curve of FVIII activity versus whatever assay is used to determine FIX levels. This standard curve is then used to define the amount or percent (%) FVIII in a patient sample using the same assay.

All vector doses led to expression of levels of FVIII sufficient to prevent bleeding and allow cessation of prophylaxis. Across the 12 subjects at 3 doses, there was a 97% reduction in annualized bleeding rate (ABR), and a 97% reduction in annualized infusion rate. The data indicate that the overall kinetics show a gradual rise to a sustained plateau of FVIII.

In the first dose cohort, FVIII levels are 14% and 15%, at 66 and 51 weeks, with no bleeding events, no elevated transaminase levels, and no use of steroids. FVIII expression has remained stable over the period of observation. Data from this low dose cohort indicate that even modest FVIII levels in the range of 15% may be adequate to prevent bleeding over a follow-up period of up to 66 weeks.

In the second dose cohort, FVIII levels are 9%, 26%, and 17% at 33, 46, and 31 weeks post infusion. The first subject in this dose cohort (Subject 3) infused a single dose of factor concentrate for a spontaneous joint bleed at day 159 and the second in this dose cohort (Subject 4) received multiple infusions for a traumatic bleed beginning at day 195. These subjects both received a course of tapering steroids, instituted at 12 and 7 weeks post vector infusion, triggered by a decline in FVIII levels, with resultant stabilization of FVIII levels. The third subject in this dose cohort (Subject 6) has had no bleeding and did not receive factor infusions nor were steroids given.

In the third dose cohort (N=7), five of seven subjects currently have FVIII levels ≥12%, with a range of 16-49%; for these subjects, the mean FVIII level beginning 12 weeks after vector infusion is 30% and the median is 22%. No bleeds have been reported among these subjects beginning 4 weeks post vector infusion.

Separately, five of the 7 at the 2×10¹² vg/kg AAV-LK03 (FVIII) vector dose received a course of steroids, initiated at time points ranging from 6 to 11 weeks after vector infusion, for one or more of the following: declining FVIII levels, rise in ALT above subject baseline, or elevated IFN-γ ELISPOTs to AAV capsid. Initiation of steroids was associated with reduction of ALT to the normal range, and extinguishing of ELISPOT signal in all cases; two subjects out of seven showed limited success in stabilizing FVIII levels, which fell to <5% possibly due to immune responses. For one of these, no bleeds have been reported through 12 weeks of follow up; the other has had 4 bleeds through 37 weeks of observation.

Overall, a favorable safety profile was observed, with only two subjects experiencing ALT elevation above the upper limit of normal. Ninety-one percent (91%) of subjects to date have experienced an ABR of ≤1 since vector infusion. All subjects experienced a rise in FVIII levels following vector infusion, but limited success in preventing declines in FVIII levels in two subjects suggests that addition of prophylactic steroids may be warranted.

Based on the hFVIII levels seen in non NHPs, and taking into account that different vector lots can have slightly different potency, it was estimated that the average FVIII levels in humans infused with SPK-8011 at a dose of 5×10¹¹ vg/kg might be approximately around 3.4%-5.8%, assuming a linear extrapolation. FVIII activity in the first subject plateaued at approximately 9.15±0.53% of normal and 13.50±0.50% in the second subject. Thus, average FVIII activity in the low dose cohort was approximately 11.3%, which is 2-4-fold higher than expected based upon studies in non-human primates.

The substantial 2-4-fold difference (depending upon the linear regression curve used) in the low dose cohort between predicted FVIII levels based on pre-clinical studies using a phylogenetically close species such as macaques and the actual results in human subjects highlights the limitations of current animal models in determining AAV vector dosages for humans. The data indicating that there was far greater FVIII activity in humans than predicted based upon the FVIII activity in NHPs administered AAV-SPK-8011 (LK03 capsid)-hFVIII was not expected.

While a universal preclinical model to determine AAV dosage in humans does not exist, previous experience in non-human primates using AAV2, AAV8 and AAV-Spk vectors to mediate liver-derived expression of coagulation factor IX indicates that macaques are a good but not perfect predictor of AAV vector efficacy in humans. More recently, chimeric “humanized” mice with livers partially repopulated with human hepatocytes have become a valuable tool to determine hepatic transduction efficacy of different viral capsids. Two independent studies have been reported that measured transduction in human hepatocytes taking advantage of this mouse model. It was reported that an approximately 10-fold difference in the percent of transduced human hepatocytes between LK03 and AAV8 (43.3±11% and 3.6±1.1% with LK03 and AAV8 vector infusion, respectively was observed (Lisowski L, et al. Nature 506:382-6 (2014)).

In sum, infusion of SPK-8011 in 12 patients with severe or moderately severe Hemophilia A resulted in safe, durable, dose-dependent FVIII activity associated with 97% reduction in ABR and 97% in recombinant FVIII usage for a period of up to 66 weeks post-gene transfer.

### Example 9

### TTR Promoter

The characterization of the transthyretin (TTR) promoter was originally described in Costa and Grayson 1991, Nucleic Acids Research 19(15): 4139-4145. The TTR promoter sequence was a modified sequence, from TATTTGTGTAG (SEQ ID NO: 30) to TATTGACTTAG (SEQ ID NO: 31).

TTR promoter with 4 nucleotide mutation (TTRmut),
SEQ ID NO: 22
GTCTGTCTGCACATTTCGTAGAGCGAGTGTTCCGATACTCTAATCTCCCT
AGGCAAGGTTCATATTGACTTAGGTTACTTATTCTCCTTTTGTTGACTAA
GTCAATAATCAGAATCAGCAGGTTTGGAGTCAGCTTGGCAGGGATCAGCA
GCCTGGGTTGGAAGGAGGGGGTATAAAAGCCCCTTCACCAGGAGAAGCCG
TCACACAGATCCACAAGCTCCT

### Example 10

### CpG Reduced FVIII Encoding Transgene Constructs and Exemplary AAV Capsids.

FVIII encoding CpG reduced nucleic acid variant X01
(SEQ ID NO: 1)
atgcagattg agctgtctac ctgcttcttc ctgtgcctgc tgaggttctg cttctctgct
accaggaggt actacctggg ggctgtggag ctgagctggg attacatgca gtctgacctg
ggggagctgc ctgtggatgc caggtttccc cccagggtgc ccaagagctt ccccttcaat
acctctgtgg tgtataagaa gaccctgttt gtggagttca ctgatcatct gttcaacatt
gctaaaccca ggcccccctg gatggggctg ctgggcccta ccatccaggc tgaggtgtat
gacactgtgg tgatcactct gaagaacatg gctagccatc ctgtgtctct gcatgctgtg
ggggtgagct actggaaggc ttctgagggg gctgagtatg atgatcagac tagccagagg
gagaaggagg atgacaaggt gttccctggg ggctctcaca cctatgtctg gcaggtgctg
aaggagaatg gccccatggc ctctgatcct ctgtgtctga cctatagcta cctgagccat
gtggacctgg tgaaggacct gaactctggc ctgattgggg ccctgctggt gtgtagggag
gggagcctgg ccaaggagaa gacccagacc ctgcacaagt tcattctgct gtttgctgtg
tttgatgagg gcaagagctg gcattctgaa accaagaaca gcctgatgca ggacagggat
gctgcctctg ctagggcctg gcccaagatg cacactgtga atgggtatgt caataggtct
ctgcctggcc tgattggctg ccacaggaag tctgtgtact ggcatgtgat tgggatgggc
accacccctg aggtgcacag catctttctg gagggccaca ccttcctggt gaggaatcac
agacaggcca gcctggagat cagccccatc accttcctga ctgcccagac cctgctgatg
gacctgggcc agtttctgct gttctgccac atctctagcc accagcatga tggcatggag
gcctatgtga aggtggactc ctgccctgag gagccccagc tgaggatgaa gaataatgag
gaggctgagg actatgatga tgacctgact gactctgaga tggatgtggt gagatttgat
gatgacaatt ctcccagctt cattcagatc aggtctgtgg ccaagaagca tcccaagacc
tgggtgcact acattgctgc tgaggaggag gactgggact atgcccccct ggtgctggcc
cctgatgaca ggagctataa gagccagtac ctgaataatg gcccccagag gattgggagg
aagtataaga aggtgaggtt catggcctat actgatgaaa ccttcaagac cagagaggcc
atccagcatg agtctgggat cctggggccc ctgctgtatg gggaggtggg ggacaccctg
ctgatcatct tcaagaacca ggccagcagg ccctacaaca tctaccctca tggcatcact
gatgtgaggc ctctgtacag cagaaggctg cccaaggggg tgaagcatct gaaggacttc
cccattctgc ctggggagat tttcaagtac aagtggactg tgactgtgga ggatggccca
accaagtctg accctaggtg cctgactagg tactacagca gctttgtgaa tatggagagg
gacctggcct ctggcctgat tggccccctg ctgatctgct acaaggagtc tgtggatcag
aggggcaacc agatcatgtc tgacaagagg aatgtgatcc tgttctctgt gtttgatgag
aacaggagct ggtacctgac tgagaacatt cagaggtttc tgcccaaccc tgctggggtg
cagctggagg accctgaatt ccaggcctct aacatcatgc acagcattaa tggctatgtg
tttgacagcc tgcagctgtc tgtgtgcctg catgaggtgg cctactggta cattctgagc
attggggccc agactgactt cctgtctgtg ttcttctctg gctacacctt taagcacaag
atggtgtatg aggataccct gaccctgttt cctttctctg gggagactgt gttcatgagc
atggagaacc ctggcctgtg gatcctgggc tgccacaact ctgacttcag gaacaggggg
atgactgctc tgctgaaggt gagcagctgt gataagaaca ctggggacta ctatgaggac
agctatgagg acatctctgc ctatctgctg agcaagaata atgctattga gcccaggagc
ttctctcaga acccccctgt gctgaagagg caccagaggg agatcaccag aactactctg
cagtctgacc aggaggagat tgactatgat gacaccatct ctgtggagat gaagaaggag
gattttgata tttatgatga ggatgaaaac cagagcccca ggagctttca gaagaagact
aggcactatt tcattgctgc tgtggagagg ctgtgggact atggcatgtc ttctagcccc
catgtgctga ggaacagggc ccagtctggc tctgtgcccc agttcaagaa ggtggtgttc
caggagttca ctgatggcag cttcactcag cccctgtaca ggggggagct gaatgagcac
ctggggctgc tgggccctta tatcagggct gaggtggagg ataacatcat ggtgaccttc
aggaaccagg ccagcaggcc ctacagcttc tactctagcc tgatcagcta tgaggaggac
cagaggcagg gggctgagcc caggaagaac tttgtgaagc ccaatgagac caagacttat
ttctggaagg tgcagcacca tatggccccc accaaggatg agtttgattg caaagcctgg
gcctacttct ctgatgtgga cctggagaag gatgtgcact ctgggctgat tggccccctg
ctggtgtgcc acaccaacac tctgaaccct gcccatggca ggcaggtgac tgtgcaggag
tttgccctgt tcttcaccat ctttgatgag actaagagct ggtacttcac tgagaacatg
gagaggaact gcagggcccc ctgcaatatc cagatggagg accccacctt taaggaaaat
tataggtttc atgccattaa tggctacatc atggacaccc tgcctggcct ggtgatggcc
caggaccaga ggatcaggtg gtacctgctg agcatgggca gcaatgagaa cattcacagc
atccacttct ctggccatgt gttcactgtg aggaagaagg aggagtacaa gatggccctg
tataatctgt accctggggt gtttgagact gtggagatgc tgcccagcaa ggctggcatc
tggagggtgg agtgcctgat tggggagcac ctgcatgctg gcatgagcac cctgttcctg
gtgtattcta acaagtgtca gacccccctg ggcatggcct ctggccatat cagggacttc
cagatcactg cctctggcca gtatgggcag tgggccccca agctggccag gctgcattac
tctggcagca tcaatgcctg gagcaccaag gagccattca gctggattaa ggtggacctg
ctggctccaa tgattatcca tggcatcaag acccaggggg ccaggcagaa gtttagcagc
ctgtacatct ctcagtttat catcatgtac tctctggatg gcaaaaagtg gcagacctac
aggggcaatt ctactggcac tctgatggtg ttctttggca atgtggacag ctctgggatc
aagcacaaca tctttaaccc ccctatcatt gccaggtaca ttaggctgca ccccacccat
tacagcatca ggagcaccct gaggatggag ctgatgggct gtgatctgaa cagctgcagc
atgcccctgg gcatggagag caaggctatc tctgatgccc agattactgc cagcagctac
ttcaccaata tgtttgccac ctggagcccc agcaaggcca ggctgcacct gcagggcagg
tctaatgcct ggaggcccca ggtgaacaac cccaaggagt ggctgcaggt ggacttccag
aagaccatga aggtgactgg ggtgaccacc cagggggtga agagcctgct gactagcatg
tatgtgaagg agttcctgat cagcagcagc caggatggcc atcagtggac cctgttcttc
cagaatggca aggtgaaggt gttccagggc aatcaggaca gcttcacccc tgtggtgaac
agcctggacc cccccctgct gaccagatac ctgaggatcc acccccagag ctgggtgcat
cagattgccc tgaggatgga ggtgctgggg tgtgaggccc aggacctgta ctga
FVIII encoding CpG reduced nucleic acid variant X02
(SEQ ID NO: 2)
atgcagattg agctgtctac ctgctttttc ctgtgtctgc tgaggttctg cttctctgcc
actaggaggt actacctggg ggctgtggag ctgtcttggg attacatgca gtctgatctg
ggggagctgc ctgtggatgc caggtttcct cccagggtgc ccaagtcttt ccccttcaat
acctctgtgg tgtataagaa gaccctgttt gtggagttta ctgatcacct gttcaacatt
gccaagccca ggcccccttg gatgggcctg ctggggccca ccatccaggc tgaggtgtat
gacactgtgg tgatcaccct gaagaacatg gcctctcacc ctgtgagcct gcatgctgtg
ggggtgagct actggaaggc ctctgagggg gctgagtatg atgaccagac cagccagagg
gagaaggagg atgataaggt gttccctggg gggagccaca cttatgtgtg gcaggtgctg
aaggagaatg gcccaatggc ctctgatccc ctgtgcctga cctattctta cctgagccat
gtggacctgg tgaaggacct gaactctggc ctgattgggg ccctgctggt gtgcagggag
ggctctctgg ctaaggagaa gacccagacc ctgcacaagt tcatcctgct gtttgctgtg
tttgatgagg ggaagagctg gcactctgag accaagaaca gcctgatgca ggacagggat
gctgcctctg ccagggcctg gcccaaaatg cacactgtga atggctatgt gaataggagc
ctgcctggcc tgattggctg ccacaggaag tctgtgtatt ggcatgtgat tggcatgggc
accacccctg aggtgcactc tatcttcctg gagggccata ctttcctggt gaggaatcat
aggcaggcca gcctggagat tagccccatt acctttctga ctgcccagac cctgctgatg
gacctgggcc agttcctgct gttttgccac atcagctctc accagcatga tggcatggag
gcctatgtga aggtggatag ctgccctgag gagccccagc tgaggatgaa gaacaatgag
gaggctgagg attatgatga tgatctgact gattctgaaa tggatgtggt gaggtttgat
gatgacaata gcccctcttt catccagatc aggtctgtgg ccaagaagca tcctaagacc
tgggtgcact acattgctgc tgaggaggag gactgggact atgctcccct ggtgctggcc
cctgatgaca ggtcttacaa gagccagtac ctgaacaatg gcccccagag aattgggagg
aagtataaga aggtgagatt catggcttac actgatgaga ccttcaagac tagggaggcc
atccagcatg agtctggcat tctgggcccc ctgctgtatg gggaggtggg ggacaccctg
ctgatcatct tcaagaacca ggcctctagg ccctacaata tttaccccca tgggatcact
gatgtgaggc ccctgtacag caggaggctg cctaaggggg tgaagcatct gaaggacttc
cccatcctgc ctggggagat cttcaagtat aagtggactg tgactgtgga agatggcccc
accaagtctg accctaggtg cctgaccagg tactactctt cttttgtgaa catggagagg
gacctggcct ctggcctgat tggccccctg ctgatctgct acaaggagtc tgtggaccag
agggggaacc agattatgtc tgacaagagg aatgtgattc tgttctctgt gtttgatgag
aacaggagct ggtatctgac tgagaacatc cagaggttcc tgcccaatcc tgctggggtg
cagctggagg accctgagtt ccaggccagc aacatcatgc acagcatcaa tgggtatgtg
tttgattctc tgcagctgtc tgtgtgcctg catgaggtgg cctactggta catcctgagc
attggggctc agactgattt cctgtctgtg ttcttttctg gctacacctt taagcataag
atggtgtatg aggacactct gaccctgttt cccttctctg gggagactgt gtttatgagc
atggagaacc ctggcctgtg gatcctgggc tgccacaact ctgatttcag gaacaggggc
atgactgctc tgctgaaggt gtcttcttgt gacaagaaca ctggggacta ttatgaggac
agctatgagg acatctctgc ctacctgctg agcaagaaca atgctattga gcccagatct
ttcagccaga acccccctgt gctgaagagg caccagaggg agatcactag gaccaccctg
cagtctgacc aggaggagat tgactatgat gacactatct ctgtggagat gaagaaggag
gactttgata tctatgatga ggatgagaac cagtctccca ggagcttcca gaaaaagacc
aggcactact tcattgctgc tgtggagagg ctgtgggact atggcatgtc ttctagcccc
catgtgctga ggaacagggc ccagtctggg tctgtgcccc agttcaagaa ggtggtgttc
caggagttca ctgatgggag cttcacccag cctctgtaca ggggggagct gaatgagcac
ctggggctgc tgggccctta tattagggct gaggtggagg acaacatcat ggtgactttc
aggaatcagg cctctaggcc ctatagcttc tacagctctc tgatcagcta tgaggaggat
cagaggcagg gggctgagcc caggaagaac tttgtgaagc ccaatgagac caagacctac
ttctggaagg tgcagcacca catggctcct accaaggatg agtttgactg caaggcctgg
gcctactttt ctgatgtgga cctggagaag gatgtgcact ctggcctgat tggccccctg
ctggtgtgtc ataccaacac cctgaaccct gcccatggca ggcaggtgac tgtgcaggag
tttgccctgt tcttcaccat ctttgatgag accaagagct ggtactttac tgagaacatg
gagaggaatt gcagagcccc ttgcaacatc cagatggagg acccaacctt caaagagaac
tacaggttcc atgccatcaa tgggtacatc atggacaccc tgcctggcct ggtgatggct
caggaccaga ggatcaggtg gtatctgctg agcatgggca gcaatgagaa tatccatagc
attcacttct ctggccatgt gttcactgtg aggaagaagg aggagtacaa gatggccctg
tataacctgt accctggggt gtttgagact gtggagatgc tgccaagcaa ggctgggatt
tggagggtgg agtgcctgat tggggagcac ctgcatgctg gcatgtctac cctgttcctg
gtgtactcca ataagtgcca gacccccctg ggcatggcct ctggccacat cagggacttc
cagatcactg cctctggcca gtatgggcag tgggccccaa agctggccag gctgcactat
tctgggagca tcaatgcttg gagcaccaag gagcctttca gctggattaa ggtggatctg
ctggccccca tgatcattca tggcatcaaa acccaggggg ctagacagaa gttttctagc
ctgtacatca gccagttcat catcatgtac agcctggatg gcaagaagtg gcagacttac
aggggcaata gcactggcac cctgatggtg ttttttggca atgtggacag ctctggcatc
aagcacaaca tctttaaccc ccccattatt gccaggtata tcaggctgca tcccacccac
tattctatta ggtctactct gagaatggag ctgatgggct gtgacctgaa cagctgtagc
atgcccctgg ggatggagag caaggctatc tctgatgccc agatcactgc cagctcttat
ttcaccaata tgtttgccac ctggtctccc tctaaggcca ggctgcacct gcagggcagg
agcaatgctt ggaggcccca ggtgaataac cccaaggagt ggctgcaggt ggacttccag
aagaccatga aggtgactgg ggtgactacc cagggggtga agtctctgct gactagcatg
tatgtgaagg agttcctgat cagcagcagc caggatgggc atcagtggac tctgttcttc
cagaatggca aggtgaaggt cttccagggg aaccaggata gcttcactcc tgtggtgaac
tctctggacc cccccctgct gactaggtat ctgaggatcc acccccagag ctgggtgcac
cagattgccc tgaggatgga ggtgctgggc tgtgaggccc aggacctgta ttga
FVIII encoding CpG reduced nucleic acid variant X03
(SEQ ID NO: 3)
atgcagattg aactgtctac ttgtttcttc ctgtgcctgc tgaggttttg cttctctgct
actaggaggt actatctggg ggctgtggag ctgtcttggg actatatgca gtctgacctg
ggggagctgc ctgtggatgc taggtttccc cccagggtgc ccaagagctt cccctttaac
acctctgtgg tgtataagaa gactctgttt gtggagttca ctgaccatct gttcaacatt
gccaagccaa ggcccccctg gatgggcctg ctgggcccca ccatccaggc tgaggtgtat
gacactgtgg tgattactct gaagaacatg gccagccatc ctgtgagcct gcatgctgtg
ggggtgtctt actggaaggc ctctgagggg gctgagtatg atgaccagac ctctcagagg
gagaaggagg atgacaaggt gttccctggg ggctctcata cctatgtgtg gcaggtcctg
aaggagaatg ggcccatggc ctctgacccc ctgtgcctga cctactctta tctgtctcat
gtggacctgg tgaaggacct gaactctggc ctgattgggg ccctgctggt gtgcagggag
ggcagcctgg ctaaggagaa gacccagact ctgcacaagt tcatcctgct gtttgctgtg
tttgatgagg gcaagagctg gcactctgag accaagaaca gcctgatgca ggacagggat
gctgcctctg ctagggcctg gcccaagatg cacactgtga atgggtatgt gaacaggagc
ctgccaggcc tgattggctg ccataggaag tctgtgtatt ggcatgtgat tgggatgggg
actacccctg aggtccacag cattttcctg gaggggcata cctttctggt gaggaaccac
aggcaggcct ctctggagat ctctcccatt actttcctga ctgcccagac cctgctgatg
gacctgggcc agttcctgct gttctgccac atcagcagcc accagcatga tggcatggag
gcctatgtga aggtggatag ctgccctgag gagccccagc tgaggatgaa aaacaatgag
gaggctgagg attatgatga tgacctgact gattctgaga tggatgtggt gaggtttgat
gatgataaca gccccagctt catccagatt aggtctgtgg ccaagaagca tcccaagacc
tgggtgcact acattgctgc tgaggaggag gattgggact atgctcctct ggtgctggcc
cctgatgaca ggagctacaa gagccagtac ctgaataatg gcccccagag gattggcagg
aagtataaga aggtgaggtt catggcctac actgatgaga cctttaagac cagggaggcc
atccagcatg aatctgggat cctgggcccc ctgctgtatg gggaggtggg ggacaccctg
ctgattatct ttaagaacca ggctagcagg ccctacaaca tttaccccca tggcattact
gatgtgaggc ccctgtacag caggaggctg cccaaggggg tgaagcacct gaaggatttc
cccattctgc ctggggagat ctttaagtac aaatggactg tgactgtgga ggatggccct
actaagtctg atcccaggtg tctgaccaga tactacagca gctttgtgaa tatggagagg
gacctggctt ctggcctgat tggccccctg ctgatctgct acaaggagtc tgtggaccag
aggggcaatc agattatgtc tgacaagagg aatgtgatcc tgttctctgt gtttgatgag
aacagaagct ggtacctgac tgagaacatc cagaggttcc tgcccaaccc tgctggggtg
cagctggagg accctgagtt ccaggctagc aatatcatgc acagcattaa tggctatgtg
tttgacagcc tgcagctgtc tgtgtgcctg catgaggtgg cctattggta cattctgagc
attggggccc agactgattt cctgtctgtg ttcttttctg gctacacctt caagcacaag
atggtgtatg aggatactct gaccctgttt cccttctctg gggagactgt gttcatgagc
atggagaacc ctggcctgtg gatcctgggc tgtcacaact ctgacttcag gaacaggggc
atgactgccc tgctgaaggt gagctcttgt gataagaaca ctggggacta ctatgaggac
tcttatgagg acatctctgc ctacctgctg agcaagaaca atgctattga gcccaggagc
ttctctcaga atccccctgt gctgaagagg catcagaggg agatcactag gactaccctg
cagtctgacc aggaagagat tgactatgat gacaccatct ctgtggaaat gaagaaggag
gactttgata tctatgatga ggatgaaaac cagagcccca ggagcttcca gaagaagacc
aggcattact tcattgctgc tgtggagagg ctgtgggact atgggatgag ctcttctccc
catgtgctga ggaatagggc tcagtctggc tctgtcccac agttcaagaa ggtggtgttt
caggagttca ctgatggcag cttcactcag cccctgtaca ggggggagct gaatgagcat
ctgggcctgc tggggcccta catcagggct gaggtggagg ataacattat ggtgactttc
aggaaccagg cctctaggcc ctacagcttc tacagcagcc tgatcagcta tgaggaggac
cagaggcagg gggctgagcc caggaagaac tttgtgaagc ccaatgagac taagacctat
ttctggaagg tgcagcatca catggctccc actaaagatg agtttgactg caaggcctgg
gcctacttct ctgatgtgga tctggagaag gatgtgcatt ctgggctgat tggccctctg
ctggtctgcc atactaacac cctgaatcct gcccatggca ggcaggtgac tgtgcaggag
tttgccctgt tctttaccat ctttgatgag accaagtctt ggtacttcac tgagaacatg
gagaggaact gcagggcccc ctgtaacatc cagatggagg accccacctt taaggagaac
tacaggttcc atgccatcaa tggctacatc atggacactc tgcctggcct ggtgatggcc
caggaccaga ggatcaggtg gtacctgctg tctatgggct ctaatgagaa cattcattct
atccacttct ctggccatgt gtttactgtg aggaagaagg aggagtacaa gatggccctg
tacaatctgt accctggggt gtttgaaact gtggagatgc tgccctctaa ggctggcatc
tggagggtgg agtgcctgat tggggaacac ctgcatgctg gcatgagcac cctgttcctg
gtctatagca ataagtgcca gacccccctg gggatggcct ctgggcatat cagagacttc
cagatcactg cctctggcca gtatggccag tgggccccca agctggccag gctgcactac
tctggcagca ttaatgcctg gagcaccaag gagcccttct cttggatcaa ggtggacctg
ctggctccca tgatcatcca tgggatcaag acccaggggg ccaggcagaa gttcagcagc
ctgtacatct ctcagttcat catcatgtac tctctggatg gcaagaagtg gcagacctac
aggggcaata gcactgggac cctgatggtg ttctttggga atgtggacag ctctggcatc
aagcacaata tcttcaaccc ccccatcatt gccaggtaca tcagactgca ccccactcat
tacagcatca ggagcactct gaggatggag ctgatgggct gtgacctgaa tagctgctct
atgcccctgg gcatggagag caaggccatt tctgatgccc agattactgc ctcttcttac
ttcactaata tgtttgccac ctggagcccc agcaaggcca ggctgcatct gcaggggagg
agcaatgcct ggaggcccca ggtgaacaac cccaaggagt ggctgcaggt ggacttccag
aagactatga aggtgactgg ggtgaccact cagggggtga agagcctgct gaccagcatg
tatgtgaagg agttcctgat ctcttctagc caggatgggc accagtggac cctgtttttc
cagaatggga aggtgaaggt gtttcagggc aatcaggaca gctttactcc tgtggtgaac
agcctggacc cccccctgct gactaggtac ctgaggattc acccccagag ctgggtgcac
cagattgccc tgaggatgga ggtgctgggc tgtgaggccc aggatctgta ctga
FVIII encoding CpG reduced nucleic acid variant X04
(SEQ ID NO: 4)
atgcagattg agctgtctac ctgcttcttt ctgtgcctgc tgaggttctg tttctctgcc
actaggaggt attatctggg ggctgtggag ctgtcctggg actacatgca gtctgatctg
ggggagctgc ctgtggatgc caggttccct cccagggtgc ccaagtcttt ccctttcaat
acctctgtgg tgtacaagaa gactctgttt gtggagttta ctgatcacct gtttaacatt
gccaagccca ggcccccctg gatggggctg ctgggcccca ccatccaggc tgaggtgtat
gacactgtgg tgattactct gaagaatatg gcttctcacc ctgtgagcct gcatgctgtg
ggggtgagct actggaaggc ctctgagggg gctgagtatg atgaccagac cagccagagg
gagaaggagg atgacaaggt gttccctggg ggcagccaca cttatgtgtg gcaggtgctg
aaggagaatg gcccaatggc ctctgacccc ctgtgcctga cctacagcta tctgagccat
gtggatctgg tgaaggatct gaactctggc ctgattgggg ccctgctggt gtgcagggag
ggctctctgg ccaaggagaa gactcagact ctgcacaagt tcatcctgct gtttgctgtg
tttgatgagg gcaagagctg gcactctgag accaagaact ctctgatgca ggatagggat
gctgcttctg ccagggcctg gcccaagatg cacactgtga atgggtatgt gaataggagc
ctgcctgggc tgattgggtg tcacaggaag tctgtgtact ggcatgtgat tggcatgggc
accactcctg aggtgcacag catctttctg gagggccaca cttttctggt gaggaatcac
aggcaggcca gcctggagat cagccccatc accttcctga ctgcccagac cctgctgatg
gatctgggcc agttcctgct gttttgccat atcagcagcc atcagcatga tgggatggag
gcttatgtga aggtggactc ttgccctgag gagcctcagc tgaggatgaa gaataatgaa
gaggctgagg actatgatga tgatctgact gactctgaga tggatgtggt gaggtttgat
gatgacaaca gccccagctt tatccagatt aggtctgtgg ccaagaagca ccccaagacc
tgggtgcatt acattgctgc tgaggaagag gattgggact atgcccccct ggtgctggcc
cctgatgaca ggagctacaa gtctcagtac ctgaacaatg gccctcagag gattggcagg
aagtacaaga aggtgaggtt catggcttac actgatgaga ccttcaagac cagggaggcc
attcagcatg aatctgggat cctgggcccc ctgctgtatg gggaggtggg ggacaccctg
ctgattattt tcaagaacca ggccagcagg ccctacaaca tttatcctca tggcattact
gatgtgagac ccctgtacag caggaggctg cctaaggggg tgaagcacct gaaggacttc
cccatcctgc ctggggagat cttcaagtac aagtggactg tgactgtgga ggatggcccc
actaagtctg accccaggtg cctgactagg tactactcca gctttgtgaa catggagagg
gacctggcct ctggcctgat tggccccctg ctgatctgct acaaggagtc tgtggatcag
aggggcaacc agatcatgtc tgacaagaga aatgtgatcc tgttctctgt gtttgatgag
aataggtctt ggtacctgac tgagaacatc cagaggtttc tgcctaatcc tgctggggtg
cagctggagg atcctgagtt ccaggcctct aacattatgc acagcatcaa tgggtatgtg
tttgacagcc tgcagctgtc tgtgtgcctg catgaggtgg cctactggta catcctgagc
attggggccc agactgactt tctgtctgtg ttcttctctg gctacacctt taagcataag
atggtgtatg aggacaccct gactctgttc cccttctctg gggagactgt gttcatgagc
atggagaacc caggcctgtg gatcctgggc tgccacaact ctgatttcag gaataggggc
atgactgccc tgctgaaggt gagcagctgt gataagaaca ctggggacta ttatgaggat
agctatgagg acatctctgc ctacctgctg agcaagaaca atgccattga gcccaggagc
ttcagccaga atcctcctgt gctgaagagg caccagaggg agatcaccag gaccaccctg
cagtctgatc aggaggagat tgactatgat gacactatct ctgtggagat gaagaaggag
gactttgaca tctatgatga ggatgagaat cagagcccca ggagcttcca gaagaagact
agacactact ttattgctgc tgtggagagg ctgtgggact atggcatgag ctcttctccc
catgtgctga gaaacagggc ccagtctggc tctgtgcccc agttcaagaa ggtggtcttc
caggagttca ctgatggctc tttcacccag cctctgtata gaggggagct gaatgagcac
ctgggcctgc tgggccctta catcagggct gaggtggagg acaatatcat ggtgaccttc
aggaaccagg ctagcaggcc ctactctttc tacagcagcc tgatcagcta tgaggaggac
cagaggcagg gggctgagcc taggaagaat tttgtgaagc ccaatgagac caagacctac
ttctggaagg tgcagcacca catggctccc actaaggatg agtttgactg caaggcctgg
gcctactttt ctgatgtgga cctggagaag gatgtgcatt ctggcctgat tggccccctg
ctggtctgcc acaccaatac tctgaaccct gctcatggga gacaggtgac tgtgcaggag
tttgccctgt tcttcaccat ctttgatgag accaagtcct ggtactttac tgagaacatg
gagaggaatt gcagggcccc ttgcaacatc cagatggagg accccacctt caaggaaaat
tataggttcc atgccatcaa tggctacatc atggacaccc tgcctggcct ggtgatggcc
caggaccaga ggatcaggtg gtatctgctg tctatgggct ctaatgagaa catccacagc
atccatttct ctggccatgt gttcactgtg aggaagaagg aggagtataa gatggctctg
tacaacctgt accctggggt ctttgagact gtggagatgc tgcccagcaa ggctggcatt
tggagggtgg agtgcctgat tggggaacac ctgcatgctg ggatgagcac cctgttcctg
gtgtactcta acaagtgcca gaccccactg ggcatggctt ctggccacat cagggatttc
cagattactg cctctggcca gtatggccag tgggctccca agctggctag gctgcactac
tctgggagca tcaatgcctg gtctactaag gagcctttct cttggatcaa agtggacctg
ctggccccta tgatcatcca tgggatcaag actcaggggg ccaggcagaa gttcagcagc
ctgtacatct ctcagttcat cattatgtac agcctggatg gcaagaagtg gcagacctac
aggggcaaca gcactggcac cctgatggtg ttctttggga atgtggacag ctctgggatt
aagcacaaca tctttaaccc ccccatcatt gccaggtata tcaggctgca ccctacccac
tacagcatta ggagcaccct gaggatggag ctgatgggct gtgacctgaa cagctgcagc
atgcccctgg ggatggagag caaggccatt tctgatgctc agatcactgc ttctagctac
ttcactaaca tgtttgccac ctggtctccc agcaaggcta gactgcacct gcaggggagg
agcaatgcct ggaggcccca ggtgaataat cccaaggagt ggctgcaggt ggatttccag
aaaaccatga aggtgactgg ggtgactacc cagggggtga agtctctgct gaccagcatg
tatgtgaagg agttcctgat cagcagcagc caggatgggc atcagtggac cctgttcttt
cagaatggga aggtgaaggt gtttcagggc aatcaggaca gcttcacccc tgtggtgaac
agcctggacc cccccctgct gaccaggtac ctgaggatcc acccccagag ctgggtgcat
cagattgccc tgaggatgga ggtgctgggc tgtgaggccc aggacctgta ctga
FVIII encoding CpG reduced nucleic acid variant X05
(SEQ ID NO: 5)
atgcagattg agctgtctac ttgcttcttc ctgtgcctgc tgaggttctg cttctctgcc
actaggaggt attacctggg ggctgtggag ctgagctggg actatatgca gtctgacctg
ggggagctgc ctgtggatgc caggtttcct cccagggtgc ctaagagctt ccccttcaac
acctctgtgg tgtacaagaa gactctgttt gtggagttta ctgatcatct gttcaacatt
gccaagccca ggcctccttg gatggggctg ctgggcccca ccatccaggc tgaggtgtat
gacactgtgg tgattaccct gaagaatatg gccagccatc ctgtgagcct gcatgctgtg
ggggtgagct attggaaggc ctctgagggg gctgagtatg atgatcagac tagccagagg
gagaaggagg atgacaaggt gttccctggg gggagccata cctatgtgtg gcaggtgctg
aaggagaatg gccccatggc ctctgaccct ctgtgcctga cttatagcta cctgagccat
gtggatctgg tgaaggacct gaactctggc ctgattgggg ccctgctggt gtgcagggag
ggcagcctgg ccaaggagaa gactcagacc ctgcacaagt tcatcctgct gtttgctgtg
tttgatgagg ggaagtcctg gcactctgag actaagaaca gcctgatgca ggatagggat
gctgcttctg ccagggcctg gcctaagatg cacactgtga atggctatgt gaataggagc
ctgcctggcc tgattggctg ccataggaag tctgtgtact ggcatgtgat tgggatgggc
accacccctg aggtgcactc tattttcctg gagggccata ctttcctggt gaggaaccat
aggcaggcca gcctggagat cagccccatc actttcctga ctgcccagac tctgctgatg
gacctgggcc agttcctgct gttctgccac atcagcagcc atcagcatga tggcatggag
gcttatgtga aggtggacag ctgccctgag gagcctcagc tgaggatgaa gaataatgag
gaggctgagg actatgatga tgacctgact gactctgaga tggatgtggt gaggtttgat
gatgacaact ctccctcttt catccagatc aggtctgtgg ccaagaagca ccctaagacc
tgggtgcact acattgctgc tgaggaggag gattgggact atgcccccct ggtgctggcc
ccagatgaca ggagctacaa gtcccagtac ctgaacaatg gcccccagag gattggcagg
aagtacaaga aggtgaggtt catggcttat actgatgaga ctttcaagac cagggaggcc
atccagcatg agtctggcat cctgggccct ctgctgtatg gggaggtggg ggacaccctg
ctgattatct tcaagaacca ggcttctagg ccctacaata tctaccctca tggcatcact
gatgtgaggc ccctgtacag caggaggctg cccaaggggg tgaagcatct gaaggatttc
cccatcctgc ctggggagat ctttaagtat aagtggactg tgactgtgga ggatggcccc
actaagtctg accccaggtg cctgaccagg tattacagca gctttgtgaa catggagagg
gatctggctt ctgggctgat tggccccctg ctgatctgct acaaggagtc tgtggaccag
aggggcaacc agatcatgtc tgacaagagg aatgtgatcc tgttctctgt gtttgatgag
aataggagct ggtacctgac tgagaacatc cagaggtttc tgcccaatcc tgctggggtg
cagctggagg atcctgagtt tcaggcctct aatatcatgc acagcatcaa tggctatgtg
tttgactctc tgcagctgtc tgtgtgcctg catgaggtgg cctattggta catcctgagc
attggggccc agactgactt tctgtctgtg tttttttctg gctacacctt caagcacaag
atggtgtatg aggatactct gactctgttc cctttttctg gggagactgt gttcatgtct
atggagaacc ctgggctgtg gattctgggc tgccacaatt ctgacttcag gaacagaggc
atgactgctc tgctgaaggt gagcagctgt gacaagaaca ctggggacta ctatgaggac
tcttatgagg acatttctgc ctacctgctg agcaagaaca atgccattga gcccagaagc
ttttctcaga acccccctgt gctgaagagg caccagaggg agatcaccag gaccaccctg
cagtctgacc aggaggagat tgactatgat gatactattt ctgtggagat gaagaaggag
gactttgaca tctatgatga ggatgagaac cagagcccca ggtctttcca gaagaagact
aggcactact ttattgctgc tgtggagagg ctgtgggact atgggatgtc tagctctcct
catgtgctga ggaacagggc ccagtctggc tctgtgcccc agtttaaaaa ggtggtgttc
caggaattca ctgatggcag ctttacccag cctctgtaca ggggggagct gaatgagcac
ctggggctgc tggggcctta cattagggct gaggtggagg acaacatcat ggtgaccttc
aggaatcagg ccagcaggcc ctactctttc tacagcagcc tgatctctta tgaggaggac
cagaggcagg gggctgaacc caggaagaac tttgtgaagc ccaatgagac caagacctac
ttctggaagg tgcagcacca catggctccc accaaggatg agtttgattg caaggcctgg
gcttacttct ctgatgtgga tctggagaag gatgtgcact ctgggctgat tggccccctg
ctggtgtgcc acaccaacac tctgaaccct gcccatggca gacaggtgac tgtgcaggag
tttgccctgt tcttcactat ctttgatgag actaagagct ggtacttcac tgagaacatg
gagaggaatt gcagggcccc ttgcaacatc cagatggagg accccacctt taaggagaac
tacaggtttc atgccattaa tggctacatc atggacaccc tgcctggcct ggtgatggcc
caggaccaga ggatcaggtg gtacctgctg tctatgggga gcaatgagaa catccacagc
attcacttct ctggccatgt gttcactgtg aggaagaagg aggagtacaa gatggccctg
tacaacctgt accctggggt gtttgagact gtggagatgc tgcccagcaa ggctgggatc
tggagggtgg agtgcctgat tggggagcac ctgcatgctg ggatgagcac cctgttcctg
gtgtatagca acaagtgcca gacccccctg ggcatggcct ctggccacat cagagacttt
cagattactg cctctggcca gtatgggcag tgggccccca agctggccag gctgcactat
tctggctcta ttaatgcctg gagcactaag gagcccttca gctggattaa ggtggacctg
ctggctccca tgatcatcca tggcatcaag actcaggggg ccaggcagaa gttctcttct
ctgtacatca gccagttcat tatcatgtac tccctggatg gcaagaagtg gcagacctat
aggggcaaca gcactggcac cctgatggtg ttctttggga atgtggacag ctctggcatc
aagcataata tcttcaatcc ccccatcatt gctaggtaca tcaggctgca ccccacccac
tactctatta ggtctaccct gaggatggag ctgatgggct gtgacctgaa cagctgcagc
atgcctctgg gcatggagag caaagccatc tctgatgccc agatcactgc cagcagctac
tttaccaaca tgtttgctac ttggagcccc agcaaggcca ggctgcacct gcaggggagg
tctaatgcct ggaggcccca ggtgaacaac cccaaggagt ggctgcaggt ggacttccag
aagactatga aggtgactgg ggtgaccacc cagggggtga agagcctgct gacctctatg
tatgtgaagg agttcctgat tagcagcagc caggatggcc accagtggac cctgtttttc
cagaatggga aggtgaaggt gtttcagggg aaccaggaca gcttcactcc tgtggtgaac
tctctggacc cccccctgct gaccaggtat ctgaggatcc accctcagag ctgggtgcac
cagattgccc tgaggatgga ggtgctgggc tgtgaggccc aggacctgta ctga
FVIII encoding CpG reduced nucleic acid variant X06
(SEQ ID NO: 6)
atgcagattg agctgagcac ctgcttcttc ctgtgcctgc tgaggttttg cttctctgcc
accaggaggt actacctggg ggctgtggag ctgagctggg attacatgca gtctgacctg
ggggagctgc ctgtggatgc caggttccct cccagggtgc ccaagtcttt ccccttcaac
acttctgtgg tgtacaagaa gaccctgttt gtggagttta ctgaccacct gttcaacatt
gccaagccca ggcctccctg gatgggcctg ctgggcccca ccattcaggc tgaggtgtat
gacactgtgg tcatcaccct gaaaaatatg gctagccacc ctgtgtctct gcatgctgtg
ggggtgagct actggaaggc ctctgagggg gctgagtatg atgaccagac tagccagagg
gagaaggagg atgacaaggt gttccctggg ggcagccaca cttatgtgtg gcaggtgctg
aaagagaatg gccccatggc ttctgatccc ctgtgtctga cctatagcta cctgagccat
gtggatctgg tgaaggacct gaactctggc ctgattgggg ccctgctggt gtgcagggag
ggcagcctgg ctaaggagaa gacccagacc ctgcataagt tcatcctgct gtttgctgtg
tttgatgagg gcaagagctg gcactctgag actaagaaca gcctgatgca ggatagggat
gctgcttctg ccagggcctg gcccaagatg cacactgtga atgggtatgt gaacaggagc
ctgcctggcc tgattggctg ccataggaag tctgtctatt ggcatgtgat tggcatgggc
actactcctg aggtgcacag catctttctg gagggccaca ccttcctggt gaggaaccac
aggcaggcca gcctggagat ctctcccatc actttcctga ctgctcagac cctgctgatg
gacctgggcc agttcctgct gttctgtcac atctctagcc accagcatga tggcatggag
gcctatgtga aggtggatag ctgccctgag gaaccccagc tgaggatgaa gaacaatgag
gaggctgagg attatgatga tgatctgact gattctgaga tggatgtggt gaggtttgat
gatgacaatt ctcctagctt cattcagatc agatctgtgg ccaaaaagca tcctaagact
tgggtgcatt atattgctgc tgaggaggag gattgggatt atgcccccct ggtgctggct
cctgatgata ggagctacaa gtctcagtac ctgaataatg ggccccagag gattggcagg
aagtacaaga aggtgaggtt catggcctac actgatgaga ccttcaagac cagggaggcc
attcagcatg agtctgggat tctggggccc ctgctgtatg gggaggtggg ggataccctg
ctgatcattt tcaagaacca ggccagcagg ccctacaaca tctaccccca tgggattact
gatgtgaggc ccctgtactc taggaggctg cctaaggggg tgaagcacct gaaggatttt
cctatcctgc ctggggaaat cttcaagtac aagtggactg tgactgtgga ggatggcccc
actaagtctg atcccaggtg tctgaccagg tattatagct cttttgtgaa catggagagg
gatctggcct ctgggctgat tggccctctg ctgatctgct acaaggagtc tgtggaccag
aggggcaacc agatcatgtc tgacaagagg aatgtgatcc tgttctctgt gtttgatgag
aacaggagct ggtatctgac tgagaacatc cagaggtttc tgcccaatcc tgctggggtg
cagctggagg atcctgagtt ccaggctagc aacatcatgc acagcatcaa tgggtatgtg
tttgacagcc tgcagctgtc tgtgtgtctg catgaggtgg cctactggta tatcctgtct
attggggccc agactgactt cctgtctgtg tttttttctg ggtatacttt taagcacaag
atggtgtatg aggacaccct gactctgttc cccttctctg gggagactgt gtttatgagc
atggagaacc ctggcctgtg gatcctgggc tgccacaatt ctgacttcag gaataggggg
atgactgccc tgctgaaggt gagcagctgt gataagaata ctggggacta ctatgaggac
tcttatgagg acatttctgc ctatctgctg tctaagaaca atgccattga acccaggagc
ttctctcaga acccccctgt gctgaagagg caccagaggg aaatcaccag aactactctg
cagtctgatc aggaggaaat tgactatgat gacactattt ctgtggagat gaagaaggag
gactttgaca tctatgatga ggatgagaac cagagcccaa ggagcttcca gaagaagact
aggcactact tcattgctgc tgtggagagg ctgtgggact atggcatgag cagcagcccc
catgtgctga gaaacagggc ccagtctggg tctgtgcccc agttcaagaa ggtggtgttc
caggagttca ctgatgggag cttcacccag cccctgtata ggggggagct gaatgagcac
ctgggcctgc tgggccccta tattagggct gaggtggagg acaacatcat ggtgaccttc
aggaatcagg cctctaggcc ctacagcttc tacagcagcc tgattagcta tgaggaggat
cagaggcagg gggctgaacc caggaagaac tttgtgaagc ccaatgagac caagacctat
ttctggaagg tgcagcatca catggccccc accaaggatg agtttgactg caaggcctgg
gcctacttct ctgatgtgga tctggagaag gatgtgcact ctggcctgat tggccccctg
ctggtgtgcc acaccaacac cctgaaccct gctcatggca ggcaggtgac tgtgcaggag
tttgccctgt tcttcaccat ctttgatgag actaagtctt ggtacttcac tgagaatatg
gagaggaatt gcagggcccc ctgcaatatt cagatggaag accccacctt caaggagaat
tacaggttcc atgccattaa tggctacatc atggataccc tgcctggcct ggtgatggcc
caggatcaga ggatcaggtg gtacctgctg agcatgggca gcaatgagaa catccactct
atccacttct ctggccatgt gtttactgtg aggaagaagg aggagtataa gatggccctg
tacaacctgt accctggggt ctttgagact gtggagatgc tgccttctaa ggctggcatt
tggagggtgg agtgcctgat tggggaacac ctgcatgctg gcatgtctac cctgttcctg
gtgtacagca ataagtgcca gacccccctg ggcatggcct ctgggcatat cagggatttc
cagatcactg cctctggcca gtatggccag tgggccccaa agctggctag gctgcactac
tctgggagca tcaatgcctg gagcactaag gagcccttca gctggatcaa ggtggacctg
ctggccccca tgattatcca tgggattaag actcaggggg ccaggcagaa gttcagcagc
ctgtacatca gccagttcat tatcatgtac agcctggatg gcaagaagtg gcagacctat
aggggcaact ctactgggac cctgatggtg ttctttggga atgtggatag ctctgggatc
aagcacaata tcttcaaccc ccccatcatt gccaggtata tcaggctgca ccccacccac
tacagcatta ggtctaccct gaggatggag ctgatgggct gtgatctgaa cagctgtagc
atgcctctgg gcatggagtc taaggccatt tctgatgccc agattactgc tagcagctac
ttcaccaaca tgtttgccac ctggtctccc agcaaggcca ggctgcatct gcagggcagg
tctaatgctt ggaggcccca ggtgaacaac ccaaaggagt ggctgcaggt ggatttccag
aagactatga aggtgactgg ggtgaccact cagggggtga agtctctgct gacctctatg
tatgtgaagg agttcctgat ctctagcagc caggatggcc atcagtggac cctgttcttc
cagaatggca aggtgaaagt gttccagggc aatcaggata gcttcactcc agtggtgaac
agcctggatc cccctctgct gactaggtac ctgaggatcc acccccagag ctgggtgcac
cagattgccc tgaggatgga ggtgctgggc tgtgaggccc aggacctgta ctga
FVIII encoding CpG reduced nucleic acid variant X07
(SEQ ID NO: 7)
atgcagattg agctgagcac ctgcttcttc ctgtgtctgc tgaggttctg cttctctgcc
accaggaggt attacctggg ggctgtggag ctgagctggg actatatgca gtctgacctg
ggggagctgc ctgtggatgc taggttcccc cccagggtgc ccaagagctt cccctttaac
acttctgtgg tgtacaagaa gaccctgttt gtggagttca ctgaccacct gttcaacatt
gccaagccca ggcccccctg gatggggctg ctggggccca ccatccaggc tgaggtgtat
gacactgtgg tgatcaccct gaagaacatg gccagccacc ctgtgagcct gcatgctgtg
ggggtgagct actggaaggc ttctgagggg gctgagtatg atgaccagac tagccagagg
gagaaggagg atgacaaggt gtttcctggg ggcagccata cctatgtgtg gcaggtgctg
aaggagaatg gccccatggc ctctgacccc ctgtgcctga cctacagcta cctgtctcat
gtggacctgg tgaaggacct gaactctggc ctgattgggg ctctgctggt gtgtagggag
ggcagcctgg ctaaggaaaa gacccagacc ctgcataagt ttatcctgct gtttgctgtg
tttgatgagg gcaagagctg gcactctgag accaagaaca gcctgatgca ggatagggat
gctgcctctg ccagggcttg gcctaagatg cacactgtga atgggtatgt gaataggagc
ctgcctggcc tgattggctg ccacaggaag tctgtgtact ggcatgtgat tgggatgggc
accacccctg aggtccatag catcttcctg gagggccaca ctttcctggt gaggaaccac
agacaggcct ctctggagat ctctcccatc accttcctga ctgctcagac tctgctgatg
gacctgggcc agttcctgct gttttgccat attagcagcc accagcatga tgggatggag
gcctatgtga aggtggatag ctgccctgag gagcctcagc tgaggatgaa gaacaatgag
gaggctgaag actatgatga tgacctgact gattctgaga tggatgtggt gaggtttgat
gatgacaata gccccagctt cattcagatc aggtctgtgg ccaagaaaca ccccaagacc
tgggtgcact acattgctgc tgaggaagag gactgggact atgctcccct ggtgctggcc
cctgatgata ggtcttataa gagccagtac ctgaacaatg ggccccagag gattggcagg
aagtacaaga aggtgaggtt catggcctac actgatgaaa ccttcaaaac cagggaggcc
attcagcatg agtctggcat cctgggccct ctgctgtatg gggaggtggg ggacaccctg
ctgatcatct tcaagaacca ggccagcagg ccctacaaca tctatcctca tggcatcact
gatgtgaggc ccctgtacag caggaggctg cccaaggggg tgaagcacct gaaagacttc
cccatcctgc ctggggagat ctttaagtat aagtggactg tgactgtgga ggatggccct
accaagtctg accccaggtg tctgaccagg tactattcta gctttgtgaa catggagagg
gacctggcct ctggcctgat tgggcccctg ctgatctgct acaaggagtc tgtggaccag
aggggcaacc agatcatgtc tgacaagagg aatgtgatcc tgttttctgt gtttgatgag
aataggagct ggtacctgac tgagaacatc cagaggtttc tgcccaatcc tgctggggtg
cagctggagg atcctgagtt ccaggccagc aatatcatgc atagcatcaa tggctatgtg
tttgacagcc tgcagctgtc tgtgtgcctg catgaggtgg cctactggta catcctgagc
attggggccc agactgactt tctgtctgtg ttcttttctg gctatacctt caagcacaag
atggtgtatg aggataccct gaccctgttc cccttctctg gggagactgt gttcatgagc
atggagaatc ctgggctgtg gatcctgggg tgccacaact ctgattttag gaacaggggg
atgactgccc tgctgaaggt gtctagctgt gataagaaca ctggggacta ctatgaggac
agctatgagg acatttctgc ttatctgctg tctaagaata atgccattga gcccagaagc
ttcagccaga atccccctgt gctgaagaga catcagaggg agatcaccag aactaccctg
cagtctgatc aggaggagat tgactatgat gacactatct ctgtggagat gaagaaggag
gactttgaca tctatgatga ggatgagaat cagtctccca ggagctttca gaagaagacc
agacattact tcattgctgc tgtggagagg ctgtgggact atggcatgag ctctagccct
catgtgctga ggaacagggc ccagtctggc tctgtgcccc agttcaagaa ggtggtgttc
caggaattca ctgatggcag cttcacccag cccctgtaca ggggggagct gaatgagcac
ctgggcctgc tggggcctta tatcagggct gaggtggagg ataatattat ggtgactttc
aggaaccagg ccagcaggcc ctactctttc tatagcagcc tgatctctta tgaggaggat
cagaggcagg gggctgagcc taggaagaac tttgtgaagc ccaatgagac taagacctac
ttctggaagg tccagcacca catggcccct accaaggatg agtttgactg caaggcctgg
gcctatttct ctgatgtgga tctggagaag gatgtccatt ctgggctgat tggccccctg
ctggtgtgcc acactaacac tctgaatcct gcccatggca ggcaggtgac tgtccaggag
tttgccctgt tcttcactat ctttgatgag accaagagct ggtactttac tgagaacatg
gagaggaact gcagagctcc ttgcaatatt cagatggagg accccacctt caaggagaat
tacaggttcc atgccattaa tgggtacatc atggacaccc tgcctggcct ggtgatggct
caggaccaga ggatcaggtg gtacctgctg agcatgggct ctaatgagaa tatccacagc
atccacttct ctgggcatgt gttcactgtg aggaagaagg aggagtacaa gatggctctg
tataatctgt accctggggt gtttgaaact gtggagatgc tgccctctaa ggctggcatc
tggagggtgg agtgcctgat tggggagcac ctgcatgctg gcatgagcac cctgttcctg
gtgtacagca acaagtgcca gacccccctg ggcatggcct ctggccacat cagggacttc
cagatcactg cctctggcca gtatggccag tgggccccca agctggccag gctgcactat
tctggcagca tcaatgcctg gagcaccaag gagcccttca gctggatcaa ggtggacctg
ctggccccca tgatcattca tggcatcaag acccaggggg ccaggcagaa gttcagctct
ctgtacatct ctcagttcat catcatgtac tctctggatg ggaagaagtg gcagacctac
aggggcaaca gcactggcac cctgatggtg ttctttggga atgtggactc ttctggcatc
aagcacaaca tcttcaatcc ccccatcatt gctaggtata ttaggctgca tcccacccac
tacagcatca ggtctaccct gaggatggag ctgatgggct gtgacctgaa ctcttgcagc
atgcccctgg gcatggagtc taaggccatc tctgatgccc agattactgc cagcagctac
ttcaccaaca tgtttgccac ctggagcccc tctaaggcca ggctgcatct gcaggggagg
agcaatgcct ggaggcctca ggtgaacaac cccaaggagt ggctgcaggt ggatttccag
aagaccatga aggtgactgg ggtgaccacc cagggggtca agagcctgct gaccagcatg
tatgtgaagg agttcctgat cagcagcagc caggatggcc accagtggac tctgttcttt
cagaatggga aggtgaaggt gtttcagggc aatcaggact ctttcacccc tgtggtgaac
agcctggacc cccccctgct gaccagatac ctgaggatcc acccccagtc ttgggtgcat
cagattgccc tgaggatgga ggtgctgggc tgtgaggctc aggatctgta ctga
FVIII encoding CpG reduced nucleic acid variant X08
(SEQ ID NO: 8)
atgcagattg agctgagcac ttgctttttt ctgtgcctgc tgaggttttg tttttctgcc
accaggaggt actacctggg ggctgtggag ctgagctggg actatatgca gtctgatctg
ggggagctgc ctgtggatgc caggttcccc cccagggtgc ccaagtcttt tcccttcaac
acctctgtgg tgtataagaa gaccctgttt gtggagttca ctgaccacct gttcaacatt
gctaagccta ggcccccctg gatgggcctg ctgggcccta ccattcaggc tgaggtgtat
gacactgtgg tgatcaccct gaagaacatg gccagccatc ctgtgagcct gcatgctgtg
ggggtctctt actggaaggc ctctgagggg gctgagtatg atgaccagac cagccagaga
gagaaggagg atgacaaggt cttccctggg ggctctcaca cctatgtgtg gcaggtgctg
aaggaaaatg gccccatggc ctctgacccc ctgtgcctga cctacagcta tctgagccat
gtggatctgg tgaaggacct gaattctggc ctgattgggg ccctgctggt gtgcagggag
ggcagcctgg ccaaggagaa gacccagacc ctgcacaagt ttatcctgct gtttgctgtg
tttgatgagg gcaagtcttg gcactctgag actaagaaca gcctgatgca ggacagggat
gctgcctctg ccagggcctg gcccaagatg cacactgtga atggctatgt gaacaggagc
ctgcctgggc tgattggctg ccacaggaag tctgtgtact ggcatgtgat tggcatgggc
accacccctg aggtgcacag catcttcctg gaaggccaca ctttcctggt gaggaaccat
aggcaggcca gcctggagat cagccctatc accttcctga ctgcccagac cctgctgatg
gatctggggc agttcctgct gttctgccac atctctagcc accagcatga tgggatggag
gcctatgtga aggtggacag ctgcccagag gagcctcagc tgaggatgaa aaacaatgaa
gaggctgagg attatgatga tgatctgact gactctgaga tggatgtggt gagatttgat
gatgacaata gccctagctt tattcagatc aggtctgtgg ctaagaagca ccccaagacc
tgggtgcatt acattgctgc tgaggaggag gactgggatt atgctcctct ggtgctggcc
cctgatgata ggagctacaa gagccagtac ctgaataatg gccctcagag gattggcagg
aagtacaaga aggtgaggtt catggcttac actgatgaga ccttcaagac tagggaggcc
atccagcatg agtctgggat cctggggccc ctgctgtatg gggaggtggg ggacaccctg
ctgatcatct tcaagaacca ggctagcagg ccttacaaca tctatcccca tgggatcact
gatgtgagac ctctgtacag caggaggctg cccaaggggg tcaagcatct gaaagacttc
cccatcctgc ctggggagat ctttaagtat aagtggactg tgactgtgga ggatgggccc
accaagtctg accccaggtg cctgaccagg tattacagca gctttgtgaa catggagagg
gatctggcct ctgggctgat tggccccctg ctgatctgtt acaaggaatc tgtggatcag
aggggcaatc agatcatgtc tgacaagagg aatgtgatcc tgttctctgt gtttgatgag
aataggtctt ggtacctgac tgaaaacatc cagaggttcc tgcccaaccc tgctggggtc
cagctggagg atcctgagtt ccaggctagc aacatcatgc acagcatcaa tgggtatgtg
tttgatagcc tgcagctgtc tgtgtgcctg catgaggtgg cctactggta catcctgtct
attggggccc agactgactt cctgtctgtg ttcttttctg gctacacctt caagcacaag
atggtgtatg aggacaccct gaccctgttc cccttctctg gggagactgt ctttatgagc
atggagaacc ctgggctgtg gatcctgggc tgccacaact ctgatttcag gaataggggc
atgactgctc tgctgaaggt gagctcttgt gacaagaaca ctggggatta ctatgaggac
agctatgagg acatttctgc ctacctgctg agcaagaaca atgccattga gcctaggagc
tttagccaga atcctcctgt cctgaagagg caccagaggg agatcaccag gaccaccctg
cagtctgacc aggaggagat tgactatgat gataccatct ctgtggagat gaagaaggag
gactttgaca tctatgatga ggatgagaat cagtctccca ggagcttcca gaagaagacc
aggcactatt tcattgctgc tgtggagagg ctgtgggact atggcatgag cagctctcct
catgtgctga ggaatagggc tcagtctggc tctgtgcccc agttcaagaa agtggtgttt
caggagttca ctgatggctc tttcacccag cctctgtata ggggggagct gaatgagcac
ctggggctgc tgggccccta tatcagggct gaggtggagg ataacatcat ggtgaccttc
aggaaccagg cctctaggcc ctacagcttc tatagcagcc tgatcagcta tgaggaggac
cagaggcagg gggctgagcc caggaagaac tttgtgaagc ccaatgagac caagacttac
ttctggaagg tgcagcatca catggccccc accaaggatg agtttgactg taaggcctgg
gcctacttct ctgatgtgga tctggagaag gatgtgcact ctggcctgat tggccccctg
ctggtgtgcc ataccaatac tctgaaccct gctcatggca ggcaggtgac tgtgcaggag
tttgctctgt tcttcactat ctttgatgag accaagtctt ggtatttcac tgagaatatg
gagaggaact gcagggcccc ctgcaacatc cagatggagg accccacctt taaggagaac
tataggtttc atgccatcaa tggctacatc atggacaccc tgcctggcct ggtgatggcc
caggatcaga ggatcaggtg gtacctgctg agcatggggt ctaatgagaa catccacagc
atccacttct ctggccatgt gtttactgtg agaaagaagg aggagtacaa gatggctctg
tacaatctgt accctggggt ctttgagact gtggagatgc tgcctagcaa ggctgggatc
tggagggtgg agtgcctgat tggggaacat ctgcatgctg ggatgtctac tctgttcctg
gtgtacagca acaagtgcca gacccccctg ggcatggctt ctggccatat cagggacttt
cagattactg cctctgggca gtatggccag tgggccccca agctggctag gctgcattat
tctggcagca tcaatgcctg gtctactaag gagcccttca gctggatcaa ggtggatctg
ctggccccca tgatcatcca tggcatcaag acccaggggg ccaggcagaa gtttagctct
ctgtacatta gccagttcat catcatgtac agcctggatg ggaagaagtg gcagacctac
aggggcaatt ctactggcac cctgatggtg ttctttggca atgtggacag ctctggcatc
aagcacaaca tctttaaccc ccctatcatt gctaggtaca tcaggctgca tcccacccat
tacagcatca ggagcaccct gaggatggag ctgatgggct gtgacctgaa ctcttgcagc
atgcccctgg gcatggagag caaggccatt tctgatgccc agattactgc cagcagctac
ttcactaaca tgtttgccac ctggtctccc agcaaggcca ggctgcacct gcagggcagg
agcaatgcct ggaggcccca ggtgaacaac cccaaggagt ggctgcaggt ggatttccag
aagaccatga aggtgactgg ggtgaccacc cagggggtga agagcctgct gactagcatg
tatgtgaagg agttcctgat cagctctagc caggatggcc accagtggac tctgtttttc
cagaatggca aggtgaaggt gttccagggc aaccaggact ctttcactcc tgtggtgaac
agcctggacc cccccctgct gaccaggtat ctgaggattc acccccagtc ttgggtgcat
cagattgccc tgaggatgga ggtgctgggc tgtgaggccc aggatctgta ctga
FVIII encoding CpG reduced nucleic acid variant X09
(SEQ ID NO: 9)
atgcagattg agctgagcac ctgcttcttc ctgtgtctgc tgagattttg cttttctgcc
actaggaggt attacctggg ggctgtggag ctgtcttggg actacatgca gtctgatctg
ggggagctgc ctgtggatgc caggttccca cctagggtgc ctaagagctt tcccttcaat
acctctgtgg tgtacaagaa gaccctgttt gtggagttca ctgaccacct gttcaacatt
gccaagccta ggcccccctg gatgggcctg ctgggcccta ccatccaggc tgaagtgtat
gacactgtgg tgatcaccct gaagaacatg gccagccacc ctgtgagcct gcatgctgtg
ggggtgtctt actggaaggc ctctgagggg gctgagtatg atgatcagac cagccagagg
gagaaggaag atgacaaggt gttccctggg ggcagccaca cctatgtctg gcaggtgctg
aaggagaatg gccccatggc ctctgatccc ctgtgcctga cctactctta cctgagccat
gtggacctgg tgaaggatct gaattctggc ctgattgggg ccctgctggt gtgcagggag
ggcagcctgg ccaaggagaa gacccagacc ctgcataagt tcatcctgct gtttgctgtg
tttgatgaag ggaagagctg gcactctgag actaagaaca gcctgatgca ggacagggat
gctgcttctg ccagggcctg gcccaagatg cacactgtga atggctatgt gaatagaagc
ctgcctggcc tgattgggtg ccacaggaag tctgtgtact ggcatgtgat tgggatgggc
actacccctg aggtgcatag catcttcctg gaaggccata ccttcctggt gaggaatcat
aggcaggctt ctctggaaat ttctcccatc actttcctga ctgctcagac cctgctgatg
gacctgggcc agttcctgct gttctgccac atcagctctc accagcatga tgggatggag
gcctatgtga aggtggacag ctgtcctgag gagccccagc tgaggatgaa gaacaatgag
gaggctgagg actatgatga tgacctgact gactctgaga tggatgtggt caggtttgat
gatgacaata gcccctcttt catccagatc aggtctgtgg ccaagaagca ccccaagact
tgggtgcact acattgctgc tgaggaggag gattgggatt atgcccctct ggtgctggcc
cctgatgaca ggagctataa gtctcagtac ctgaataatg gcccccagag gattgggagg
aagtataaga aggtgaggtt tatggcctac actgatgaga ccttcaagac cagggaggcc
atccagcatg agtctggcat cctgggcccc ctgctgtatg gggaggtggg ggataccctg
ctgatcatct tcaagaacca ggcctctagg ccctacaata tctaccctca tggcatcact
gatgtgagac ccctgtatag caggaggctg cctaaggggg tgaagcacct gaaggacttc
cccatcctgc ctggggagat cttcaagtat aagtggactg tgactgtgga ggatggcccc
accaagtctg accccaggtg cctgaccagg tattacagct cttttgtgaa catggagagg
gatctggcct ctgggctgat tggcccactg ctgatctgct acaaggagtc tgtggatcag
aggggcaatc agatcatgtc tgacaagagg aatgtgatcc tgttttctgt gtttgatgaa
aataggtctt ggtatctgac tgagaacatc cagaggtttc tgcccaatcc tgctggggtg
cagctggagg atcctgagtt tcaggcctct aatatcatgc attctatcaa tggctatgtg
tttgacagcc tgcagctgtc tgtgtgcctg catgaggtgg cctactggta catcctgagc
attggggctc agactgactt cctgtctgtg ttcttttctg gctatacttt caagcacaag
atggtgtatg aggacactct gaccctgttc cccttctctg gggagactgt gttcatgtct
atggaaaatc ctgggctgtg gattctgggc tgccacaatt ctgacttcag gaataggggg
atgactgccc tgctgaaggt gtctagctgt gataagaaca ctggggatta ctatgaggac
tcttatgaag atatctctgc ctatctgctg agcaagaaca atgccattga gcccaggagc
ttcagccaga acccccctgt gctgaagagg caccagaggg agatcaccag gaccactctg
cagtctgatc aggaggagat tgactatgat gacactatct ctgtggagat gaagaaggag
gattttgaca tttatgatga ggatgagaac cagtctccca ggagcttcca gaagaagacc
aggcattact ttattgctgc tgtggagagg ctgtgggact atgggatgag cagctctcct
catgtgctga ggaacagggc ccagtctggg tctgtgcccc agttcaagaa ggtggtgttc
caggagttca ctgatgggag cttcacccag cccctgtata ggggggagct gaatgagcac
ctgggcctgc tgggccccta catcagggct gaggtggagg ataatatcat ggtgaccttc
aggaaccagg ctagcaggcc ttacagcttt tacagcagcc tgatctctta tgaagaagac
cagaggcagg gggctgagcc caggaagaat tttgtgaagc ctaatgagac caagacttat
ttttggaagg tgcagcatca catggctcct accaaggatg agtttgactg caaggcctgg
gcctactttt ctgatgtgga tctggagaag gatgtgcact ctggcctgat tggccctctg
ctggtgtgcc atactaacac tctgaaccct gcccatggga ggcaggtgac tgtgcaggag
tttgccctgt tcttcactat ttttgatgag accaagtctt ggtatttcac tgagaacatg
gagaggaact gcagggctcc ctgcaacatc cagatggaag accccacctt caaggagaac
tataggttcc atgccatcaa tgggtacatc atggataccc tgcctggcct ggtgatggcc
caggatcaga ggattaggtg gtatctgctg agcatgggct ctaatgagaa catccacagc
atccatttct ctggccatgt gttcactgtg aggaagaagg aggagtacaa gatggctctg
tacaacctgt atcctggggt gtttgagact gtggagatgc tgcccagcaa ggctggcatc
tggagggtgg aatgcctgat tggggagcac ctgcatgctg gcatgagcac tctgttcctg
gtgtatagca acaagtgcca gacccccctg ggcatggcct ctggccatat cagggatttc
cagatcactg cttctggcca gtatggccag tgggccccca agctggccag gctgcactat
tctggcagca tcaatgcctg gagcactaag gagccttttt cttggatcaa ggtggacctg
ctggccccta tgattattca tggcatcaag acccaggggg ccaggcagaa gttctctagc
ctgtacatct ctcagttcat cattatgtat agcctggatg gcaagaagtg gcagacctac
aggggcaata gcactggcac cctgatggtg ttttttggga atgtggactc ttctgggatc
aagcacaaca tctttaaccc ccccatcatt gccaggtata ttaggctgca ccccacccac
tacagcatca ggagcaccct gaggatggag ctgatgggct gtgatctgaa ttcttgctct
atgcccctgg gcatggagag caaggccatc tctgatgccc agatcactgc cagctcttac
ttcaccaaca tgtttgccac ctggtctcct agcaaggcca ggctgcatct gcagggcagg
agcaatgcct ggaggcccca ggtgaacaac cccaaggagt ggctgcaggt ggacttccag
aagaccatga aggtgactgg ggtgaccact cagggggtga agagcctgct gacctctatg
tatgtgaagg agttcctgat cagcagcagc caggatggcc accagtggac tctgttcttc
cagaatggga aggtgaaggt gttccagggc aaccaggata gctttacccc tgtggtgaac
agcctggacc ctcctctgct gaccagatac ctgaggatcc atcctcagag ctgggtgcac
cagattgccc tgaggatgga ggtgctgggc tgtgaggccc aggatctgta ctga
FVIII encoding CpG reduced nucleic acid variant X10
(SEQ ID NO: 10)
atgcagattg agctgagcac ttgcttcttc ctgtgcctgc tgaggttctg cttttctgct
actaggaggt actacctggg ggctgtggag ctgagctggg attacatgca gtctgacctg
ggggagctgc cagtggatgc caggttcccc cccagggtgc ccaagtcttt tcctttcaac
acctctgtgg tgtacaagaa gaccctgttt gtggagttca ctgaccacct gttcaacatt
gccaagccca ggcccccctg gatggggctg ctggggccca ccatccaggc tgaggtgtat
gacactgtgg tgattaccct gaagaacatg gctagccacc ctgtgagcct gcatgctgtg
ggggtgagct attggaaggc ctctgagggg gctgagtatg atgatcagac cagccagagg
gaaaaggagg atgacaaggt gttccctggg ggcagccata cttatgtgtg gcaggtgctg
aaggagaatg ggcccatggc ctctgacccc ctgtgcctga cttacagcta tctgagccat
gtggacctgg tgaaggatct gaactctggc ctgattgggg ctctgctggt gtgcagggag
ggcagcctgg ctaaggagaa gactcagact ctgcataagt tcatcctgct gtttgctgtg
tttgatgaag gcaagagctg gcactctgag accaagaact ctctgatgca ggatagggat
gctgcctctg ccagggcttg gcccaagatg cacactgtga atggctatgt gaacaggagc
ctgcctggcc tgattgggtg ccacaggaag tctgtgtact ggcatgtgat tggcatgggc
accacccctg aggtgcacag cattttcctg gagggccaca ccttcctggt gaggaatcac
aggcaggcca gcctggagat cagccccatc accttcctga ctgcccagac cctgctgatg
gacctggggc agtttctgct gttctgccac atcagcagcc atcagcatga tggcatggag
gcctatgtga aggtggactc ttgccctgag gagccccagc tgaggatgaa gaacaatgag
gaggctgagg attatgatga tgacctgact gactctgaga tggatgtggt gaggtttgat
gatgacaata gccccagctt catccagatt aggtctgtgg ccaagaagca ccctaagacc
tgggtgcact acattgctgc tgaggaggag gattgggatt atgcccccct ggtgctggct
cctgatgaca ggtcttataa gagccagtac ctgaacaatg ggccccagag gattggcagg
aagtacaaga aggtgaggtt catggcttac actgatgaga ccttcaagac tagggaggcc
atccagcatg agtctggcat cctgggcccc ctgctgtatg gggaggtggg ggataccctg
ctgatcatct tcaagaacca ggccagcagg ccctacaaca tttaccctca tggcatcact
gatgtgaggc ccctgtacag caggagactg cccaaggggg tgaagcacct gaaggatttt
cccattctgc ctggggagat cttcaagtac aagtggactg tgactgtgga ggatggcccc
accaagtctg atcccaggtg cctgactagg tactactctt cttttgtgaa tatggagagg
gatctggcct ctggcctgat tggccccctg ctgatctgct acaaggagtc tgtggaccag
aggggcaacc agatcatgtc tgacaagagg aatgtgatcc tgttctctgt gtttgatgag
aataggagct ggtacctgac tgagaatatc cagaggttcc tgcctaatcc tgctggggtc
cagctggagg atcctgagtt ccaggctagc aacattatgc acagcatcaa tggctatgtg
tttgattctc tgcagctgtc tgtgtgcctg catgaggtgg cttactggta catcctgtct
attggggccc agactgattt cctgtctgtg ttcttctctg gctacacttt caagcataag
atggtgtatg aggataccct gaccctgttc cccttctctg gggagactgt gttcatgtct
atggagaacc ctggcctgtg gatcctgggc tgtcataact ctgacttcag aaacaggggc
atgactgccc tgctgaaggt gagcagctgt gacaagaaca ctggggacta ctatgaggac
agctatgagg atatctctgc ttatctgctg agcaagaata atgccattga gcccaggagc
ttcagccaga acccccctgt gctgaagagg caccagaggg agatcactag gactaccctg
cagtctgatc aggaggagat tgactatgat gacaccatct ctgtggagat gaagaaggag
gactttgaca tctatgatga ggatgagaac cagtccccca ggtctttcca gaagaagacc
aggcactact tcattgctgc tgtggagagg ctgtgggact atggcatgag ctctagcccc
catgtgctga ggaacagggc tcagtctggc tctgtgcccc agttcaagaa ggtggtcttc
caggagttca ctgatggctc ttttacccag cctctgtaca gaggggagct gaatgagcac
ctgggcctgc tgggccccta catcagggct gaggtggagg ataatatcat ggtgaccttc
agaaaccagg cctctaggcc ctacagcttc tacagcagcc tgatctctta tgaggaggat
cagaggcagg gggctgagcc caggaagaac tttgtgaagc ccaatgagac caagacctac
ttctggaagg tgcagcacca tatggcccct actaaggatg agtttgactg caaggcctgg
gcttattttt ctgatgtgga cctggagaag gatgtgcact ctgggctgat tggccccctg
ctggtgtgcc acaccaacac cctgaaccct gcccatggca ggcaggtgac tgtgcaggag
tttgccctgt tcttcactat ctttgatgag accaagagct ggtacttcac tgagaacatg
gagagaaatt gtagggctcc ctgcaatatc cagatggagg accccacctt caaagaaaat
tacagattcc atgccatcaa tgggtacatc atggataccc tgcctgggct ggtgatggct
caggaccaga ggatcaggtg gtacctgctg agcatggggt ctaatgagaa catccactct
atccatttct ctggccatgt gttcactgtg agaaagaagg aggagtataa gatggctctg
tacaacctgt acccaggggt gtttgagact gtggaaatgc tgcccagcaa agctgggatc
tggagggtgg agtgcctgat tggggagcac ctgcatgctg gcatgtctac cctgttcctg
gtgtacagca acaagtgcca gactcccctg ggcatggcct ctgggcacat cagggatttt
cagatcactg cctctggcca gtatggccag tgggccccca agctggccag gctgcactac
tctggcagca ttaatgcttg gagcactaag gagcccttca gctggatcaa ggtggatctg
ctggccccca tgatcatcca tggcatcaag acccaggggg ccaggcagaa gttctctagc
ctgtacattt ctcagttcat catcatgtac agcctggatg ggaagaagtg gcagacctac
agggggaaca gcactgggac cctgatggtg ttctttggca atgtggatag ctctggcatc
aagcacaata tcttcaatcc ccccattatt gccaggtaca ttaggctgca tcctactcac
tactctatta ggagcaccct gaggatggag ctgatggggt gtgacctgaa cagctgttct
atgcccctgg gcatggagtc taaggctatc tctgatgccc agatcactgc cagcagctac
ttcactaata tgtttgccac ctggagccct agcaaggcca gactgcacct gcagggcagg
agcaatgcct ggaggcccca ggtgaacaac cccaaggagt ggctgcaggt ggacttccag
aagaccatga aggtgactgg ggtgaccact cagggggtga agagcctgct gaccagcatg
tatgtgaagg agttcctgat cagcagcagc caggatggcc accagtggac cctgttcttc
cagaatggga aggtgaaggt gttccagggc aaccaggact ctttcacccc tgtggtgaac
agcctggatc ctcccctgct gaccaggtac ctgaggatcc acccccagag ctgggtgcac
cagattgctc tgaggatgga agtgctgggc tgtgaggccc aggatctgta ctga
FVIII encoding CpG reduced nucleic acid variant X11
(SEQ ID NO: 11)
atgcagattg agctgagcac ctgcttcttc ctgtgcctgc tgaggttttg cttctctgct
accaggaggt actacctggg ggctgtggag ctgagctggg actatatgca gtctgacctg
ggggagctgc ctgtggatgc taggttccct cccagggtgc ccaagagctt cccctttaat
acctctgtgg tgtacaagaa aaccctgttt gtggagttca ctgaccatct gttcaacatt
gccaagccca ggcccccttg gatgggcctg ctgggcccca ccattcaggc tgaggtgtat
gacactgtgg tcattaccct gaagaacatg gcttctcacc ctgtgagcct gcatgctgtg
ggggtgagct actggaaggc ctctgagggg gctgagtatg atgaccagac cagccagagg
gagaaggagg atgataaggt gttccctggg ggcagccaca cctatgtgtg gcaggtgctg
aaggagaatg gccccatggc ctctgatccc ctgtgcctga cctactctta tctgtctcat
gtggacctgg tgaaggacct gaactctggc ctgattgggg ctctgctggt gtgcagggag
ggctctctgg ccaaggagaa gacccagacc ctgcacaagt ttattctgct gtttgctgtc
tttgatgagg gcaagagctg gcattctgag accaagaaca gcctgatgca ggacagggat
gctgcctctg ccagggcctg gcccaaaatg cacactgtga atggctatgt gaacaggagc
ctgcctggcc tgattggctg ccacaggaag tctgtgtact ggcatgtgat tggcatgggc
accacccctg aggtgcacag catcttcctg gagggccaca cctttctggt gaggaatcac
aggcaggcca gcctggagat tagccccatc accttcctga ctgcccagac cctgctgatg
gacctgggcc agttcctgct gttctgccac atcagcagcc accagcatga tggcatggag
gcctatgtga aggtggatag ctgccctgag gagccccagc tgaggatgaa aaacaatgag
gaggctgagg attatgatga tgacctgact gactctgaga tggatgtggt gaggtttgat
gatgacaata gccccagctt tattcagatt aggtctgtgg ctaagaagca ccccaagact
tgggtgcact acattgctgc tgaggaggag gattgggact atgcccctct ggtcctggcc
cctgatgata ggtcttacaa gagccagtat ctgaacaatg gcccccagag gattggcagg
aagtacaaga aggtgaggtt catggcctac actgatgaga cctttaagac cagggaggcc
attcagcatg agtctgggat cctgggcccc ctgctgtatg gggaggtggg ggacactctg
ctgatcatct tcaagaacca ggccagcagg ccttataaca tctaccctca tgggatcact
gatgtgaggc ccctgtactc tagaaggctg cccaaggggg tcaagcacct gaaggatttt
cccatcctgc ctggggagat tttcaagtac aagtggactg tgactgtgga ggatggcccc
accaagtctg accctaggtg cctgaccagg tactacagct cttttgtgaa catggagagg
gacctggcct ctggcctgat tggccctctg ctgatttgct acaaggagtc tgtggaccag
aggggcaacc agatcatgtc tgacaagagg aatgtgatcc tgttttctgt gtttgatgag
aacaggtctt ggtacctgac tgagaacatc cagaggttcc tgcctaaccc agctggggtg
cagctggagg atcctgagtt ccaggccagc aatattatgc atagcattaa tggctatgtg
tttgatagcc tgcagctgtc tgtgtgcctg catgaggtgg cctactggta catcctgagc
attggggccc agactgactt tctgtctgtg ttcttctctg gctacacctt caagcataag
atggtgtatg aggacaccct gactctgttc cctttttctg gggagactgt gtttatgagc
atggagaatc ctggcctgtg gatcctgggc tgccataatt ctgacttcag gaacaggggc
atgactgccc tgctgaaagt gagcagctgt gacaagaata ctggggacta ctatgaagac
agctatgagg acatctctgc ctacctgctg agcaagaaca atgccattga gcccaggagc
ttcagccaga accccccagt gctgaagagg caccagagag agatcaccag gactaccctg
cagtctgacc aggaggagat tgactatgat gacaccattt ctgtggagat gaagaaggag
gactttgaca tttatgatga ggatgagaat cagagcccca ggagcttcca gaagaagact
aggcactatt ttattgctgc tgtggagagg ctgtgggact atggcatgag cagctctccc
catgtgctga ggaatagggc ccagtctggc tctgtgcctc agttcaagaa ggtggtgttc
caggagttca ctgatggcag ctttacccag cccctgtata ggggggagct gaatgagcac
ctgggcctgc tgggccccta tatcagggct gaggtggagg acaatattat ggtgaccttt
aggaaccagg ccagcaggcc ctactctttc tatagcagcc tgatcagcta tgaggaggac
cagaggcagg gggctgagcc caggaagaat tttgtgaagc ctaatgagac caagacctac
ttctggaagg tgcagcatca catggccccc accaaggatg agtttgactg caaggcttgg
gcctatttct ctgatgtgga cctggagaag gatgtgcact ctggcctgat tggccccctg
ctggtgtgcc acactaacac tctgaatcct gcccatggca ggcaggtgac tgtgcaggag
tttgccctgt tcttcaccat ctttgatgag accaagagct ggtacttcac tgagaacatg
gagaggaact gcagggcccc ctgcaacatc cagatggagg atcccacctt caaggagaac
tacaggtttc atgccatcaa tggctacatc atggacactc tgcctggcct ggtgatggcc
caggatcaga ggatcaggtg gtacctgctg agcatgggct ctaatgagaa tatccatagc
atccacttct ctggccatgt gttcactgtc aggaagaagg aggagtacaa gatggctctg
tataatctgt accctggggt gtttgagact gtggagatgc tgcccagcaa ggctggcatc
tggagggtgg agtgcctgat tggggagcac ctgcatgctg ggatgagcac cctgtttctg
gtgtactcta acaagtgcca gacccccctg ggcatggcct ctgggcacat cagggatttc
cagatcactg cttctggcca gtatggccag tgggccccca agctggccag gctgcactac
tctggcagca tcaatgcctg gtctaccaag gagccctttt cttggattaa ggtggacctg
ctggccccca tgatcatcca tggcatcaag acccaggggg ccaggcagaa gttcagcagc
ctgtacatca gccagttcat catcatgtac agcctggatg gcaaaaagtg gcagacctac
aggggcaata gcactgggac tctgatggtg ttctttggca atgtggacag ctctgggatc
aagcacaata tcttcaaccc tcccatcatt gctaggtaca tcaggctgca ccccacccac
tatagcatca ggtctaccct gaggatggag ctgatgggct gtgacctgaa ctcttgcagc
atgcccctgg gcatggagtc caaagctatc tctgatgccc agattactgc cagcagctac
ttcaccaaca tgtttgccac ctggtctccc tctaaggcca ggctgcacct gcagggcagg
agcaatgcct ggaggcccca ggtgaacaat cccaaggagt ggctgcaggt ggatttccag
aaaactatga aggtgactgg ggtgaccacc cagggggtga agtctctgct gaccagcatg
tatgtgaagg agttcctgat ctcttctagc caggatggcc accagtggac tctgttcttc
cagaatggca aggtgaaggt gttccagggc aaccaggaca gcttcacccc tgtggtgaac
tctctggatc cccccctgct gaccaggtac ctgaggattc atccccagag ctgggtgcac
cagattgctc tgagaatgga ggtgctgggg tgtgaggctc aggacctgta ttga
FVIII encoding CpG reduced nucleic acid variant X12
(SEQ ID NO: 12)
atgcagattg agctgtctac ttgttttttt ctgtgcctgc tgaggttctg cttctctgcc
accaggaggt attacctggg ggctgtggag ctgagctggg attacatgca gtctgatctg
ggggagctgc ctgtggatgc caggttcccc cccagggtgc ccaagagctt ccccttcaac
acctctgtgg tgtataagaa gaccctgttt gtggagttca ctgatcatct gtttaacatt
gccaagccca ggcccccctg gatgggcctg ctgggcccaa ctatccaggc tgaggtgtat
gacactgtgg tcatcaccct gaagaatatg gccagccatc ctgtgagcct gcatgctgtg
ggggtgagct actggaaggc ctctgagggg gctgagtatg atgaccagac cagccagagg
gagaaggagg atgacaaggt gttccctggg ggcagccaca cctatgtgtg gcaggtgctg
aaggagaatg gccccatggc ctctgacccc ctgtgcctga cttatagcta cctgtctcat
gtggacctgg tgaaggacct gaactctggc ctgattgggg ccctgctggt ctgtagggaa
ggcagcctgg ccaaggagaa gacccagacc ctgcacaagt ttattctgct gtttgctgtg
tttgatgaag gcaagagctg gcactctgag accaagaatt ctctgatgca ggatagggat
gctgcctctg ccagggcctg gcccaagatg catactgtga atggctatgt gaacagaagc
ctgcctggcc tgattggctg ccataggaag tctgtgtatt ggcatgtgat tgggatgggc
actacccctg aagtgcacag cattttcctg gagggccaca ctttcctggt gaggaaccac
aggcaggcct ctctggagat cagccccatt actttcctga ctgcccagac cctgctgatg
gatctgggcc agttcctgct gttctgccac atctctagcc accagcatga tggcatggag
gcctatgtga aggtggacag ctgccctgag gagccccagc tgaggatgaa gaataatgag
gaggctgagg attatgatga tgacctgact gactctgaga tggatgtggt gaggtttgat
gatgataata gccccagctt catccagatc aggtctgtgg ccaagaagca tcccaagacc
tgggtgcact atattgctgc tgaagaggag gactgggact atgcccctct ggtgctggct
cctgatgaca ggagctataa gagccagtat ctgaacaatg ggccccagag gattgggagg
aagtacaaga aggtgaggtt catggcctac actgatgaga cctttaagac cagggaggcc
atccagcatg agtctggcat tctggggccc ctgctgtatg gggaggtggg ggacactctg
ctgatcattt tcaagaacca ggccagcagg ccctacaata tttaccccca tggcatcact
gatgtgaggc ccctgtacag caggaggctg cccaaggggg tgaagcacct gaaggacttc
cccatcctgc ctggggagat cttcaagtac aagtggactg tgactgtgga ggatggccct
accaagtctg accctaggtg tctgactagg tactacagca gctttgtgaa catggagaga
gacctggctt ctggcctgat tggccccctg ctgatctgct acaaggagtc tgtggatcag
aggggcaacc agattatgtc tgataagagg aatgtcatcc tgttctctgt gtttgatgag
aacaggagct ggtatctgac tgagaacatt cagaggttcc tgcccaaccc tgctggggtg
cagctggagg accctgagtt ccaggccagc aacatcatgc attctattaa tggctatgtg
tttgacagcc tgcagctgtc tgtgtgcctg catgaggtgg cctactggta catcctgagc
attggggccc agactgactt tctgtctgtg tttttctctg ggtacacctt caagcacaag
atggtctatg aggacaccct gaccctgttc cccttttctg gggaaactgt gtttatgagc
atggagaacc ctgggctgtg gatcctgggc tgccacaact ctgactttag gaataggggc
atgactgccc tgctgaaggt gagcagctgt gacaagaata ctggggatta ctatgaggac
agctatgagg atatctctgc ctacctgctg agcaagaaca atgccattga gcctaggagc
ttcagccaga acccccctgt gctgaagagg caccagaggg agatcaccag gaccaccctg
cagtctgatc aggaggagat tgactatgat gacaccatct ctgtggagat gaagaaggag
gactttgata tttatgatga ggatgagaac cagagcccca ggagcttcca gaagaagacc
aggcactatt tcattgctgc tgtggagagg ctgtgggact atggcatgag ctctagcccc
catgtgctga ggaacagggc ccagtctggc tctgtgcccc agttcaagaa ggtggtgttc
caggaattta ctgatggcag ctttacccag cccctgtaca gaggggagct gaatgagcac
ctgggcctgc tgggccccta catcagggct gaggtggagg ataatatcat ggtgaccttt
aggaaccagg cctctaggcc ctattctttt tacagcagcc tgatcagcta tgaggaggac
cagaggcagg gggctgagcc taggaagaac tttgtgaagc ccaatgagac caagacctac
ttttggaaag tgcagcacca catggccccc actaaggatg agtttgattg caaggcctgg
gcctatttct ctgatgtgga cctggagaag gatgtgcact ctggcctgat tggccccctg
ctggtgtgcc acaccaacac tctgaaccct gcccatggca ggcaggtgac tgtgcaggag
tttgccctgt tctttaccat ctttgatgag actaagagct ggtatttcac tgagaacatg
gagaggaact gcagagcccc ttgcaacatc cagatggagg accctacctt caaggagaac
tataggttcc atgccatcaa tgggtacatc atggataccc tgcctggcct ggtgatggct
caggaccaga ggatcaggtg gtacctgctg agcatgggga gcaatgagaa cattcatagc
atccacttct ctgggcatgt gttcactgtg aggaagaagg aggagtataa gatggccctg
tacaacctgt accctggggt gtttgagact gtggagatgc tgcccagcaa ggctggcatc
tggagggtgg agtgcctgat tggggagcac ctgcatgctg gcatgagcac tctgttcctg
gtgtacagca acaagtgcca gacccccctg ggcatggcct ctggccacat cagggacttc
cagattactg cctctgggca gtatgggcag tgggccccca agctggccag gctgcactac
tctgggtcta tcaatgcttg gagcaccaag gagcctttca gctggatcaa ggtggatctg
ctggccccca tgatcattca tgggatcaag acccaggggg ccaggcagaa gttcagcagc
ctgtatattt ctcagttcat catcatgtat tctctggatg gcaaaaagtg gcagacctat
agagggaaca gcactgggac cctgatggtg ttttttggca atgtggatag ctctggcatc
aagcacaata tcttcaaccc ccccattatt gccaggtaca tcaggctgca ccccacccac
tactctatca ggagcaccct gaggatggag ctgatgggct gtgatctgaa cagctgctct
atgcctctgg ggatggaaag caaggccatc tctgatgccc agatcactgc cagcagctat
ttcaccaata tgtttgccac ttggagccct agcaaggcta ggctgcatct gcagggcagg
tctaatgcct ggaggcccca ggtgaacaac cccaaggagt ggctgcaggt ggacttccag
aagactatga aagtgactgg ggtgaccacc cagggggtga aaagcctgct gaccagcatg
tatgtgaagg agttcctgat tagcagcagc caggatggcc accagtggac cctgttcttc
cagaatggga aggtgaaggt gtttcagggc aatcaggata gcttcacccc agtggtgaac
agcctggacc cccccctgct gaccaggtac ctgaggatcc acccccagag ctgggtgcac
cagattgccc tgaggatgga ggtgctgggc tgtgaggccc aggatctgta ctga
FVIII encoding CpG reduced nucleic acid variant X13
(SEQ ID NO: 13)
atgcagattg agctgagcac ctgctttttc ctgtgcctgc tgaggttctg cttctctgct
accaggaggt actacctggg ggctgtggag ctgtcttggg attacatgca gtctgacctg
ggggagctgc ctgtggatgc caggtttccc cccagggtgc ccaagtcttt cccctttaac
acctctgtgg tgtataagaa gactctgttt gtggagttca ctgatcacct gttcaatatt
gccaagccca ggcccccttg gatgggcctg ctgggcccca ctatccaggc tgaggtgtat
gacactgtgg tcatcaccct gaagaacatg gccagccacc ctgtgagcct gcatgctgtg
ggggtgagct actggaaggc ctctgagggg gctgagtatg atgaccagac cagccagagg
gagaaggagg atgacaaggt gttcccaggg gggtctcaca cttatgtgtg gcaggtgctg
aaggagaatg ggcccatggc ctctgaccct ctgtgcctga cttatagcta cctgtctcat
gtggatctgg tgaaggacct gaactctggc ctgattgggg ccctgctggt gtgcagggag
gggagcctgg ccaaggagaa gacccagacc ctgcacaagt tcatcctgct gtttgctgtg
tttgatgagg ggaagagctg gcactctgag accaagaata gcctgatgca ggacagggat
gctgcttctg ctagggcctg gcctaagatg cacactgtga atggctatgt gaacaggagc
ctgcctggcc tgattgggtg tcacaggaag tctgtgtact ggcatgtgat tggcatgggg
actactccag aagtgcacag catcttcctg gaggggcaca ccttcctggt gaggaatcac
aggcaggcca gcctggagat ttctcccatc actttcctga ctgcccagac cctgctgatg
gatctggggc agttcctgct gttctgccac atcagcagcc atcagcatga tgggatggag
gcctatgtga aggtggacag ctgccctgag gagcctcagc tgaggatgaa gaacaatgag
gaggctgagg actatgatga tgatctgact gactctgaga tggatgtggt gaggtttgat
gatgacaact ctcccagctt catccagatc aggtctgtgg ccaagaagca ccccaagacc
tgggtgcact acattgctgc tgaggaggag gattgggatt atgctcccct ggtgctggct
cctgatgata ggagctacaa gagccagtat ctgaataatg ggccccagag gattggcagg
aagtataaga aggtgaggtt catggcctac actgatgaga cctttaagac cagggaggct
attcagcatg agtctggcat cctgggcccc ctgctgtatg gggaggtggg ggacaccctg
ctgatcattt tcaagaacca ggccagcagg ccctataaca tctatcccca tgggatcact
gatgtgaggc ccctgtactc taggaggctg cccaaggggg tcaagcacct gaaggacttc
cccatcctgc ctggggagat cttcaagtac aagtggactg tgactgtgga ggatggcccc
actaagtctg accccaggtg cctgactagg tactacagca gctttgtgaa catggagaga
gatctggcct ctggcctgat tggccccctg ctgatctgct acaaagagtc tgtggatcag
aggggcaacc agatcatgtc tgacaagagg aatgtgatcc tgttctctgt gtttgatgag
aacagaagct ggtacctgac tgagaacatt cagaggtttc tgcccaaccc tgctggggtc
cagctggagg accctgagtt tcaggccagc aacatcatgc acagcatcaa tgggtatgtg
tttgacagcc tgcagctgtc tgtgtgcctg catgaggtgg cctactggta tatcctgagc
attggggccc agactgattt cctgtctgtg ttcttctctg gctacacttt caagcacaag
atggtgtatg aggataccct gaccctgttc cctttctctg gggaaactgt gttcatgagc
atggagaacc ctgggctgtg gatcctgggg tgccacaatt ctgatttcag gaacagaggc
atgactgctc tgctgaaggt gtctagctgt gacaagaaca ctggggacta ctatgaggac
agctatgagg acatctctgc ctacctgctg agcaagaaca atgctattga acccaggtct
ttcagccaga acccccctgt gctgaagagg caccagaggg agatcactag gaccaccctg
cagtctgatc aggaggagat tgactatgat gacaccatct ctgtggagat gaagaaggag
gactttgaca tctatgatga ggatgagaat cagtctccca ggagcttcca gaagaagact
aggcattact tcattgctgc tgtggagagg ctgtgggact atggcatgag ctctagccct
catgtgctga ggaacagggc ccagtctggc tctgtgcccc agttcaagaa ggtggtgttt
caggagttca ctgatggcag cttcacccag cccctgtaca ggggggagct gaatgagcat
ctgggcctgc tgggccccta catcagggct gaggtggagg acaacatcat ggtgaccttc
agaaatcagg ctagcaggcc ctacagcttc tacagcagcc tgatctctta tgaggaggac
cagaggcagg gggctgagcc caggaagaac tttgtgaagc ccaatgagac caagacctat
ttctggaagg tgcagcacca catggccccc accaaggatg agtttgattg caaggcctgg
gcctacttct ctgatgtgga cctggagaag gatgtgcatt ctgggctgat tggccctctg
ctggtgtgcc acaccaacac cctgaatcct gcccatggca ggcaggtgac tgtgcaggag
tttgccctgt tctttactat ctttgatgag accaagtctt ggtattttac tgagaacatg
gagaggaact gcagggcccc ctgcaacatc cagatggagg accccacctt caaggagaac
tacagattcc atgccatcaa tggctacatt atggacactc tgcctggcct ggtgatggcc
caggaccaga ggatcaggtg gtacctgctg tctatgggca gcaatgagaa cattcactct
atccacttct ctgggcatgt gttcactgtg aggaagaagg aggagtacaa gatggccctg
tacaacctgt accctggggt gtttgagact gtggagatgc tgcctagcaa ggctgggatc
tggagggtgg agtgcctgat tggggagcac ctgcatgctg gcatgtctac cctgttcctg
gtgtacagca acaagtgcca gacccccctg ggcatggcct ctggccacat cagagatttt
cagatcactg cctctggcca gtatggccag tgggctccta agctggccag gctgcactac
tctggcagca tcaatgcctg gagcaccaag gagcccttta gctggatcaa ggtggacctg
ctggccccca tgatcatcca tggcatcaag actcaggggg ccaggcagaa gttctctagc
ctgtacatta gccagttcat catcatgtat agcctggatg gcaagaagtg gcagacctac
aggggcaaca gcactgggac cctgatggtg ttctttggga atgtggacag ctctgggatc
aagcacaata tcttcaaccc ccccattatt gccaggtata ttaggctgca ccccactcac
tacagcatta ggagcaccct gaggatggag ctgatgggct gtgatctgaa cagctgcagc
atgcccctgg gcatggagtc taaggccatc tctgatgccc agatcactgc cagctcttac
ttcaccaaca tgtttgccac ttggagcccc agcaaggcca ggctgcacct gcagggcagg
agcaatgcct ggaggcccca ggtgaacaac cccaaggagt ggctgcaggt ggatttccag
aagactatga aggtgactgg ggtgaccact cagggggtga agagcctgct gactagcatg
tatgtgaagg agttcctgat cagctctagc caggatggcc accagtggac cctgttcttt
cagaatggca aggtgaaggt gttccagggc aaccaggact ctttcacccc tgtggtgaat
tctctggacc ctcccctgct gactaggtat ctgaggattc atccccagag ctgggtgcat
cagattgccc tgaggatgga ggtgctgggc tgtgaggccc aggacctgta ttga
FVIII encoding CpG reduced nucleic acid variant X14
(SEQ ID NO: 14)
atgcagattg agctgagcac ctgcttcttc ctgtgcctgc tgaggttttg cttttctgcc
actaggaggt actacctggg ggctgtggag ctgtcttggg attacatgca gtctgacctg
ggggagctgc cagtggatgc caggttcccc ccaagggtgc ccaagtcttt tcccttcaat
acctctgtgg tgtacaagaa gaccctgttt gtggagttta ctgatcatct gtttaacatt
gccaagccca ggcccccctg gatggggctg ctgggcccca ccatccaggc tgaggtgtat
gatactgtgg tgattaccct gaagaatatg gccagccatc ctgtgtctct gcatgctgtg
ggggtgtctt attggaaggc ctctgagggg gctgagtatg atgatcagac cagccagagg
gagaaggagg atgataaggt gttccctggg ggctctcaca cctatgtgtg gcaggtgctg
aaggagaatg ggcctatggc ctctgaccca ctgtgcctga cttacagcta tctgagccat
gtggacctgg tgaaggacct gaactctggg ctgattgggg ccctgctggt gtgcagggag
ggcagcctgg ccaaggagaa gactcagacc ctgcacaagt tcatcctgct gtttgctgtg
tttgatgagg gcaagtcttg gcactctgag accaagaaca gcctgatgca ggatagggat
gctgcctctg ccagggcctg gcccaagatg cacactgtga atggctatgt gaacaggtct
ctgcctggcc tgattggctg ccacaggaag tctgtgtact ggcatgtgat tggcatgggc
accacccctg aggtgcatag cattttcctg gagggccaca ccttcctggt gaggaaccac
aggcaggcta gcctggagat cagccccatc actttcctga ctgcccagac cctgctgatg
gacctgggcc agttcctgct gttctgccac atctctagcc accagcatga tggcatggag
gcctatgtga aggtggactc ttgtcctgag gagccccagc tgaggatgaa gaacaatgag
gaggctgagg attatgatga tgatctgact gattctgaga tggatgtggt gaggtttgat
gatgacaaca gcccctcttt catccagatc aggtctgtgg ccaagaagca ccccaagacc
tgggtgcact acattgctgc tgaggaggag gattgggatt atgcccccct ggtgctggcc
cctgatgaca ggagctataa gtctcagtac ctgaacaatg gcccccagag aattggcagg
aagtacaaga aggtgaggtt catggcctat actgatgaga ccttcaaaac cagggaggcc
attcagcatg agtctggcat cctggggccc ctgctgtatg gggaggtggg ggacaccctg
ctgatcatct tcaagaacca ggctagcagg ccttacaaca tctaccccca tgggatcact
gatgtgaggc ccctgtacag caggaggctg cctaaggggg tgaagcacct gaaggacttt
cccattctgc ctggggagat cttcaagtat aagtggactg tgactgtgga ggatgggccc
accaagtctg accccaggtg cctgactagg tactactcta gctttgtgaa catggagagg
gacctggcct ctgggctgat tggccccctg ctgatctgtt acaaggagtc tgtggaccag
aggggcaacc agatcatgtc tgataagagg aatgtgatcc tgttctctgt gtttgatgag
aacaggagct ggtacctgac tgagaacatc cagagattcc tgcccaaccc tgctggggtg
cagctggagg atcctgagtt ccaggccagc aacatcatgc attctatcaa tgggtatgtg
tttgatagcc tgcagctgtc tgtgtgtctg catgaggtgg cctactggta cattctgagc
attggggccc agactgactt cctgtctgtg ttcttctctg gctacacttt caaacacaag
atggtgtatg aggacaccct gaccctgttc cccttctctg gggagactgt gtttatgagc
atggagaacc ctgggctgtg gattctgggc tgccacaact ctgacttcag aaacaggggc
atgactgccc tgctgaaggt gtcttcttgt gataagaaca ctggggacta ttatgaagac
agctatgagg acatctctgc ctacctgctg agcaagaata atgctattga gcccaggtct
ttctctcaga acccccctgt gctgaagagg caccagaggg agatcaccag gaccaccctg
cagtctgatc aggaggagat tgactatgat gacactattt ctgtggagat gaagaaggaa
gactttgata tctatgatga ggatgagaac cagagcccta ggagcttcca gaagaagact
aggcattact tcattgctgc tgtggagagg ctgtgggact atggcatgag cagcagcccc
catgtgctga ggaatagggc tcagtctggc tctgtgcctc agttcaagaa ggtggtgttc
caggaattca ctgatggcag cttcactcag cccctgtaca ggggggagct gaatgagcac
ctggggctgc tgggccctta catcagggct gaggtggagg acaatatcat ggtgaccttt
aggaaccagg cctctaggcc ttacagcttc tactctagcc tgatctctta tgaagaggac
cagaggcagg gggctgagcc caggaagaac tttgtgaagc ccaatgagac taagacttac
ttctggaagg tgcagcacca catggctccc accaaggatg agtttgactg caaggcttgg
gcctacttct ctgatgtgga cctggagaag gatgtgcact ctgggctgat tgggcccctg
ctggtgtgcc acactaacac tctgaatcct gcccatggca gacaggtgac tgtgcaggag
tttgccctgt tttttaccat ctttgatgag actaagtctt ggtacttcac tgagaacatg
gagaggaact gcagggcccc ctgcaacatc cagatggagg atcccacctt caaggagaac
tacaggtttc atgccatcaa tggctacatc atggacaccc tgcctggcct ggtgatggct
caggaccaga ggattaggtg gtatctgctg agcatgggca gcaatgagaa tatccactct
atccacttct ctgggcatgt gttcactgtg aggaagaagg aggagtacaa gatggccctg
tataacctgt atcctggggt gtttgagact gtggagatgc tgcccagcaa ggctggcatc
tggagagtgg agtgcctgat tggggagcac ctgcatgctg gcatgagcac tctgtttctg
gtgtatagca acaagtgtca gacccctctg ggcatggcct ctgggcacat tagggacttt
cagatcactg cttctggcca gtatgggcag tgggctccca agctggccag gctgcactat
tctggcagca ttaatgcctg gagcaccaag gagcctttca gctggatcaa ggtggacctg
ctggccccca tgatcatcca tgggatcaag acccaggggg ctaggcagaa gttcagcagc
ctgtacatca gccagtttat catcatgtat tctctggatg gcaagaagtg gcagacctac
aggggcaatt ctactggcac tctgatggtg ttctttggga atgtggatag ctctgggatc
aagcataata tcttcaatcc ccccattatt gctaggtata tcaggctgca ccccacccac
tatagcatca ggagcaccct gaggatggag ctgatggggt gtgacctgaa cagctgcagc
atgcccctgg gcatggagag caaggctatt tctgatgccc agatcactgc cagcagctac
tttactaata tgtttgccac ctggagcccc agcaaggcca gactgcacct gcagggcagg
tctaatgcct ggaggcctca ggtgaataac cccaaggagt ggctgcaggt ggacttccag
aaaaccatga aggtgactgg ggtgactacc cagggggtga agtctctgct gaccagcatg
tatgtgaagg agttcctgat ctcttctagc caggatggcc accagtggac cctgttcttt
cagaatggga aggtgaaggt cttccagggc aaccaggata gcttcacccc tgtggtgaat
agcctggatc ctcctctgct gaccaggtat ctgaggatcc acccccagag ctgggtgcat
cagattgccc tgaggatgga ggtgctgggc tgtgaggctc aggacctgta ctga
FVIII encoding CpG reduced nucleic acid variant X15
(SEQ ID NO: 15)
atgcagattg agctgagcac ctgtttcttc ctgtgcctgc tgaggttctg tttctctgcc
actaggaggt actacctggg ggctgtggag ctgagctggg actatatgca gtctgacctg
ggggagctgc ctgtggatgc caggttcccc cccagggtgc ctaagagctt ccccttcaat
acttctgtgg tgtacaagaa gactctgttt gtggagttta ctgaccacct gttcaacatt
gctaagccca ggcctccctg gatggggctg ctgggcccca ccatccaggc tgaggtgtat
gatactgtgg tgattaccct gaagaacatg gcctctcatc cagtgagcct gcatgctgtg
ggggtgagct actggaaggc ctctgaaggg gctgagtatg atgaccagac cagccagagg
gagaaggagg atgacaaggt gttccctggg ggcagccaca cctatgtgtg gcaggtgctg
aaggagaatg gcccaatggc ctctgacccc ctgtgcctga cttatagcta cctgagccat
gtggatctgg tgaaggacct gaattctggc ctgattgggg ccctgctggt gtgcagagag
ggctctctgg ctaaggagaa gacccagact ctgcacaagt tcatcctgct gtttgctgtg
tttgatgagg gcaagagctg gcactctgag actaagaata gcctgatgca ggacagggat
gctgcttctg ccagggcctg gcccaagatg catactgtga atggctatgt gaacaggagc
ctgcctggcc tgattggctg tcacaggaaa tctgtctact ggcatgtgat tgggatgggc
actacccctg aggtgcactc tatcttcctg gagggccata ccttcctggt gaggaaccac
aggcaggcca gcctggagat ctctcccatt accttcctga ctgcccagac cctgctgatg
gatctgggcc agttcctgct gttctgccac atcagcagcc accagcatga tgggatggag
gcttatgtga aggtggatag ctgccctgag gagccccagc tgaggatgaa gaacaatgag
gaggctgagg actatgatga tgacctgact gactctgaga tggatgtggt gaggtttgat
gatgacaact ctcccagctt tattcagatc aggtctgtgg ctaagaagca ccccaagact
tgggtgcact acattgctgc tgaggaggag gactgggact atgcccctct ggtgctggct
cctgatgaca ggtcttacaa gtctcagtac ctgaataatg gccctcagag gattggcagg
aagtacaaga aggtgaggtt catggcctac actgatgaga ccttcaagac cagggaggcc
atccagcatg agtctggcat cctgggcccc ctgctgtatg gggaggtggg ggataccctg
ctgatcatct tcaagaatca ggccagcagg ccctacaaca tctaccccca tggcatcact
gatgtgaggc cactgtacag caggaggctg cccaaggggg tgaagcatct gaaggacttc
cccattctgc ctggggagat cttcaagtac aaatggactg tgactgtgga ggatggccct
accaagtctg accccaggtg tctgaccagg tactacagca gctttgtgaa tatggagagg
gacctggcct ctggcctgat tggccccctg ctgatctgct acaaggagtc tgtggaccag
aggggcaatc agatcatgtc tgataagagg aatgtgattc tgttctctgt gtttgatgag
aacaggagct ggtacctgac tgagaacatc cagaggttcc tgcccaatcc tgctggggtg
cagctggagg accctgagtt ccaggccagc aatatcatgc acagcatcaa tggctatgtc
tttgacagcc tgcagctgtc tgtgtgcctg catgaggtgg cttactggta tattctgagc
attggggccc agactgattt cctgtctgtg ttcttttctg gctatacctt taagcacaag
atggtgtatg aggacaccct gaccctgttc cccttctctg gggagactgt gttcatgtct
atggagaacc ctgggctgtg gatcctgggc tgccacaact ctgacttcag gaacaggggg
atgactgccc tgctgaaggt gtctagctgt gataagaaca ctggggacta ttatgaggac
agctatgagg acatctctgc ttacctgctg agcaagaaca atgccattga gcccaggtct
ttcagccaga atccccctgt gctgaagagg catcagaggg agatcaccag gaccaccctg
cagtctgatc aggaggagat tgattatgat gacactatct ctgtggaaat gaagaaggag
gactttgaca tctatgatga ggatgagaac cagagcccca ggagcttcca gaagaagacc
aggcactact tcattgctgc tgtggagagg ctgtgggatt atggcatgag cagctctccc
catgtgctga ggaacagagc ccagtctggc tctgtgcctc agttcaagaa ggtggtcttc
caggagttca ctgatggctc tttcacccag cccctgtaca ggggggagct gaatgagcac
ctgggcctgc tggggcccta cattagggct gaggtggagg ataacatcat ggtgactttc
agaaaccagg ccagcaggcc ttacagcttt tactcttctc tgattagcta tgaggaggat
cagaggcagg gggctgagcc taggaagaac tttgtgaagc ccaatgagac caagacctat
ttctggaagg tgcagcacca catggctccc actaaggatg agtttgactg caaggcttgg
gcctacttct ctgatgtgga cctggagaag gatgtgcact ctggcctgat tgggcccctg
ctggtgtgcc acaccaacac cctgaaccct gcccatggca ggcaggtgac tgtgcaggag
tttgccctgt tcttcaccat ctttgatgag actaagagct ggtacttcac tgagaacatg
gagaggaact gcagggcccc ctgcaacatc cagatggagg accccacctt caaggagaat
tacaggttcc atgccatcaa tggctacatt atggacaccc tgcctggcct ggtgatggcc
caggatcaga ggatcaggtg gtatctgctg agcatgggct ctaatgagaa catccacagc
atccacttct ctggccatgt gtttactgtg aggaagaagg aggaatacaa gatggctctg
tataacctgt accctggggt gtttgagact gtggagatgc tgcccagcaa ggctgggatc
tggagggtgg agtgcctgat tggggagcac ctgcatgctg ggatgagcac cctgttcctg
gtgtatagca ataagtgcca gacccccctg ggcatggctt ctggccacat cagggatttc
cagatcactg cttctggcca gtatggccag tgggctccca agctggctag gctgcattac
tctgggtcta tcaatgcctg gagcactaag gagcccttca gctggatcaa ggtggacctg
ctggccccca tgatcattca tggcatcaag acccaggggg ctaggcagaa gttcagcagc
ctgtacatca gccagttcat cattatgtac agcctggatg gcaagaagtg gcagacttac
aggggcaata gcactgggac tctgatggtg ttctttggca atgtggactc ttctggcatc
aagcacaaca tcttcaaccc tcccatcatt gccaggtaca ttaggctgca ccctacccac
tactctatca ggagcaccct gaggatggag ctgatggggt gtgatctgaa ctcttgcagc
atgcctctgg gcatggaaag caaagccatc tctgatgccc agatcactgc ctctagctat
ttcaccaata tgtttgccac ctggagccct agcaaggcca ggctgcacct gcagggcaga
tctaatgcct ggaggcccca ggtgaacaat cccaaggagt ggctgcaggt ggacttccag
aagaccatga aggtgactgg ggtgaccact cagggggtga agagcctgct gactagcatg
tatgtgaagg agttcctgat ctcttctagc caggatggcc accagtggac cctgttcttc
cagaatggca aggtgaaagt gttccagggc aaccaggata gcttcactcc tgtggtgaac
tctctggacc ctcccctgct gactaggtac ctgaggattc atccccagag ctgggtgcac
cagattgccc tgaggatgga ggtgctgggc tgtgaggccc aggatctgta ctga
FVIII encoding CpG reduced nucleic acid variant X16
(SEQ ID NO: 16)
atgcagattg agctgagcac ctgcttcttc ctgtgcctgc tgaggttctg cttctctgcc
accaggaggt actacctggg ggctgtggag ctgtcttggg actatatgca gtctgacctg
ggggagctgc cagtggatgc caggttcccc cccagggtgc ccaagagctt tcctttcaac
acttctgtgg tgtacaagaa gaccctgttt gtggagttca ctgaccacct gttcaatatt
gctaagccca ggccaccctg gatgggcctg ctgggcccta ccattcaggc tgaggtgtat
gacactgtgg tgattactct gaagaatatg gccagccacc ctgtgagcct gcatgctgtg
ggggtgtctt actggaaggc ctctgagggg gctgagtatg atgatcagac ttctcagagg
gagaaggagg atgataaggt gttccctggg ggctctcaca cttatgtgtg gcaggtgctg
aaggagaatg gccccatggc ttctgatcca ctgtgcctga cctactctta cctgagccat
gtggacctgg tgaaggacct gaactctggc ctgattgggg ccctgctggt gtgcagggag
ggcagcctgg ccaaggagaa gacccagacc ctgcataagt tcatcctgct gtttgctgtg
tttgatgagg ggaagagctg gcactctgag accaagaatt ctctgatgca ggacagggat
gctgcctctg ccagggcctg gcctaagatg cacactgtga atggctatgt gaacaggtct
ctgcctggcc tgattggctg ccacaggaag tctgtgtact ggcatgtgat tggcatgggc
actacccctg aggtgcacag cattttcctg gagggccaca ccttcctggt caggaaccat
aggcaggcct ctctggagat cagccccatc actttcctga ctgcccagac cctgctgatg
gacctgggcc agttcctgct gttctgccac attagcagcc accagcatga tggcatggag
gcctatgtga aggtggactc ttgccctgag gagccccagc tgaggatgaa gaacaatgag
gaagctgagg attatgatga tgacctgact gactctgaga tggatgtggt gaggtttgat
gatgacaaca gccccagctt catccagatc aggtctgtgg ccaagaagca ccccaagacc
tgggtgcact acattgctgc tgaggaggag gattgggact atgctcccct ggtgctggct
cctgatgata ggagctacaa gtctcagtac ctgaataatg gcccccagag gattggcagg
aagtacaaga aggtgaggtt catggcctac actgatgaga ccttcaagac cagagaggct
atccagcatg agtctgggat cctggggccc ctgctgtatg gggaggtggg ggacaccctg
ctgatcatct tcaagaacca ggccagcaga ccctacaaca tctaccccca tgggatcact
gatgtgaggc ccctgtacag caggaggctg cctaaggggg tgaagcacct gaaggacttc
cccatcctgc ctggggagat cttcaagtat aagtggactg tgactgtgga ggatgggccc
accaagtctg accctaggtg cctgactagg tactactcta gctttgtgaa catggagagg
gacctggcct ctggcctgat tggccccctg ctgatttgct acaaggagtc tgtggatcag
aggggcaatc agatcatgtc tgacaagagg aatgtgatcc tgttctctgt gtttgatgag
aataggtctt ggtacctgac tgagaacatc cagaggttcc tgcctaatcc tgctggggtg
cagctggagg accctgagtt tcaggccagc aacatcatgc acagcatcaa tggctatgtg
tttgactctc tgcagctgtc tgtgtgcctg catgaggtgg cttactggta tatcctgagc
attggggctc agactgactt cctgtctgtg ttcttttctg gctacacttt taagcacaag
atggtgtatg aggacaccct gaccctgttc cccttttctg gggagactgt gttcatgtct
atggagaacc ctgggctgtg gattctgggc tgtcacaact ctgacttcag aaacaggggc
atgactgccc tgctgaaggt gtctagctgt gacaagaata ctggggacta ctatgaggac
agctatgagg acatttctgc ctatctgctg agcaagaaca atgccattga gcccaggagc
ttttctcaga atccccctgt gctgaagagg caccagagag agatcaccag gaccactctg
cagtctgatc aggaggagat tgattatgat gacactatct ctgtggagat gaagaaagag
gactttgata tctatgatga ggatgagaat cagtctccca ggagcttcca gaagaagact
agacactact tcattgctgc tgtggagagg ctgtgggact atggcatgag ctctagccct
catgtgctga ggaacagggc ccagtctggg tctgtgcccc agttcaagaa ggtggtgttc
caggagttca ctgatggcag ctttacccag cccctgtata ggggggagct gaatgagcat
ctgggcctgc tgggccccta tattagggct gaagtggagg acaacatcat ggtgaccttt
aggaaccagg ccagcaggcc ctacagcttt tacagcagcc tgattagcta tgaggaggat
cagagacagg gggctgagcc caggaagaac tttgtgaagc ccaatgagac caagacctac
ttctggaagg tgcagcacca catggcccct accaaggatg agtttgactg caaggcctgg
gcttacttct ctgatgtgga cctggagaaa gatgtgcact ctggcctgat tgggcccctg
ctggtgtgcc acaccaacac cctgaaccct gcccatggga ggcaggtgac tgtgcaggag
tttgccctgt ttttcaccat ctttgatgag accaagagct ggtacttcac tgagaacatg
gagaggaact gcagggcccc ctgtaacatc cagatggagg atcctacttt caaggagaac
tacaggttcc atgccattaa tgggtacatc atggacaccc tgcctgggct ggtgatggcc
caggatcaga ggattaggtg gtatctgctg tctatgggct ctaatgagaa catccactct
atccacttct ctggccatgt gttcactgtg aggaagaagg aggagtacaa gatggccctg
tacaacctgt accctggggt gtttgaaact gtggagatgc tgccctctaa agctgggatc
tggagggtgg agtgcctgat tggggagcac ctgcatgctg gcatgagcac cctgttcctg
gtgtacagca ataagtgcca gactcccctg ggcatggctt ctgggcacat cagggatttc
cagatcactg cctctggcca gtatggccag tgggccccca agctggctag gctgcactac
tctggcagca tcaatgcctg gagcaccaag gagcccttct cttggattaa ggtggacctg
ctggctccca tgatcattca tggcatcaag acccaggggg ccaggcagaa gttttctagc
ctgtatatta gccagttcat catcatgtat agcctggatg ggaagaagtg gcagacctac
agggggaata gcactggcac cctgatggtg ttttttggca atgtggattc ttctggcatc
aagcataaca tcttcaatcc ccctatcatt gccaggtaca ttaggctgca tcccacccat
tactctatca ggagcaccct gaggatggag ctgatggggt gtgatctgaa cagctgtagc
atgcccctgg gcatggagtc caaggctatc tctgatgccc agatcactgc cagcagctac
ttcaccaaca tgtttgccac ctggagcccc agcaaggcca ggctgcacct gcagggcagg
tctaatgcct ggaggcccca ggtgaacaat cccaaggagt ggctgcaggt ggacttccag
aagactatga aggtgactgg ggtgaccact cagggggtga agagcctgct gaccagcatg
tatgtgaagg agttcctgat ctcttctagc caggatgggc atcagtggac cctgtttttt
cagaatggca aagtgaaggt gtttcagggg aatcaggaca gctttacccc tgtggtgaac
agcctggatc ctcctctgct gactagatac ctgaggatcc acccccagag ctgggtccac
cagattgctc tgaggatgga ggtgctgggg tgtgaggctc aggacctgta ctga
FVIII encoding CpG reduced nucleic acid variant X17
(SEQ ID NO: 17)
atgcagattg agctgagcac ctgcttcttt ctgtgcctgc tgaggttctg cttctctgcc
accaggaggt actacctggg ggctgtggaa ctgagctggg actatatgca gtctgacctg
ggggagctgc ctgtggatgc caggttcccc cccagggtgc ccaagtcttt cccctttaac
acttctgtgg tgtacaagaa gaccctgttt gtggagttta ctgaccacct gttcaatatt
gccaagccca ggcccccctg gatgggcctg ctgggcccaa ccatccaggc tgaggtgtat
gatactgtgg tgatcaccct gaagaacatg gccagccacc ctgtgagcct gcatgctgtg
ggggtgagct attggaaggc ttctgagggg gctgagtatg atgaccagac tagccagagg
gagaaggagg atgacaaggt gttccctggg gggtctcata cctatgtgtg gcaggtgctg
aaggagaatg gccccatggc ctctgacccc ctgtgcctga cctattctta cctgagccat
gtggacctgg tcaaggacct gaactctggc ctgattgggg ctctgctggt gtgcagggag
ggcagcctgg ccaaggagaa gactcagact ctgcataagt tcatcctgct gtttgctgtg
tttgatgagg gcaagagctg gcactctgag accaagaact ctctgatgca ggatagggat
gctgcctctg ccagggcctg gcccaagatg cacactgtga atggctatgt gaataggtct
ctgcctggcc tgattggctg ccataggaag tctgtgtact ggcatgtgat tggcatgggc
actacccctg aggtgcactc tatcttcctg gaggggcaca ccttcctggt gaggaaccac
aggcaggcca gcctggagat ctctcccatc accttcctga ctgcccagac tctgctgatg
gacctgggcc agttcctgct gttctgccat atcagcagcc accagcatga tggcatggag
gcctatgtga aggtggacag ctgcccagag gaaccccagc tgaggatgaa gaacaatgag
gaggctgagg actatgatga tgacctgact gactctgaga tggatgtggt gaggtttgat
gatgacaaca gccccagctt tattcagatc aggtctgtgg ccaagaagca ccccaagacc
tgggtgcact acattgctgc tgaggaggag gactgggatt atgcccccct ggtgctggcc
cctgatgaca ggtcttacaa gtctcagtac ctgaacaatg gcccccagag gattgggagg
aagtacaaga aggtgaggtt catggcctac actgatgaga ccttcaagac cagggaggcc
atccagcatg agtctggcat cctggggccc ctgctgtatg gggaggtggg ggataccctg
ctgattatct tcaagaacca ggctagcagg ccctataaca tctaccccca tggcattact
gatgtgaggc ccctgtactc taggagactg cccaaggggg tgaagcacct gaaagacttc
cccatcctgc ctggggagat cttcaagtat aagtggactg tgactgtgga ggatggcccc
actaagtctg accccaggtg cctgaccagg tattacagca gctttgtgaa tatggagagg
gatctggctt ctggcctgat tgggcctctg ctgatttgct acaaggagtc tgtggatcag
agggggaacc agattatgtc tgacaagagg aatgtgattc tgttctctgt gtttgatgag
aacaggagct ggtacctgac tgagaatatc cagaggttcc tgcctaatcc tgctggggtg
cagctggagg accctgagtt ccaggctagc aacattatgc acagcatcaa tggctatgtg
tttgacagcc tgcagctgtc tgtgtgcctg catgaggtgg cttactggta cattctgtct
attggggccc agactgactt cctgtctgtg ttcttctctg gctacacctt caagcacaag
atggtgtatg aggacactct gaccctgttc cccttctctg gggagactgt gttcatgagc
atggagaatc ctgggctgtg gattctgggg tgccacaact ctgatttcag gaacaggggc
atgactgccc tgctgaaggt gagcagctgt gacaagaaca ctggggatta ttatgaggac
agctatgagg acatttctgc ctacctgctg agcaagaaca atgccattga gcctaggagc
ttcagccaga atccccctgt gctgaagaga caccagaggg agatcactag gaccactctg
cagtctgatc aggaggagat tgactatgat gacaccattt ctgtggagat gaagaaggag
gactttgata tttatgatga ggatgagaac cagagcccca gaagcttcca gaagaagacc
aggcactact tcattgctgc tgtggagagg ctgtgggatt atggcatgtc ttctagcccc
catgtgctga ggaacagggc tcagtctggc tctgtgcctc agttcaagaa ggtggtgttc
caggagttca ctgatgggag cttcacccag cctctgtaca ggggggagct gaatgaacat
ctgggcctgc tggggcccta catcagggct gaggtggagg ataatatcat ggtgactttc
aggaatcagg cctctaggcc ctacagcttc tactctagcc tgatcagcta tgaggaggac
cagaggcagg gggctgagcc taggaagaat tttgtgaaac ccaatgagac caagacctac
ttttggaagg tgcagcacca catggcccct accaaggatg agtttgactg taaggcctgg
gcctacttct ctgatgtgga cctggagaag gatgtgcatt ctgggctgat tggccccctg
ctggtgtgcc acaccaacac cctgaaccct gcccatggca ggcaggtgac tgtgcaggag
tttgccctgt tcttcaccat ctttgatgag actaagagct ggtatttcac tgagaacatg
gagaggaact gtagggctcc ctgcaacatc cagatggagg atccaacttt caaggagaac
tacaggttcc atgccatcaa tggctacatc atggacaccc tgcctggcct ggtgatggcc
caggaccaga ggattaggtg gtacctgctg agcatgggct ctaatgagaa catccactct
atccacttct ctggccatgt gtttactgtg aggaagaagg aggagtacaa gatggctctg
tacaacctgt accctggggt gtttgagact gtggagatgc tgcctagcaa ggctggcatt
tggagagtgg agtgtctgat tggggagcac ctgcatgctg ggatgtctac cctgttcctg
gtgtactcta acaagtgcca gacccccctg gggatggctt ctgggcacat cagagatttt
cagattactg cttctgggca gtatggccag tgggctccca agctggccag actgcattac
tctggctcta ttaatgcttg gagcaccaag gagcctttca gctggatcaa ggtggacctg
ctggctccca tgatcatcca tggcattaag actcaggggg ctaggcagaa gttcagcagc
ctgtatattt ctcagtttat tatcatgtat tctctggatg gcaagaagtg gcagacttac
aggggcaaca gcactggcac cctgatggtg ttctttggca atgtggacag ctctgggatc
aagcataaca tcttcaaccc ccccattatt gccaggtaca tcaggctgca ccccacccac
tattctatca ggagcactct gaggatggag ctgatggggt gtgacctgaa cagctgctct
atgcccctgg gcatggagag caaggccatc tctgatgccc agatcactgc cagctcttat
ttcaccaaca tgtttgccac ctggagcccc agcaaggcca ggctgcacct gcagggcaga
agcaatgcct ggaggcccca ggtgaacaat cctaaggagt ggctgcaggt ggacttccag
aagactatga aggtgactgg ggtgactacc cagggggtga agagcctgct gaccagcatg
tatgtgaagg agttcctgat tagcagcagc caggatgggc atcagtggac cctgttcttc
cagaatggga aggtgaaggt gttccagggc aatcaggaca gcttcacccc tgtggtgaac
agcctggacc cccccctgct gaccaggtac ctgaggatcc atccccagag ctgggtgcac
cagattgctc tgagaatgga ggtgctgggc tgtgaggccc aggacctgta ttga
FVIII encoding CpG reduced nucleic acid variant X18
(SEQ ID NO: 18)
atgcagattg agctgtctac ctgttttttt ctgtgcctgc tgaggttctg cttctctgct
accaggaggt attatctggg ggctgtggag ctgagctggg actacatgca gtctgacctg
ggggagctgc ctgtggatgc caggtttcct cccagggtgc ctaagagctt ccccttcaac
acctctgtgg tgtacaagaa gactctgttt gtggagttca ctgaccacct gttcaacatt
gccaagccca ggcccccctg gatggggctg ctgggcccca ctatccaggc tgaggtgtat
gatactgtgg tgattaccct gaagaacatg gcctctcacc ctgtgtctct gcatgctgtg
ggggtgagct actggaaggc ttctgagggg gctgaatatg atgatcagac ctctcagagg
gagaaggagg atgacaaggt gtttcctggg ggcagccaca cctatgtgtg gcaggtgctg
aaggagaatg ggcccatggc ctctgatccc ctgtgcctga cctacagcta cctgagccat
gtggacctgg tgaaggacct gaactctggc ctgattgggg ccctgctggt gtgcagggag
ggcagcctgg ccaaggaaaa gacccagacc ctgcataagt tcatcctgct gtttgctgtg
tttgatgagg gcaagtcttg gcactctgag accaagaaca gcctgatgca ggacagggat
gctgcctctg ctagggcctg gcccaagatg cacactgtga atgggtatgt gaacagatct
ctgcctggcc tgattggctg ccacaggaag tctgtgtact ggcatgtgat tggcatgggg
accacccctg aggtgcatag catcttcctg gaggggcaca ccttcctggt gagaaatcat
aggcaggcca gcctggagat tagccccatc accttcctga ctgcccagac cctgctgatg
gacctgggcc agttcctgct gttctgccac atttctagcc accagcatga tggcatggag
gcctatgtga aggtggatag ctgccctgaa gagccccagc tgaggatgaa gaacaatgag
gaggctgagg attatgatga tgatctgact gactctgaga tggatgtggt gaggtttgat
gatgacaaca gccccagctt catccagatc aggtctgtgg ccaagaagca ccctaagacc
tgggtgcact acattgctgc tgaagaggag gactgggact atgcccccct ggtgctggcc
ccagatgaca ggtcttacaa gagccagtac ctgaataatg gcccccagag gattgggagg
aagtataaga aagtgaggtt catggcttac actgatgaga cctttaagac tagggaggcc
attcagcatg agtctgggat tctgggccct ctgctgtatg gggaggtggg ggacaccctg
ctgatcattt tcaagaacca ggccagcagg ccctataata tttatcccca tgggattact
gatgtcaggc ccctgtacag caggaggctg cctaaggggg tgaagcacct gaaggacttc
cccattctgc ctggggagat cttcaagtat aagtggactg tgactgtgga ggatggcccc
accaagtctg atcctaggtg cctgaccagg tactatagca gctttgtgaa catggagagg
gacctggctt ctggcctgat tggccccctg ctgatctgct acaaggaatc tgtggaccag
aggggcaacc agattatgtc tgacaagagg aatgtgatcc tgttttctgt gtttgatgag
aataggagct ggtatctgac tgagaacatc cagaggttcc tgcccaatcc tgctggggtg
cagctggagg accctgagtt ccaggcttct aacatcatgc atagcatcaa tgggtatgtg
tttgactctc tgcagctgtc tgtgtgcctg catgaggtgg cctattggta catcctgagc
attggggccc agactgactt cctgtctgtg ttcttctctg gctacacctt caagcacaag
atggtgtatg aggacaccct gaccctgttc cctttctctg gggagactgt gttcatgagc
atggagaacc ctggcctgtg gattctgggc tgccataatt ctgacttcag aaacaggggc
atgactgctc tgctgaaggt gagcagctgt gacaagaata ctggggacta ctatgaggac
tcttatgagg atatttctgc ctacctgctg agcaagaaca atgctattga gcccaggagc
ttcagccaga acccccctgt cctgaagagg catcagaggg agatcactag gaccaccctg
cagtctgatc aggaggagat tgactatgat gacactatct ctgtggaaat gaagaaggag
gactttgata tctatgatga ggatgagaac cagagcccca ggtctttcca gaagaagacc
aggcactact tcattgctgc tgtggagagg ctgtgggact atggcatgtc tagcagcccc
catgtgctga ggaacagagc ccagtctggc tctgtgcccc agttcaagaa ggtggtgttt
caggagttca ctgatgggag cttcactcag cccctgtata ggggggagct gaatgagcat
ctgggcctgc tggggcccta catcagggct gaggtggagg ataacatcat ggtgaccttc
aggaaccagg ccagcaggcc ctactctttc tactcttctc tgatcagcta tgaggaggat
cagaggcagg gggctgagcc taggaagaac tttgtcaagc ctaatgagac taagacctac
ttttggaagg tgcagcacca catggctccc actaaggatg agtttgattg caaggcctgg
gcctacttct ctgatgtgga cctggagaag gatgtgcact ctggcctgat tggccccctg
ctggtgtgtc acaccaatac cctgaaccct gcccatggca ggcaggtcac tgtgcaggag
tttgccctgt ttttcactat ctttgatgag actaagtctt ggtacttcac tgagaacatg
gaaaggaatt gcagggctcc ctgcaacatc cagatggagg accccacctt caaggagaac
tacaggtttc atgccatcaa tggctacatc atggacaccc tgcctggcct ggtgatggct
caggatcaga ggattaggtg gtatctgctg agcatgggca gcaatgagaa catccacagc
atccactttt ctggccatgt gttcactgtg aggaagaagg aggagtacaa gatggctctg
tacaatctgt accctggggt gtttgagact gtggagatgc tgcccagcaa ggctgggatc
tggagggtgg agtgcctgat tggggaacac ctgcatgctg gcatgtctac cctgttcctg
gtgtactcta acaagtgcca gactcccctg ggcatggcct ctgggcacat cagggacttc
cagatcactg cctctgggca gtatggccag tgggccccta agctggctag gctgcattac
tctggcagca tcaatgcctg gagcaccaag gagcccttca gctggatcaa ggtggacctg
ctggccccta tgatcatcca tggcatcaag acccaggggg ccagacagaa gttctcttct
ctgtacatct ctcagttcat catcatgtac tctctggatg gcaagaagtg gcagacctac
agggggaatt ctactggcac tctgatggtg ttctttggga atgtggatag ctctgggatc
aagcataata ttttcaaccc ccccattatt gctaggtaca tcaggctgca cccaacccac
tactctatta ggtctaccct gaggatggag ctgatgggct gtgacctgaa ctcttgtagc
atgcccctgg gcatggagag caaggctatc tctgatgccc agatcactgc cagcagctac
tttaccaaca tgtttgctac ttggagcccc agcaaggcca ggctgcacct gcagggcagg
agcaatgcct ggaggcccca ggtgaacaac cccaaggagt ggctgcaggt ggattttcag
aagaccatga aggtgactgg ggtgaccact cagggggtga aaagcctgct gactagcatg
tatgtgaagg agtttctgat cagcagctct caggatggcc atcagtggac cctgttcttc
cagaatggca aggtgaaggt gttccagggc aaccaggata gcttcacccc tgtggtgaat
agcctggacc cccccctgct gaccaggtac ctgaggatcc atccccagag ctgggtgcac
cagattgccc tgaggatgga ggtgctgggc tgtgaagccc aggacctgta ctga
Wild-type factor VIII-BDD cDNA
(SEQ ID NO: 19)
ATGCAAATAG AGCTCTCCAC CTGCTTCTTT CTGTGCCTTT TGCGATTCTG CTTTAGTGCC
ACCAGAAGAT ACTACCTGGG TGCAGTGGAA CTGTCATGGG ACTATATGCA AAGTGATCTC
GGTGAGCTGC CTGTGGACGC AAGATTTCCT CCTAGAGTGC CAAAATCTTT TCCATTCAAC
ACCTCAGTCG TGTACAAAAA GACTCTGTTT GTAGAATTCA CGGATCACCT TTTCAACATC
GCTAAGCCAA GGCCACCCTG GATGGGTCTG CTAGGTCCTA CCATCCAGGC TGAGGTTTAT
GATACAGTGG TCATTACACT TAAGAACATG GCTTCCCATC CTGTCAGTCT TCATGCTGTT
GGTGTATCCT ACTGGAAAGC TTCTGAGGGA GCTGAATATG ATGATCAGAC CAGTCAAAGG
GAGAAAGAAG ATGATAAAGT CTTCCCTGGT GGAAGCCATA CATATGTCTG GCAGGTCCTG
AAAGAGAATG GTCCAATGGC CTCTGACCCA CTGTGCCTTA CCTACTCATA TCTTTCTCAT
GTGGACCTGG TAAAAGACTT GAATTCAGGC CTCATTGGAG CCCTACTAGT ATGTAGAGAA
GGGAGTCTGG CCAAGGAAAA GACACAGACC TTGCACAAAT TTATACTACT TTTTGCTGTA
TTTGATGAAG GGAAAAGTTG GCACTCAGAA ACAAAGAACT CCTTGATGCA GGATAGGGAT
GCTGCATCTG CTCGGGCCTG GCCTAAAATG CACACAGTCA ATGGTTATGT AAACAGGTCT
CTGCCAGGTC TGATTGGATG CCACAGGAAA TCAGTCTATT GGCATGTGAT TGGAATGGGC
ACCACTCCTG AAGTGCACTC AATATTCCTC GAAGGTCACA CATTTCTTGT GAGGAACCAT
CGCCAGGCGT CCTTGGAAAT CTCGCCAATA ACTTTCCTTA CTGCTCAAAC ACTCTTGATG
GACCTTGGAC AGTTTCTACT GTTTTGTCAT ATCTCTTCCC ACCAACATGA TGGCATGGAA
GCTTATGTCA AAGTAGACAG CTGTCCAGAG GAACCCCAAC TACGAATGAA AAATAATGAA
GAAGCGGAAG ACTATGATGA TGATCTTACT GATTCTGAAA TGGATGTGGT CAGGTTTGAT
GATGACAACT CTCCTTCCTT TATCCAAATT CGCTCAGTTG CCAAGAAGCA TCCTAAAACT
TGGGTACATT ACATTGCTGC TGAAGAGGAG GACTGGGACT ATGCTCCCTT AGTCCTCGCC
CCCGATGACA GAAGTTATAA AAGTCAATAT TTGAACAATG GCCCTCAGCG GATTGGTAGG
AAGTACAAAA AAGTCCGATT TATGGCATAC ACAGATGAAA CCTTTAAGAC TCGTGAAGCT
ATTCAGCATG AATCAGGAAT CTTGGGACCT TTACTTTATG GGGAAGTTGG AGACACACTG
TTGATTATAT TTAAGAATCA AGCAAGCAGA CCATATAACA TCTACCCTCA CGGAATCACT
GATGTCCGTC CTTTGTATTC AAGGAGATTA CCAAAAGGTG TAAAACATTT GAAGGATTTT
CCAATTCTGC CAGGAGAAAT ATTCAAATAT AAATGGACAG TGACTGTAGA AGATGGGCCA
ACTAAATCAG ATCCTCGGTG CCTGACCCGC TATTACTCTA GTTTCGTTAA TATGGAGAGA
GATCTAGCTT CAGGACTCAT TGGCCCTCTC CTCATCTGCT ACAAAGAATC TGTAGATCAA
AGAGGAAACC AGATAATGTC AGACAAGAGG AATGTCATCC TGTTTTCTGT ATTTGATGAG
AACCGAAGCT GGTACCTCAC AGAGAATATA CAACGCTTTC TCCCCAATCC AGCTGGAGTG
CAGCTTGAGG ATCCAGAGTT CCAAGCCTCC AACATCATGC ACAGCATCAA TGGCTATGTT
TTTGATAGTT TGCAGTTGTC AGTTTGTTTG CATGAGGTGG CATACTGGTA CATTCTAAGC
ATTGGAGCAC AGACTGACTT CCTTTCTGTC TTCTTCTCTG GATATACCTT CAAACACAAA
ATGGTCTATG AAGACACACT CACCCTATTC CCATTCTCAG GAGAAACTGT CTTCATGTCG
ATGGAAAACC CAGGTCTATG GATTCTGGGG TGCCACAACT CAGACTTTCG GAACAGAGGC
ATGACCGCCT TACTGAAGGT TTCTAGTTGT GACAAGAACA CTGGTGATTA TTACGAGGAC
AGTTATGAAG ATATTTCAGC ATACTTGCTG AGTAAAAACA ATGCCATTGA ACCAAGAAGC
TTCTCCCAAA ACCCACCAGT CTTGAAACGC CATCAACGGG AAATAACTCG TACTACTCTT
CAGTCAGATC AAGAGGAAAT TGACTATGAT GATACCATAT CAGTTGAAAT GAAGAAGGAA
GATTTTGACA TTTATGATGA GGATGAAAAT CAGAGCCCCC GCAGCTTTCA AAAGAAAACA
CGACACTATT TTATTGCTGC AGTGGAGAGG CTCTGGGATT ATGGGATGAG TAGCTCCCCA
CATGTTCTAA GAAACAGGGC TCAGAGTGGC AGTGTCCCTC AGTTCAAGAA AGTTGTTTTC
CAGGAATTTA CTGATGGCTC CTTTACTCAG CCCTTATACC GTGGAGAACT AAATGAACAT
TTGGGACTCC TGGGGCCATA TATAAGAGCA GAAGTTGAAG ATAATATCAT GGTAACTTTC
AGAAATCAGG CCTCTCGTCC CTATTCCTTC TATTCTAGCC TTATTTCTTA TGAGGAAGAT
CAGAGGCAAG GAGCAGAACC TAGAAAAAAC TTTGTCAAGC CTAATGAAAC CAAAACTTAC
TTTTGGAAAG TGCAACATCA TATGGCACCC ACTAAAGATG AGTTTGACTG CAAAGCCTGG
GCTTATTTCT CTGATGTTGA CCTGGAAAAA GATGTGCACT CAGGCCTGAT TGGACCCCTT
CTGGTCTGCC ACACTAACAC ACTGAACCCT GCTCATGGGA GACAAGTGAC AGTACAGGAA
TTTGCTCTGT TTTTCACCAT CTTTGATGAG ACCAAAAGCT GGTACTTCAC TGAAAATATG
GAAAGAAACT GCAGGGCTCC CTGCAATATC CAGATGGAAG ATCCCACTTT TAAAGAGAAT
TATCGCTTCC ATGCAATCAA TGGCTACATA ATGGATACAC TACCTGGCTT AGTAATGGCT
CAGGATCAAA GGATTCGATG GTATCTGCTC AGCATGGGCA GCAATGAAAA CATCCATTCT
ATTCATTTCA GTGGACATGT GTTCACCGTA CGAAAAAAAG AGGAGTATAA AATGGCACTG
TACAATCTCT ATCCAGGTGT TTTTGAGACA GTGGAAATGT TACCATCCAA AGCTGGAATT
TGGCGGGTGG AATGCCTTAT TGGCGAGCAT CTACATGCTG GGATGAGCAC ACTTTTTCTG
GTGTACAGCA ATAAGTGTCA GACTCCCCTG GGAATGGCTT CTGGACACAT TAGAGATTTT
CAGATTACAG CTTCAGGACA ATATGGACAG TGGGCCCCAA AGCTGGCCAG ACTTCATTAT
TCCGGATCAA TCAATGCCTG GAGCACCAAG GAGCCCTTTT CTTGGATCAA GGTGGATCTG
TTGGCACCAA TGATTATTCA CGGCATCAAG ACCCAGGGTG CCCGTCAGAA GTTCTCCAGC
CTCTACATCT CTCAGTTTAT CATCATGTAT AGTCTTGATG GGAAGAAGTG GCAGACTTAT
CGAGGAAATT CCACTGGAAC CTTAATGGTC TTCTTTGGCA ATGTGGATTC ATCTGGGATA
AAACACAATA TTTTTAACCC TCCAATTATT GCTCGATACA TCCGTTTGCA CCCAACTCAT
TATAGCATTC GCAGCACTCT TCGCATGGAG TTGATGGGCT GTGATTTAAA TAGTTGCAGC
ATGCCATTGG GAATGGAGAG TAAAGCAATA TCAGATGCAC AGATTACTGC TTCATCCTAC
TTTACCAATA TGTTTGCCAC CTGGTCTCCT TCAAAAGCTC GACTTCACCT CCAAGGGAGG
AGTAATGCCT GGAGACCTCA GGTGAATAAT CCAAAAGAGT GGCTGCAAGT GGACTTCCAG
AAGACAATGA AAGTCACAGG AGTAACTACT CAGGGAGTAA AATCTCTGCT TACCAGCATG
TATGTGAAGG AGTTCCTCAT CTCCAGCAGT CAAGATGGCC ATCAGTGGAC TCTCTTTTTT
CAGAATGGCA AAGTAAAGGT TTTTCAGGGA AATCAAGACT CCTTCACACC TGTGGTGAAC
TCTCTAGACC CACCGTTACT GACTCGCTAC CTTCGAATTC ACCCCCAGAG TTGGGTGCAC
CAGATTGCCC TGAGGATGGA GGTTCTGGGC TGCGAGGCAC AGGACCTCTA CTGA
V3 factor VIII cDNA
(SEQ ID NO: 20)
ATGCAGATTGAGCTGAGCACCTGCTTCTTCCTGTGCCTGCTGAGGTTCTGCTTCTCTGCCACCAGGAGATA
CTACCTGGGGGCTGTGGAGCTGAGCTGGGACTACATGCAGTCTGACCTGGGGGAGCTGCCTGTGGATGCCA
GGTTCCCCCCCAGAGTGCCCAAGAGCTTCCCCTTCAACACCTCTGTGGTGTACAAGAAGACCCTGTTTGTG
GAGTTCACTGACCACCTGTTCAACATTGCCAAGCCCAGGCCCCCCTGGATGGGCCTGCTGGGCCCCACCAT
CCAGGCTGAGGTGTATGACACTGTGGTGATCACCCTGAAGAACATGGCCAGCCACCCTGTGAGCCTGCATG
CTGTGGGGGTGAGCTACTGGAAGGCCTCTGAGGGGGCTGAGTATGATGACCAGACCAGCCAGAGGGAGAAG
GAGGATGACAAGGTGTTCCCTGGGGGCAGCCACACCTATGTGTGGCAGGTGCTGAAGGAGAATGGCCCCAT
GGCCTCTGACCCCCTGTGCCTGACCTACAGCTACCTGAGCCATGTGGACCTGGTGAAGGACCTGAACTCTG
GCCTGATTGGGGCCCTGCTGGTGTGCAGGGAGGGCAGCCTGGCCAAGGAGAAGACCCAGACCCTGCACAAG
TTCATCCTGCTGTTTGCTGTGTTTGATGAGGGCAAGAGCTGGCACTCTGAAACCAAGAACAGCCTGATGCA
GGACAGGGATGCTGCCTCTGCCAGGGCCTGGCCCAAGATGCACACTGTGAATGGCTATGTGAACAGGAGCC
TGCCTGGCCTGATTGGCTGCCACAGGAAGTCTGTGTACTGGCATGTGATTGGCATGGGCACCACCCCTGAG
GTGCACAGCATCTTCCTGGAGGGCCACACCTTCCTGGTCAGGAACCACAGGCAGGCCAGCCTGGAGATCAG
CCCCATCACCTTCCTGACTGCCCAGACCCTGCTGATGGACCTGGGCCAGTTCCTGCTGTTCTGCCACATCA
GCAGCCACCAGCATGATGGCATGGAGGCCTATGTGAAGGTGGACAGCTGCCCTGAGGAGCCCCAGCTGAGG
ATGAAGAACAATGAGGAGGCTGAGGACTATGATGATGACCTGACTGACTCTGAGATGGATGTGGTGAGGTT
TGATGATGACAACAGCCCCAGCTTCATCCAGATCAGGTCTGTGGCCAAGAAGCACCCCAAGACCTGGGTGC
ACTACATTGCTGCTGAGGAGGAGGACTGGGACTATGCCCCCCTGGTGCTGGCCCCTGATGACAGGAGCTAC
AAGAGCCAGTACCTGAACAATGGCCCCCAGAGGATTGGCAGGAAGTACAAGAAGGTCAGGTTCATGGCCTA
CACTGATGAAACCTTCAAGACCAGGGAGGCCATCCAGCATGAGTCTGGCATCCTGGGCCCCCTGCTGTATG
GGGAGGTGGGGGACACCCTGCTGATCATCTTCAAGAACCAGGCCAGCAGGCCCTACAACATCTACCCCCAT
GGCATCACTGATGTGAGGCCCCTGTACAGCAGGAGGCTGCCCAAGGGGGTGAAGCACCTGAAGGACTTCCC
CATCCTGCCTGGGGAGATCTTCAAGTACAAGTGGACTGTGACTGTGGAGGATGGCCCCACCAAGTCTGACC
CCAGGTGCCTGACCAGATACTACAGCAGCTTTGTGAACATGGAGAGGGACCTGGCCTCTGGCCTGATTGGC
CCCCTGCTGATCTGCTACAAGGAGTCTGTGGACCAGAGGGGCAACCAGATCATGTCTGACAAGAGGAATGT
GATCCTGTTCTCTGTGTTTGATGAGAACAGGAGCTGGTACCTGACTGAGAACATCCAGAGGTTCCTGCCCA
ACCCTGCTGGGGTGCAGCTGGAGGACCCTGAGTTCCAGGCCAGCAACATCATGCACAGCATCAATGGCTAT
GTGTTTGACAGCCTGCAGCTGTCTGTGTGCCTGCATGAGGTGGCCTACTGGTACATCCTGAGCATTGGGGC
CCAGACTGACTTCCTGTCTGTGTTCTTCTCTGGCTACACCTTCAAGCACAAGATGGTGTATGAGGACACCC
TGACCCTGTTCCCCTTCTCTGGGGAGACTGTGTTCATGAGCATGGAGAACCCTGGCCTGTGGATTCTGGGC
TGCCACAACTCTGACTTCAGGAACAGGGGCATGACTGCCCTGCTGAAAGTCTCCAGCTGTGACAAGAACAC
TGGGGACTACTATGAGGACAGCTATGAGGACATCTCTGCCTACCTGCTGAGCAAGAACAATGCCATTGAGC
CCAGGAGCTTCAGCCAGAACAGCAGGCACCCCAGCACCAGGCAGAAGCAGTTCAATGCCACCACCATCCCT
GAGAATGACATAGAGAAGACAGACCCATGGTTTGCCCACCGGACCCCCATGCCCAAGATCCAGAATGTGAG
CAGCTCTGACCTGCTGATGCTGCTGAGGCAGAGCCCCACCCCCCATGGCCTGAGCCTGTCTGACCTGCAGG
AGGCCAAGTATGAAACCTTCTCTGATGACCCCAGCCCTGGGGCCATTGACAGCAACAACAGCCTGTCTGAG
ATGACCCACTTCAGGCCCCAGCTGCACCACTCTGGGGACATGGTGTTCACCCCTGAGTCTGGCCTGCAGCT
GAGGCTGAATGAGAAGCTGGGCACCACTGCTGCCACTGAGCTGAAGAAGCTGGACTTCAAAGTCTCCAGCA
CCAGCAACAACCTGATCAGCACCATCCCCTCTGACAACCTGGCTGCTGGCACTGACAACACCAGCAGCCTG
GGCCCCCCCAGCATGCCTGTGCACTATGACAGCCAGCTGGACACCACCCTGTTTGGCAAGAAGAGCAGCCC
CCTGACTGAGTCTGGGGGCCCCCTGAGCCTGTCTGAGGAGAACAATGACAGCAAGCTGCTGGAGTCTGGCC
TGATGAACAGCCAGGAGAGCAGCTGGGGCAAGAATGTGAGCACCAGGAGCTTCCAGAAGAAGACCAGGCAC
TACTTCATTGCTGCTGTGGAGAGGCTGTGGGACTATGGCATGAGCAGCAGCCCCCATGTGCTGAGGAACAG
GGCCCAGTCTGGCTCTGTGCCCCAGTTCAAGAAGGTGGTGTTCCAGGAGTTCACTGATGGCAGCTTCACCC
AGCCCCTGTACAGAGGGGAGCTGAATGAGCACCTGGGCCTGCTGGGCCCCTACATCAGGGCTGAGGTGGAG
GACAACATCATGGTGACCTTCAGGAACCAGGCCAGCAGGCCCTACAGCTTCTACAGCAGCCTGATCAGCTA
TGAGGAGGACCAGAGGCAGGGGGCTGAGCCCAGGAAGAACTTTGTGAAGCCCAATGAAACCAAGACCTACT
TCTGGAAGGTGCAGCACCACATGGCCCCCACCAAGGATGAGTTTGACTGCAAGGCCTGGGCCTACTTCTCT
GATGTGGACCTGGAGAAGGATGTGCACTCTGGCCTGATTGGCCCCCTGCTGGTGTGCCACACCAACACCCT
GAACCCTGCCCATGGCAGGCAGGTGACTGTGCAGGAGTTTGCCCTGTTCTTCACCATCTTTGATGAAACCA
AGAGCTGGTACTTCACTGAGAACATGGAGAGGAACTGCAGGGCCCCCTGCAACATCCAGATGGAGGACCCC
ACCTTCAAGGAGAACTACAGGTTCCATGCCATCAATGGCTACATCATGGACACCCTGCCTGGCCTGGTGAT
GGCCCAGGACCAGAGGATCAGGTGGTACCTGCTGAGCATGGGCAGCAATGAGAACATCCACAGCATCCACT
TCTCTGGCCATGTGTTCACTGTGAGGAAGAAGGAGGAGTACAAGATGGCCCTGTACAACCTGTACCCTGGG
GTGTTTGAGACTGTGGAGATGCTGCCCAGCAAGGCTGGCATCTGGAGGGTGGAGTGCCTGATTGGGGAGCA
CCTGCATGCTGGCATGAGCACCCTGTTCCTGGTGTACAGCAACAAGTGCCAGACCCCCCTGGGCATGGCCT
CTGGCCACATCAGGGACTTCCAGATCACTGCCTCTGGCCAGTATGGCCAGTGGGCCCCCAAGCTGGCCAGG
CTGCACTACTCTGGCAGCATCAATGCCTGGAGCACCAAGGAGCCCTTCAGCTGGATCAAGGTGGACCTGCT
GGCCCCCATGATCATCCATGGCATCAAGACCCAGGGGGCCAGGCAGAAGTTCAGCAGCCTGTACATCAGCC
AGTTCATCATCATGTACAGCCTGGATGGCAAGAAGTGGCAGACCTACAGGGGCAACAGCACTGGCACCCTG
ATGGTGTTCTTTGGCAATGTGGACAGCTCTGGCATCAAGCACAACATCTTCAACCCCCCCATCATTGCCAG
ATACATCAGGCTGCACCCCACCCACTACAGCATCAGGAGCACCCTGAGGATGGAGCTGATGGGCTGTGACC
TGAACAGCTGCAGCATGCCCCTGGGCATGGAGAGCAAGGCCATCTCTGATGCCCAGATCACTGCCAGCAGC
TACTTCACCAACATGTTTGCCACCTGGAGCCCCAGCAAGGCCAGGCTGCACCTGCAGGGCAGGAGCAATGC
CTGGAGGCCCCAGGTCAACAACCCCAAGGAGTGGCTGCAGGTGGACTTCCAGAAGACCATGAAGGTGACTG
GGGTGACCACCCAGGGGGTGAAGAGCCTGCTGACCAGCATGTATGTGAAGGAGTTCCTGATCAGCAGCAGC
CAGGATGGCCACCAGTGGACCCTGTTCTTCCAGAATGGCAAGGTGAAGGTGTTCCAGGGCAACCAGGACAG
CTTCACCCCTGTGGTGAACAGCCTGGACCCCCCCCTGCTGACCAGATACCTGAGGATTCACCCCCAGAGCT
GGGTGCACCAGATTGCCCTGAGGATGGAGGTGCTGGGCTGTGAGGCCCAGGACCTGTACTGA
CO3 factor VIII cDNA
(SEQ ID NO: 21)
atgcagattg agctgtcaac ttgctttttc ctgtgcctgc tgagattttg tttttccgct
actagaagat actacctggg ggctgtggaa ctgtcttggg attacatgca gagtgacctg
ggagagctgc cagtggacgc acgatttcca cctagagtcc ctaaatcatt ccccttcaac
accagcgtgg tctataagaa aacactgttc gtggagttta ctgatcacct gttcaacatc
gctaagcctc ggccaccctg gatgggactg ctgggaccaa caatccaggc agaggtgtac
gacaccgtgg tcattacact gaaaaacatg gcctcacacc ccgtgagcct gcatgctgtg
ggcgtcagct actggaaggc ttccgaaggg gcagagtatg acgatcagac ttcccagaga
gaaaaagagg acgataaggt gtttcctggc gggtctcata cctatgtgtg gcaggtcctg
aaagagaatg gccccatggc ttccgaccct ctgtgcctga cctactctta tctgagtcac
gtggacctgg tcaaggatct gaacagcgga ctgatcggag cactgctggt gtgtagggaa
gggagcctgg ctaaggagaa aacccagaca ctgcataagt tcattctgct gttcgccgtg
tttgacgaag gaaaatcatg gcacagcgag acaaagaata gtctgatgca ggaccgggat
gccgcttcag ccagagcttg gcccaaaatg cacactgtga acggctacgt caatcgctca
ctgcctggac tgatcggctg ccaccgaaag agcgtgtatt ggcatgtcat cggaatgggc
accacacctg aagtgcactc cattttcctg gaggggcata cctttctggt ccgcaaccac
cgacaggcct ccctggagat ctctccaatt accttcctga cagctcagac tctgctgatg
gatctgggac agttcctgct gttttgccac atcagctccc accagcatga tggcatggag
gcctacgtga aagtggacag ctgtcccgag gaacctcagc tgaggatgaa gaacaatgag
gaagctgaag actatgacga tgacctgacc gactccgaga tggatgtggt ccgattcgat
gacgataaca gcccctcctt tatccagatt agatctgtgg ccaagaaaca ccctaagaca
tgggtccatt acatcgcagc cgaggaagag gactgggatt atgcaccact ggtgctggca
ccagacgatc gatcctacaa atctcagtat ctgaacaatg gaccacagcg gattggcaga
aagtacaaga aagtgaggtt catggcttat accgatgaaa ccttcaagac tcgcgaagca
atccagcacg agagcgggat tctgggacca ctgctgtacg gagaagtggg ggacaccctg
ctgatcattt ttaagaacca ggccagcagg ccttacaata tctatccaca tggaattaca
gatgtgcgcc ctctgtacag ccggagactg ccaaagggcg tcaaacacct gaaggacttc
ccaatcctgc ccggggaaat ttttaagtat aaatggactg tcaccgtcga ggatggcccc
actaagagcg accctaggtg cctgacccgc tactattcta gtttcgtgaa tatggaaagg
gatctggcca gcggactgat cggcccactg ctgatttgtt acaaagagag cgtggatcag
agaggcaacc agatcatgtc cgacaagagg aatgtgattc tgttcagtgt ctttgacgaa
aaccggtcat ggtatctgac cgagaacatc cagagattcc tgcctaatcc agccggagtg
cagctggaag atcctgagtt tcaggcttct aacatcatgc atagtattaa tggctacgtg
ttcgacagtc tgcagctgtc agtgtgtctg cacgaggtcg cttactggta tatcctgagc
attggagcac agacagattt cctgagcgtg ttcttttccg gctacacttt taagcataaa
atggtgtatg aggacacact gactctgttc cccttcagcg gcgaaaccgt gtttatgtcc
atggagaatc ccgggctgtg gatcctggga tgccacaaca gcgatttcag gaatcgcggg
atgactgccc tgctgaaagt gtcaagctgt gacaagaaca ccggagacta ctatgaagat
tcatacgagg acatcagcgc atatctgctg tccaaaaaca atgccattga acccaggtct
tttagtcaga atcctccagt gctgaagagg caccagcgcg agatcacccg cactaccctg
cagagtgatc aggaagagat cgactacgac gatacaattt ctgtggaaat gaagaaagag
gacttcgata tctatgacga agatgagaac cagagtcctc gatcattcca gaagaaaacc
cggcattact ttattgctgc agtggagcgc ctgtgggatt atggcatgtc ctctagtcct
cacgtgctgc gaaatcgggc ccagtcaggg agcgtcccac agttcaagaa agtggtcttc
caggagttta cagacggatc ctttactcag ccactgtacc ggggcgaact gaacgagcac
ctggggctgc tgggacccta tatcagagct gaagtggagg ataacattat ggtcaccttc
agaaatcagg catctaggcc ttacagtttt tattcaagcc tgatctctta cgaagaggac
cagaggcagg gagcagaacc acgaaaaaac ttcgtgaagc ctaatgagac caaaacatac
ttttggaagg tgcagcacca tatggcccca acaaaagacg aattcgattg caaggcatgg
gcctattttt ctgacgtgga tctggagaag gacgtccaca gtggcctgat cgggccactg
ctggtgtgtc atactaacac cctgaatccc gcacacggca ggcaggtcac tgtccaggaa
ttcgccctgt tctttaccat ctttgatgag acaaaaagct ggtacttcac cgaaaacatg
gagcgaaatt gccgggctcc atgtaatatt cagatggaag accccacatt caaggagaac
taccgctttc atgccatcaa tgggtatatt atggatactc tgcccggact ggtcatggct
caggaccaga gaatcaggtg gtacctgctg agcatggggt ccaacgagaa tatccactca
attcatttca gcggacacgt gtttactgtc cggaagaaag aagagtataa aatggccctg
tacaacctgt atcccggcgt gttcgaaacc gtcgagatgc tgcctagcaa ggcagggatc
tggagagtgg aatgcctgat tggggagcac ctgcatgccg gaatgtctac cctgtttctg
gtgtacagta ataagtgtca gacacccctg gggatggctt ccggacatat ccgggatttc
cagattaccg catctggaca gtacggccag tgggccccta agctggctag actgcactat
tccgggtcta tcaacgcttg gtccacaaaa gagcctttct cttggattaa ggtggacctg
ctggcaccaa tgatcattca tggcatcaaa actcaggggg ccaggcagaa gttctcctct
ctgtacatct cacagtttat catcatgtac agcctggatg gcaagaaatg gcagacatac
cgcggcaata gcacagggac tctgatggtg ttctttggca acgtggacag ttcagggatc
aagcacaaca ttttcaatcc ccctatcatt gctagataca tcaggctgca cccaacccat
tattctattc gaagtacact gcggatggaa ctgatggggt gcgatctgaa cagttgttca
atgcccctgg gaatggagtc caaggcaatc tctgacgccc agattaccgc tagctcctac
ttcactaata tgtttgctac ctggagcccc tccaaagcac gactgcatct gcagggacga
agcaacgcat ggcgaccaca ggtgaacaat cccaaggagt ggctgcaggt cgattttcag
aaaactatga aggtgaccgg agtcacaact cagggcgtga aaagtctgct gacctcaatg
tacgtcaagg agttcctgat ctctagttca caggacggcc accagtggac actgttcttt
cagaacggaa aggtgaaagt cttccagggc aatcaggatt cctttacacc tgtggtcaac
tctctggacc cacccctgct gactcgctac ctgcgaatcc acccacagtc ctgggtgcat
cagattgcac tgagaatgga agtcctgggc tgcgaggccc aggacctgta ttga
Full length cassette including mutated TTR promoter (TTRmut), synthetic
intron, CpG reduced factor VIII cDNA, poly A and ITRs
(SEQ ID NO: 23)
cctgcaggca gctgcgcgct cgctcgctca ctgaggccgc ccgggcaaag cccgggcgtc
gggcgacctt tggtcgcccg gcctcagtga gcgagcgagc gcgcagagag ggagtggcca
actccatcac taggggttcc tacgcgtgtc tgtctgcaca tttcgtagag cgagtgttcc
gatactctaa tctccctagg caaggttcat attgacttag gttacttatt ctccttttgt
tgactaagtc aataatcaga atcagcaggt ttggagtcag cttggcaggg atcagcagcc
tgggttggaa ggagggggta taaaagcccc ttcaccagga gaagccgtca cacagatcca
caagctcctg ctagcaggta agtgccgtgt gtggttcccg cgggcctggc ctctttacgg
gttatggccc ttgcgtgcct tgaattactg acactgacat ccactttttc tttttctcca
caggtttaaa cgccaccatg cagattgagc tgagcacctg cttcttcctg tgtctgctga
ggttctgctt ctctgccacc aggaggtatt acctgggggc tgtggagctg agctgggact
atatgcagtc tgacctgggg gagctgcctg tggatgctag gttccccccc agggtgccca
agagcttccc ctttaacact tctgtggtgt acaagaagac cctgtttgtg gagttcactg
accacctgtt caacattgcc aagcccaggc ccccctggat ggggctgctg gggcccacca
tccaggctga ggtgtatgac actgtggtga tcaccctgaa gaacatggcc agccaccctg
tgagcctgca tgctgtgggg gtgagctact ggaaggcttc tgagggggct gagtatgatg
accagactag ccagagggag aaggaggatg acaaggtgtt tcctgggggc agccatacct
atgtgtggca ggtgctgaag gagaatggcc ccatggcctc tgaccccctg tgcctgacct
acagctacct gtctcatgtg gacctggtga aggacctgaa ctctggcctg attggggctc
tgctggtgtg tagggagggc agcctggcta aggaaaagac ccagaccctg cataagttta
tcctgctgtt tgctgtgttt gatgagggca agagctggca ctctgagacc aagaacagcc
tgatgcagga tagggatgct gcctctgcca gggcttggcc taagatgcac actgtgaatg
ggtatgtgaa taggagcctg cctggcctga ttggctgcca caggaagtct gtgtactggc
atgtgattgg gatgggcacc acccctgagg tccatagcat cttcctggag ggccacactt
tcctggtgag gaaccacaga caggcctctc tggagatctc tcccatcacc ttcctgactg
ctcagactct gctgatggac ctgggccagt tcctgctgtt ttgccatatt agcagccacc
agcatgatgg gatggaggcc tatgtgaagg tggatagctg ccctgaggag cctcagctga
ggatgaagaa caatgaggag gctgaagact atgatgatga cctgactgat tctgagatgg
atgtggtgag gtttgatgat gacaatagcc ccagcttcat tcagatcagg tctgtggcca
agaaacaccc caagacctgg gtgcactaca ttgctgctga ggaagaggac tgggactatg
ctcccctggt gctggcccct gatgataggt cttataagag ccagtacctg aacaatgggc
cccagaggat tggcaggaag tacaagaagg tgaggttcat ggcctacact gatgaaacct
tcaaaaccag ggaggccatt cagcatgagt ctggcatcct gggccctctg ctgtatgggg
aggtggggga caccctgctg atcatcttca agaaccaggc cagcaggccc tacaacatct
atcctcatgg catcactgat gtgaggcccc tgtacagcag gaggctgccc aagggggtga
agcacctgaa agacttcccc atcctgcctg gggagatctt taagtataag tggactgtga
ctgtggagga tggccctacc aagtctgacc ccaggtgtct gaccaggtac tattctagct
ttgtgaacat ggagagggac ctggcctctg gcctgattgg gcccctgctg atctgctaca
aggagtctgt ggaccagagg ggcaaccaga tcatgtctga caagaggaat gtgatcctgt
tttctgtgtt tgatgagaat aggagctggt acctgactga gaacatccag aggtttctgc
ccaatcctgc tggggtgcag ctggaggatc ctgagttcca ggccagcaat atcatgcata
gcatcaatgg ctatgtgttt gacagcctgc agctgtctgt gtgcctgcat gaggtggcct
actggtacat cctgagcatt ggggcccaga ctgactttct gtctgtgttc ttttctggct
ataccttcaa gcacaagatg gtgtatgagg ataccctgac cctgttcccc ttctctgggg
agactgtgtt catgagcatg gagaatcctg ggctgtggat cctggggtgc cacaactctg
attttaggaa cagggggatg actgccctgc tgaaggtgtc tagctgtgat aagaacactg
gggactacta tgaggacagc tatgaggaca tttctgctta tctgctgtct aagaataatg
ccattgagcc cagaagcttc agccagaatc cccctgtgct gaagagacat cagagggaga
tcaccagaac taccctgcag tctgatcagg aggagattga ctatgatgac actatctctg
tggagatgaa gaaggaggac tttgacatct atgatgagga tgagaatcag tctcccagga
gctttcagaa gaagaccaga cattacttca ttgctgctgt ggagaggctg tgggactatg
gcatgagctc tagccctcat gtgctgagga acagggccca gtctggctct gtgccccagt
tcaagaaggt ggtgttccag gaattcactg atggcagctt cacccagccc ctgtacaggg
gggagctgaa tgagcacctg ggcctgctgg ggccttatat cagggctgag gtggaggata
atattatggt gactttcagg aaccaggcca gcaggcccta ctctttctat agcagcctga
tctcttatga ggaggatcag aggcaggggg ctgagcctag gaagaacttt gtgaagccca
atgagactaa gacctacttc tggaaggtcc agcaccacat ggcccctacc aaggatgagt
ttgactgcaa ggcctgggcc tatttctctg atgtggatct ggagaaggat gtccattctg
ggctgattgg ccccctgctg gtgtgccaca ctaacactct gaatcctgcc catggcaggc
aggtgactgt ccaggagttt gccctgttct tcactatctt tgatgagacc aagagctggt
actttactga gaacatggag aggaactgca gagctccttg caatattcag atggaggacc
ccaccttcaa ggagaattac aggttccatg ccattaatgg gtacatcatg gacaccctgc
ctggcctggt gatggctcag gaccagagga tcaggtggta cctgctgagc atgggctcta
atgagaatat ccacagcatc cacttctctg ggcatgtgtt cactgtgagg aagaaggagg
agtacaagat ggctctgtat aatctgtacc ctggggtgtt tgaaactgtg gagatgctgc
cctctaaggc tggcatctgg agggtggagt gcctgattgg ggagcacctg catgctggca
tgagcaccct gttcctggtg tacagcaaca agtgccagac ccccctgggc atggcctctg
gccacatcag ggacttccag atcactgcct ctggccagta tggccagtgg gcccccaagc
tggccaggct gcactattct ggcagcatca atgcctggag caccaaggag cccttcagct
ggatcaaggt ggacctgctg gcccccatga tcattcatgg catcaagacc cagggggcca
ggcagaagtt cagctctctg tacatctctc agttcatcat catgtactct ctggatggga
agaagtggca gacctacagg ggcaacagca ctggcaccct gatggtgttc tttgggaatg
tggactcttc tggcatcaag cacaacatct tcaatccccc catcattgct aggtatatta
ggctgcatcc cacccactac agcatcaggt ctaccctgag gatggagctg atgggctgtg
acctgaactc ttgcagcatg cccctgggca tggagtctaa ggccatctct gatgcccaga
ttactgccag cagctacttc accaacatgt ttgccacctg gagcccctct aaggccaggc
tgcatctgca ggggaggagc aatgcctgga ggcctcaggt gaacaacccc aaggagtggc
tgcaggtgga tttccagaag accatgaagg tgactggggt gaccacccag ggggtcaaga
gcctgctgac cagcatgtat gtgaaggagt tcctgatcag cagcagccag gatggccacc
agtggactct gttctttcag aatgggaagg tgaaggtgtt tcagggcaat caggactctt
tcacccctgt ggtgaacagc ctggaccccc ccctgctgac cagatacctg aggatccacc
cccagtcttg ggtgcatcag attgccctga ggatggaggt gctgggctgt gaggctcagg
atctgtactg agcggccgca ataaaagatc agagctctag agatctgtgt gttggttttt
tgtgtaggaa cccctagtga tggagttggc cactccctct ctgcgcgctc gctcgctcac
tgaggccggg cgaccaaagg tcgcccgacg cccgggcttt gcccgggcgg cctcagtgag
cgagcgagcg cgcagctgcc tgcagg
Full length plasmid including mutated TTR promoter (TTRmut), synthetic
intron, CpG reduced factor VIII cDNA, poly A and ITRs
(SEQ ID NO: 24)
cctgcaggca gctgcgcgct cgctcgctca ctgaggccgc ccgggcaaag cccgggcgtc
gggcgacctt tggtcgcccg gcctcagtga gcgagcgagc gcgcagagag ggagtggcca
actccatcac taggggttcc tacgcgtgtc tgtctgcaca tttcgtagag cgagtgttcc
gatactctaa tctccctagg caaggttcat attgacttag gttacttatt ctccttttgt
tgactaagtc aataatcaga atcagcaggt ttggagtcag cttggcaggg atcagcagcc
tgggttggaa ggagggggta taaaagcccc ttcaccagga gaagccgtca cacagatcca
caagctcctg ctagcaggta agtgccgtgt gtggttcccg cgggcctggc ctctttacgg
gttatggccc ttgcgtgcct tgaattactg acactgacat ccactttttc tttttctcca
caggtttaaa cgccaccatg cagattgagc tgagcacctg cttcttcctg tgtctgctga
ggttctgctt ctctgccacc aggaggtatt acctgggggc tgtggagctg agctgggact
atatgcagtc tgacctgggg gagctgcctg tggatgctag gttccccccc agggtgccca
agagcttccc ctttaacact tctgtggtgt acaagaagac cctgtttgtg gagttcactg
accacctgtt caacattgcc aagcccaggc ccccctggat ggggctgctg gggcccacca
tccaggctga ggtgtatgac actgtggtga tcaccctgaa gaacatggcc agccaccctg
tgagcctgca tgctgtgggg gtgagctact ggaaggcttc tgagggggct gagtatgatg
accagactag ccagagggag aaggaggatg acaaggtgtt tcctgggggc agccatacct
atgtgtggca ggtgctgaag gagaatggcc ccatggcctc tgaccccctg tgcctgacct
acagctacct gtctcatgtg gacctggtga aggacctgaa ctctggcctg attggggctc
tgctggtgtg tagggagggc agcctggcta aggaaaagac ccagaccctg cataagttta
tcctgctgtt tgctgtgttt gatgagggca agagctggca ctctgagacc aagaacagcc
tgatgcagga tagggatgct gcctctgcca gggcttggcc taagatgcac actgtgaatg
ggtatgtgaa taggagcctg cctggcctga ttggctgcca caggaagtct gtgtactggc
atgtgattgg gatgggcacc acccctgagg tccatagcat cttcctggag ggccacactt
tcctggtgag gaaccacaga caggcctctc tggagatctc tcccatcacc ttcctgactg
ctcagactct gctgatggac ctgggccagt tcctgctgtt ttgccatatt agcagccacc
agcatgatgg gatggaggcc tatgtgaagg tggatagctg ccctgaggag cctcagctga
ggatgaagaa caatgaggag gctgaagact atgatgatga cctgactgat tctgagatgg
atgtggtgag gtttgatgat gacaatagcc ccagcttcat tcagatcagg tctgtggcca
agaaacaccc caagacctgg gtgcactaca ttgctgctga ggaagaggac tgggactatg
ctcccctggt gctggcccct gatgataggt cttataagag ccagtacctg aacaatgggc
cccagaggat tggcaggaag tacaagaagg tgaggttcat ggcctacact gatgaaacct
tcaaaaccag ggaggccatt cagcatgagt ctggcatcct gggccctctg ctgtatgggg
aggtggggga caccctgctg atcatcttca agaaccaggc cagcaggccc tacaacatct
atcctcatgg catcactgat gtgaggcccc tgtacagcag gaggctgccc aagggggtga
agcacctgaa agacttcccc atcctgcctg gggagatctt taagtataag tggactgtga
ctgtggagga tggccctacc aagtctgacc ccaggtgtct gaccaggtac tattctagct
ttgtgaacat ggagagggac ctggcctctg gcctgattgg gcccctgctg atctgctaca
aggagtctgt ggaccagagg ggcaaccaga tcatgtctga caagaggaat gtgatcctgt
tttctgtgtt tgatgagaat aggagctggt acctgactga gaacatccag aggtttctgc
ccaatcctgc tggggtgcag ctggaggatc ctgagttcca ggccagcaat atcatgcata
gcatcaatgg ctatgtgttt gacagcctgc agctgtctgt gtgcctgcat gaggtggcct
actggtacat cctgagcatt ggggcccaga ctgactttct gtctgtgttc ttttctggct
ataccttcaa gcacaagatg gtgtatgagg ataccctgac cctgttcccc ttctctgggg
agactgtgtt catgagcatg gagaatcctg ggctgtggat cctggggtgc cacaactctg
attttaggaa cagggggatg actgccctgc tgaaggtgtc tagctgtgat aagaacactg
gggactacta tgaggacagc tatgaggaca tttctgctta tctgctgtct aagaataatg
ccattgagcc cagaagcttc agccagaatc cccctgtgct gaagagacat cagagggaga
tcaccagaac taccctgcag tctgatcagg aggagattga ctatgatgac actatctctg
tggagatgaa gaaggaggac tttgacatct atgatgagga tgagaatcag tctcccagga
gctttcagaa gaagaccaga cattacttca ttgctgctgt ggagaggctg tgggactatg
gcatgagctc tagccctcat gtgctgagga acagggccca gtctggctct gtgccccagt
tcaagaaggt ggtgttccag gaattcactg atggcagctt cacccagccc ctgtacaggg
gggagctgaa tgagcacctg ggcctgctgg ggccttatat cagggctgag gtggaggata
atattatggt gactttcagg aaccaggcca gcaggcccta ctctttctat agcagcctga
tctcttatga ggaggatcag aggcaggggg ctgagcctag gaagaacttt gtgaagccca
atgagactaa gacctacttc tggaaggtcc agcaccacat ggcccctacc aaggatgagt
ttgactgcaa ggcctgggcc tatttctctg atgtggatct ggagaaggat gtccattctg
ggctgattgg ccccctgctg gtgtgccaca ctaacactct gaatcctgcc catggcaggc
aggtgactgt ccaggagttt gccctgttct tcactatctt tgatgagacc aagagctggt
actttactga gaacatggag aggaactgca gagctccttg caatattcag atggaggacc
ccaccttcaa ggagaattac aggttccatg ccattaatgg gtacatcatg gacaccctgc
ctggcctggt gatggctcag gaccagagga tcaggtggta cctgctgagc atgggctcta
atgagaatat ccacagcatc cacttctctg ggcatgtgtt cactgtgagg aagaaggagg
agtacaagat ggctctgtat aatctgtacc ctggggtgtt tgaaactgtg gagatgctgc
cctctaaggc tggcatctgg agggtggagt gcctgattgg ggagcacctg catgctggca
tgagcaccct gttcctggtg tacagcaaca agtgccagac ccccctgggc atggcctctg
gccacatcag ggacttccag atcactgcct ctggccagta tggccagtgg gcccccaagc
tggccaggct gcactattct ggcagcatca atgcctggag caccaaggag cccttcagct
ggatcaaggt ggacctgctg gcccccatga tcattcatgg catcaagacc cagggggcca
ggcagaagtt cagctctctg tacatctctc agttcatcat catgtactct ctggatggga
agaagtggca gacctacagg ggcaacagca ctggcaccct gatggtgttc tttgggaatg
tggactcttc tggcatcaag cacaacatct tcaatccccc catcattgct aggtatatta
ggctgcatcc cacccactac agcatcaggt ctaccctgag gatggagctg atgggctgtg
acctgaactc ttgcagcatg cccctgggca tggagtctaa ggccatctct gatgcccaga
ttactgccag cagctacttc accaacatgt ttgccacctg gagcccctct aaggccaggc
tgcatctgca ggggaggagc aatgcctgga ggcctcaggt gaacaacccc aaggagtggc
tgcaggtgga tttccagaag accatgaagg tgactggggt gaccacccag ggggtcaaga
gcctgctgac cagcatgtat gtgaaggagt tcctgatcag cagcagccag gatggccacc
agtggactct gttctttcag aatgggaagg tgaaggtgtt tcagggcaat caggactctt
tcacccctgt ggtgaacagc ctggaccccc ccctgctgac cagatacctg aggatccacc
cccagtcttg ggtgcatcag attgccctga ggatggaggt gctgggctgt gaggctcagg
atctgtactg agcggccgca ataaaagatc agagctctag agatctgtgt gttggttttt
tgtgtaggaa cccctagtga tggagttggc cactccctct ctgcgcgctc gctcgctcac
tgaggccggg cgaccaaagg tcgcccgacg cccgggcttt gcccgggcgg cctcagtgag
cgagcgagcg cgcagctgcc tgcaggggca gcttgaagga aatactaagg caaaggtact
gcaagtgctc gcaacattcg cttatgcgga ttattgccgt agtgccgcga cgccgggggc
aagatgcaga gattgccatg gtacaggccg tgcggttgat attgccaaaa cagagctgtg
ggggagagtt gtcgagaaag agtgcggaag atgcaaaggc gtcggctatt caaggatgcc
agcaagcgca gcatatcgcg ctgtgacgat gctaatccca aaccttaccc aacccacctg
gtcacgcact gttaagccgc tgtatgacgc tctggtggtg caatgccaca aagaagagtc
aatcgcagac aacattttga atgcggtcac acgttagcag catgattgcc acggatggca
acatattaac ggcatgatat tgacttattg aataaaattg ggtaaatttg actcaacgat
gggttaattc gctcgttgtg gtagtgagat gaaaagaggc ggcgcttact accgattccg
cctagttggt cacttcgacg tatcgtctgg aactccaacc atcgcaggca gagaggtctg
caaaatgcaa tcccgaaaca gttcgcaggt aatagttaga gcctgcataa cggtttcggg
attttttata tctgcacaac aggtaagagc attgagtcga taatcgtgaa gagtcggcga
gcctggttag ccagtgctct ttccgttgtg ctgaattaag cgaataccgg aagcagaacc
ggatcaccaa atgcgtacag gcgtcatcgc cgcccagcaa cagcacaacc caaactgagc
cgtagccact gtctgtcctg aattcattag taatagttac gctgcggcct tttacacatg
accttcgtga aagcgggtgg caggaggtcg cgctaacaac ctcctgccgt tttgcccgtg
catatcggtc acgaacaaat ctgattacta aacacagtag cctggatttg ttctatcagt
aatcgacctt attcctaatt aaatagagca aatcccctta ttgggggtaa gacatgaaga
tgccagaaaa acatgacctg ttggccgcca ttctcgcggc aaaggaacaa ggcatcgggg
caatccttgc gtttgcaatg gcgtaccttc gcggcagata taatggcggt gcgtttacaa
aaacagtaat cgacgcaacg atgtgcgcca ttatcgccta gttcattcgt gaccttctcg
acttcgccgg actaagtagc aatctcgctt atataacgag cgtgtttatc ggctacatcg
gtactgactc gattggttcg cttatcaaac gcttcgctgc taaaaaagcc ggagtagaag
atggtagaaa tcaataatca acgtaaggcg ttcctcgata tgctggcgtg gtcggaggga
actgataacg gacgtcagaa aaccagaaat catggttatg acgtcattgt aggcggagag
ctatttactg attactccga tcaccctcgc aaacttgtca cgctaaaccc aaaactcaaa
tcaacaggcg ccggacgcta ccagcttctt tcccgttggt gggatgccta ccgcaagcag
cttggcctga aagacttctc tccgaaaagt caggacgctg tggcattgca gcagattaag
gagcgtggcg ctttacctat gattgatcgt ggtgatatcc gtcaggcaat cgaccgttgc
agcaatatct gggcttcact gccgggcgct ggttatggtc agttcgagca taaggctgac
agcctgattg caaaattcaa agaagcgggc ggaacggtca gagagattga tgtatgagca
gagtcaccgc gattatctcc gctctggtta tctgcatcat cgtctgcctg tcatgggctg
ttaatcatta ccgtgataac gccattacct acaaagccca gcgcgacaaa aatgccagag
aactgaagct ggcgaacgcg gcaattactg acatgcagat gcgtcagcgt gatgttgctg
cgctcgatgc aaaatacacg aaggagttag ctgatgctaa agctgaaaat gatgctctgc
gtgatgatgt tgccgctggt cgtcgtcggt tgcacatcaa agcagtctgt cagtcagtgc
gtgaagccac caccgcctcc ggcgtggata atgcagcctc cccccgactg gcagacaccg
ctgaacggga ttatttcacc ctcagagaga ggctgatcac tatgcaaaaa caactggaag
gaacccagaa gtatattaat gagcagtgca gatagagttg cccatatcga tgggcaactc
atgcaattat tgtgagcaat acacacgcgc ttccagcgga gtataaatgc ctaaagtaat
aaaaccgagc aatccattta cgaatgtttg ctgggtttct gttttaacaa cattttctgc
gccgccacaa attttggctg catcgacagt tttcttctgc ccaattccag aaacgaagaa
atgatgggtg atggtttcct ttggtgctac tgctgccggt ttgttttgaa cagtaaacgt
ctgttgagca catcctgtaa taagcagggc cagcgcagta gcgagtagca tttttttcat
ggtgttattc ccgatgcttt ttgaagttcg cagaatcgta tgtgtagaaa attaaacaaa
ccctaaacaa tgagttgaaa tttcatattg ttaatattta ttaatgtatg tcaggtgcga
tgaatcgtca ttgtattccc ggattaacta tgtccacagc cctgacgggg aacttctctg
cgggagtgtc cgggaataat taaaacgatg cacacagggt ttagcgcgta cacgtattgc
attatgccaa cgccccggtg ctgacacgga agaaaccgga cgttatgatt tagcgtggaa
agatttgtgt agtgttctga atgctctcag taaatagtaa tgaattatca aaggtatagt
aatatctttt atgttcatgg atatttgtaa cccatcggaa aactcctgct ttagcaagat
tttccctgta ttgctgaaat gtgatttctc ttgatttcaa cctatcatag gacgtttcta
taagatgcgt gtttcttgag aatttaacat ttacaacctt tttaagtcct tttattaaca
cggtgttatc gttttctaac acgatgtgaa tattatctgt ggctagatag taaatataat
gtgagacgtt gtgacgtttt agttcagaat aaaacaattc acagtctaaa tcttttcgca
cttgatcgaa tatttcttta aaaatggcaa cctgagccat tggtaaaacc ttccatgtga
tacgagggcg cgtagtttgc attatcgttt ttatcgtttc aatctggtct gacctccttg
tgttttgttg atgatttatg tcaaatatta ggaatgtttt cacttaatag tattggttgc
gtaacaaagt gcggtcctgc tggcattctg gagggaaata caaccgacag atgtatgtaa
ggccaacgtg ctcaaatctt catacagaaa gatttgaagt aatattttaa ccgctagatg
aagagcaagc gcatggagcg acaaaatgaa taaagaacaa tctgctgatg atccctccgt
ggatctgatt cgtgtaaaaa atatgcttaa tagcaccatt tctatgagtt accctgatgt
tgtaattgca tgtatagaac ataaggtgtc tctggaagca ttcagagcaa ttgaggcagc
gttggtgaag cacgataata atatgaagga ttattccctg gtggttgact gatcaccata
actgctaatc attcaaacta tttagtctgt gacagagcca acacgcagtc tgtcactgtc
aggaaagtgg taaaactgca actcaattac tgcaatgccc tcgtaattaa gtgaatttac
aatatcgtcc tgttcggagg gaagaacgcg ggatgttcat tcttcatcac ttttaattga
tgtatatgct ctcttttctg acgttagtct ccgacggcag gcttcaatga cccaggctga
gaaattcccg gacccttttt gctcaagagc gatgttaatt tgttcaatca tttggttagg
aaagcggatg ttgcgggttg ttgttctgcg ggttctgttc ttcgttgaca tgaggttgcc
ccgtattcag tgtcgctgat ttgtattgtc tgaagttgtt tttacgttaa gttgatgcag
atcaattaat acgatacctg cgtcataatt gattatttga cgtggtttga tggcctccac
gcacgttgtg atatgtagat gataatcatt atcactttac gggtcctttc cggtgatccg
acaggttacg gggcggcgac ctgcctgatg cggtattttc tccttacgca tctgtgcggt
atttcacacc gcatacgtca aagcaaccat agtacgcgcc ctgtagcggc gcattaagcg
cggcgggtgt ggtggttacg cgcagcgtga ccgctacact tgccagcgcc ttagcgcccg
ctcctttcgc tttcttccct tcctttctcg ccacgttcgc cggctttccc cgtcaagctc
taaatcgggg gctcccttta gggttccgat ttagtgcttt acggcacctc gaccccaaaa
aacttgattt gggtgatggt tcacgtagtg ggccatcgcc ctgatagacg gtttttcgcc
ctttgacgtt ggagtccacg ttctttaata gtggactctt gttccaaact ggaacaacac
tcaactctat ctcgggctat tcttttgatt tagacctgca ggcatgcaag cttggcactg
gccgtcgttt tacaacgtcg tgactgggaa aaccctggcg ttacccaact taatcgcctt
gcagcacatc cccctttcgc cagctggcgt aatagcgaag aggcccgcac cgatcgccct
tcccaacagt tgcgcagcct gaatggcgaa tgcgatttat tcaacaaagc cgccgtcccg
tcaagtcagc gtaatgctct gccagtgtta caaccaatta accaattctg attagaaaaa
ctcatcgagc atcaaatgaa actgcaattt attcatatca ggattatcaa taccatattt
ttgaaaaagc cgtttctgta atgaaggaga aaactcaccg aggcagttcc ataggatggc
aagatcctgg tatcggtctg cgattccgac tcgtccaaca tcaatacaac ctattaattt
cccctcgtca aaaataaggt tatcaagtga gaaatcacca tgagtgacga ctgaatccgg
tgagaatggc aaaagcttat gcatttcttt ccagacttgt tcaacaggcc agccattacg
ctcgtcatca aaatcactcg catcaaccaa accgttattc attcgtgatt gcgcctgagc
gagacgaaat acgcgatcgc tgttaaaagg acaattacaa acaggaatcg aatgcaaccg
gcgcaggaac actgccagcg catcaacaat attttcacct gaatcaggat attcttctaa
tacctggaat gctgttttcc cggggatcgc agtggtgagt aaccatgcat catcaggagt
acggataaaa tgcttgatgg tcggaagagg cataaattcc gtcagccagt ttagtctgac
catctcatct gtaacatcat tggcaacgct acctttgcca tgtttcagaa acaactctgg
cgcatcgggc ttcccataca atcgatagat tgtcgcacct gattgcccga cattatcgcg
agcccattta tacccatata aatcagcatc catgttggaa tttaatcgcg gcttcgagca
agacgtttcc cgttgaatat ggctcataac accccttgta ttactgttta tgtaagcaga
cagttttatt gttcatgatg atatattttt atcttgtgca atgtaacatc agagattttg
agacacaacg tggctttgtt gaataaatcg aacttttgct gagttgaagg atcagatcac
gcatcttccc gacaacgcag accgttccgt ggcaaagcaa aagttcaaaa tcaccaactg
gtccacctac aacaaagctc tcatcaaccg tggctccctc actttctggc tggatgatgg
ggcgattcag gcctggtatg agtcagcaac accttcttca cgaggcagac ctctcgacgg
agttccactg agcgtcagac cccgtagaaa agatcaaagg atcttcttga gatccttttt
ttctgcgcgt aatctgctgc ttgcaaacaa aaaaaccacc gctaccagcg gtggtttgtt
tgccggatca agagctacca actctttttc cgaaggtaac tggcttcagc agagcgcaga
taccaaatac tgttcttcta gtgtagccgt agttaggcca ccacttcaag aactctgtag
caccgcctac atacctcgct ctgctaatcc tgttaccagt ggctgctgcc agtggcgata
agtcgtgtct taccgggttg gactcaagac gatagttacc ggataaggcg cagcggtcgg
gctgaacggg gggttcgtgc acacagccca gcttggagcg aacgacctac accgaactga
gatacctaca gcgtgagcta tgagaaagcg ccacgcttcc cgaagggaga aaggcggaca
ggtatccggt aagcggcagg gtcggaacag gagagcgcac gagggagctt ccagggggaa
acgcctggta tctttatagt cctgtcgggt ttcgccacct ctgacttgag cgtcgatttt
tgtgatgctc gtcagggggg cggagcctat ggaaaaacgc cagcaacgcg gcctttttac
ggttcctggc cttttgctgg ccttttgctc acatgt
FVIII-BDD encoded by X01-X18 nucleic acid sequences. SQ sequence
bold/underlined
(SEQ ID NO: 25)
MQIELSTCFFLCLLRFCFSATRRYYLGAVELSWDYMQSDLGELPVDARFPPRVPKSFPFNTS
VVYKKTLFVEFTDHLFNIAKPRPPWMGLLGPTIQAEVYDTVVITLKNMASHPVSLHAVGVSY
WKASEGAEYDDQTSQREKEDDKVFPGGSHTYVWQVLKENGPMASDPLCLTYSYLSHVDLVKD
LNSGLIGALLVCREGSLAKEKTQTLHKFILLFAVFDEGKSWHSETKNSLMQDRDAASARAWP
KMHTVNGYVNRSLPGLIGCHRKSVYWHVIGMGTTPEVHSIFLEGHTFLVRNHRQASLEISPI
TFLTAQTLLMDLGQFLLFCHISSHQHDGMEAYVKVDSCPEEPQLRMKNNEEAEDYDDDLTDS
EMDVVRFDDDNSPSFIQIRSVAKKHPKTWVHYIAAEEEDWDYAPLVLAPDDRSYKSQYLNNG
PQRIGRKYKKVRFMAYTDETFKTREAIQHESGILGPLLYGEVGDTLLIIFKNQASRPYNIYP
HGITDVRPLYSRRLPKGVKHLKDFPILPGEIFKYKWTVTVEDGPTKSDPRCLTRYYSSFVNM
ERDLASGLIGPLLICYKESVDQRGNQIMSDKRNVILFSVFDENRSWYLTENIQRFLPNPAGV
QLEDPEFQASNIMHSINGYVFDSLQLSVCLHEVAYWYILSIGAQTDFLSVFFSGYTFKHKMV
YEDTLTLFPFSGETVFMSMENPGLWILGCHNSDFRNRGMTALLKVSSCDKNTGDYYEDSYED
ISAYLLSKNNAIEPRSFSQNPPVLKRHQREITRTTLQSDQEEIDYDDTISVEMKKEDFDIYD
EDENQSPRSFQKKTRHYFIAAVERLWDYGMSSSPHVLRNRAQSGSVPQFKKVVFQEFTDGSF
TQPLYRGELNEHLGLLGPYIRAEVEDNIMVTFRNQASRPYSFYSSLISYEEDQRQGAEPRKN
FVKPNETKTYFWKVQHHMAPTKDEFDCKAWAYFSDVDLEKDVHSGLIGPLLVCHTNTLNPAH
GRQVTVQEFALFFTIFDETKSWYFTENMERNCRAPCNIQMEDPTFKENYRFHAINGYIMDTL
PGLVMAQDQRIRWYLLSMGSNENIHSIHFSGHVFTVRKKEEYKMALYNLYPGVFETVEMLPS
KAGIWRVECLIGEHLHAGMSTLFLVYSNKCQTPLGMASGHIRDFQITASGQYGQWAPKLARL
HYSGSINAWSTKEPFSWIKVDLLAPMIIHGIKTQGARQKFSSLYISQFIIMYSLDGKKWQTY
RGNSTGTLMVFFGNVDSSGIKHNIFNPPIIARYIRLHPTHYSIRSTLRMELMGCDLNSCSMP
LGMESKAISDAQITASSYFTNMFATWSPSKARLHLQGRSNAWRPQVNNPKEWLQVDFQKTMK
VTGVITQGVKSLLTSMYVKEFLISSSQDGHQWTLFFQNGKVKVFQGNQDSFTPVVNSLDPPL
LTRYLRIHPQSWVHQIALRMEVLGCEAQDLY
Wild-type FVIII with BDD
(SEQ ID NO: 26)
MQIELSTCFFLCLLRFCFSATRRYYLGAVELSWDYMQSDLGELPVDARFPPRVPKSF
PFNTSVVYKKTLFVEFTDHLFNIAKPRPPWMGLLGPTIQAEVYDTVVITLKNMASHPVSLHA
VGVSYWKASEGAEYDDQTSQREKEDDKVFPGGSHTYVWQVLKENGPMASDPLCLTYSYLSHV
DLVKDLNSGLIGALLVCREGSLAKEKTQTLHKFILLFAVFDEGKSWHSETKNSLMQDRDAAS
ARAWPKMHTVNGYVNRSLPGLIGCHRKSVYWHVIGMGTTPEVHSIFLEGHTFLVRNHRQASL
EISPITFLTAQTLLMDLGQFLLFCHISSHQHDGMEAYVKVDSCPEEPQLRMKNNEEAEDYDD
DLTDSEMDVVRFDDDNSPSFIQIRSVAKKHPKTWVHYIAAEEEDWDYAPLVLAPDDRSYKSQ
YLNNGPQRIGRKYKKVRFMAYTDETFKTREAIQHESGILGPLLYGEVGDTLLIIFKNQASRP
YNIYPHGITDVRPLYSRRLPKGVKHLKDFPILPGEIFKYKWTVTVEDGPTKSDPRCLTRYYS
SFVNMERDLASGLIGPLLICYKESVDQRGNQIMSDKRNVILFSVFDENRSWYLTENIQRFLP
NPAGVQLEDPEFQASNIMHSINGYVFDSLQLSVCLHEVAYWYILSIGAQTDFLSVFFSGYTF
KHKMVYEDTLTLFPFSGETVFMSMENPGLWILGCHNSDFRNRGMTALLKVSSCDKNTGDYYE
DSYEDISAYLLSKNNAIEPRSFSQNSRHPSTRQKQFNATTIPENDIEKTDPWFAHRTPMPKI
QNVSSSDLLMLLRQSPTPHGLSLSDLQEAKYETFSDDPSPGAIDSNNSLSEMTHFRPQLHHS
GDMVFTPESGLQLRLNEKLGTTAATELKKLDFKVSSTSNNLISTIPSDNLAAGTDNTSSLGP
PSMPVHYDSQLDTTLFGKKSSPLTESGGPLSLSEENNDSKLLESGLMNSQESSWGKNVSSTE
SGRLFKGKRAHGPALLTKDNALFKVSISLLKTNKTSNNSATNRKTHIDGPSLLIENSPSVWQ
NILESDTEFKKVTPLIHDRMLMDKNATALRLNHMSNKTTSSKNMEMVQQKKEGPIPPDAQNP
DMSFFKMLFLPESARWIQRTHGKNSLNSGQGPSPKQLVSLGPEKSVEGQNFLSEKNKVVVGK
GEFTKDVGLKEMVFPSSRNLFLTNLDNLHENNTHNQEKKIQEEIEKKETLIQENVVLPQIHT
VTGTKNFMKNLFLLSTRQNVEGSYDGAYAPVLQDFRSLNDSTNRTKKHTAHFSKKGEEENLE
GLGNQTKQIVEKYACTTRISPNTSQQNFVTQRSKRALKQFRLPLEETELEKRIIVDDTSTQW
SKNMKHLTPSTLTQIDYNEKEKGAITQSPLSDCLTRSHSIPQANRSPLPIAKVSSFPSIRPI
YLTRVLFQDNSSHLPAASYRKKDSGVQESSHFLQGAKKNNLSLAILTLEMTGDQREVGSLGT
SATNSVTYKKVENTVLPKPDLPKTSGKVELLPKVHIYQKDLFPTETSNGSPGHLDLVEGSLL
QGTEGAIKWNEANRPGKVPFLRVATESSAKTPSKLLDPLAWDNHYGTQIPKEEWKSQEKSPE
KTAFKKKDTILSLNACESNHAIAAINEGQNKPEIEVTWAKQGRTERLCSQNPPVLKRHQREI
TRTTLQSDQEEIDYDDTISVEMKKEDFDIYDEDENQSPRSFQKKTRHYFIAAVERLWDYGMS
SSPHVLRNRAQSGSVPQFKKVVFQEFTDGSFTQPLYRGELNEHLGLLGPYIRAEVEDNIMVT
FRNQASRPYSFYSSLISYEEDQRQGAEPRKNFVKPNETKTYFWKVQHHMAPTKDEFDCKAWA
YFSDVDLEKDVHSGLIGPLLVCHTNTLNPAHGRQVTVQEFALFFTIFDETKSWYFTENMERN
CRAPCNIQMEDPTFKENYRFHAINGYIMDTLPGLVMAQDQRIRWYLLSMGSNENIHSIHFSG
HVFTVRKKEEYKMALYNLYPGVFETVEMLPSKAGIWRVECLIGEHLHAGMSTLFLVYSNKCQ
TPLGMASGHIRDFQITASGQYGQWAPKLARLHYSGSINAWSTKEPFSWIKVDLLAPMIIHGI
KTQGARQKFSSLYISQFIIMYSLDGKKWQTYRGNSTGTLMVFFGNVDSSGIKHNIFNPPIIA
RYIRLHPTHYSIRSTLRMELMGCDLNSCSMPLGMESKAISDAQITASSYFTNMFATWSPSKA
RLHLQGRSNAWRPQVNNPKEWLQVDFQKTMKVTGVTTQGVKSLLTSMYVKEFLISSSQDGHQ
WTLFFQNGKVKVFQGNQDSFTPVVNSLDPPLLTRYLRIHPQSWVHQIALRMEVLGCEAQDLY
AAV-LK03 VP1 Capsid
(SEQ ID NO: 27)
MAADGYLPDWLEDNLSEGIREWWALQPGAPKPKANQQHQDNARGLVLPGYKYLGPGNGLDKG
EPVNAADAAALEHDKAYDQQLKAGDNPYLKYNHADAEFQERLKEDTSFGGNLGRAVFQAKKR
LLEPLGLVEEAAKTAPGKKRPVDQSPQEPDSSSGVGKSGKQPARKRLNFGQTGDSESVPDPQ
PLGEPPAAPTSLGSNTMASGGGAPMADNNEGADGVGNSSGNWHCDSQWLGDRVITTSTRTWA
LPTYNNHLYKQISSQSGASNDNHYFGYSTPWGYFDFNRFHCHFSPRDWQRLINNNWGFRPKK
LSFKLFNIQVKEVTQNDGTTTIANNLTSTVQVFTDSEYQLPYVLGSAHQGCLPPFPADVFMV
PQYGYLTLNNGSQAVGRSSFYCLEYFPSQMLRTGNNFQFSYTFEDVPFHSSYAHSQSLDRLM
NPLIDQYLYYLNRTQGTTSGTTNQSRLLFSQAGPQSMSLQARNWLPGPCYRQQRLSKTANDN
NNSNFPWTAASKYHLNGRDSLVNPGPAMASHKDDEEKFFPMHGNLIFGKEGTTASNAELDNV
MITDEEEIRTTNPVATEQYGTVANNLQSSNTAPTTRTVNDQGALPGMVWQDRDVYLQGPIWA
KIPHTDGHFHPSPLMGGFGLKHPPPQIMIKNTPVPANPPTTFSPAKFASFITQYSTGQVSVE
IEWELQKENSKRWNPEIQYTSNYNKSVNVDFTVDTNGVYSEPRPIGTRYLTRPL
AAV-SPK VP1 Capsid (SEQ ID NO: 28) used in AAV-SPK-8005 and AAV-SPK-hFIX
MAADGYLPDWLEDNLSEGIREWWDLKPGAPKPKANQQKQDNGRGLVLPGYKYLGPFNGLDKGEPVNAADAA
ALEHDKAYDQQLQAGDNPYLRYNHADAEFQERLQEDTSFGGNLGRAVFQAKKRVLEPLGLVESPVKTAPGK
KRPVEPSPQRSPDSSTGIGKKGQQPAKKRLNFGQTGDSESVPDPQPIGEPPAAPSGVGPNTMAAGGGAPMA
DNNEGADGVGSSSGNWHCDSTWLGDRVITTSTRTWALPTYNNHLYKQISNGTSGGSTNDNTYFGYSTPWGY
FDFNRFHCHFSPRDWQRLINNNWGFRPKRLNFKLFNIQVKEVTQNEGTKTIANNLTSTIQVFTDSEYQLPY
VLGSAHQGCLPPFPADVFMIPQYGYLTLNNGSQAVGRSSFYCLEYFPSQMLRTGNNFEFSYNFEDVPFHSS
YAHSQSLDRLMNPLIDQYLYYLSRTQSTGGTAGTQQLLFSQAGPNNMSAQAKNWLPGPCYRQQRVSTTLSQ
NNNSNFAWTGATKYHLNGRDSLVNPGVAMATHKDDEERFFPSSGVLMFGKQGAGKDNVDYSSVMLTSEEEI
KTTNPVATEQYGVVADNLQQQNAAPIVGAVNSQGALPGMVWQNRDVYLQGPIWAKIPHTDGNFHPSPLMGG
FGLKHPPPQILIKNTPVPADPPTTFNQAKLASFITQYSTGQVSVEIEWELQKENSKRWNPEIQYTSNYYKS
TNVDFAVNTEGTYSEPRPIGTRYLTRNL

Percent Identity Matrix of hFVIII Vectors (WT, CO3, X09, X02, X06, X08, X15, X05,
X18, X14, X01, X12, X04, X11, X07, X03, X16, X13, X17 and X10)

| | hFVIII | hFVIII | hFVIII | hFVIII | hFVIII | hFVIII | hFVIII | hFVIII | hFVIII | hFVIII | hFVIII | hFVIII |
|---|---|---|---|---|---|---|---|---|---|---|---|---|
| | WT | CO3 | X09 | X02 | X06 | X08 | X15 | X05 | X18 | X14 | X01 | X12 |
| hFVIII | | 77.2 | 79.5 | 79.1 | 79.3 | 79.2 | 79.3 | 79.1 | 79 | 79.6 | 79.6 | 79.4 |
| WT | | | | | | | | | | | | |
| hFVIII | 77.2 | | 81.9 | 81.9 | 81.5 | 81.3 | 81.6 | 81.6 | 81.2 | 81.4 | 81.1 | 81.1 |
| CO3 | | | | | | | | | | | | |
| hFVIII | 79.5 | 81.9 | | 91.5 | 91.4 | 91.8 | 92 | 91.8 | 91 | 91.4 | 91.5 | 91.5 |
| X09 | | | | | | | | | | | | |
| hFVIII | 79.1 | 81.9 | 91.5 | | 91.4 | 91.3 | 92 | 92.1 | 92.2 | 91.7 | 92 | 91.9 |
| X02 | | | | | | | | | | | | |
| hFVIII | 79.3 | 81.5 | 91.4 | 91.4 | | 91.8 | 91.9 | 91.8 | 91.5 | 91.8 | 92.3 | 91.7 |
| X06 | | | | | | | | | | | | |
| hFVIII | 79.2 | 81.3 | 91.8 | 91.3 | 91.8 | | 91.8 | 91.5 | 91.5 | 91.8 | 92.2 | 91.5 |
| X08 | | | | | | | | | | | | |
| hFVIII | 79.3 | 81.6 | 92 | 92 | 91.9 | 91.8 | | 92.2 | 91.6 | 91.7 | 92.3 | 92.1 |
| X15 | | | | | | | | | | | | |
| hFVIII | 79.1 | 81.6 | 91.8 | 92.1 | 91.8 | 91.5 | 92.2 | | 92.5 | 91.9 | 92.7 | 92.4 |
| X05 | | | | | | | | | | | | |
| hFVIII | 79 | 81.2 | 91 | 92.2 | 91.5 | 91.5 | 91.6 | 92.5 | | 91.6 | 93 | 92.1 |
| X18 | | | | | | | | | | | | |
| hFVIII | 79.6 | 81.4 | 91.4 | 91.7 | 91.8 | 91.8 | 91.7 | 91.9 | 91.6 | | 93 | 92 |
| X14 | | | | | | | | | | | | |
| hFVIII | 79.6 | 81.1 | 91.5 | 92 | 92.3 | 92.2 | 92.3 | 92.7 | 93 | 93 | | 93.4 |
| X01 | | | | | | | | | | | | |
| hFVIII | 79.4 | 81.1 | 91.5 | 91.9 | 91.7 | 91.5 | 92.1 | 92.4 | 92.1 | 92 | 93.4 | |
| X12 | | | | | | | | | | | | |
| hFVIII | 79.4 | 81.3 | 91.7 | 91.9 | 91.8 | 92.3 | 92.2 | 92.1 | 91.5 | 91.6 | 92.3 | 92 |
| X04 | | | | | | | | | | | | |
| hFVIII | 79.4 | 81.7 | 91.7 | 92 | 92 | 92.5 | 92.5 | 91.5 | 91.8 | 91.8 | 92.5 | 92 |
| X11 | | | | | | | | | | | | |
| hFVIII | 79.2 | 81.8 | 92.2 | 91.5 | 91.5 | 92 | 92 | 92.1 | 91.7 | 91.3 | 92.6 | 92.4 |
| X07 | | | | | | | | | | | | |
| hFVIII | 79.4 | 81.6 | 91.5 | 91 | 91.4 | 91.7 | 92.1 | 91.6 | 91.4 | 91.8 | 92.5 | 92.4 |
| X03 | | | | | | | | | | | | |
| hFVIII | 79.1 | 81.9 | 92.1 | 91.5 | 91.7 | 91.4 | 92.2 | 91.7 | 91.1 | 92.3 | 92.2 | 91.7 |
| X16 | | | | | | | | | | | | |
| hFVIII | 79 | 81.8 | 91.8 | 92.3 | 92.4 | 92.3 | 92.3 | 92.3 | 91.8 | 92.2 | 92.6 | 92.4 |
| X13 | | | | | | | | | | | | |
| hFVIII | 79.6 | 82.1 | 91.1 | 91.9 | 91.6 | 91.6 | 92.5 | 91.9 | 91.8 | 91.8 | 92.4 | 92.6 |
| X17 | | | | | | | | | | | | |
| hFVIII | 79.3 | 82.2 | 91.6 | 92.1 | 91.8 | 91.9 | 92 | 92 | 92 | 92 | 92.1 | 92.6 |
| X10 | | | | | | | | | | | | |
| | | | | | hFVIII | hFVIII | hFVIII | hFVIII | hFVIII | hFVIII | hFVIII | hFVIII |
| | | | | | X04 | X11 | X07 | X03 | X16 | X13 | X17 | X10 |
| | | | | hFVIII | 79.4 | 79.4 | 79.2 | 79.4 | 79.1 | 79 | 79.6 | 79.3 |
| | | | | WT | | | | | | | | |
| | | | | hFVIII | 81.3 | 81.7 | 81.8 | 81.6 | 81.9 | 81.8 | 82.1 | 82.2 |
| | | | | CO3 | | | | | | | | |
| | | | | hFVIII | 91.7 | 91.7 | 92.2 | 91.5 | 92.1 | 91.8 | 91.1 | 91.6 |
| | | | | X09 | | | | | | | | |
| | | | | hFVIII | 91.9 | 92 | 91.5 | 91 | 91.5 | 92.3 | 91.9 | 92.1 |
| | | | | X02 | | | | | | | | |
| | | | | hFVIII | 91.8 | 92 | 91.5 | 91.4 | 91.7 | 92.4 | 91.6 | 91.8 |
| | | | | X06 | | | | | | | | |
| | | | | hFVIII | 92.3 | 92.5 | 92 | 91.7 | 91.4 | 92.3 | 91.6 | 91.9 |
| | | | | X08 | | | | | | | | |
| | | | | hFVIII | 92.2 | 92.5 | 92 | 92.1 | 92.2 | 92.3 | 92.5 | 92 |
| | | | | X15 | | | | | | | | |
| | | | | hFVIII | 92.1 | 91.5 | 92.1 | 91.6 | 91.7 | 92.3 | 91.9 | 92 |
| | | | | X05 | | | | | | | | |
| | | | | hFVIII | 91.5 | 91.8 | 91.7 | 91.4 | 91.1 | 91.8 | 91.8 | 92 |
| | | | | X18 | | | | | | | | |
| | | | | hFVIII | 91.6 | 91.8 | 91.3 | 91.8 | 92.3 | 92.2 | 91.8 | 92 |
| | | | | X14 | | | | | | | | |
| | | | | hFVIII | 92.3 | 92.5 | 92.6 | 92.5 | 92.2 | 92.6 | 92.4 | 92.1 |
| | | | | X01 | | | | | | | | |
| | | | | hFVIII | 92 | 92 | 92.4 | 92.4 | 91.7 | 92.4 | 92.6 | 92.6 |
| | | | | X12 | | | | | | | | |
| | | | | hFVIII | | 92.6 | 92 | 91.5 | 91.5 | 92 | 91.9 | 92.5 |
| | | | | X04 | | | | | | | | |
| | | | | hFVIII | 92.6 | | 92.6 | 92 | 91.9 | 92.3 | 91.8 | 91.9 |
| | | | | X11 | | | | | | | | |
| | | | | hFVIII | 92 | 92.6 | | 92.1 | 92 | 92.4 | 91.9 | 92.7 |
| | | | | X07 | | | | | | | | |
| | | | | hFVIII | 91.5 | 92 | 92.1 | | 92 | 92.7 | 92.1 | 91.6 |
| | | | | X03 | | | | | | | | |
| | | | | hFVIII | 91.5 | 91.9 | 92 | 92 | | 92.4 | 92 | 92.8 |
| | | | | X16 | | | | | | | | |
| | | | | hFVIII | 92 | 92.3 | 92.4 | 92.7 | 92.4 | | 92.4 | 92.8 |
| | | | | X13 | | | | | | | | |
| | | | | hFVIII | 91.9 | 91.8 | 91.9 | 92.1 | 92 | 92.4 | | 92.9 |
| | | | | X17 | | | | | | | | |
| | | | | hFVIII | 92.5 | 91.9 | 92.7 | 91.6 | 92.8 | 92.8 | 92.9 | |
| | | | | X10 | | | | | | | | |

Certain Definitions/Abbreviations Used
BDD: all or at least part of B domain (BD) deleted
FVIII-BDD: FVIII with B domain deletion
SQ: SFSQNPPVLKRHQR (SEQ ID NO: 29)
FVIII/SQ: FVIII with SQ
FVIIIX01-X18: CpG reduced FVIII encoding nucleic acid variants, set forth as SEQ ID Nos:1-18, respectively.
TTRmut: TTR promoter with 4 mutations, from TAmGTGTAG to TATTGACTTAG
CO3: codon optimized FVIII nucleic acid variant, set forth as SEQ ID NO: 21
NHP: Non human primate
ALT: Alanine aminotransferase
D-dimer: A protein fragment from the break down of a blood clot
SPK-8005: AAV capsid (SEQ ID NO: 28) + TTRmut-hFVIII-X07; also referred to as AAV-SPK- 8005
SPK-8011: AAV LKO3 capsid (SEQ ID NO: 27) + TTRmut-hFVIII-X07; also referred to as AAV-SPK-8011

While certain of the embodiments of the invention have been described and specifically exemplified above, it is not intended that the invention be limited to such embodiments. Various modifications may be made thereto without departing from the scope and spirit of the invention, as set forth in the following claims.

1-140. (canceled)

141. A method of treating a human patient having hemophilia A comprising administering to said patient a recombinant adeno-associated virus (rAAV) vector comprising:
a) an expression cassette comprising 5′ to 3′: (i) a 5′ AAV2 ITR, (ii) a promoter comprising the sequence of SEQ ID NO: 22; (iii) a nucleic acid variant sequence encoding a Factor VIII having a B domain deletion, wherein said nucleic acid variant sequence comprises a nucleic acid sequence having a sequence identity of at least 95% to SEQ ID NO: 7, (iv) a polyadenylation sequence; and (v) a 3′ AAV2 ITR,
wherein said promoter is operably linked to said nucleic acid variant sequence; and
b) an LK03 capsid encapsidating said expression cassette, wherein said LK03 capsid comprises the amino acid sequence of SEQ ID NO: 27;
wherein the rAAV vector is administered to said patient at a dose of 2×10¹¹ to 9×10¹¹ vector genomes per kilogram (vg/kg), inclusive.

142. The method of claim 141, wherein said expression cassette comprises 5′ to 3′: said 5′ AAV2 ITR, said promoter sequence, a synthetic intron, said variant Factor VIII encoding sequence, said polyadenylation signal and said 3′ AAV2 ITR.

143. The method of claim 142, wherein said synthetic intron is derived from human elongation factor EF-1 alpha and comprises the intron sequence provided in SEQ ID NO: 23; and said polyadenylation signal is a rabbit beta globin polyA signal sequence comprising the polyA signal sequence provided in SEQ ID NO: 23.

144. The method of claim 143, wherein said expression cassette comprises a Kozak consensus sequence.

145. The method of claim 144, wherein said nucleic acid variant sequence has a sequence identity of at least 99% to SEQ ID NO:7.

146. The method of claim 145, wherein said nucleic acid variant sequence has no more than 4 CpGs.

147. The method of claim 146, wherein said nucleic acid variant sequence sequence has no CpGs.

148. The method of claim 147, wherein said FVIII comprising a B domain deletion comprises an amino acid sequence at least 95% identical to a wild type human FVIII comprising a B domain deletion.

149. The method of claim 148, wherein said Factor VIII having a B domain deletion comprises a sequence at least 95% identical to SEQ ID NO: 25.

150. The method of claim 149, wherein said Factor VIII having a B domain deletion comprises the amino acid sequence of SEQ ID NO: 25.

151. The method of claim 143, wherein said nucleic acid variant sequence comprises a sequence having a sequence identity of at least 99.5% to SEQ ID NO:7 and has zero CpGs, and said Factor VIII having a B domain deletion comprises a sequence at least 99% identical to SEQ ID NO: 25.

152. The method of claim 151, wherein said expression cassette further comprises a Kozak consensus sequence.

153. The method of claim 147, wherein said rAAV vector is administered to said patient at a dose of 5×10¹¹ vector genomes per kilogram (vg/kg).

154. The method of claim 149, wherein said rAAV vector is administered to said patient at a dose of 5×10¹¹ vector genomes per kilogram (vg/kg).

155. The method of claim 150, wherein said rAAV vector is administered to said patient at a dose of 5×10¹¹ vector genomes per kilogram (vg/kg).

156. The method of claim 151, wherein said rAAV vector is administered to said patient at a dose of 5×10¹¹ vector genomes per kilogram (vg/kg).

157. The method of claim 152, wherein said rAAV vector is administered to said patient at a dose of 5×10¹¹ vector genomes per kilogram (vg/kg).

158. The method of claim 156, wherein said patient has mild hemophilia A.

159. The method of claim 157, wherein said patient has moderate hemophilia A.

160. The method of claim 151, wherein said patient prior to rAAV vector administration is receiving on-demand therapy, has a FVIII baseline level of 1-2% of normal A, and has experienced >10 bleeding events over the previous 12 months.

161. The method of claim 151, wherein an immunosuppressive agent is not added 2-4 days after vector administration.

162. The method of claim 151, wherein a steroid is not used within 51 weeks of administration.

163. The method of claim 151, further comprising the use of an immunosuppressive agent.

164. The method of claim 151, wherein the variant Factor VIII level plateaus at approximately 9.15%±0.53% or 13.50%±0.50% of normal Factor VIII.

165. The method of claim 151, wherein said patent does not undergo a spontaneous bleed within one year of rAAV administration.

166. The method of claim 152, wherein said patient has mild hemophilia A.

167. The method of claim 152, wherein said patient has moderate hemophilia A.

168. The method of claim 152, wherein said patient has severe hemophilia A.

169. The method of claim 152, wherein said patient prior to rAAV vector administration is receiving on-demand therapy, has a FVIII baseline level of 1-2% of normal A, and has experienced >10 bleeding events over the previous 12 months.

170. The method of claim 152, wherein an immunosuppressive agent is not added 2-4 days after vector administration.

171. The method of claim 152, wherein a steroid is not used within 51 weeks of administration.

172. The method of claim 152, further comprising the use of an immunosuppressive agent.

173. The method of claim 152, wherein the variant Factor VIII level plateaus at approximately 9.15%±0.53% or 13.50%±0.50% of normal Factor VIII.

174. The method of claim 152, wherein said patent does not undergo a spontaneous bleed within one year of rAAV administration.
